(19) United States
(12) Patent Application Publication
(10) Pub. No.: US 2023/0404963 A1
(43) Pub. Date: Dec. 21, 2023

(54) COMBINATIONS OF METAP2 INHIBITORS AND CD4/6 INHIBITORS FOR THE TREATMENT OF CANCER

(71) Applicant: SynDevRx, Inc., Cambridge, MA (US)

(72) Inventors: Peter CORNELIUS, Framingham, MA (US); James SHANAHAN, Cambridge, MA (US); Bradley J. CARVER, Cambridge, MA (US)

(21) Appl. No.: 18/036,565

(22) PCT Filed: Nov. 10, 2021
(86) PCT No.: PCT/US2021/058775

§ 371 Date: May 11, 2023

Related U.S. Application Data
(60) Provisional application No. 63/166,060, filed on Mar. 25, 2021.
(60) Provisional application No. 63/112,217, filed on Nov. 11, 2020.

(51) Int. Cl. A61K 31/336; A61K 31/519; A61K 31/506; A61P 35/00

(52) U.S. Cl. CPC A61K 31/336; A61K 31/519; A61K 31/506; A61P 35/00

(57) ABSTRACT

The present disclosure is directed to combinations of MetAP2 inhibitors and CDK4/6 inhibitors for the treatment and prevention of cancer.

### CROSS REFERENCE TO RELATED APPLICATIONS

This application claims priority to, and the benefit of, U.S. Provisional Application No. 63/112,217, filed on Nov. 11, 2020, and U.S. Provisional Application No. 63/166,060, filed on Mar. 25, 2021. The contents of each of the aforementioned patent applications are incorporated herein by reference in their entireties.

### BACKGROUND

Inhibitors of cyclin-dependent kinases CDK4 and CDK6, referred to herein as CDK4/6 inhibitors, are used for the treatment of breast cancers, for example, metastatic, hormone receptor (HR)-positive, human epidermal growth factor 2 (HER2)-negative (HR+HER2−) breast cancer. Treatment resistance to drugs from this class, such as palbociclib, the first CDK4/6 inhibitor to be approved as a cancer therapy by the Federal Drug Administration (FDA), as well as other CDK4/6 inhibitors (e.g., abemaciclib, ribociclib), has been reported to be mediated through a number of factors including upregulation of CDK2 and Cyclins D and F, increased autophagy, through the Akt protein, via changes to the estrogen receptor, and other mechanisms. Moreover, there is currently still only limited demonstrated improvement in overall survival (OS) in taking palbociclib or other CDK4/6 inhibitors, as resistance against these agents emerges in a majority of patients, resulting in eventual disease progression. Another proposed mediator of CDK4/6 resistance is through increased intracellular recycling of proteins (autophagy), which allows cells to replicate at an accelerated pace. Thus, there is a need in the art for compositions and methods that attenuate treatment resistance and boost the efficacy of CDK4/6 inhibitors. The present disclosure presents combinations of MetAP2 inhibitors and CDK4/6 inhibitors for the treatment of cancer.

### SUMMARY

The present disclosure provides combinations comprising at least one MetAP2 inhibitor, or a pharmaceutically acceptable salt thereof, and at least one CDK 4/6 inhibitor, or a pharmaceutically acceptable salt thereof, for use in treating a cancer.

The present disclosure provides methods of treating cancer in a subject in need thereof, the method comprising administering to the subject at least one therapeutically effective amount of at least one MetAP2 inhibitor, or a pharmaceutically acceptable salt thereof, and at least one therapeutically effective amount of at least one CDK4/6 inhibitor, or a pharmaceutically acceptable salt thereof.

The present disclosure provides a MetAP2 inhibitor, or a pharmaceutically acceptable salt thereof, for use in a method of treating a cancer, wherein the method further comprises administration of at least one CDK4/6 inhibitor or a pharmaceutically acceptable salt thereof.

The present disclosure provides a CDK4/6 inhibitor, or a pharmaceutically acceptable salt thereof, for use in a method of treating a cancer, wherein the method further comprises administration of at least one MetAP2 inhibitor or a pharmaceutically acceptable salt thereof.

In some aspects, an at least one MetAP2 inhibitor, or pharmaceutically acceptable salt thereof and an at least one CDK4/6 inhibitor, or pharmaceutically acceptable salt thereof, are administered concurrently or in temporal proximity.

The present disclosure provides a method of treating cancer in a subject in need thereof, the method comprising administering to the subject at least one therapeutically effective amount of at least one MetAP2 inhibitor, or a pharmaceutically acceptable salt thereof, and at least one therapeutically effective amount of at least one CDK4/6 inhibitor, or a pharmaceutically acceptable salt thereof.

The present disclosure provides a combination of at least one MetAP2 inhibitor, or a pharmaceutically acceptable salt thereof in combination with at least one CDK 4/6 inhibitor, or a pharmaceutically acceptable salt thereof, for use in the manufacture of a medicament for the treatment of a cancer.

The present disclosure provides a combination of at least one MetAP2 inhibitor, or a pharmaceutically acceptable salt thereof, and at least one CDK4/6 inhibitor or a pharmaceutically acceptable salt thereof, for use in treating a cancer.

The present disclosure provides a combination therapy comprising at least one therapeutically effective amount of at least one MetAP2 inhibitor, or a pharmaceutically acceptable salt thereof and at least one therapeutically effective amount of at least one CDK4/6 inhibitor, or a pharmaceutically acceptable salt thereof.

The present disclosure provides a pharmaceutical composition comprising at least one therapeutically effective amount of at least one MetAP2 inhibitor, or a pharmaceutically acceptable salt thereof and at least one therapeutically effective amount of at least one CDK4/6 inhibitor, or a pharmaceutically acceptable salt thereof.

The present disclosure provides a kit comprising at least one therapeutically effective amount of at least one MetAP2 inhibitor, or a pharmaceutically acceptable salt thereof and at least one therapeutically effective amount of at least one CDK4/6 inhibitor, or a pharmaceutically acceptable salt thereof.

The present disclosure provides a method of treating cancer in a subject in need thereof, the method comprising administering to the subject in need thereof at least one therapeutically effective amount of the combination therapy of claim 4, the pharmaceutical composition of claim 5 or the kit of claim 6.

A MetAP2 inhibitor can be a compound represented by Formula (I):

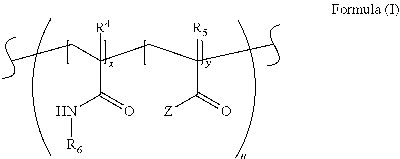

wherein, independently for each occurrence,
R₄ is H or C₁-C₆ alkyl;
R₅ is H or C₁-C₆ alkyl;
R₆ is C₂-C₆ hydroxyalkyl;
Z is —NH-AA₁-AA₂-AA₃-AA₄-AA₅-AA₆-C(O)-L or —NH-AA₁-AA₂-AA₃-AA₄-AA₅-AA₆-C(O)-Q-X—Y—C(O)—W;
AA₁ is glycine, alanine, or H₂N(CH₂)ₘCO₂H, wherein m is 2, 3, 4 or 5;
AA₂ is a bond, or alanine, cysteine, aspartic acid, glutamic acid, phenylalanine, glycine, histidine, isoleucine, lysine, leucine, methionine, asparagine, proline, glutamine, arginine, serine, threonine, valine, tryptophan, or tyrosine;
AA₃ is a bond, or alanine, cysteine, aspartic acid, glutamic acid, phenylalanine, glycine, histidine, isoleucine, lysine, leucine, methionine, asparagine, proline, glutamine, arginine, serine, threonine, valine, tryptophan, or tyrosine;
AA₄ is a bond, or alanine, cysteine, aspartic acid, glutamic acid, phenylalanine, glycine, histidine, isoleucine, lysine, leucine, methionine, asparagine, proline, glutamine, arginine, serine, threonine, valine, tryptophan, or tyrosine;
AA₅ is a bond, or glycine, valine, tyrosine, tryptophan, phenylalanine, methionine, leucine, isoleucine, or asparagine;
AA₆ is a bond, or alanine, asparagine, citrulline, glutamine, glycine, leucine, methionine, phenylalanine, serine, threonine, tryptophan, tyrosine, valine, or H₂N(CH₂)ₘCO₂H, wherein m is 2, 3, 4 or 5; L is —OH, —O-succinimide, —O-sulfosuccinimide, alkoxy, aryloxy, acyloxy, aroyloxy, alkoxycarbonyloxy, aryloxycarbonyloxy, —NH₂, —NH(C₂-C₆ hydroxyalkyl), halide or perfluoroalkyloxy;
Q is NR, O, or S;
X is M-(C(R)₂)ₚ-M-J-M-(C(R)₂)ₚ-M-V;
M is a bond, or C(O);
J is a bond, or ((CH₂)_{q}Q)ᵣ, C₅-C₈ cycloalkyl, aryl, heteroaryl, NR, O, or S;
Y is NR, O, or S;
R is H or alkyl;

V is a bond or

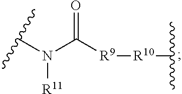

R⁹ is alkyl, aryl, aralkyl, or a bond; or R⁹ taken together with Y forms a heterocyclic ring;
R¹⁰ is amido or a bond;
R¹¹ is H or alkyl;
W is a MetAP2 inhibitor moiety or alkyl;
x is in the range of 1 to about 450;
y is in the range of 1 to about 30;
n is in the range of 1 to about 100;
p is 0 to 20;
q is 2 or 3;
r is 1, 2, 3, 4, 5, or 6;
or a pharmaceutically acceptable salt, prodrug, metabolite, analog or derivative thereof.

A MetAP2 inhibitor can be

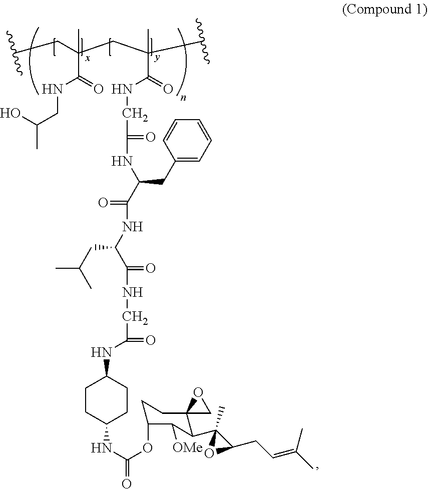

or a pharmaceutically acceptable salt, prodrug, metabolite, analog or derivative thereof.

A MetAP2 inhibitor can be

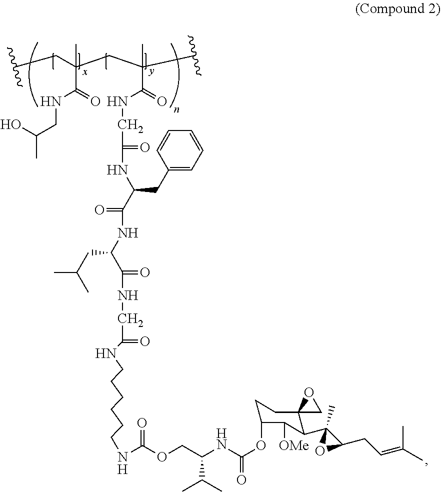

or a pharmaceutically acceptable salt, prodrug, metabolite, analog or derivative thereof.

A MetAP2 inhibitor can be

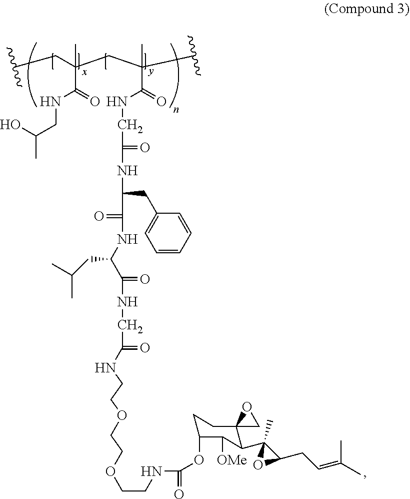

or a pharmaceutically acceptable salt, prodrug, metabolite, analog or derivative thereof.

A MetAP2 inhibitor can be:

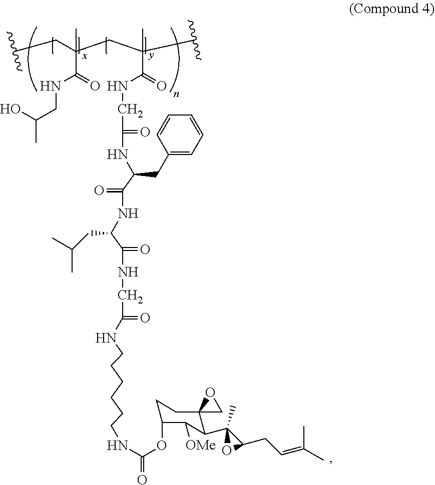

or a pharmaceutically acceptable salt, analog, derivative, salt or ester thereof.

In some embodiments, X can be in the range of 1 to about 450. In some embodiments, Y can be in the range of 1 to about 30. In some embodiments, n can be in the range of 1 to about 100.

In some embodiments, the MetAP2 inhibitor can be

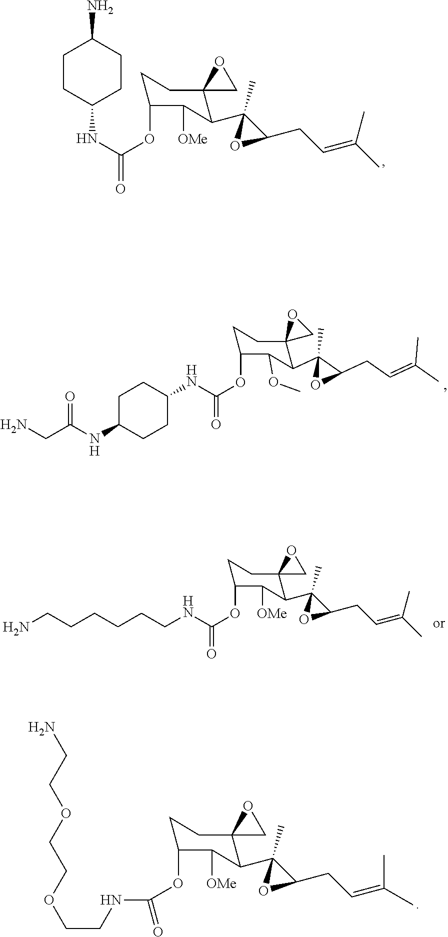

In some embodiments, the MetAP2 inhibitor can be

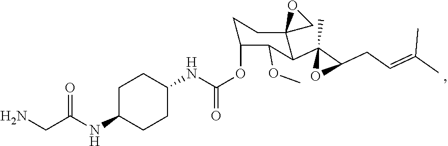

In some embodiments, R₄ can be methyl. In some embodiments, R₅ can be methyl. In some embodiments, R₆ can be 2-hydroxypropyl.

In some embodiments, Z can be —NH-AA₆-C(O)-Q-X—Y—C(O)—W. In some embodiments, AA₆ can be glycine.

In some embodiments, Z can be —NH-AA₅-AA₆-C(O)—X—Y—C(O)—W. In some embodiments, AA₅ can be leucine and AA₆ can be glycine. In some embodiments, AA₅ can be valine and AA₆ can be glycine. In some embodiments, AA₅ can be phenylalanine and AA₆ can be glycine. In some embodiments, AA₅ can be glycine and AA₆ can be glycine.

In some embodiments, Z can be —NH-AA₃-AA₄-AA₅-AA₆-C(O)-Q-X—Y—C(O)—W. In some embodiments, AA₅ can be leucine and each of AA₃, AA₄, or AA₆ can be glycine. In some embodiments, AA₅ can be valine and each of AA₃, AA₄, or AA₆ can be glycine. In some embodiments, AA₅ can be phenylalanine and each of AA₃, AA₄, or AA₆ can be glycine. In some embodiments, AA₃ can be glycine, AA₄ can be phenylalanine, AA₅ can be leucine and AA₆ can be glycine. In some embodiments, each of AA₃, AA₄, AA₅ and AA₆ can be glycine.

In some embodiments, -Q-X—Y can be

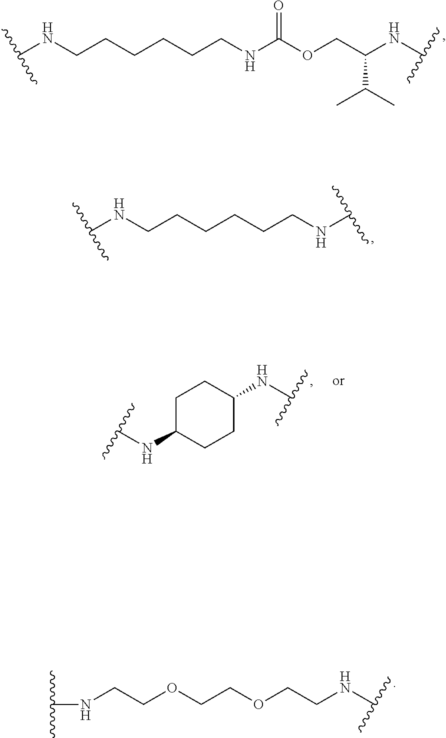 [SMILES: *NCCCCCCNC(=O)OC[C@H](N*)C(C)C ; *NCCCCCCN* ; *N[C@H]1CC[C@H](N*)CC1 ; *NCCOCCOCCN*]

In some embodiments, W can be

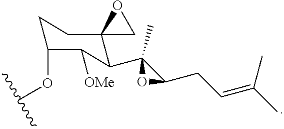

In some embodiments, the ratio of x to y can be in the range of about 30:1 to about 3:1. In some embodiments, the ratio of x to y can be about 11:1.

A CDK4/6 inhibitor can be selected from palbociclib, abemaciclib, ribociclib, trilaciclib, SHR-6390, FCN-437c, lerociclib, milciclib, PF-06873600, XZP-3287, zotiraciclib, BEBT-209, BPI-16350, CS-3002, fadraciclib, HS-10342, ON-123300, PF-06842874, TQ-05510, BPI-1178, JS-101, NUV-422, AU-294, CCT-68127 ETH-155008, HEC-80797, JRP-890, JS-104, NEOS-518, PF-07104091, PF-07220060, RMC-4550, SRX-3177, VS-2370, VS-2370, or a pharmaceutically acceptable salt thereof. A CDK4/6 inhibitor can be palbociclib, or a pharmaceutically acceptable salt thereof. A CDK4/6 inhibitor can be abemaciclib, or a pharmaceutically acceptable salt thereof. A CDK4/6 inhibitor can be ribociclib, or a pharmaceutically acceptable salt thereof.

A MetAP2 inhibitor can be for administration by subcutaneous injection. A CDK4/6 inhibitor can be for oral administration.

A cancer can be a carcinoma, a lymphoma, a blastoma, a sarcoma, a leukemia, a brain cancer, a breast cancer, a blood cancer, a bone cancer, a lung cancer, a skin cancer, a liver cancer, an ovarian cancer, a bladder cancer, a renal cancer, a kidney cancer, a gastric cancer, a thyroid cancer, a pancreatic cancer, an esophageal cancer, a prostate cancer, a cervical cancer, a uterine cancer, a stomach cancer, a soft tissue cancer, a laryngeal cancer, a small intestine cancer, a testicular cancer, an anal cancer, a vulvar cancer, a joint cancer, an oral cancer, a pharynx cancer or a colorectal cancer. A cancer can be a breast cancer. A breast cancer can be HR+HER2− breast cancer.

The present disclosure provides a method of treating breast cancer in a subject in need thereof, the method comprising administering to the subject: a) at least one therapeutically effective amount of the Compound 1:

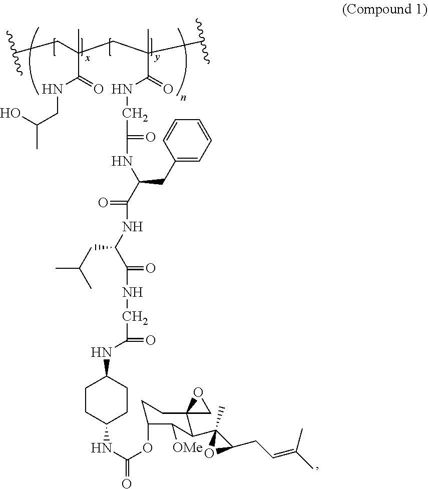

or a pharmaceutically acceptable salt, prodrug, metabolite, analog or derivative thereof, wherein x is in the range of 1 to about 450, y is in the range of 1 to about 30, and n is in the range of 1 to about 100; and b) at least one therapeutically effective amount of palbociclib, or a pharmaceutically acceptable salt thereof.

The present disclosure provides a method of treating breast cancer in a subject in need thereof, the method comprising administering to the subject: a) at least one therapeutically effective amount of the Compound 1:

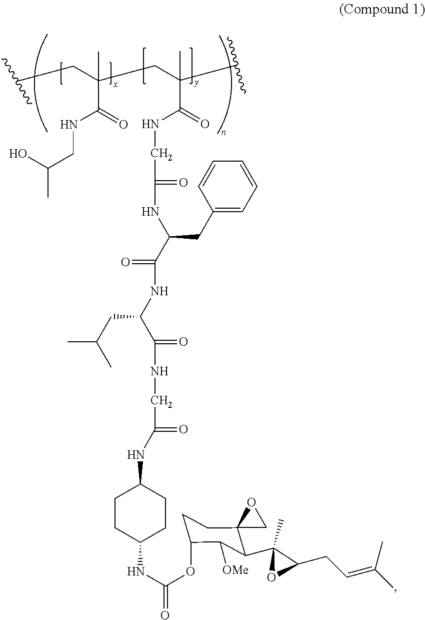

or a pharmaceutically acceptable salt, prodrug, metabolite, analog or derivative thereof, wherein x is in the range of 1 to about 450, y is in the range of 1 to about 30, and n is in the range of 1 to about 100; and b) at least one therapeutically effective amount of ribociclib, or a pharmaceutically acceptable salt thereof.

Any of the above aspects, or any other aspect described herein, can be combined with any other aspect.

Unless otherwise defined, all technical and scientific terms used herein have the same meaning as commonly understood by one of ordinary skill in the art to which this disclosure belongs. In the Specification, the singular forms also include the plural unless the context clearly dictates otherwise; as examples, the terms “a,” “an,” and “the” are understood to be singular or plural and the term “or” is understood to be inclusive. By way of example, “an element” means one or more element. Throughout the specification the word “comprising,” or variations such as “comprises” or “comprising,” will be understood to imply the inclusion of a stated element, integer or step, or group of elements, integers or steps, but not the exclusion of any other element, integer or step, or group of elements, integers or steps. About can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Although methods and materials similar or equivalent to those described herein can be used in the practice or testing of the present disclosure, suitable methods and materials are described below. All publications, patent applications, patents, and other references mentioned herein are incorporated by reference in their entirety. The references cited herein are not admitted to be prior art to the claimed invention. In the case of conflict, the present Specification, including definitions, will control. In addition, the materials, methods, and examples are illustrative only and are not intended to be limiting. Other features and advantages of the disclosure will be apparent from the following detailed description and claim.

### BRIEF DESCRIPTION OF THE DRAWINGS

The above and further features will be more clearly appreciated from the following detailed description when taken in conjunction with the accompanying drawings.

FIG. 1 is a graph showing the MCF tumor volume in mice over the course of treatment, wherein the mice were treated either with a vehicle control, a combination of compound 1 and palbociclib, compound 1 alone or palbociclib alone.

FIG. 2 is a graph showing the MCF tumor volume in mice at the end of treatment, wherein the mice were treated either with a vehicle control, a combination of compound 1 and palbociclib, compound 1 alone or palbociclib alone.

FIG. 3 is a graph showing the bodyweight of mice over the course of treatment, wherein the mice were treated either with a vehicle control, a combination of compound 1 and palbociclib, compound 1 alone or palbociclib alone.

FIG. 4 is a series of graphs showing the percent survival of mice over the course of treatment, wherein the mice were treated either with a vehicle control, a combination of compound 1 and palbociclib, compound 1 alone or palbociclib alone.

FIG. 5 is a graph showing the expression level of Cyclin D1 protein in tumor samples collected at the end of treatment, wherein the mice were treated either with a vehicle control, a combination of compound 1 and palbociclib, compound 1 alone or palbociclib alone.

FIG. 6 is a graph showing the expression level of Cyclin E1 protein in tumor samples collected at the end of treatment, wherein the mice were treated either with a vehicle control, a combination of compound 1 and palbociclib, compound 1 alone or palbociclib alone.

FIG. 7 is a graph showing the expression level of Cyclin E2 protein in tumor samples collected at the end of treatment, wherein the mice were treated either with a vehicle control, a combination of compound 1 and palbociclib, compound 1 alone or palbociclib alone.

FIG. 8 is a graph showing the expression level of p21 protein in tumor samples collected at the end of treatment, wherein the mice were treated either with a vehicle control, a combination of compound 1 and palbociclib, compound 1 alone or palbociclib alone.

FIG. 9 is a graph showing the expression level of CDK4 protein in tumor samples collected at the end of treatment, wherein the mice were treated either with a vehicle control, a combination of compound 1 and palbociclib, compound 1 alone or palbociclib alone.

FIG. 10 is a graph showing the expression level of CDK2 protein in tumor samples collected at the end of treatment, wherein the mice were treated either with a vehicle control, a combination of compound 1 and palbociclib, compound 1 alone or palbociclib alone.

FIG. 11 is a graph showing the expression level of Rb protein in tumor samples collected at the end of treatment, wherein the mice were treated either with a vehicle control, a combination of compound 1 and palbociclib, compound 1 alone or palbociclib alone.

FIG. 12 is a graph showing the expression level of LC3B protein in tumor samples collected at the end of treatment, wherein the mice were treated either with a vehicle control, a combination of compound 1 and palbociclib, compound 1 alone or palbociclib alone.

FIG. 13 is a graph showing the expression level of Akt protein in tumor samples collected at the end of treatment, wherein the mice were treated either with a vehicle control, a combination of compound 1 and palbociclib, compound 1 alone or palbociclib alone.

FIG. 14 is a graph showing the expression level of Phospho-Akt protein in tumor samples collected at the end of treatment, wherein the mice were treated either with a vehicle control, a combination of compound 1 and palbociclib, compound 1 alone or palbociclib alone.

FIG. 15 is a graph showing the expression level of estrogen receptor alpha (ERα)-62 kDa protein in tumor samples collected at the end of treatment, wherein the mice were treated either with a vehicle control, a combination of compound 1 and palbociclib, compound 1 alone or palbociclib alone.

FIG. 16 is a graph showing the expression level of ERα-55 kDa protein in tumor samples collected at the end of treatment, wherein the mice were treated either with a vehicle control, a combination of compound 1 and palbociclib, compound 1 alone or palbociclib alone.

FIG. 17 is a graph showing the sum of the expression levels of ERα-55 kDa protein and ERα-62 kDa protein in tumor samples collected at the end of treatment, wherein the mice were treated either with a vehicle control, a combination of compound 1 and palbociclib, compound 1 alone or palbociclib alone.

FIG. 18 is a graph showing the expression levels of PHGDH protein in tumor samples collected at the end of treatment, wherein the mice were treated either with a vehicle control, a combination of compound 1 and palbociclib, compound 1 alone or palbociclib alone.

FIG. 19 is a graph showing the amount of neutrophils in whole blood samples collected at the conclusion of the study, wherein the mice were treated either with a vehicle control, a combination of compound 1 and palbociclib, compound 1 alone or palbociclib alone.

FIG. 20 is a graph showing the expression levels of PHGDH in tumor samples collected at the end of treatment, wherein the mice were treated either with a vehicle control, a combination of compound 1 and palbociclib, compound 1 alone or palbociclib alone.

FIG. 21 is a graph showing the expression levels of PSPH in tumor samples collected at the end of treatment, wherein the mice were treated either with a vehicle control, a combination of compound 1 and palbociclib, compound 1 alone or palbociclib alone.

FIG. 22 is a graph showing the expression levels of TYMS in tumor samples collected at the end of treatment, wherein the mice were treated either with a vehicle control, a combination of compound 1 and palbociclib, compound 1 alone or palbociclib alone.

FIG. 23 is a graph showing the expression levels of MTHFD1L in tumor samples collected at the end of treatment, wherein the mice were treated either with a vehicle control, a combination of compound 1 and palbociclib, compound 1 alone or palbociclib alone.

FIG. 24 is a graph showing the expression levels of MTHFD1 in tumor samples collected at the end of treatment, wherein the mice were treated either with a vehicle control, a combination of compound 1 and palbociclib, compound 1 alone or palbociclib alone.

FIG. 25 is a graph showing the expression levels of MTHFD2 in tumor samples collected at the end of treatment, wherein the mice were treated either with a vehicle control, a combination of compound 1 and palbociclib, compound 1 alone or palbociclib alone.

FIG. 26 is a graph showing the expression levels of SHMT1 in tumor samples collected at the end of treatment, wherein the mice were treated either with a vehicle control, a combination of compound 1 and palbociclib, compound 1 alone or palbociclib alone.

FIG. 27 is a graph showing the expression levels of SHMT2 in tumor samples collected at the end of treatment, wherein the mice were treated either with a vehicle control, a combination of compound 1 and palbociclib, compound 1 alone or palbociclib alone.

FIG. 28 is a graph showing the expression levels of PIK3IP1 in tumor samples collected at the end of treatment, wherein the mice were treated either with a vehicle control, a combination of compound 1 and palbociclib, compound 1 alone or palbociclib alone.

FIG. 29 is a graph showing the expression levels of Greb1 in tumor samples collected at the end of treatment, wherein the mice were treated either with a vehicle control, a combination of compound 1 and palbociclib, compound 1 alone or palbociclib alone.

FIG. 30 is a Kaplan-Meier Plot of ER+ breast cancer patients with either high expression or low expression of PHGDH (N=5526).

FIG. 31 is a Kaplan-Meier Plot of ER+ breast cancer patients with either high expression or low expression of TYMS (N=5526).

FIG. 32 is a Kaplan-Meier Plot of ER+ breast cancer patients with either high expression or low expression of PIK3IP1 (N=5526).

FIG. 33 is a graph showing the expression levels of DHFR in tumor samples collected at the end of treatment, wherein the mice were treated either with a vehicle control, a combination of compound 1 and palbociclib, compound 1 alone or palbociclib alone.

FIG. 34 is a graph showing the expression levels of MybL2 in tumor samples collected at the end of treatment, wherein the mice were treated either with a vehicle control, a combination of compound 1 and palbociclib, compound 1 alone or palbociclib alone.

FIG. 35 is a graph showing the expression levels of BIRC5/Survivin in tumor samples collected at the end of treatment, wherein the mice were treated either with a vehicle control, a combination of compound 1 and palbociclib, compound 1 alone or palbociclib alone.

FIG. 36 is a graph showing the expression levels of Ki-67 in tumor samples collected at the end of treatment, wherein the mice were treated either with a vehicle control, a combination of compound 1 and palbociclib, compound 1 alone or palbociclib alone.

FIG. 37 is a graph showing the expression levels of CCNB1/Cyclin B1 in tumor samples collected at the end of treatment, wherein the mice were treated either with a vehicle control, a combination of compound 1 and palbociclib, compound 1 alone or palbociclib alone.

FIG. 38 is a graph showing the expression levels of SCUBE2 in tumor samples collected at the end of treatment, wherein the mice were treated either with a vehicle control, a combination of compound 1 and palbociclib, compound 1 alone or palbociclib alone.

FIG. 39 is a graph showing the expression levels of RRM2 in tumor samples collected at the end of treatment, wherein the mice were treated either with a vehicle control, a combination of compound 1 and palbociclib, compound 1 alone or palbociclib alone.

FIG. 40 is a graph showing the expression levels of PCLAF in tumor samples collected at the end of treatment, wherein the mice were treated either with a vehicle control, a combination of compound 1 and palbociclib, compound 1 alone or palbociclib alone.

FIG. 41 is a graph showing the expression levels of SLC7A5/LAT1 in tumor samples collected at the end of treatment, wherein the mice were treated either with a vehicle control, a combination of compound 1 and palbociclib, compound 1 alone or palbociclib alone.

FIG. 42 is a graph showing the expression levels of SLC3A2 in tumor samples collected at the end of treatment, wherein the mice were treated either with a vehicle control, a combination of compound 1 and palbociclib, compound 1 alone or palbociclib alone.

FIG. 43 is a graph showing the expression levels of EVL in tumor samples collected at the end of treatment, wherein the mice were treated either with a vehicle control, a combination of compound 1 and palbociclib, compound 1 alone or palbociclib alone.

FIG. 44 is a graph showing the expression levels of ANP32E in tumor samples collected at the end of treatment, wherein the mice were treated either with a vehicle control, a combination of compound 1 and palbociclib, compound 1 alone or palbociclib alone.

FIG. 45 is a graph showing the expression levels of H2AZ1 in tumor samples collected at the end of treatment, wherein the mice were treated either with a vehicle control, a combination of compound 1 and palbociclib, compound 1 alone or palbociclib alone.

FIG. 46 is a graph showing the expression levels of H2AX in tumor samples collected at the end of treatment, wherein the mice were treated either with a vehicle control, a combination of compound 1 and palbociclib, compound 1 alone or palbociclib alone.

FIG. 47 is a graph showing the MCF tumor volume in mice over the course of 14 days of treatment, wherein the mice were treated either with a vehicle control, a combination of compound 1 and ribociclib, compound 1 alone or ribociclib alone.

FIG. 48 is a graph showing the MCF tumor volume in mice at day 14 of treatment, wherein the mice were treated either with a vehicle control, a combination of compound 1 and ribociclib, compound 1 alone or ribociclib alone.

FIG. 49 is a graph showing the MCF tumor volume in mice over the course of 18 days of treatment, wherein the mice were treated either with a vehicle control, a combination of compound 1 and ribociclib, compound 1 alone or ribociclib alone.

FIG. 50 is a graph showing the MCF tumor volume in mice at day 18 of treatment, wherein the mice were treated either with a vehicle control, a combination of compound 1 and ribociclib, compound 1 alone or ribociclib alone.

### DETAILED DESCRIPTION

The present disclosure provides, inter alia, a method of treating cancer or preventing treatment resistance to cancer, comprising administering to a subject in need thereof at least one therapeutically effective amount of at least one MetAP2 inhibitor of the present disclosure, or a pharmaceutically acceptable salt thereof and at least one therapeutically effective amount of at least one CDK4/6 inhibitor, or a pharmaceutically acceptable salt thereof.

The present disclosure provides a combination therapy comprising at least one therapeutically effective amount of at least one MetAP2 inhibitor of the present disclosure, or a pharmaceutically acceptable salt thereof and at least one therapeutically effective amount of at least one CDK4/6 inhibitor, or a pharmaceutically acceptable salt thereof.

The present disclosure provides a method of treating cancer in a subject in need thereof, the method comprising administering to the subject in need thereof at least one therapeutically effective amount of the preceding combination therapy.

The present disclosure provides a method of preventing and/or mitigating treatment resistance in a subject in need thereof, the method comprising administering to the subject in need thereof at least one therapeutically effective amount of the preceding combination therapy.

The present disclosure provides a pharmaceutical composition comprising at least one therapeutically effective amount of at least one MetAP2 inhibitor of the present disclosure, or a pharmaceutically acceptable salt thereof and at least one therapeutically effective amount of at least one CDK4/6 inhibitor, or a pharmaceutically acceptable salt thereof.

The present disclosure provides a method of treating cancer in a subject in need thereof, the method comprising administering to the subject in need thereof at least one therapeutically effective amount of the preceding pharmaceutical composition.

The present disclosure provides a method of preventing and/or mitigating treatment resistance in a subject in need thereof, the method comprising administering to the subject in need thereof at least one therapeutically effective amount of the preceding pharmaceutical composition.

The present disclosure provides a kit comprising at least one therapeutically effective amount of at least one MetAP2 inhibitor of the present disclosure, or a pharmaceutically acceptable salt thereof and at least one therapeutically effective amount of at least one CDK4/6 inhibitor, or a pharmaceutically acceptable salt thereof.

The present disclosure provides a method of treating cancer a subject in need thereof, the method comprising administering to the subject in need thereof at least one therapeutically effective amount of the preceding kit.

The present disclosure provides a method of preventing and/or mitigating treatment resistance in a subject in need thereof, the method comprising administering to the subject at least one therapeutically effective amount of the preceding kit.

The present disclosure provides a method of treating cancer a subject in need thereof, the method comprising administering to the subject at least one therapeutically effective amount of at least one MetAP2 inhibitor of the present disclosure, or a pharmaceutically acceptable salt thereof, and at least one therapeutically effective amount of at least one CDK4/6 inhibitor, or a pharmaceutically acceptable salt thereof.

The present disclosure provides a method of preventing and/or mitigating treatment resistance in a subject in need thereof, the method comprising administering to the subject at least one therapeutically effective amount of at least one MetAP2 inhibitor of the present disclosure, or a pharmaceutically acceptable salt thereof, and at least one therapeutically effective amount of at least one CDK4/6 inhibitor, or a pharmaceutically acceptable salt thereof.

The present disclosure provides a use of at least one MetAP2 inhibitor of the present disclosure, or a pharmaceutically acceptable salt thereof, in combination with at least one CDK4/6 inhibitor or a pharmaceutically acceptable salt thereof, in the manufacture of a medicament for the treatment of a cancer.

The present disclosure provides a use of at least one MetAP2 inhibitor of the present disclosure, or a pharmaceutically acceptable salt thereof, in combination with at least one CDK4/6 inhibitor or a pharmaceutically acceptable salt thereof, in the manufacture of a medicament for the prevention and/or the mitigation of treatment resistance in a subject in need thereof.

The present disclosure provides a use of at least one CDK4/6 inhibitor, or a pharmaceutically acceptable salt thereof, in combination with at least one MetAP2 inhibitor of the present disclosure, or a pharmaceutically acceptable salt thereof, in the manufacture of a medicament for the treatment of a cancer.

The present disclosure provides a use of at least one CDK4/6 inhibitor, or a pharmaceutically acceptable salt thereof, in combination with at least one MetAP2 inhibitor of the present disclosure, or a pharmaceutically acceptable salt thereof, in the manufacture of a medicament for the prevention and/or the mitigation of treatment resistance in a subject in need thereof.

The present disclosure provides a combination of at least one MetAP2 inhibitor of the present disclosure, or a pharmaceutically acceptable salt thereof in combination with at least one CDK 4/6 inhibitor, or a pharmaceutically acceptable salt thereof, for use in the manufacture of a medicament for the treatment of a cancer.

The present disclosure provides a combination of at least one MetAP2 inhibitor of the present disclosure, or a pharmaceutically acceptable salt thereof in combination with at least one CDK4/6 inhibitor, or a pharmaceutically acceptable salt thereof, for use in the manufacture of a medicament for the prevention and/or the mitigation of treatment resistance in a subject in need thereof.

In some aspects of the preceding methods and uses, the treatment resistance can be resistance to treatment with a CDK4/6 inhibitor.

The present disclosure provides at least one MetAP2 inhibitor of the present disclosure, or a pharmaceutically acceptable salt thereof, for use in combination with at least one CDK4/6 inhibitor or a pharmaceutically acceptable salt thereof, in treating a cancer.

The present disclosure provides at least one MetAP2 inhibitor of the present disclosure, or a pharmaceutically acceptable salt thereof, for use in combination with at least one CDK4/6 inhibitor or a pharmaceutically acceptable salt thereof, in preventing and/or mitigating treatment resistance in a subject in need thereof.

The present disclosure provides at least one CDK4/6 inhibitor or a pharmaceutically acceptable salt thereof, for use in combination with at least one MetAP2 inhibitor of the present disclosure, or a pharmaceutically acceptable salt thereof, in treating a cancer.

The present disclosure provides at least one CDK4/6 inhibitor or a pharmaceutically acceptable salt thereof, for use in combination with at least one MetAP2 inhibitor of the present disclosure, or a pharmaceutically acceptable salt thereof, in preventing and/or mitigating treatment resistance in a subject in need thereof.

The present disclosure provides a combination of at least one MetAP2 inhibitor of the present disclosure, or a pharmaceutically acceptable salt thereof, and at least one CDK4/6 inhibitor or a pharmaceutically acceptable salt thereof, for use in treating a cancer.

The present disclosure provides a combination of at least one MetAP2 inhibitor of the present disclosure, or a pharmaceutically acceptable salt thereof, and at least one CDK4/6 inhibitor or a pharmaceutically acceptable salt thereof, for use in preventing and/or mitigating treatment resistance in a subject in need thereof.

The present disclosure provides a combination comprising at least one MetAP2 inhibitor of the present disclosure, or a pharmaceutically acceptable salt thereof, and at least one CDK4/6 inhibitor or a pharmaceutically acceptable salt thereof, for use in treating a cancer. The present disclosure provides a combination comprising at least one MetAP2 inhibitor of the present disclosure, or a pharmaceutically acceptable salt thereof, for use in treating a cancer, wherein the combination comprises at least one CDK4/6 inhibitor, or a pharmaceutically acceptable salt thereof. The present disclosure provides a combination comprising at least one CDK4/6 inhibitor, or a pharmaceutically acceptable salt thereof, for use in treating a cancer, wherein the combination comprises at least one MetAP2 inhibitor of the present disclosure, or a pharmaceutically acceptable salt thereof. In some aspects, the at least one at least one MetAP2 inhibitor of the present disclosure, or a pharmaceutically acceptable salt thereof, and that least at least one CDK4/6 inhibitor, or a pharmaceutically acceptable salt thereof can be administered concurrently, separately or sequentially. In some aspects, the at least one at least one MetAP2 inhibitor of the present disclosure, or a pharmaceutically acceptable salt thereof, and that least at least one CDK4/6 inhibitor, or a pharmaceutically acceptable salt thereof can be administered concurrently or in temporal proximity.

In some aspects of the preceding methods and uses, treatment resistance can be treatment resistance to at least one CDK4/6 inhibitor.

In some aspects, a MetAP2 inhibitor, or pharmaceutically acceptable salt thereof, and a CDK4/6 inhibitor, or pharmaceutically acceptable salt thereof, can be administered by the same administration route. In some aspects, the MetAP2 inhibitor, or pharmaceutically acceptable salt thereof, and the CDK4/6 inhibitor, or pharmaceutically acceptable salt thereof, can be administered by a different administration route.

In some aspects, the MetAP2 inhibitor, or pharmaceutically acceptable salt thereof, and the CDK4/6 inhibitor, or pharmaceutically acceptable salt thereof, can be administered concurrently.

In some aspects, the MetAP2 inhibitor, or pharmaceutically acceptable salt thereof, and the CDK4/6 inhibitor, or pharmaceutically acceptable salt thereof, can be administered in temporal proximity.

As used herein, the term “temporal proximity” refers to that administration of one therapeutic agent (e.g., a MetAP2 inhibitor compound disclosed herein) occurs within a time period before or after the administration of another therapeutic agent (e.g., palbociclib), such that the therapeutic effect of the one therapeutic agent overlaps with the therapeutic effect of the other therapeutic agent. In some embodiments, the therapeutic effect of the one therapeutic agent completely overlaps with the therapeutic effect of the other therapeutic agent. In some embodiments, “temporal proximity” means that administration of one therapeutic agent occurs within a time period before or after the administration of another therapeutic agent, such that there is a synergistic effect between the one therapeutic agent and the other therapeutic agent. “Temporal proximity” may vary according to various factors, including but not limited to, the age, gender, weight, genetic background, medical condition, disease history, and treatment history of the subject to which the therapeutic agents are to be administered; the disease or condition to be treated or ameliorated; the therapeutic outcome to be achieved; the dosage, dosing frequency, and dosing duration of the therapeutic agents; the pharmacokinetics and pharmacodynamics of the therapeutic agents; and the route(s) through which the therapeutic agents are administered. In some embodiments, “temporal proximity” means within 15 minutes, within 30 minutes, within an hour, within two hours, within four hours, within six hours, within eight hours, within 12 hours, within 18 hours, within 24 hours, within 36 hours, within 2 days, within 3 days, within 4 days, within 5 days, within 6 days, within a week, within 2 weeks, within 3 weeks, within 4 weeks, with 6 weeks, or within 8 weeks. In some embodiments, multiple administration of one therapeutic agent can occur in temporal proximity to a single administration of another therapeutic agent. In some embodiments, temporal proximity may change during a treatment cycle or within a dosing regimen.

In some aspects, the administration of a combination of at least one MetAP2 inhibitor, or pharmaceutically acceptable salt thereof, and at least one CDK4/6 inhibitor, or pharmaceutically acceptable salt thereof, can result in the decrease in the expression level of at least one protein in at least one tumor in the subject. In some aspects, the at least one protein can comprise Rb protein, CDK2 protein, CDK4 protein, cyclin E1 protein, cyclin E2 protein, Akt protein, Phospho-Akt, ERα-62, ERα-55 or any combination thereof. In some aspects, the decrease in the expression level of the at least one protein can be a decrease of at least about 5%, or at least about 10%, or at least about 15%, or at least about 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, or at least about 90%, or at least about 95%, or at least about 99%.

In some aspects, the administration of a combination of at least one MetAP2 inhibitor, or pharmaceutically acceptable salt thereof, and at least one CDK4/6 inhibitor, or pharmaceutically acceptable salt thereof, can result in the decrease in the expression level of at least one gene encoding at least one protein in at least one tumor in the subject. In some aspects, the at least one protein can comprise Rb protein, CDK2 protein, CDK4 protein, cyclin E1 protein, cyclin E2 protein, Akt protein, Phospho-Akt, ERα-62, ERα-55, PHGDH or any combination thereof. In some aspects, the decrease in the expression level of the at least one gene encoding the at least one protein can be a decrease of at least about 5%, or at least about 10%, or at least about 15%, or at least about 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, or at least about 90%, or at least about 95%, or at least about 99%.

In some aspects, the administration of a combination of at least one MetAP2 inhibitor, or pharmaceutically acceptable salt thereof, and at least one CDK4/6 inhibitor, or pharmaceutically acceptable salt thereof, can result in the decrease in the expression level of at least one gene in at least one tumor in the subject. In some aspects, the at least one gene can comprise MTHFDIL, TYMS, ALDH1L1, MTHFD1, MTHFD2, GART, SHMT1, DHFR, MTR, SHMT2 and MTFMT or any combination thereof. In some aspects, the at least one gene can comprise PHGDH, PSPH, TYMS, MTHFD1L, MTHFD1, MTHFD2, SHMT1, SHMT2, Greb1, DHFR, MybL2, BIRC5/Suvivin, Ki-67, CCNB1/Cyclin B1, RRM2, PCLAF, SLC7A5/LAT1, SLC3A2 ANP32E, H2AZ1, H2AX or any combination thereof. In some aspects, the decrease in the expression level of the at least one gene can be a decrease of at least about 5%, or at least about 10%, or at least about 15%, or at least about 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, or at least about 90%, or at least about 95%, or at least about 99%.

In some aspects, the decrease in the expression level of the at least one protein, the at least one gene encoding the at least one protein, or the at least one gene, upon administration of the combination at least one MetAP2 inhibitor, or pharmaceutically acceptable salt thereof, and at least one CDK4/6 inhibitor, or pharmaceutically acceptable salt thereof, can be a greater decrease in expression level as compared to the decrease in expression level caused by administration of the at least one MetAP2 inhibitor alone and/or the administration of the at least one CDK4/6 inhibitor alone. In some aspects, the decrease in expression level can be at least about 25%, or at least about 50%, or at least about 75%, or at least about 100%, or at least about 125%, or at least about 150%, or at least about 175%, or at least about 200% greater upon administration of the combination of the combination at least one MetAP2 inhibitor, or pharmaceutically acceptable salt thereof, and at least one CDK4/6 inhibitor as compared to the decrease in expression level caused by administration of the at least one MetAP2 inhibitor alone and/or the administration of the at least one CDK4/6 inhibitor alone.

In some aspects, the administration of a combination of at least one MetAP2 inhibitor, or pharmaceutically acceptable salt thereof, and at least one CDK4/6 inhibitor, or pharmaceutically acceptable salt thereof, can result in a reduction in the increase in the expression level of at least one protein in at least one tumor in the subject caused by the administration of the at least one MetAP2 inhibitor alone and/or the at least one CDK4/6 inhibitor alone. In some aspects, the at least one protein can comprise Rb protein, p21 protein, CDK2 protein, CDK4 protein, cyclin E1 protein, cyclin E2 protein, LC3B protein, estrogen receptor, Akt protein, or any combination thereof. In some aspects, the reduction can be a reduction of at least about 5%, or at least about 10%, or at least about 15%, or at least about 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, or at least about 90%, or at least about 95%, or at least about 99%.

In some aspects, the administration of a combination of at least one MetAP2 inhibitor, or pharmaceutically acceptable salt thereof, and at least one CDK4/6 inhibitor, or pharmaceutically acceptable salt thereof, can result an increase in expression of at least one protein that is smaller as compared to the increase in expression level caused by administration of the at least one MetAP2 inhibitor alone and/or the administration of the at least one CDK4/6 inhibitor alone. In some aspects, the at least one protein can be p²¹, LC3B or Cyclin D1. In some aspects, the increase in expression upon administration of the combination of at least one MetAP2 inhibitor, or pharmaceutically acceptable salt thereof, and at least one CDK4/6 inhibitor, or pharmaceutically acceptable salt thereof is at least about 10%, or at least about 25%, or at least about 50%, or at least about 75%, or at least about 100%, or at least about 125%, or at least about 150%, or at least about 175%, or at least about 200% less than the increase in expression upon administration of the at least one MetAP2 inhibitor alone and/or the administration of the at least one CDK4/6 inhibitor alone.

In some aspects, the administration of a combination of at least one MetAP2 inhibitor, or pharmaceutically acceptable salt thereof, and at least one CDK4/6 inhibitor, or pharmaceutically acceptable salt thereof, can attenuate the increase in expression of at least one protein as compared to the increase in expression level caused by administration of the at least one MetAP2 inhibitor alone and/or the administration of the at least one CDK4/6 inhibitor alone. In some aspects the at least one protein can be p21, Akt protein, or Cyclin D1. In some aspects, the attenuation can be an attenuation of at least about 10%, or at least about 25%, or at least about 50%, or at least about 75%, or at least about 100%.

In some aspects, the administration of a combination of at least one MetAP2 inhibitor, or pharmaceutically acceptable salt thereof, and at least one CDK4/6 inhibitor, or pharmaceutically acceptable salt thereof, can result an increase in expression of at least one protein that is no more than about 10%, or about 20%, or about 30%, or about 40%, or about 50%, or about 60%, or about 70%, or about 80%, or about 90%, or about 100%.

In some aspects, the administration of a combination of at least one MetAP2 inhibitor, or pharmaceutically acceptable salt thereof, and at least one CDK4/6 inhibitor, or pharmaceutically acceptable salt thereof, can result in the increased amount of neutrophils in a subject as compared to the amount of neutrophils in a subject that has been administered the at least one MetAP2 inhibitor alone and/or the at least one CDK4/6 inhibitor alone. In some aspects, the amount of neutrophils in a subject upon administration of the combination of at least one MetAP2 inhibitor, or pharmaceutically acceptable salt thereof, and at least one CDK4/6 inhibitor, or pharmaceutically acceptable salt thereof is at least about 5%, or at least about 10%, or at least about 15%, or at least about 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, or at least about 90%, or at least about 95%, or at least about 100%, or at least about 125%, or at least about 150%, or at least about 175%, or at least about 200%, or at least about 22%, or at least about 250%, or at least about 275%, or at least about 300%, or at least about 325%, or at least about 350%, or at least about 375%, or at least about 400%, or at least about 425%, or at least about 450% greater as compared to the amount of neutrophils in a subject that has been administered the at least one MetAP2 inhibitor alone and/or the administration of the at least one CDK4/6 inhibitor alone.

In some aspects, the administration of a combination of at least one MetAP2 inhibitor, or pharmaceutically acceptable salt thereof, and at least one CDK4/6 inhibitor, or pharmaceutically acceptable salt thereof, can result in the increase in the expression level of at least one gene. In some aspects, the at least one gene can comprise PIK3IP1, SCUBE2, EVL. In some aspects, the increase in the expression level of the at least one protein can be an increase of at least about 5%, or at least about 10%, or at least about 15%, or at least about 20%, or at least about 25%, or at least about 30%, or at least about 35%, or at least about 40%, or at least about 45%, or at least about 50%, or at least about 55%, or at least about 60%, or at least about 65%, or at least about 70%, or at least about 75%, or at least about 80%, or at least about 85%, or at least about 90%, or at least about 95%, or at least about 99%.

MetAP2 Inhibitors

Any of the MetAP2 inhibitors described herein can be used in the kits, pharmaceutical compositions, uses and methods described herein.

In some aspects, a MetAP2 inhibitor can be a compound of Formula (I), or a pharmaceutically acceptable salt, analog, derivative, salt or ester thereof, wherein Formula I is represented by:

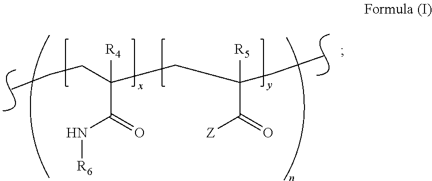

wherein, independently for each occurrence, R₄ is H or C₁-C₆ alkyl; R₅ is H or C₁-C₆ alkyl; R₆ is C₂-C₆ hydroxyalkyl; Z is —NH-AA₁-AA₂-AA₃-AA₄-AA₅-AA₆-C(O)-L or —NH-AA₁-AA₂-AA₃-AA₄-AA₅-AA₆-C(O)-Q-X—Y—C(O)—W; AA₁ is glycine, alanine, or H₂N(CH₂)ₘCO₂H, wherein m is 2, 3, 4 or 5; AA₂ is a bond, or alanine, cysteine, aspartic acid, glutamic acid, phenylalanine, glycine, histidine, isoleucine, lysine, leucine, methionine, asparagine, proline, glutamine, arginine, serine, threonine, valine, tryptophan, or tyrosine; AA₅ is a bond, or alanine, cysteine, aspartic acid, glutamic acid, phenylalanine, glycine, histidine, isoleucine, lysine, leucine, methionine, asparagine, proline, glutamine, arginine, serine, threonine, valine, tryptophan, or tyrosine; AA₄ is a bond, or alanine, cysteine, aspartic acid, glutamic acid, phenylalanine, glycine, histidine, isoleucine, lysine, leucine, methionine, asparagine, proline, glutamine, arginine, serine, threonine, valine, tryptophan, or tyrosine; AA₅ is a bond, or glycine, valine, tyrosine, tryptophan, phenylalanine, methionine, leucine, isoleucine, or asparagine; AA₅ is a bond, or alanine, asparagine, citrulline, glutamine, glycine, leucine, methionine, phenylalanine, serine, threonine, tryptophan, tyrosine, valine, or H₂N(CH₂)mCO₂H, wherein in is 2, 3, 4 or 5; L is —OH, —O-succinimide, —O-sulfosuccinimide, alkoxy, aryloxy, acyloxy, aroyloxy, alkoxycarbonyloxy, aryloxycarbonyloxy, —NH₂, —NH(C₂-C₆ hydroxyalkyl), halide or perfluoroalkyloxy; Q is NR, O, or S; X is M-(C(R)₂)ₚ-M-J-M-(C(R)₂)ₚ-M-V; M is a bond, or C(O); J is a bond, or ((CH₂)_{q}Q)ᵣ, C₅-C₈cycloalkyl, aryl, heteroaryl, NR, O, or S; Y is NR, O, or S; R is H₁ or alkyl; V is a bond or

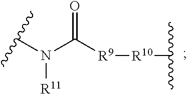

R⁹ is alkyl, aryl, aralkyl, or a bond; or R⁹ taken together with Y forms a heterocyclic ring; R¹⁰ is amido or a bond; R¹¹ is H or alkyl; W is a MetAP2 inhibitor moiety or alkyl; x is in the range of 1 to about 450; y is in the range of 1 to about 30; n is in the range of 1 to about 100; p is 0 to 20; q is 2 or 3; and r is 1, 2, 3, 4, 5, or 6. In some aspects, n is in the range of about 1 to about 90; about 1 to about 80; about 1 to about 70; about 1 to about 60; about 1 to about 55; or about 1 to about 50.

In some embodiments, R₄ is C₁-C₆ alkyl. In some embodiments, R₄ is methyl. In some embodiments, R₅ is C₁-C₆ alkyl. In some embodiments, R₅ is methyl. In some embodiments, R₆ is 2-hydroxyethyl, 2-hydroxypropyl or 3-hydroxypropyl. In some embodiments, R₆ is 2-hydroxypropyl.

In some embodiments, the compound has a molecular weight of greater than about 100 kDa. In some embodiments, the compound has a molecular weight of less than about 100 kDa. In some embodiments, the molecular weight is less than about 95 kDa. In some embodiments, the molecular weight is less than about 90 kDa. In some embodiments, the molecular weight is less than about 80 kDa. In some embodiments, the molecular weight is less than about 70 kDa. In some embodiments, the molecular weight is less than about 65 kDa. In some embodiments, the molecular weight is less than about 60 kDa. In some embodiments, the molecular weight is less than about 45 kDa. In some embodiments, the molecular weight is less than about 35 kDa.

In some embodiments, the ratio of x to y is in the range of about 100:1 to about 1:1. In some embodiments, the ratio of x to y is in the range of about 30:1 to about 3:1. In some embodiments, the ratio of x to y is in the range of about 19:2 to about 7:2. In some embodiments, the ratio of x to v is in the range of about 9:1 to about 4:1. In some embodiments, the ratio of x to y is about 11:1. In some embodiments, the ratio of x to y is about 9:1. In some embodiments, the ratio of x to y is about 4:1. In some embodiments, the ratio of x to y is about 12:1. For example, In some embodiments, the ratio of x:y is about 3:1; the ratio of x:y is about 4:1; the ratio of x:y is about 5:1; the ratio of x:y is about 6:1; the ratio of x:y is about 7:1; the ratio of x:y is about 8:1; the ratio of x:y is about 9:1 the ratio of x:y is about 10:1; the ratio of x:y is about 11:1; the ratio of x:y is about 12:1; the ratio of x:y is about 13:1; the ratio of x:y is about 14:1; the ratio of x:y is about 15:1; the ratio of x:y is about 16:1; the ratio of x:y is about 17:1; the ratio of x:y is about 18:1; the ratio of x:y is about 19:1; the ratio of x:y is about 20:1; the ratio of x:y is about 21:1; the ratio of x:y is about 22:1; the ratio of x:y is about 23:1; the ratio of x:y is about 24:1; the ratio of x:y is about 25:1; the ratio of x:y is about 26:1; the ratio of x:y is about 27:1; the ratio of x:y is about 28:1; the ratio of x:y is about 29:1; or the ratio of x:y is about 30:1.

In some embodiments, Z is —NH-AA₁-AA₂-AA₁-AA₄-AA₅-AA₆-C(O)-L. In some embodiments, L is methoxy, ethoxy, pentafluorophenyloxy, phenyloxy, acetoxy, fluoride, chloride, methoxycarbonyloxy; ethoxycarbonyloxy, phenyloxycarbonyloxy, 4-nitrophenyloxy, trifluoromethoxy, pentafluoroethoxy, or trifluoroethoxy. In some embodiments, L is 4-nitrophenyloxy.

In some embodiments, Z is —NH-AA₁-AA₂-AA₃-AA₄-AA₅-AA₆-C(O)-Q-X—Y—C(O)—W. In some embodiments, AA₁ is glycine. In some embodiments, AA₂ is glycine. In some embodiments, AA₃ is glycine. In some embodiments, AA₄ is glycine or phenylalanine. In some embodiments, AA₅ is leucine, phenylalanine, valine or tyrosine. In some embodiments, AA₆ is asparagine, citrulline, glutamine, glycine, leucine, methionine, threonine or tyrosine. In some embodiments, AA₅-AA₆ is Leu-Cit, Leu-Gin, Leu-Gly, Leu-Leu, Leu-Met, Leu-Thr, Phe-Cit, Phe-Gln, Phe-Leu, Phe-Met, Phe-Thr, Val-Asn, Val-Cit, Val-Gln, Val-Leu, Val-Met, Val-Thr, Tyr-Cit, Tyr-Leu, or Tyr-Met. In some embodiments, AA₁, AA₃ and AA₅ are glycine, valine, tyrosine, tryptophan, phenylalanine, methionine, leucine, isoleucine, or asparagine. In some embodiments, AA₂, AA₄ and AA₆ are glycine, asparagine, citrulline, glutamine, glycine, leucine, methionine, phenylalanine, threonine or tyrosine. In some embodiments, AA₂ is a bond; and AA₃ is a bond. In some embodiments, AA₁ is glycine; AA₄ is phenylalanine; AA₅ is leucine; and AA₆ is glycine.

In some embodiments, W is

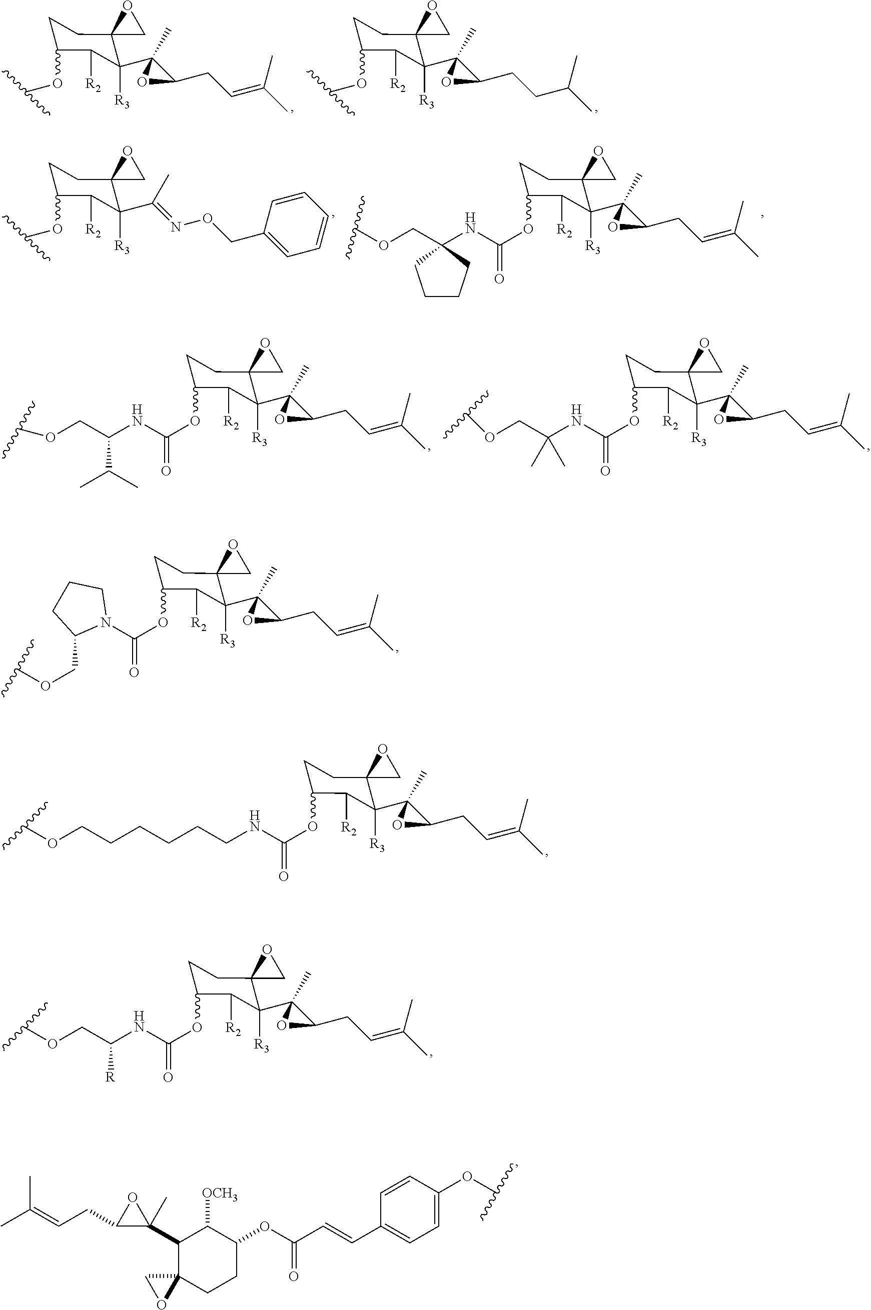

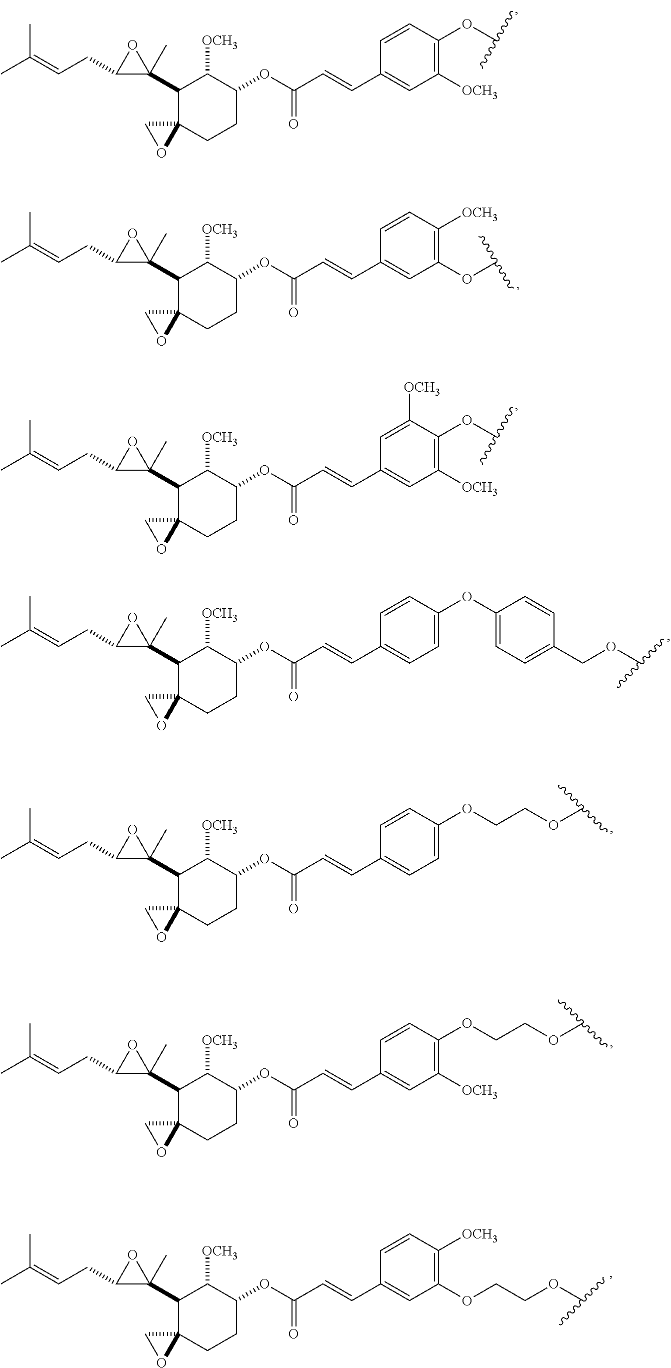

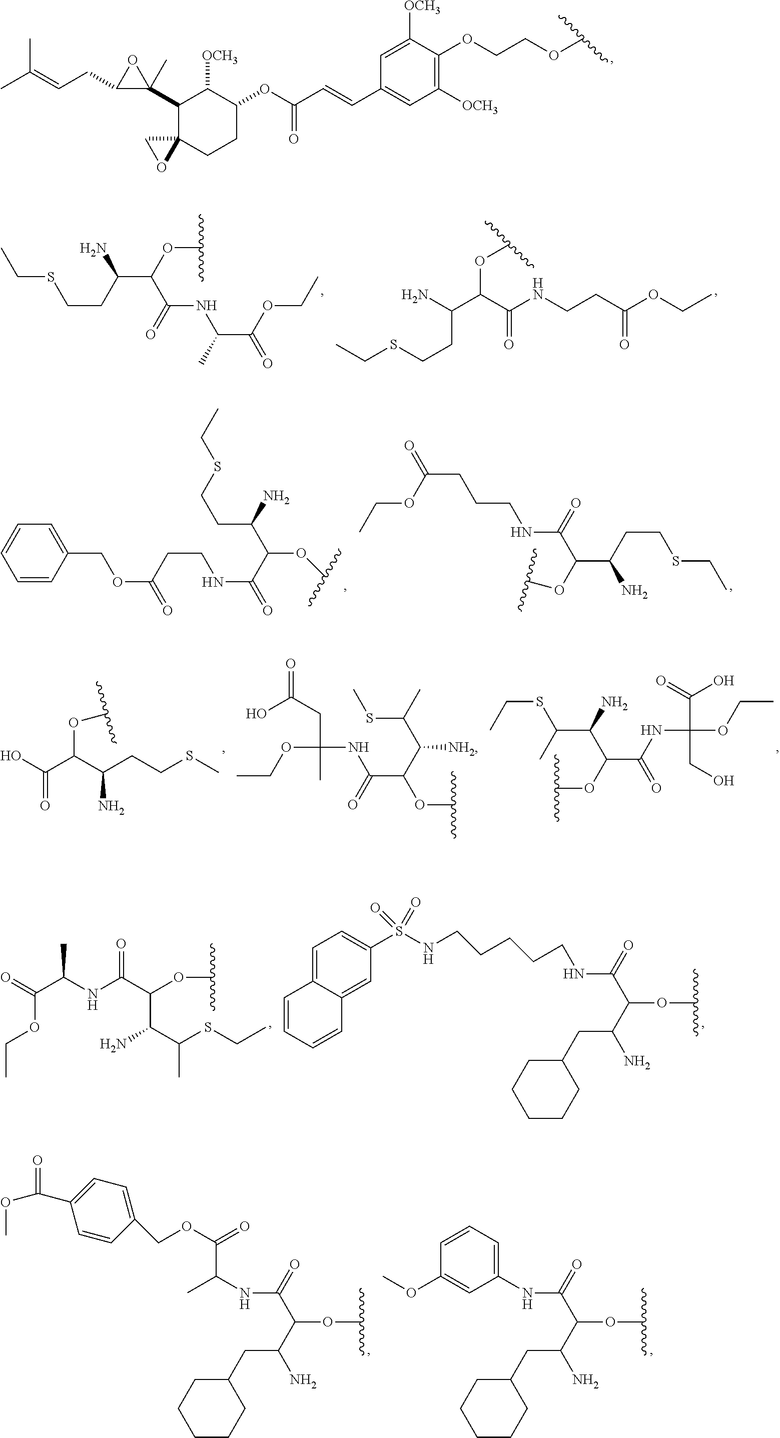

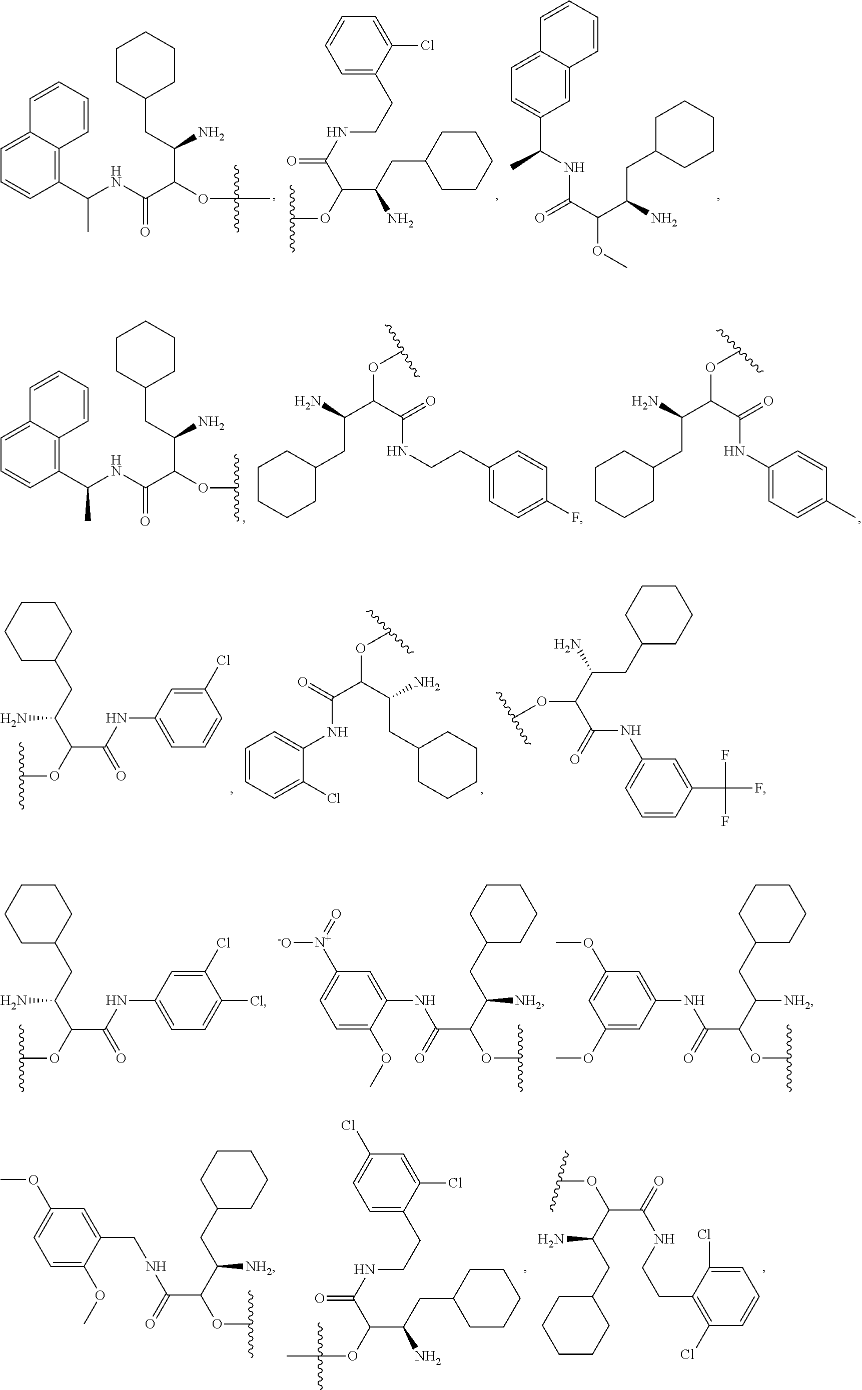

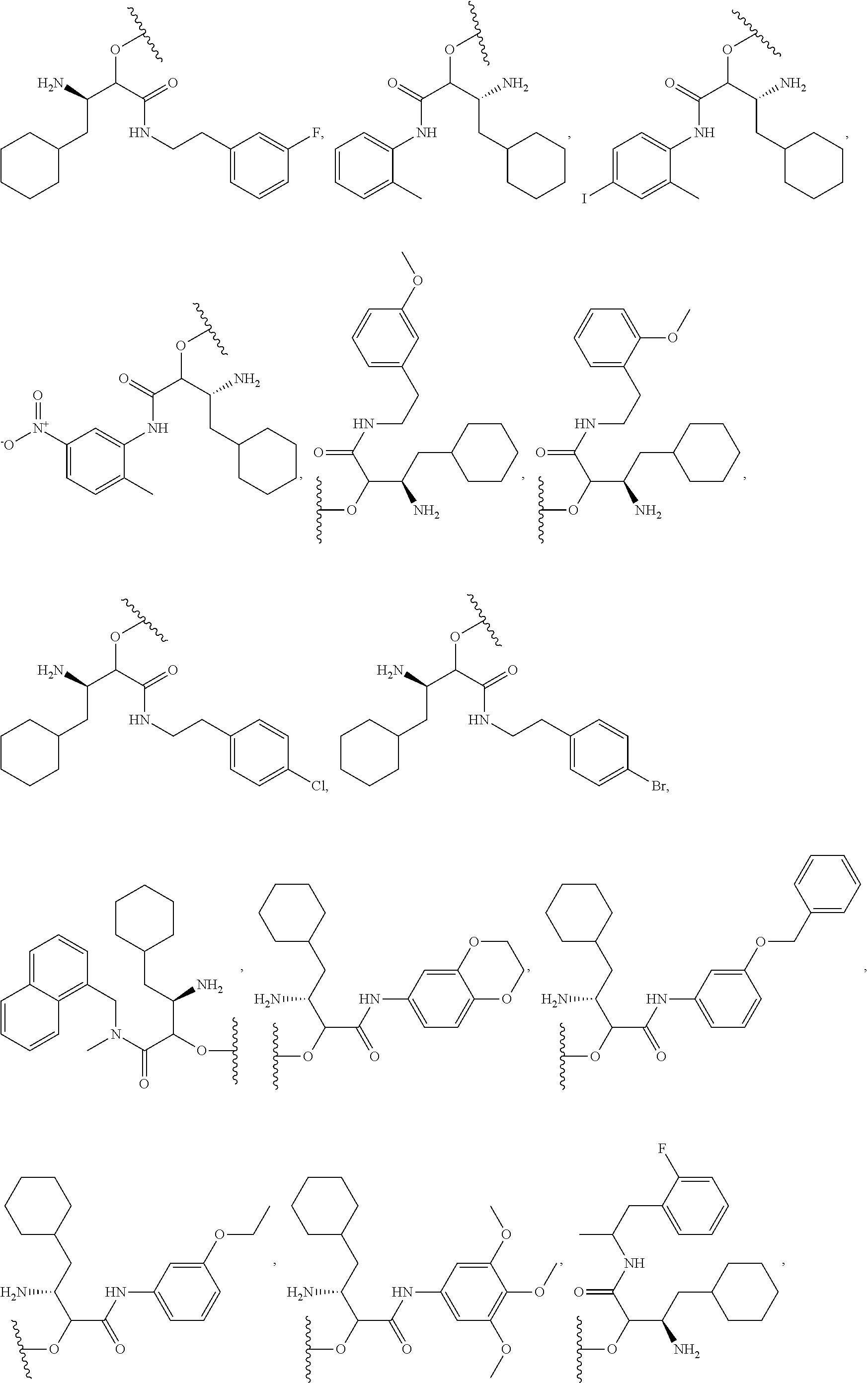

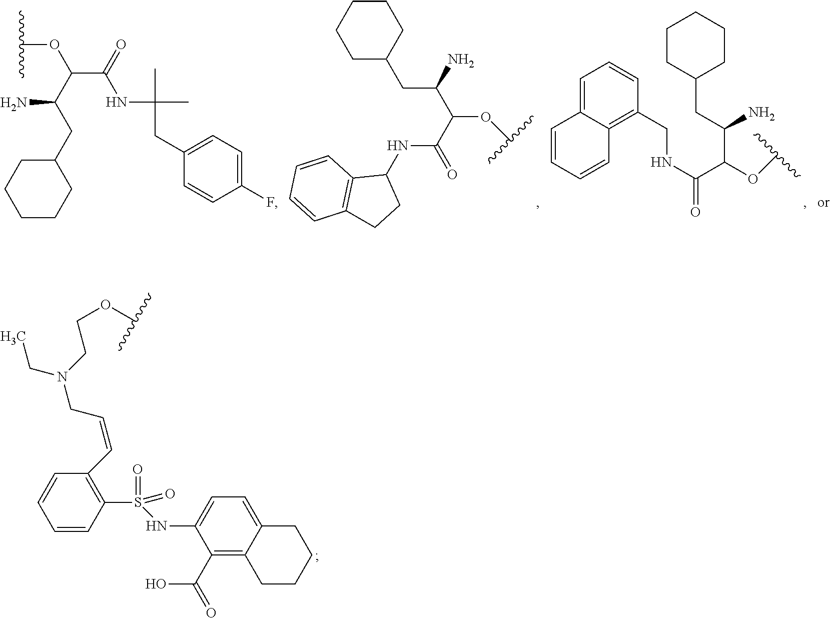

wherein R₂ is —OH or methoxy; and R₃ is H, —OH or methoxy.

In some embodiments, W is

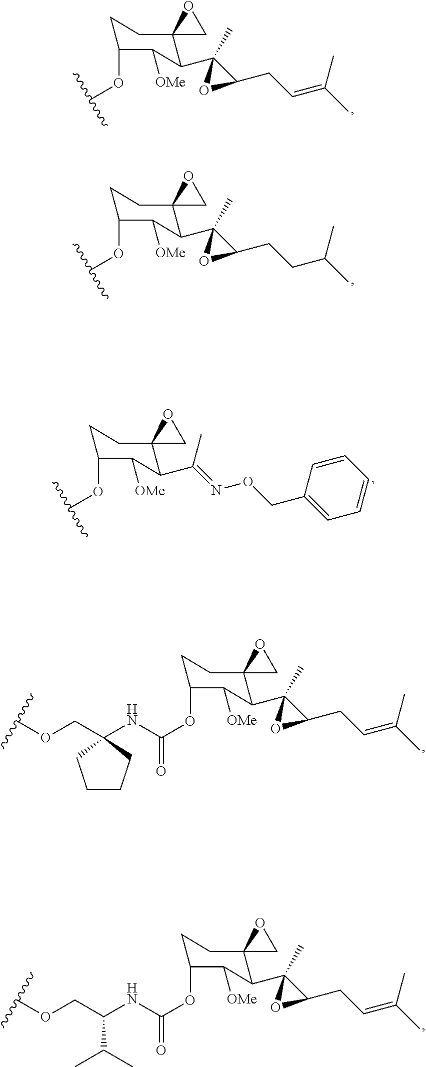

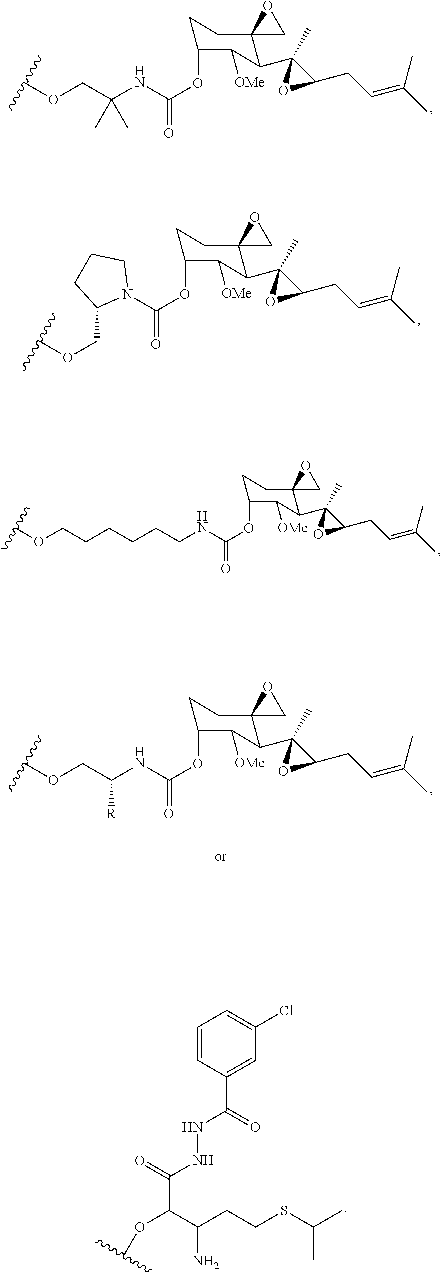

In some embodiments, W is

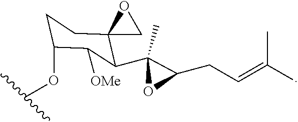

In some embodiments, Q is NR. In some embodiments, Q is S.

In some embodiments, J is NR. In some embodiments, J is ((CH₂)_{q}Q)ᵣ. In some embodiments, J is C₅-C₈ cycloalkyl. In some embodiments, J is aryl.

In some embodiments, Y is NR. In some embodiments, Y is S.

In some embodiments, -Q-X—Y— is

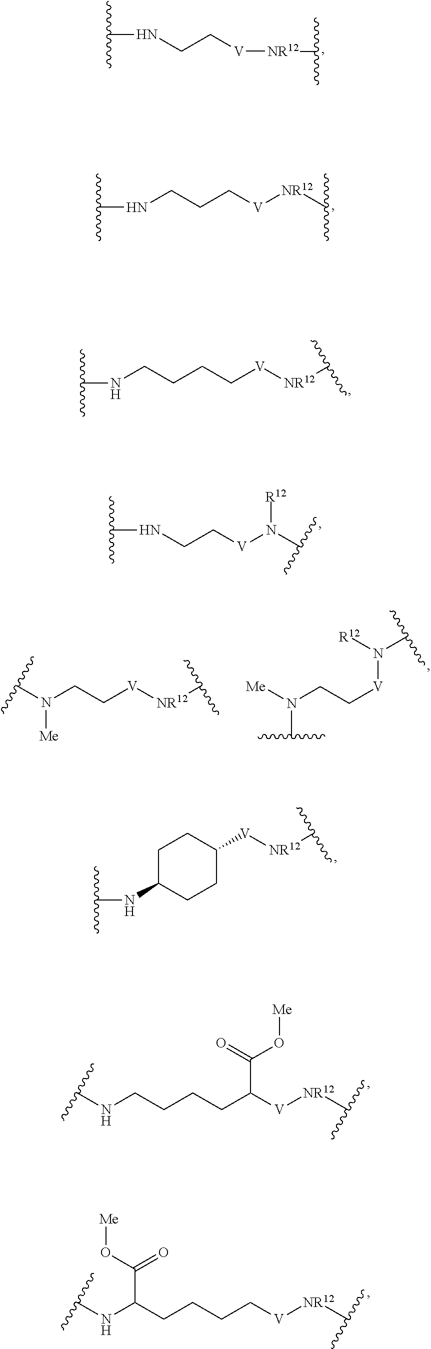

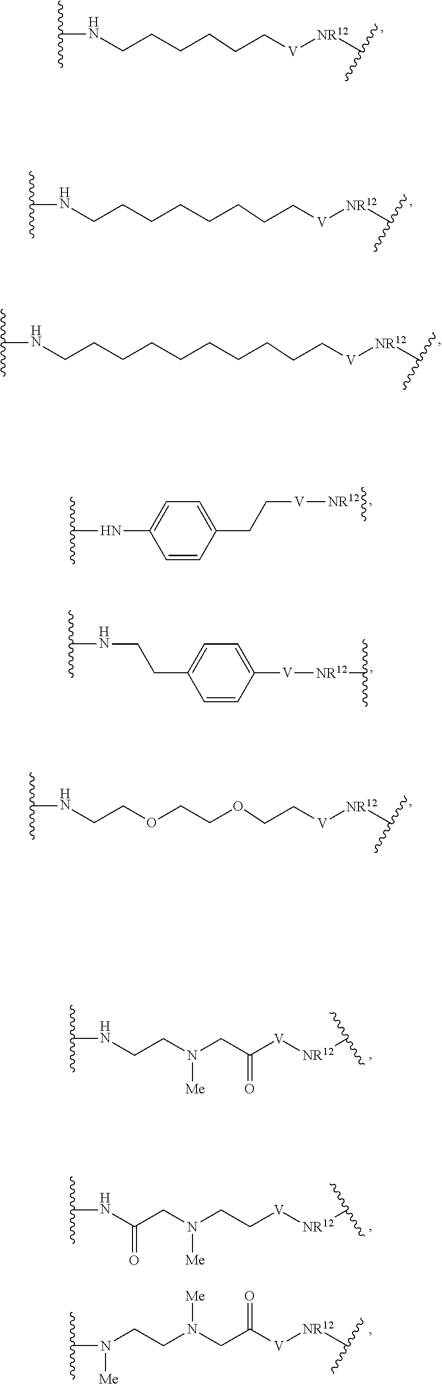

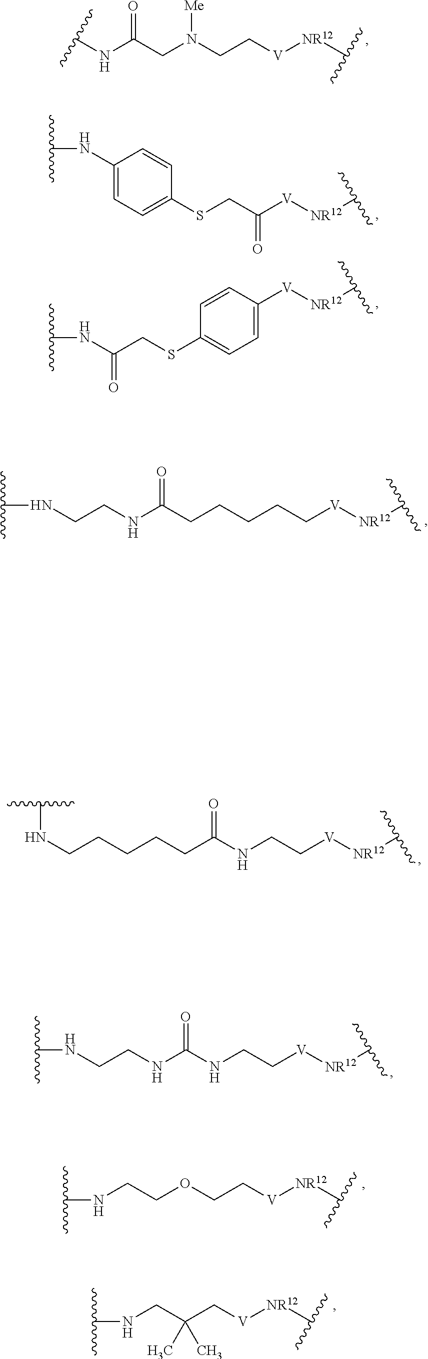

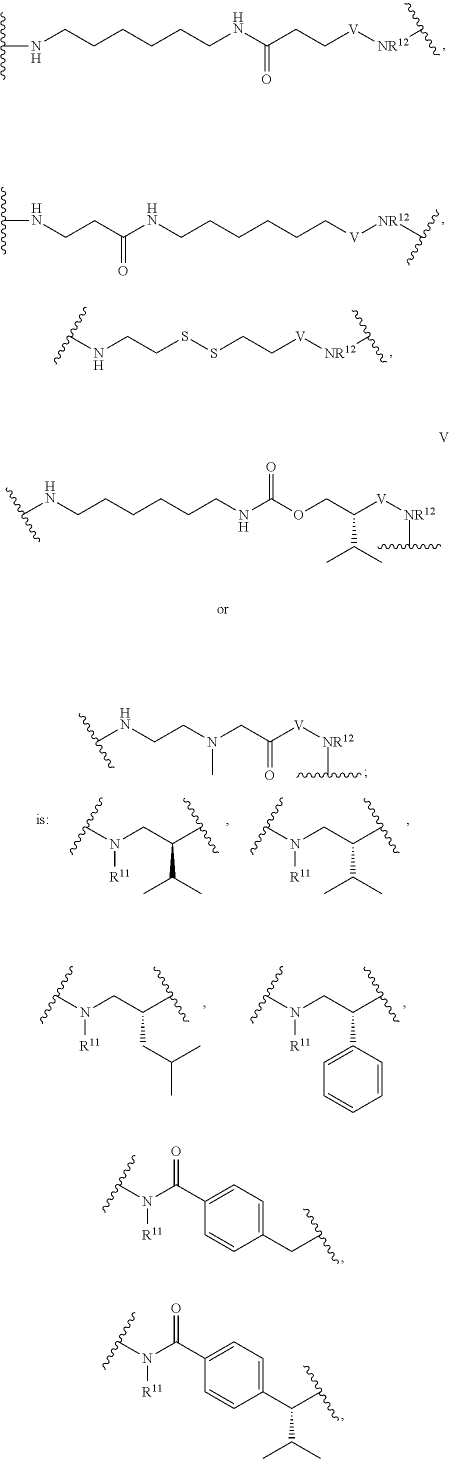

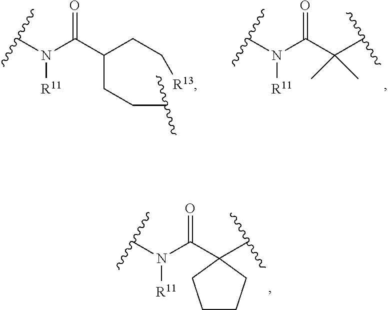

or a bond; R¹² is H or Me; or R¹² taken together with R¹⁴ forms a piperidine ring; R¹¹ is H or Me; and R¹³ taken together with R¹² forms a piperidine ring.

In some embodiments, -Q-X—Y— is

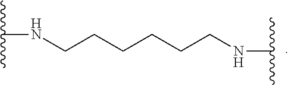 [SMILES: *NCCCCCCN*]

In some embodiments, -Q-X—Y— is

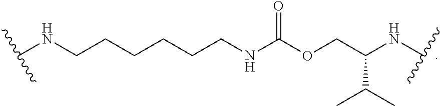 [SMILES: *NCCCCCCNC(=O)OC[C@H](N*)C(C)C]

In some embodiments, -Q-X—Y— is

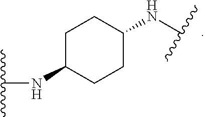 [SMILES: *N[C@H]1CC[C@H](N*)CC1]

In some embodiments, -QXY is

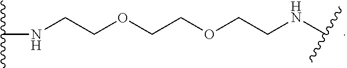 [SMILES: *NCCOCCOCCN*]

In some embodiments, -Q-X—Y— is

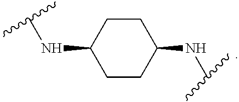 [SMILES: *N[C@H]1CC[C@@H](N*)CC1]

In some embodiments, -Q-X—Y— is

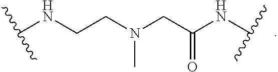 [SMILES: *NCCN(C)CC(=O)N*]

In some embodiments, R₄ and R₅ are methyl; R₆ is 2-hydroxypropyl; Z is —NH-AA₁-AA₂-AA₃-AA₄-AA₅-AA₆-C(O)-Q-X—Y—C(O)—W; AA₁ is glycine; AA₂ is a bond; AA₃ is a bond; AA₄ is phenylalanine; AA₅ is leucine; AA₆ is glycine; -Q-X—Y— is

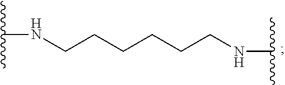 [SMILES: *NCCCCCCN*]

### and W is

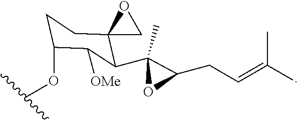

In some embodiments, R₄ and R₅ are methyl; R₆ is 2-hydroxypropyl; Z is —NH-AA₁-AA₂-AA₃-AA₄-AA₅-AA₆-C(O)-Q-X—Y—C(O)—W; AA₁ is glycine; AA₂ is a bond; AA₃ is a bond; AA₄ is phenylalanine; AA₅ is leucine; AA₆ is glycine; -Q-X—Y— is

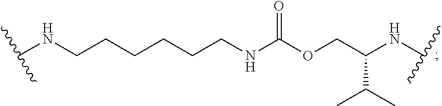 [SMILES: *NCCCCCCNC(=O)OC[C@H](N*)C(C)C]

### and W is

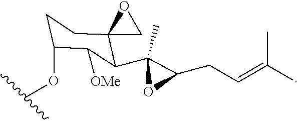

In some embodiments, R₄ and R₅ are methyl; R₆ is 2-hydroxypropyl; Z is —NH-AA₁-AA₂-AA₃-AA₄-AA₅-AA₆-C(O)-Q-X—Y—C(O)—W; AA₁ is glycine; AA₂ is a bond; AA₃ is a bond; AA₄ is phenylalanine; AA₅ is leucine; AA₆ is glycine; -Q-X—Y— is

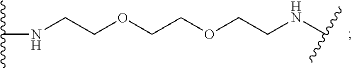 [SMILES: *NCCOCCOCCN*]

### and W is

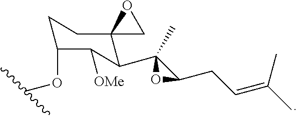

In some embodiments, R₄ and R₅ are methyl; Rh is 2-hydroxypropyl; Z is —NH-AA₁-AA₂-AA₃-AA₄-AA₅-AA₆-C(O)-Q-X—Y—C(O)—W; AA₁ is glycine; AA₂ is a bond; AA₃ is a bond; AA₄ is phenylalanine, AA₅ is leucine, AA₆ is glycine; -Q-X—Y— is

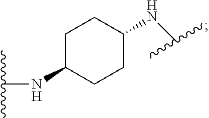 [SMILES: *N[C@H]1CC[C@H](N*)CC1]

### and W is

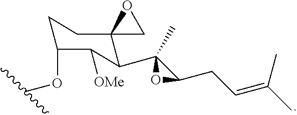

In some embodiments, -Q-X—Y— is a self-immolating linker that releases the MetAP2 inhibitor in the form of a carbamate derivative, as shown in the scheme below:

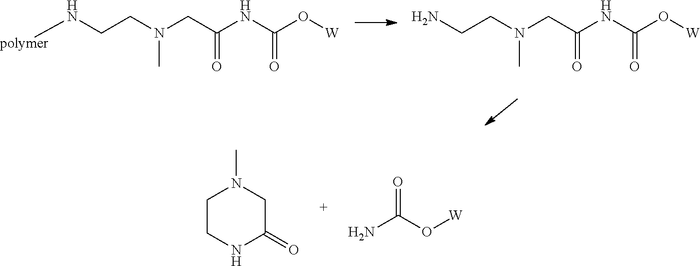

Another aspect of the present disclosure provides conjugates with linkers having the structure: Z-Q-X—Y—C(O)—W; wherein, independently for each occurrence, Z is H₂N-AA₂-AA₃-AA₄-AA₅-AA₆-C(O)— or H; AA₂ is a bond, or alanine, cysteine, aspartic acid, glutamic acid, phenylalanine, glycine, histidine, isoleucine, lysine, leucine, methionine, asparagine, proline, glutamine, arginine, serine, threonine, valine, tryptophan, or tyrosine; AA₃ is a bond, or alanine, cysteine, aspartic acid, glutamic acid, phenylalanine, glycine, histidine, isoleucine, lysine, leucine, methionine, asparagine, proline, glutamine, arginine, serine, threonine, valine, tryptophan, or tyrosine; AA₄ is a bond, or alanine, cysteine, aspartic acid, glutamic acid, phenylalanine, glycine, histidine, isoleucine, lysine, leucine, methionine, asparagine, proline, glutamine, arginine, serine, threonine, valine, tryptophan, or tyrosine; AA₅ is a bond, alanine, cysteine, glycine, isoleucine, leucine, methionine, phenylalanine, valine, tryptophan, or; AA₆ is alanine, asparagine, citrulline, glutamine, glycine, leucine, methionine, phenylalanine, serine, threonine, tryptophan, tyrosine, valine or H₂N(CH₂)ₘCO₂H, wherein m is 2, 3, 4 or 5; Q is NR, O, or S; X is M-(C(R)₂)ₚ-M-J-M-(C(R)₂)ₚ-M-V; M is a bond, or C(O); J is a bond, or ((CH₂)_{q}Q)ᵣ, C₅-C₈cycloalkyl, aryl, heteroaryl, NR, O, or S; Y is NR, O, or S; R is H or alkyl; V is a bond or

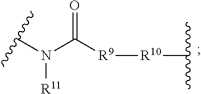

R⁹ is alkyl, aryl, aralkyl, or a bond; or Re taken together with Y forms a heterocyclic ring; R¹⁰ is amido or a bond; R¹¹ is H or alkyl; W is a MetAP2 inhibitor moiety; p is 0 to 20; q is 2 or 3; and r is 1, 2, 3, 4, 5, or 6.

In some embodiments, Z is H₂N-AA₅-AA₆-C(O)—. In some embodiments, AA₅ is alanine, cysteine, glycine, isoleucine, leucine, methionine, phenylalanine, valine, tryptophan, or tyrosine and AA₆ is glycine. In some embodiments, AA₅ is leucine and AA₆ is glycine. In some embodiments, AA₅ is valine and AA₆ is glycine. In some embodiments, AA₅ is phenylalanine and AA₆ is glycine. In some embodiments AA₅ is glycine and AA₆ is glycine. In some embodiments, AA₅ is not valine.

In some embodiments, Z is H₂N-AA₃-AA₄-AA₄-AA₅-AA₆-C(O)—. In some embodiments, AA₅ is alanine, cysteine, glycine, isoleucine, leucine, methionine, phenylalanine, valine, tryptophan, or tyrosine and each of AA₃, AA₄, or AA₆ is glycine. In some embodiments, AA₅ is leucine and each of AA₃, AA₄, or AA₆ is glycine. In some embodiments, AA₅ is valine and each of AA₃, AA₄, or AA₆ is glycine. In some embodiments, AA₅ is phenylalanine and each of AA₃, AA₄, or AA₆ is glycine. In some embodiments, AA₃ is glycine, AA₄ is phenylalanine, AA₅ is leucine and AA₆ is glycine. In some embodiments, each of AA₃, AA₄, AA₅ and AA₆ is glycine. In some embodiments, AA₅ is not valine.

In some embodiments, Z is H. In some embodiments, Z is H₂N-AA₆-C(O)—. In some embodiments, AA₆ is glycine.

In some embodiments, Q is NR. In some embodiments, M is a bond. In some embodiments, J is a bond. In some embodiments, Y is NR.

In some embodiments, W is:

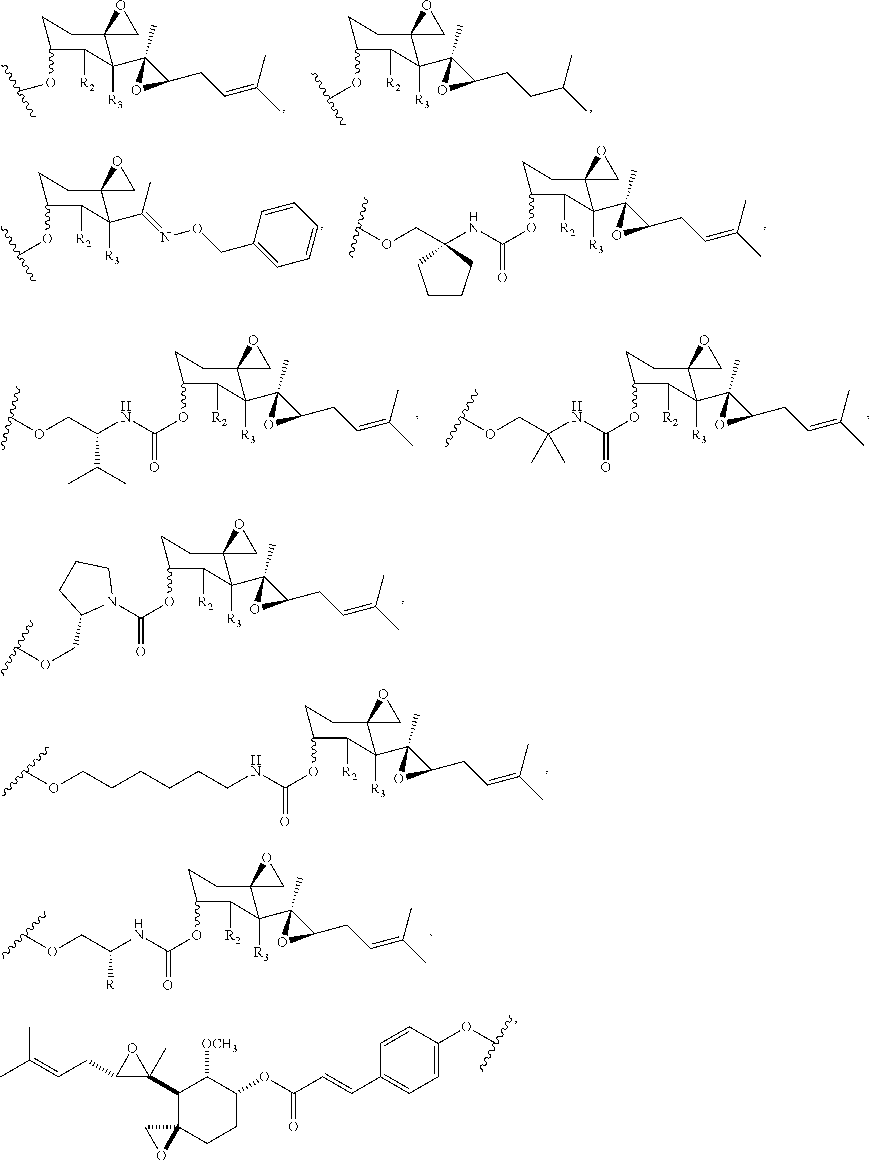

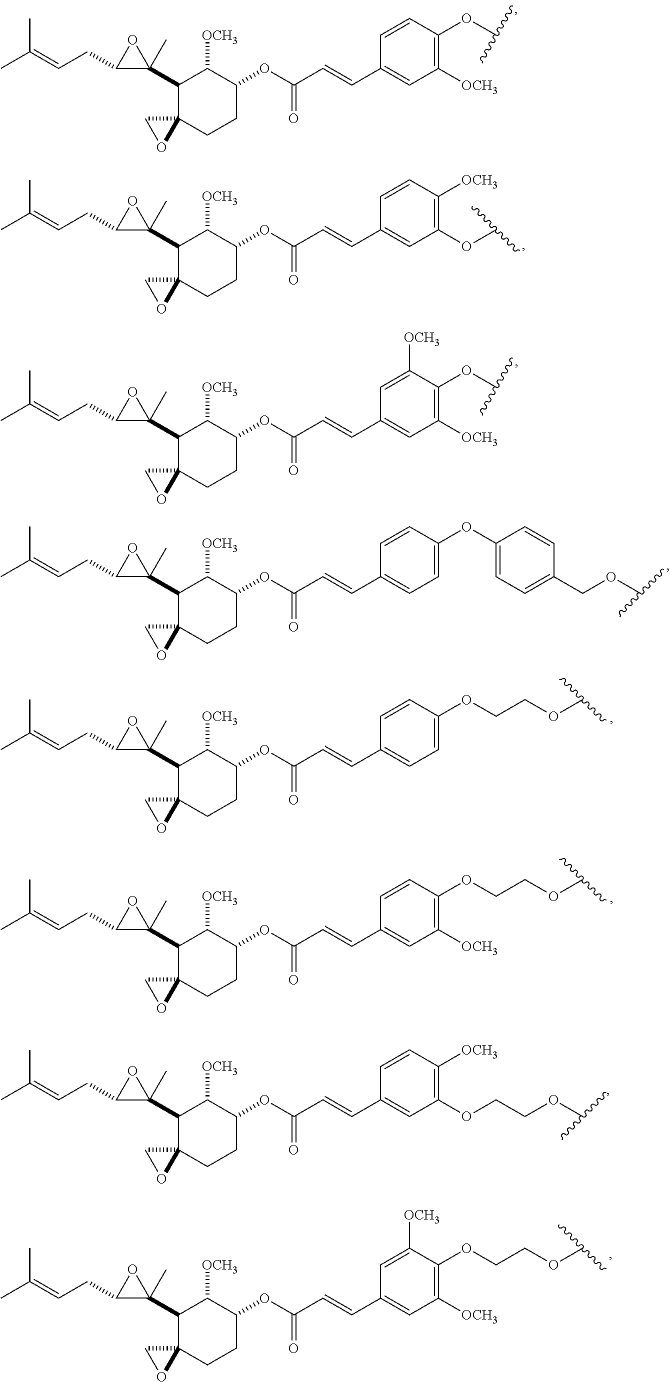

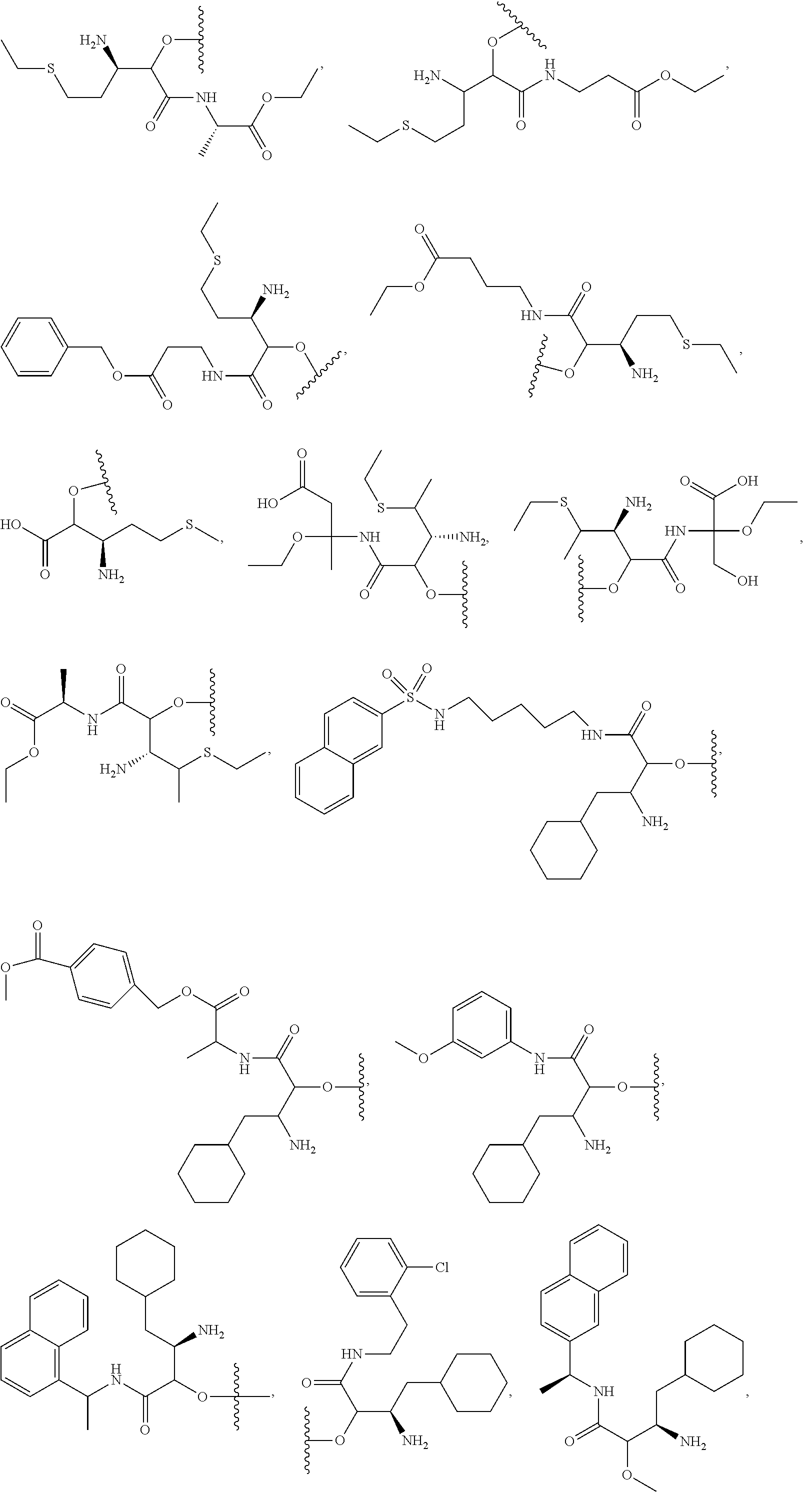

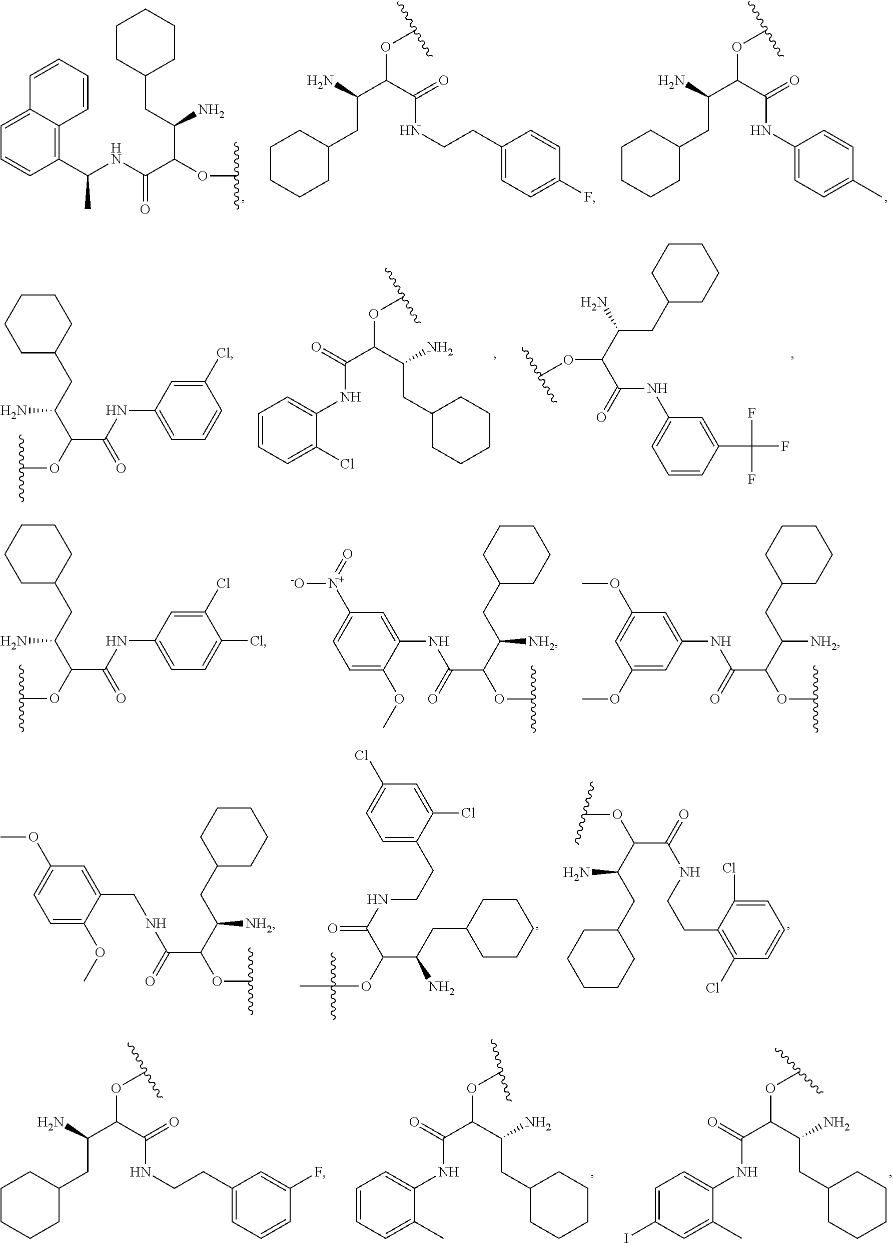

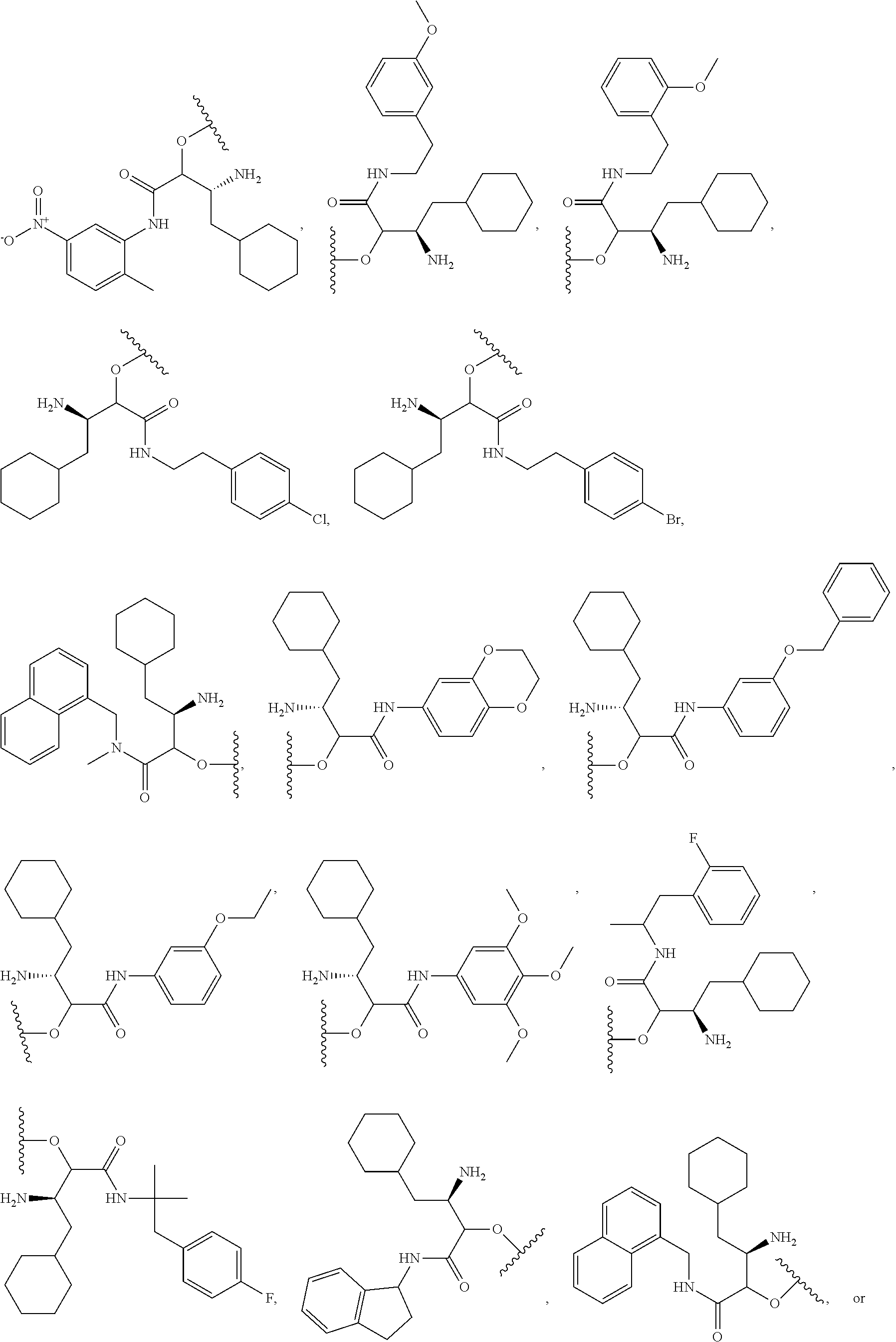

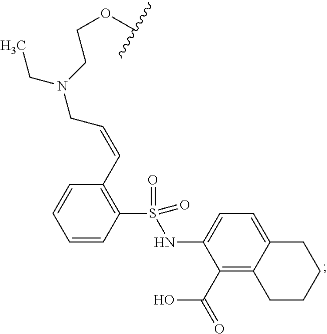

wherein R is —OH or methoxy, and R₃ is H, —OH or methoxy.

In some embodiments, W is

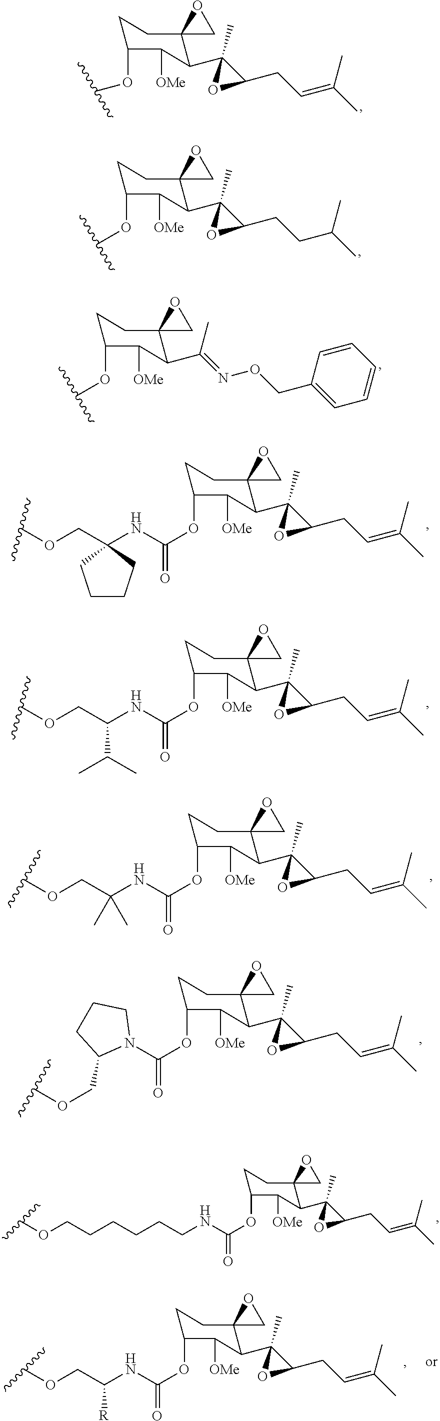

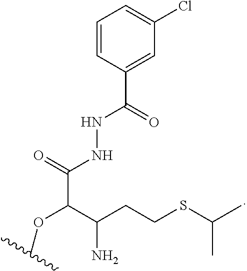 [SMILES: *OC(C(=O)NNC(=O)c1cccc(Cl)c1)C(N)CCSC(C)C]

In some embodiments, W is

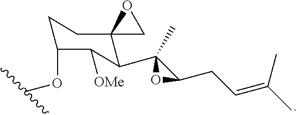

In some embodiments, -Q-X—Y— is

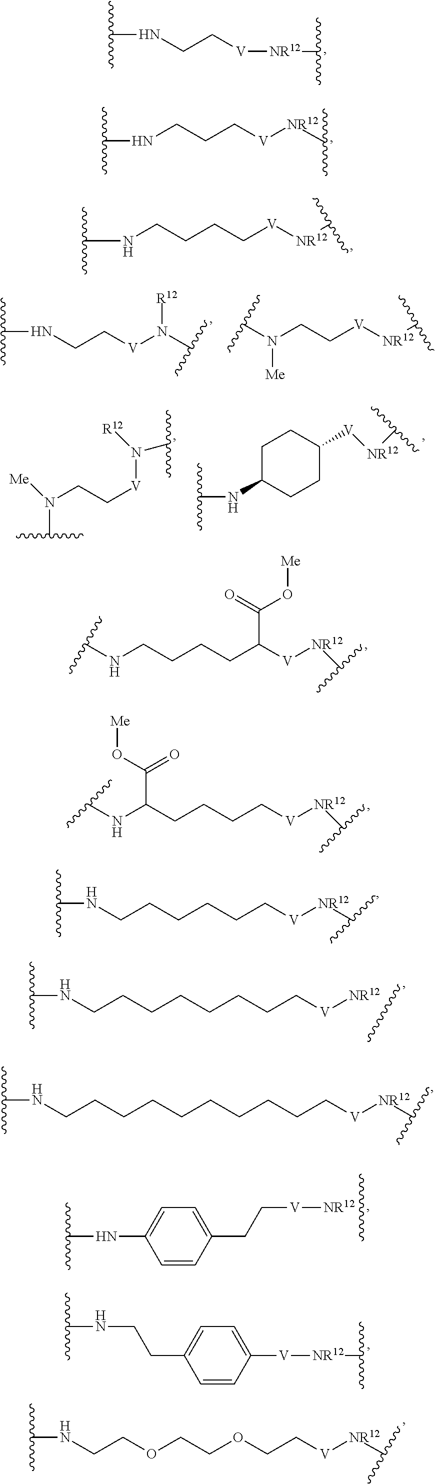

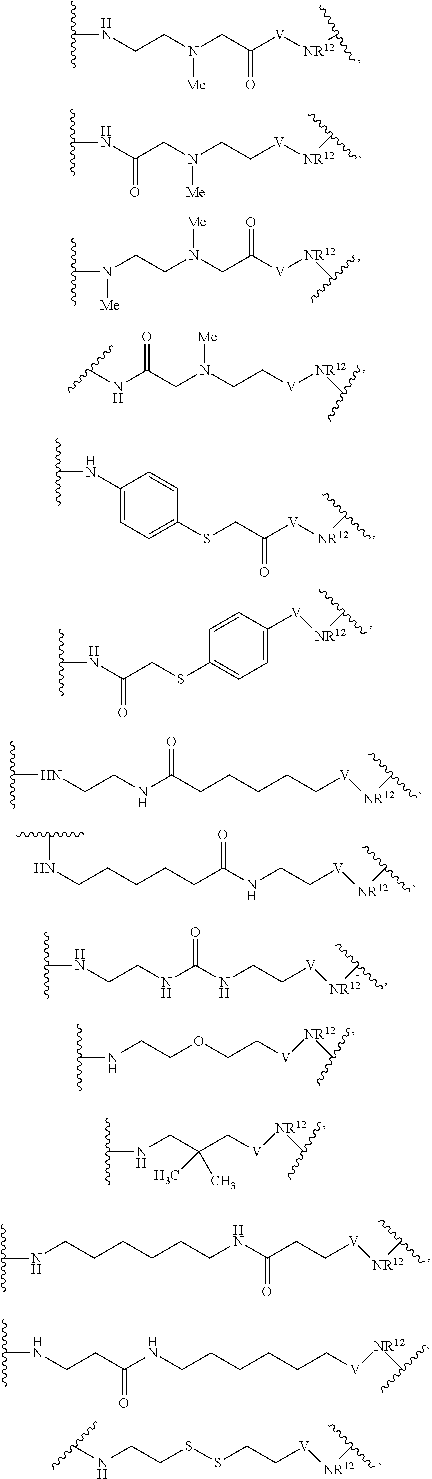

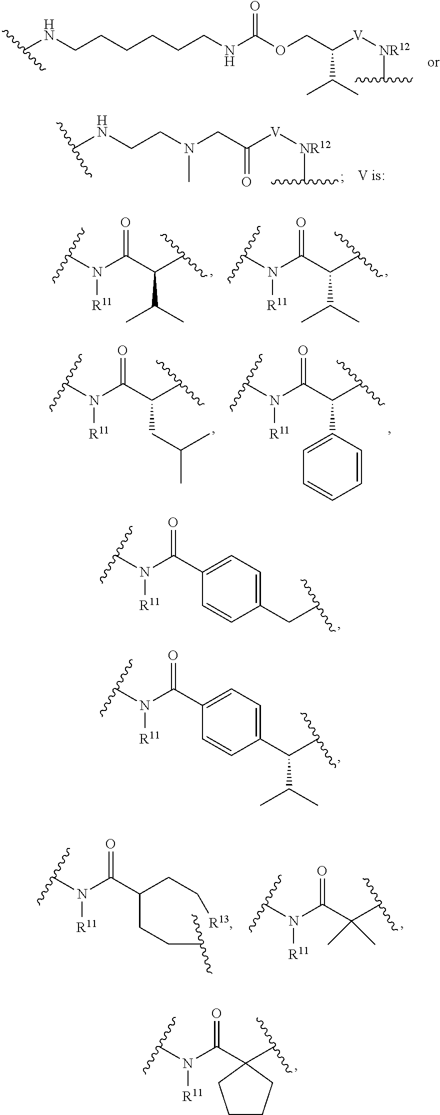

or a bond; R¹² is H or Me, or R¹² taken together with R¹⁴ forms a piperidine ring; R¹¹ is H or Me; and R¹³ taken together with R¹² forms a piperidine ring.

In some embodiments, Z is H₂N-AA₅-AA₆-C(O)—; AA₅ is leucine and AA₆ is glycine; Q-X—Y is

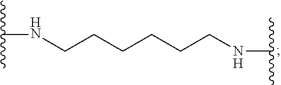 [SMILES: *NCCCCCCN*]

### and W is

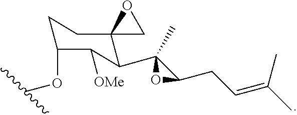

In some embodiments, Z is H₂N-AA₅-AA₆-C(O)—; AA₅ is valine and AA₅ is glycine; Q-X—Y is

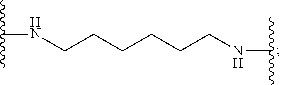 [SMILES: *NCCCCCCN*]

### and W is

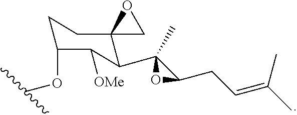

In some embodiments, Z is H₂N-AA₅-AA₆-C(O)—; AA₅ is phenylalanine and AA₆ is glycine; Q-X—Y is

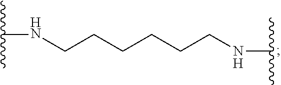 [SMILES: *NCCCCCCN*]

### and W is

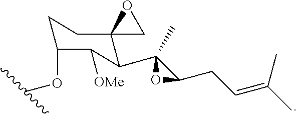

In some embodiments, Z is H₂N-AA₅-AA₆-C(O)—; AA₅ is glycine and AA₆ is glycine; Q-X—Y is

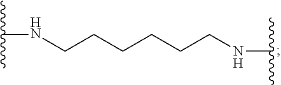 [SMILES: *NCCCCCCN*]

### and W is

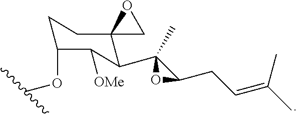

In some embodiments, Z is H₂N-AA₃-AA₄-AA₅-AA₆-C(O)—; AA₅ is leucine and each of AA₃, AA₄, or AA₆ is glycine; Q-X—Y is

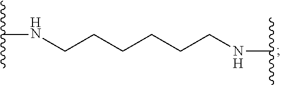 [SMILES: *NCCCCCCN*]

### and W is

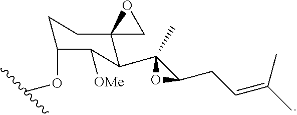

In some embodiments, Z is H₂N-AA₃-AA₄-AA₅-AA₆-C(O)—; AA₅ is valine and each of AA₃, AA₄, or AA₆ is glycine; Q-X—Y is

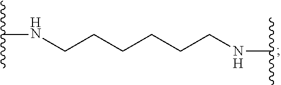 [SMILES: *NCCCCCCN*]

### and W is

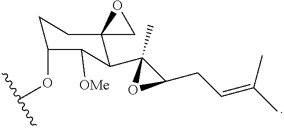

In some embodiments, Z is H₂N-AA₃-AA₄-AA₅-AA₆-C(O)—; AA₅ is phenylalanine and each of AA₃, AA₄, or AA₆ is glycine; Q-X—Y is

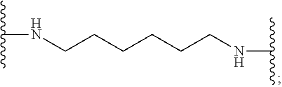 [SMILES: *NCCCCCCN*]

### and W is

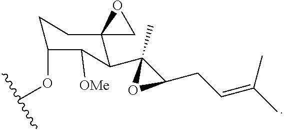

In some embodiments, Z is H₂N-AA₃-AA₄-AA₅-AA₆-C(O)—; AA₃ is glycine, AA₄ is phenylalanine, AA₅ is leucine and AA₆ is glycine; Q-X—Y is

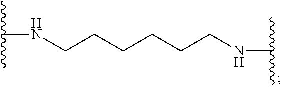 [SMILES: *NCCCCCCN*]

### and W is

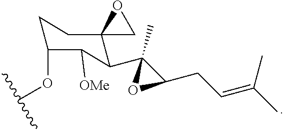

In some embodiments, Z is H₂N-AA₃-AA₄-AA₅-AA₆-C(O)—; each of AA₃, AA₄, AA₅ and AA₆ is glycine; Q-X—Y is

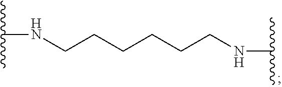 [SMILES: *NCCCCCCN*]

### and W is

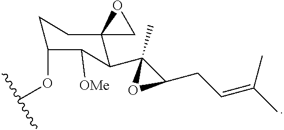

In some embodiments, Z is H₂N-AA₆-C(O)—; AA₆ is glycine; Q-X—Y is

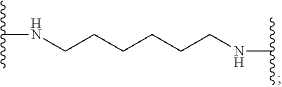 [SMILES: *NCCCCCCN*]

### and W is

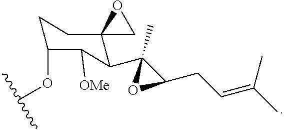

In some embodiments, Z is H; Q-X—Y is

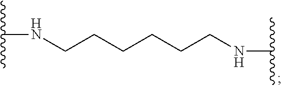 [SMILES: *NCCCCCCN*]

### and W is

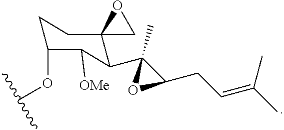

In some embodiments, Z is H₂N-AA₅-AA₆-C(O)—; AA₅ is leucine and AA₆ is glycine; Q-X—Y is

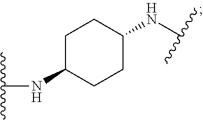 [SMILES: *N[C@H]1CC[C@H](N*)CC1]

### and W is

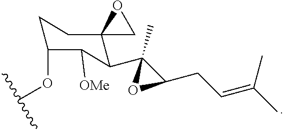

In some embodiments, Z is H₂N-AA₅-AA₆-C(O)—, AA₅ is valine and AA₆ is glycine; Q-X—Y is

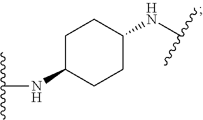 [SMILES: *N[C@H]1CC[C@H](N*)CC1]

### and W is

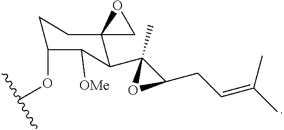

In some embodiments, Z is H₂N-AA₅-AA₆-C(O)—; AA₅ is phenylalanine and AA₆ is glycine; Q-X—Y is

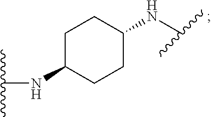 [SMILES: *N[C@H]1CC[C@H](N*)CC1]

### and W is

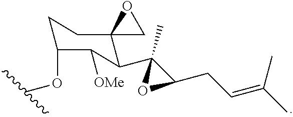

In some embodiments, Z is H₂N-AA₅-AA₆-C(O)—; AA₅ is glycine and AA₆ is glycine; Q-X—Y is

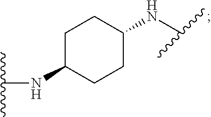 [SMILES: *N[C@H]1CC[C@H](N*)CC1]

### and W is

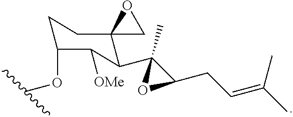

In some embodiments, Z is H₂N-AA₃-AA₄-AA₅-AA₆-C(O)—; AA₅ is leucine and each of AA₃, AA₄, or AA₆ is glycine; Q-X—Y is

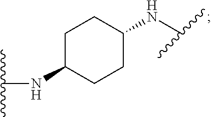 [SMILES: *N[C@H]1CC[C@H](N*)CC1]

### and W is

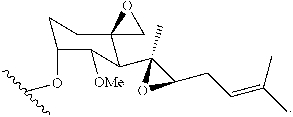

In some embodiments, Z is H₂N-AA₃-AA₄-AA₅-AA₆-C(O)—; AA₅ is valine and each of AA₃, AA₄, or AA₆ is glycine; Q-X—Y is

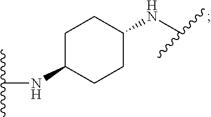 [SMILES: *N[C@H]1CC[C@H](N*)CC1]

### and W is

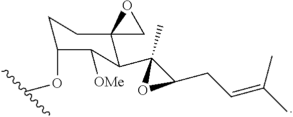

In some embodiments, Z is H₂N-AA₃-AA₄-AA₅-AA-C(O)—; AA₅ is phenylalanine and each of AA₃, AA₄, or AA₆ is glycine; Q-X—Y is

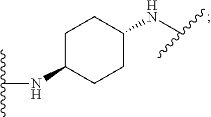 [SMILES: *N[C@H]1CC[C@H](N*)CC1]

### and W is

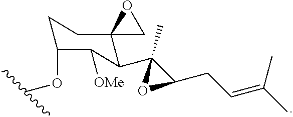

In some embodiments, Z is H₂N-AA₃-AA₄-AA₅-AA₆-C(O)—; AA₃ is glycine, AA₄ is phenylalanine, AA₅ is leucine and AA₆ is glycine, Q-X—Y is

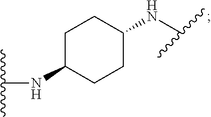 [SMILES: *N[C@H]1CC[C@H](N*)CC1]

### and W is

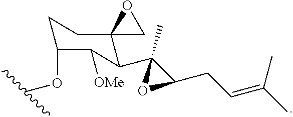

In some embodiments, Z is H₂N-AA₃-AA₄-AA₅-AA₆-C(O)—; each of AA₃, AA₄, AA₅ and AA₆ is glycine; Q-X—Y is

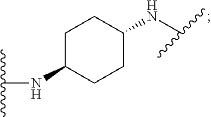 [SMILES: *N[C@H]1CC[C@H](N*)CC1]

### and W is

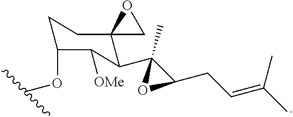

In some embodiments, Z is H₂N-AA₆-C(O)—; AA₆ is glycine; Q-X—Y is

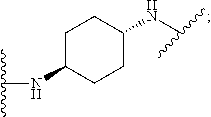 [SMILES: *N[C@H]1CC[C@H](N*)CC1]

### and W is

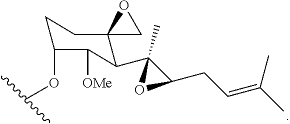

In some embodiments, Z is H; Q-X—Y is

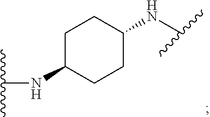 [SMILES: *N[C@H]1CC[C@H](N*)CC1]

### and W is

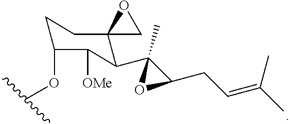

In some embodiments, Z is H₂N-AA₅-AA₆-C(O)—; AA₅ is leucine and AA₆ is glycine; Q-X—Y is

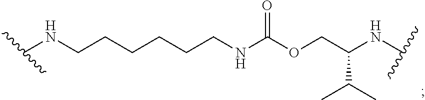 [SMILES: *NCCCCCCNC(=O)OC[C@H](N*)C(C)C]

### and W is

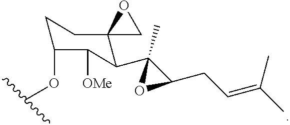

In some embodiments, Z is H₂N-AA₅-AA₆-C(O)—; AA₅ is valine and AA₆ is glycine; Q-X—Y is

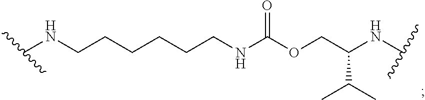 [SMILES: *NCCCCCCNC(=O)OC[C@H](N*)C(C)C]

### and W is

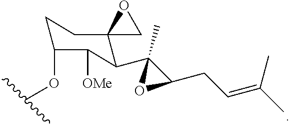

In some embodiments, Z is H₂N-AA₅-AA₆-C(O)—; AA₅ is phenylalanine and AA₆ is glycine; Q-X—Y is

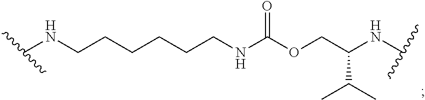 [SMILES: *NCCCCCCNC(=O)OC[C@H](N*)C(C)C]

### and W is

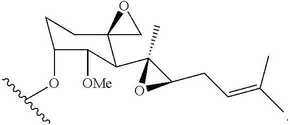

In some embodiments, Z is H₂N-AA₅-AA₆-C(O)—; AA₅ is glycine and AA₆ is glycine; Q-X—Y is

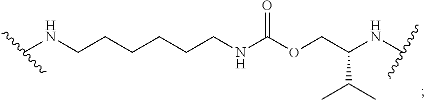 [SMILES: *NCCCCCCNC(=O)OC[C@H](N*)C(C)C]

### and W is

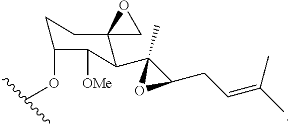

In some embodiments, Z is H₂N-AA₃-AA₄-AA₅-AA₆-C(O)—; AA₅ is leucine and each of AA₃, AA₄, or AA₆ is glycine; Q-X—Y is

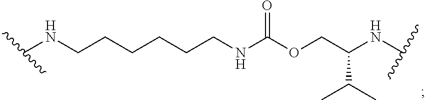 [SMILES: *NCCCCCCNC(=O)OC[C@H](N*)C(C)C]

### and W is

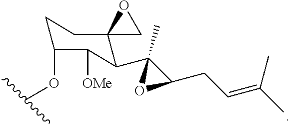

In some embodiments, Z is H₂N-AA₃-AA₄-AA₅-AA₆-C(O)—; AA₅ is valine and each of AA₃, AA₄, or AA₆ is glycine; Q-X—Y is

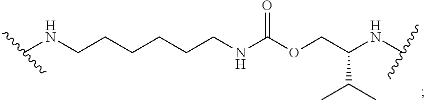 [SMILES: *NCCCCCCNC(=O)OC[C@H](N*)C(C)C]

### and W is

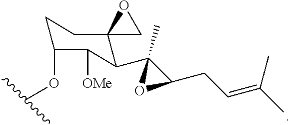

In some embodiments, Z is H₂N-AA₃-AA₄-AA₅-AA₆-C(O)—; AA₅ is phenylalanine and each of AA₃, AA₄, or AA₆ is glycine; Q-X—Y is

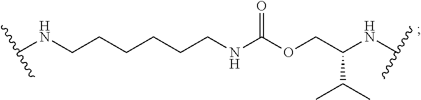 [SMILES: *NCCCCCCNC(=O)OC[C@H](N*)C(C)C]

### and W is

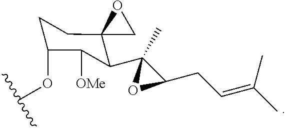

In some embodiments, Z is H₂N-AA₃-AA₄-AA₅-AA₆-C(O)—; AA₃ is glycine, AA₄ is phenylalanine, AA₅ is leucine and AA₆ is glycine; Q-X—Y is

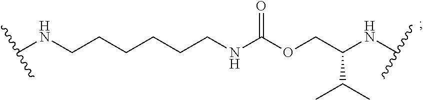 [SMILES: *NCCCCCCNC(=O)OC[C@H](N*)C(C)C]

### and W is

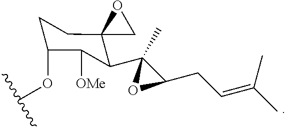

In some embodiments, Z is H₂N-AA₃-AA₄-AA₅-AA₆-C(O)—; each of AA₃, AA₄, AA₅ and AA₆ is glycine; Q-X—Y is

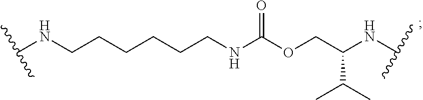 [SMILES: *NCCCCCCNC(=O)OC[C@H](N*)C(C)C]

### and W is

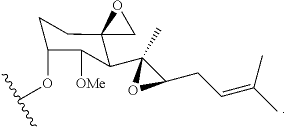

In some embodiments, Z is H₂N-AA₆-C(O)—; AA₆ is glycine; Q-X—Y is

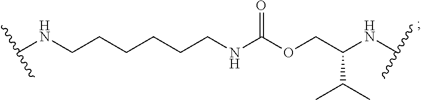 [SMILES: *NCCCCCCNC(=O)OC[C@H](N*)C(C)C]

### and W is

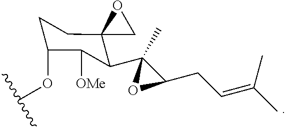

In some embodiments, Z is H; Q-X—Y is

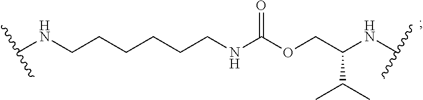 [SMILES: *NCCCCCCNC(=O)OC[C@H](N*)C(C)C]

### and W is

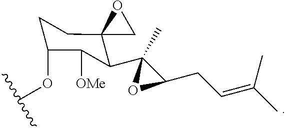

In some embodiments, Z is H₂N-AA₅-AA₆-C(O)—; AA₅ is leucine and AA₆ is glycine; Q-X—Y is

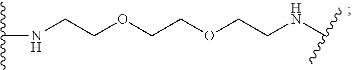 [SMILES: *NCCOCCOCCN*]

### and W is

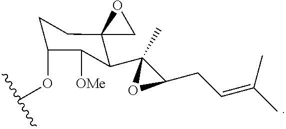

In some embodiments, Z is H₂N-AA₅-AA₆-C(O)—; AA₅ is valine and AA₆ is glycine; Q-X—Y is

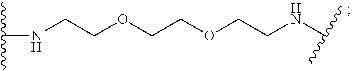 [SMILES: *NCCOCCOCCN*]

### and W is

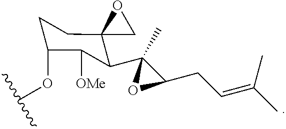

In some embodiments, Z is H₂N-AA₅-AA₆-C(O)—; AA₅ is phenylalanine and AA₆ is glycine; Q-X—Y is

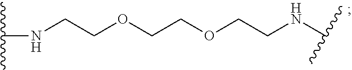 [SMILES: *NCCOCCOCCN*]

### and W is

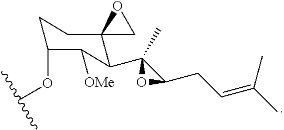

In some embodiments, Z is H₂N-AA₅-AA₆-C(O)—; AA₅ is glycine and AA₆ is glycine; Q-X—Y is

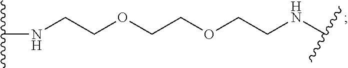 [SMILES: *NCCOCCOCCN*]

### and W is

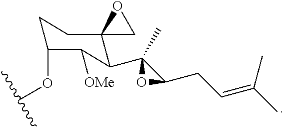

In some embodiments, Z is H₂N-AA₃-AA₄-AA₅-AA₆-C(O)—; AA₅ is leucine and each of AA₃, AA₄, or AA₆ is glycine; Q-X—Y is

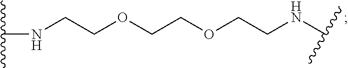 [SMILES: *NCCOCCOCCN*]

### and W is

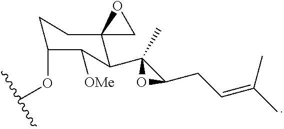

In some embodiments, Z is H₂N-AA₃-AA₄-AA₅-AA₆-C(O)—; AA₅ is valine and each of AA₃, AA₄, or AA₆ is glycine; Q-X—Y is

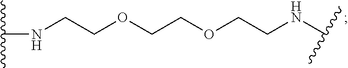 [SMILES: *NCCOCCOCCN*]

### and W is

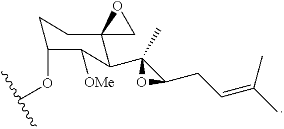

In some embodiments, Z is H₂N-AA₃-AA₄-AA₅-AA₆-C(O)—; AA₅ is phenylalanine and each of AA₃, AA₄, or AA₆ is glycine; Q-X—Y is

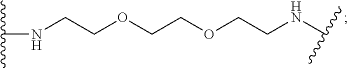 [SMILES: *NCCOCCOCCN*]

### and W is

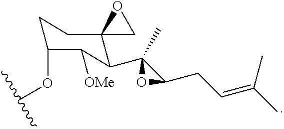

In some embodiments, Z is H₂N-AA₃-AA₄-AA₅-AA₆-C(O)—; AA₃ is glycine, AA₄ is phenylalanine, AA₅ is leucine and AA₆ is glycine; Q-X—Y is

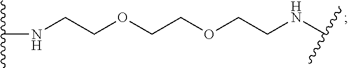 [SMILES: *NCCOCCOCCN*]

### and W is

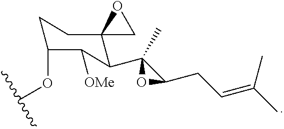

In some embodiments, Z is H₂N-AA₃-AA₄-AA₅-AA₆-C(O)—; each of AA₃, AA₄, AA₅ and AA₆ is glycine; Q-X—Y is

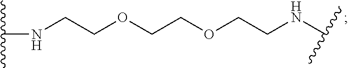 [SMILES: *NCCOCCOCCN*]

### and W is

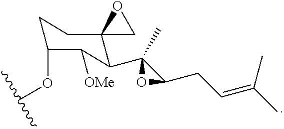

In some embodiments, Z is H₂N-AA₆-C(O)—; AA₆ is glycine; Q-X—Y is

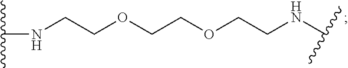 [SMILES: *NCCOCCOCCN*]

### and W is

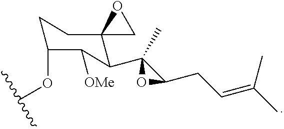

In some embodiments, Z is H; Q-X—Y is

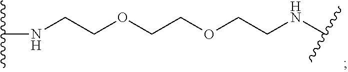 [SMILES: *NCCOCCOCCN*]

### and W is

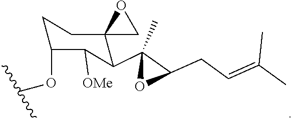

Other active moieties that may be modified to be used in conjugates of the disclosure include the following structures:

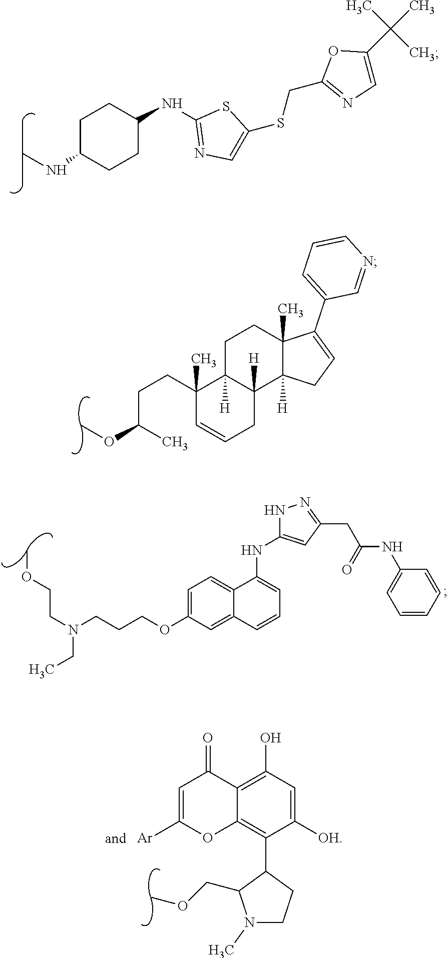

In some aspects, a MetAP2 inhibitor can be a compound represented by one or more of the formulae recited in Table 1, or a pharmaceutically acceptable salt, analog, derivative, salt or ester thereof:

TABLE 1

| Com- | |
|---|---|
| pound | |
| No. | Chemical Structure |
| 5 | 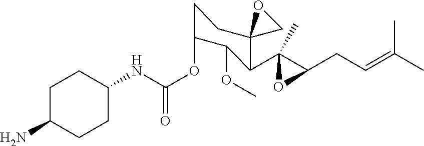 |
| 6 | 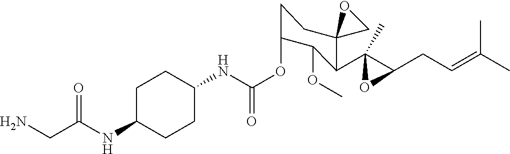 |
| 7 | 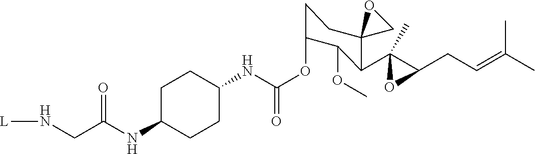 |
| 8 | 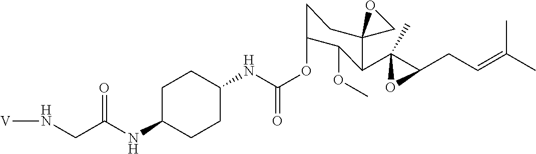 |
| 9 | 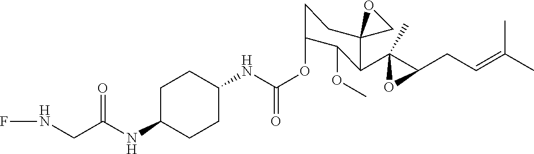 |
| 10 | 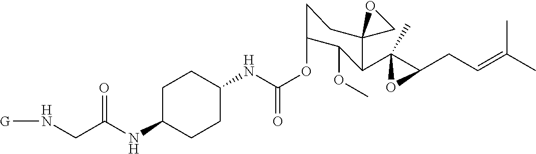 |
| 11 | 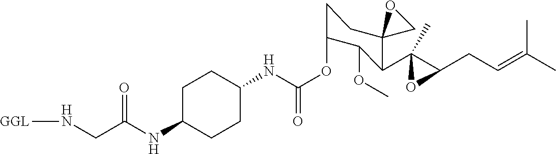 |
| 12 | 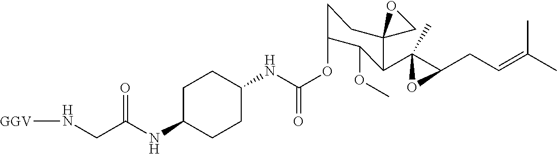 |
| 13 | 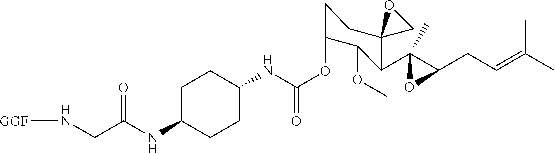 |
| 14 | 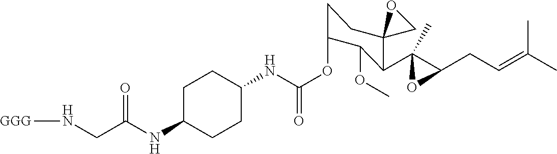 |
| 15 | 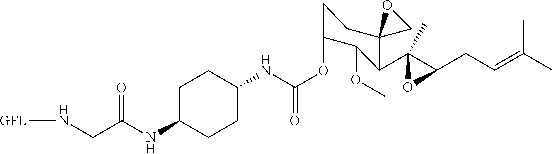 |
| 16 | 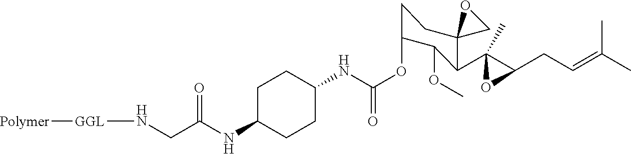 |
| 17 | 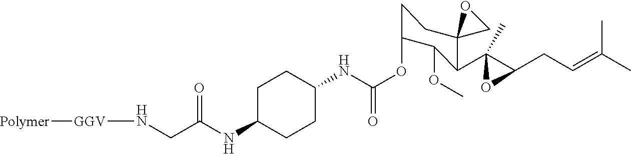 |
| 18 | 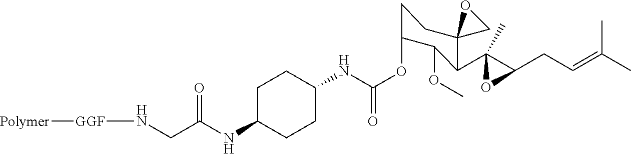 |
| 19 | 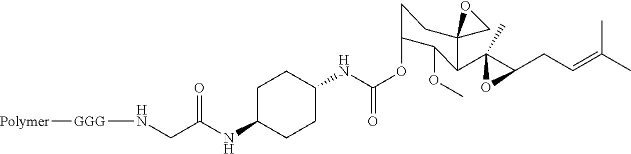 |
| 20 | 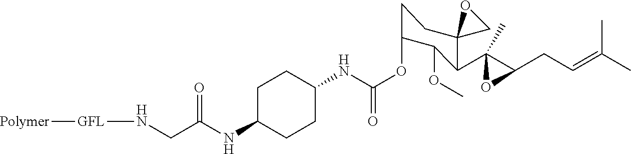 |
| 21 | 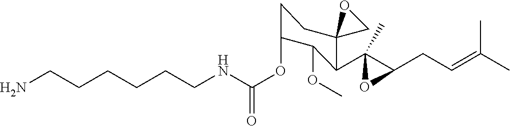 |
| 22 | 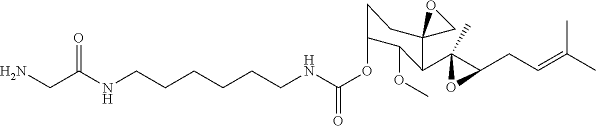 |
| 23 | 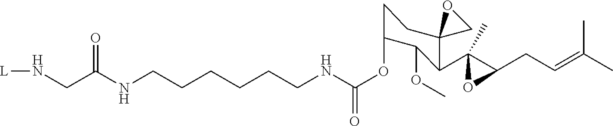 |
| 24 | 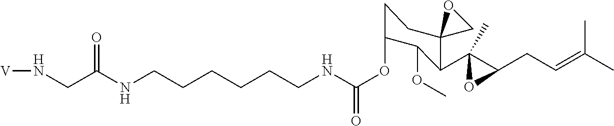 |
| 25 | 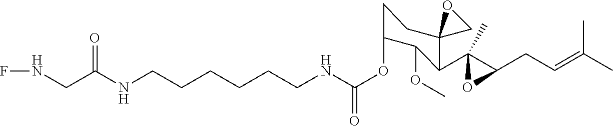 |
| 26 | 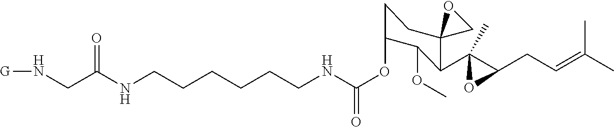 |
| 27 | 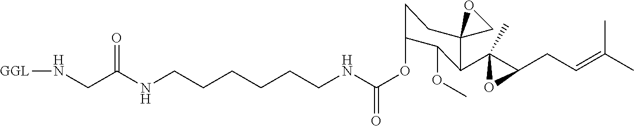 |
| 28 | 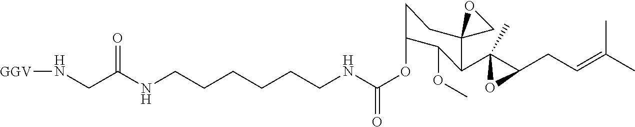 |
| 29 | 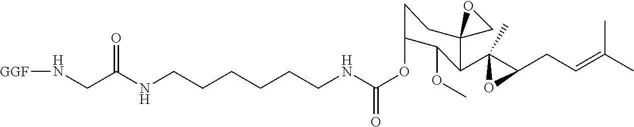 |
| 30 | 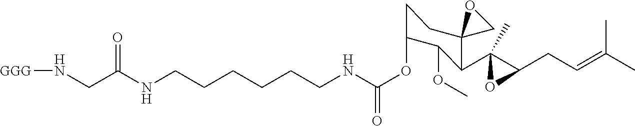 |
| 31 | 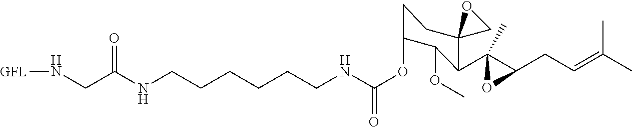 |
| 32 | 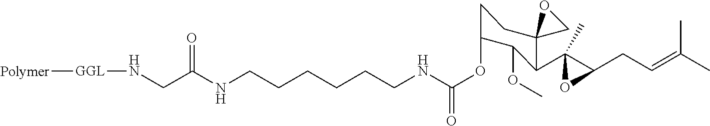 |
| 33 | 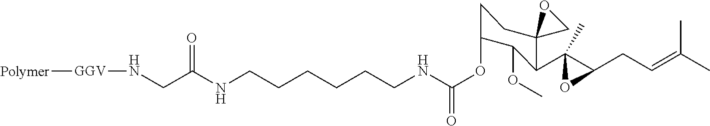 |
| 34 | 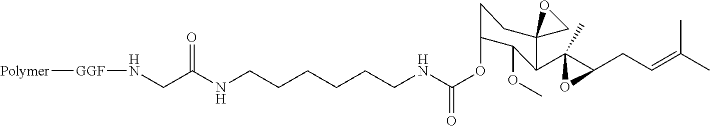 |
| 35 | 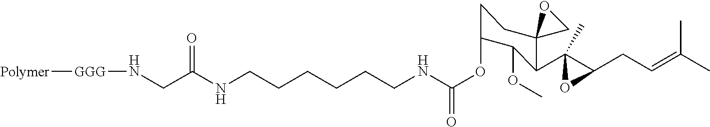 |
| 36 | 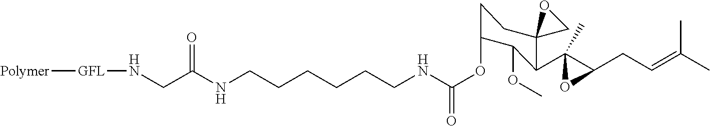 |
| 37 | 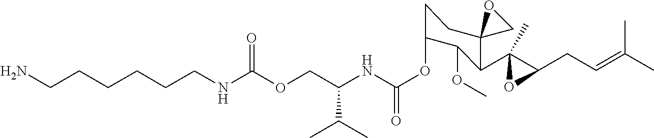 |
| 38 | 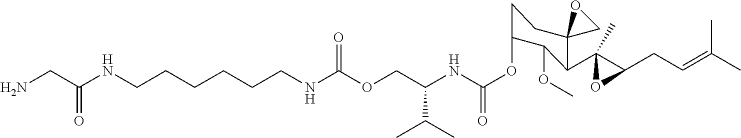 |
| 39 | 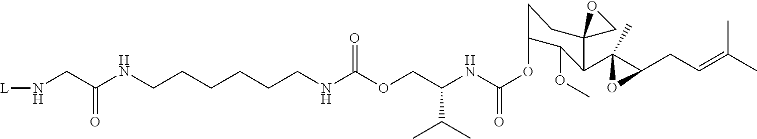 |
| 40 | 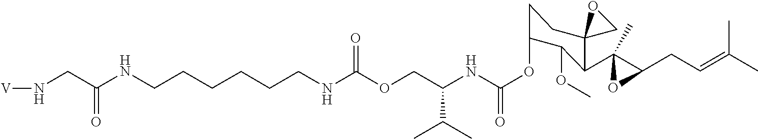 |
| 41 | 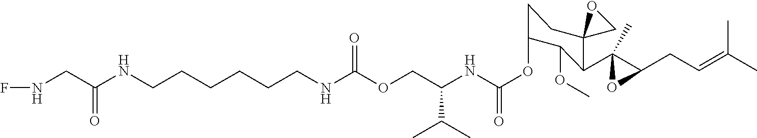 |
| 42 | 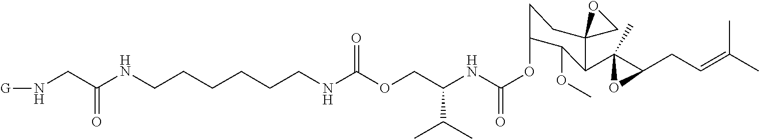 |
| 43 | 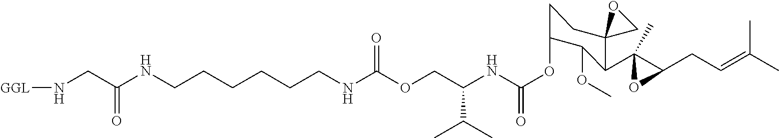 |
| 44 | 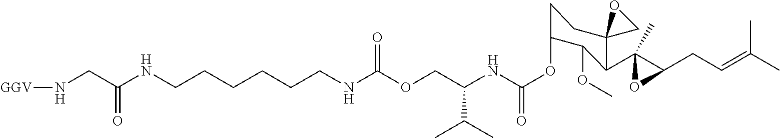 |
| 45 | 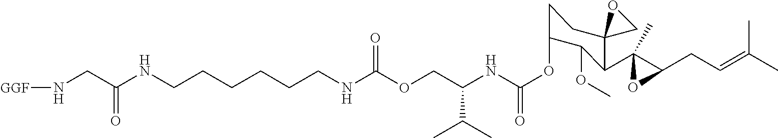 |
| 46 | 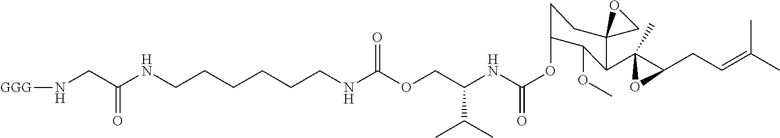 |
| 47 | 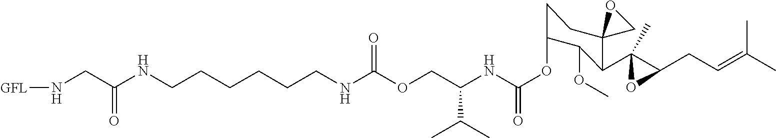 |
| 48 | 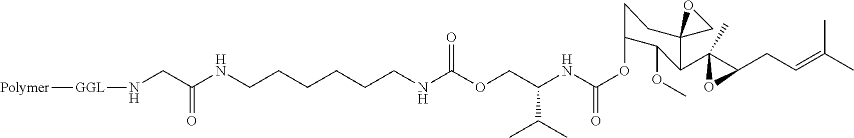 |
| 49 | 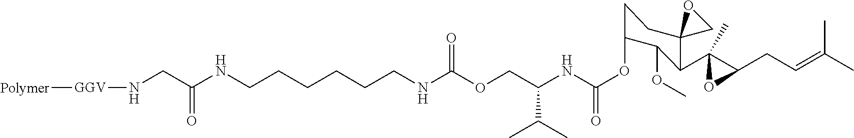 |
| 50 | 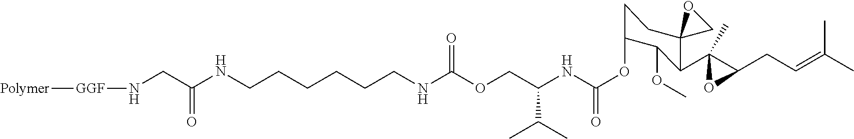 |
| 51 | 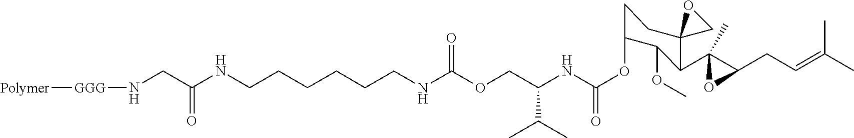 |
| 52 | 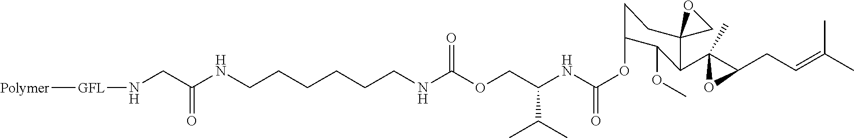 |
| 53 | 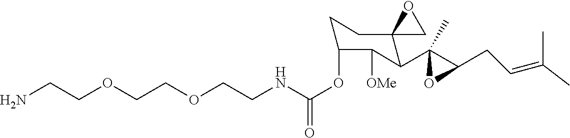 |
| 54 | 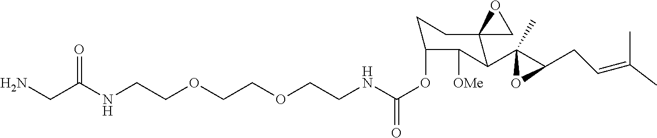 |
| 55 | 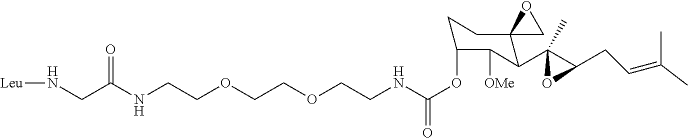 |
| 56 | 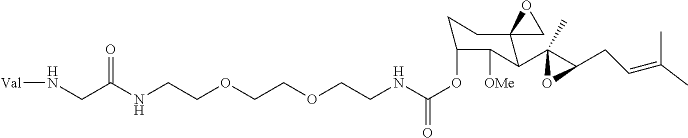 |
| 57 | 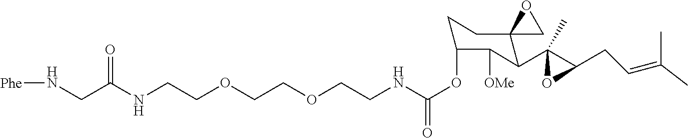 |
| 58 | 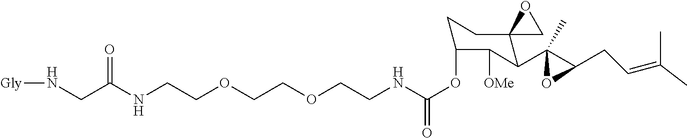 |
| 59 | 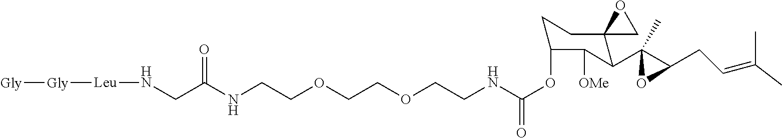 |
| 60 | 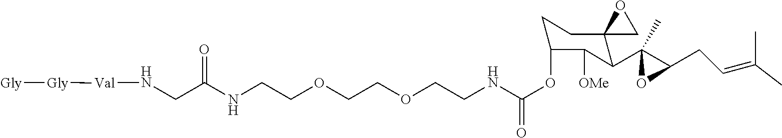 |
| 61 | 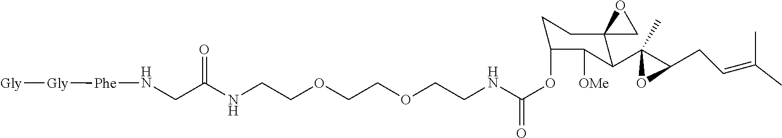 |
| 62 | 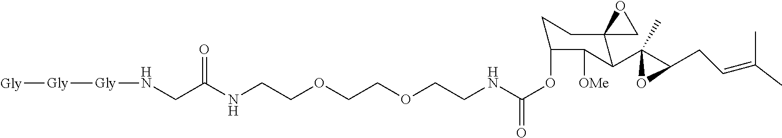 |
| 63 | 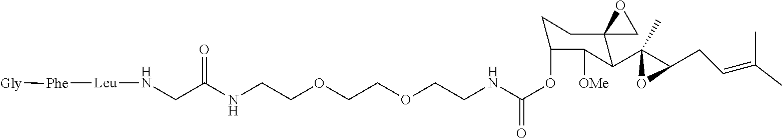 |
| 64 | 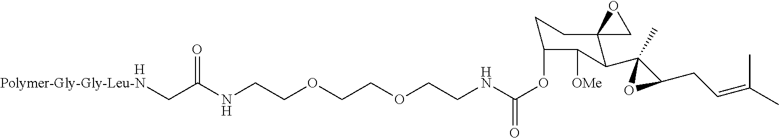 |
| 65 | 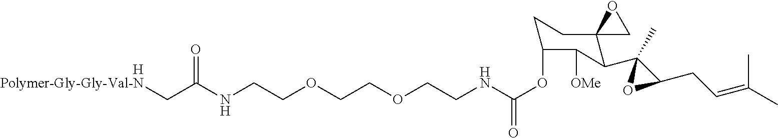 |
| 66 | 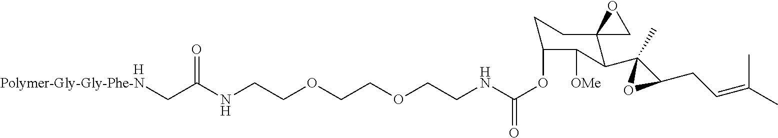 |
| 67 | 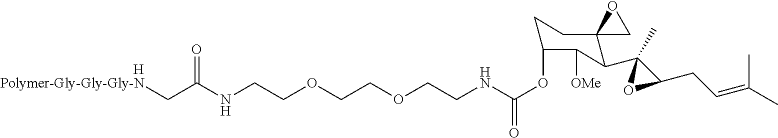 |
| 68 | 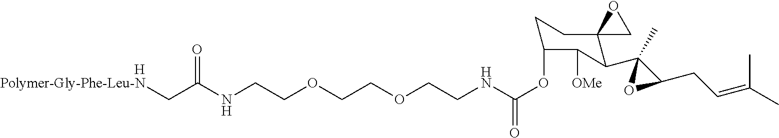 |
| 69 | 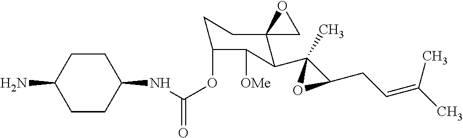 |
| 70 | 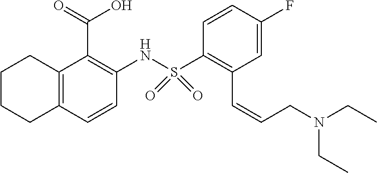 [SMILES: CCN(CC)C/C=C\c1cc(F)ccc1S(=O)(=O)Nc1ccc2c(c1C(=O)O)CCCC2] |
| 71 | 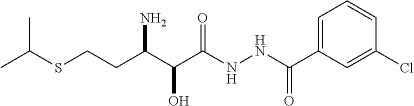 |
| 72 | 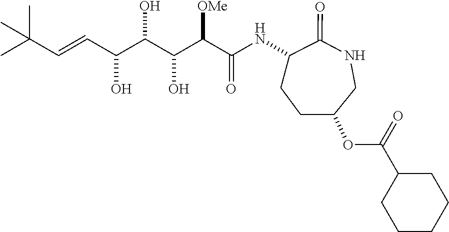 |
| 73 | 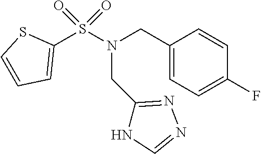 [SMILES: O=S(=O)(c1cccs1)N(Cc1ccc(F)cc1)Cc1nnc[nH]1] |
| 74 | 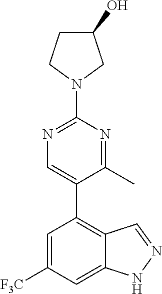 |
| 75 | 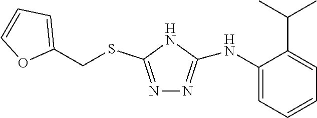 [SMILES: CC(C)c1ccccc1Nc1nnc(SCc2ccco2)[nH]1] |
| 76 | 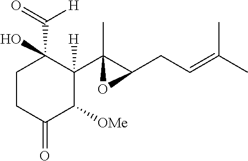 [SMILES: CO[C@@H]1C(=O)CC[C@](O)(C=O)[C@H]1[C@@]1(C)O[C@@H]1CC=C(C)C] |
| 77 | 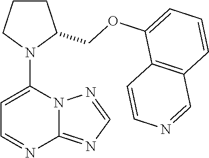 [SMILES: c1cc(OC[C@H]2CCCN2c2ccnc3ncnn23)c2ccncc2c1] |
| 78 | 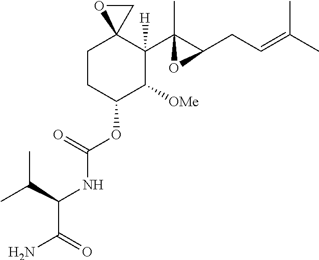 [SMILES: CO[C@@H]1[C@H](OC(=O)N[C@@H](C(N)=O)C(C)C)CC[C@]2(CO2)[C@H]1[C@@]1(C)O[C@@H]1CC=C(C)C] |
| 79 | 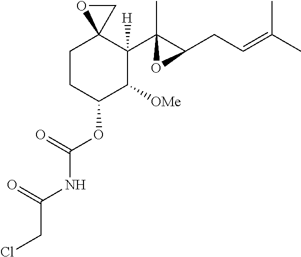 [SMILES: CO[C@@H]1[C@H](OC(=O)NC(=O)CCl)CC[C@]2(CO2)[C@H]1[C@@]1(C)O[C@@H]1CC=C(C)C] |
| 80 | 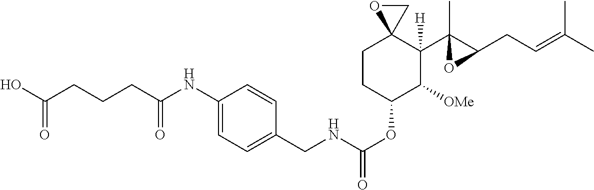 [SMILES: CO[C@@H]1[C@H](OC(=O)NCc2ccc(NC(=O)CCCC(=O)O)cc2)CC[C@]2(CO2)[C@H]1[C@@]1(C)O[C@@H]1CC=C(C)C] |
| 81 | 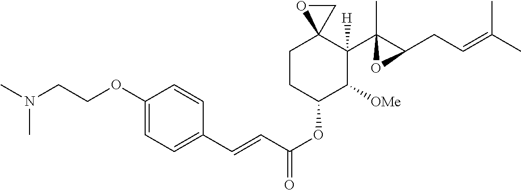 [SMILES: CO[C@@H]1[C@H](OC(=O)/C=C/c2ccc(OCCN(C)C)cc2)CC[C@]2(CO2)[C@H]1[C@@]1(C)O[C@@H]1CC=C(C)C] |
| 82 | 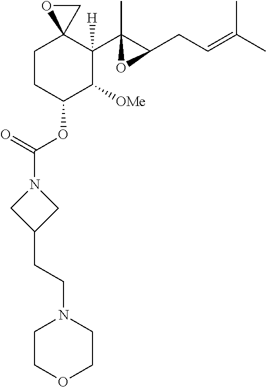 [SMILES: CO[C@@H]1[C@H](OC(=O)N2CC(CCN3CCOCC3)C2)CC[C@]2(CO2)[C@H]1[C@@]1(C)O[C@@H]1CC=C(C)C] |
| 83 | 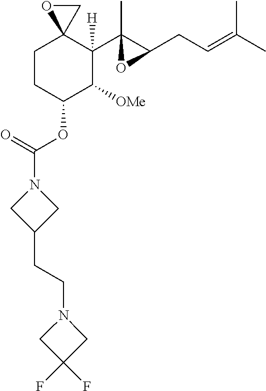 [SMILES: CO[C@@H]1[C@H](OC(=O)N2CC(CCN3CC(F)(F)C3)C2)CC[C@]2(CO2)[C@H]1[C@@]1(C)O[C@@H]1CC=C(C)C] |
| 84 | 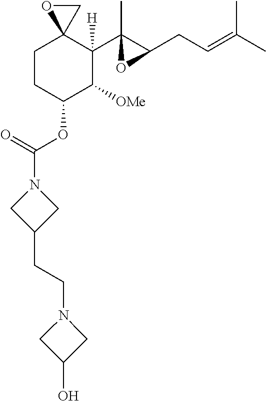 [SMILES: CO[C@@H]1[C@H](OC(=O)N2CC(CCN3CC(O)C3)C2)CC[C@]2(CO2)[C@H]1[C@@]1(C)O[C@@H]1CC=C(C)C] |
| 85 | 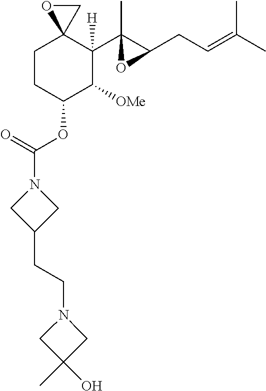 [SMILES: CO[C@@H]1[C@H](OC(=O)N2CC(CCN3CC(C)(O)C3)C2)CC[C@]2(CO2)[C@H]1[C@@]1(C)O[C@@H]1CC=C(C)C] |
| 86 | 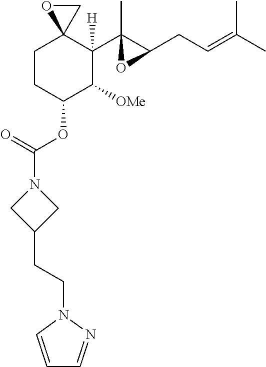 [SMILES: CO[C@@H]1[C@H](OC(=O)N2CC(CCn3cccn3)C2)CC[C@]2(CO2)[C@H]1[C@@]1(C)O[C@@H]1CC=C(C)C] |
| 87 | 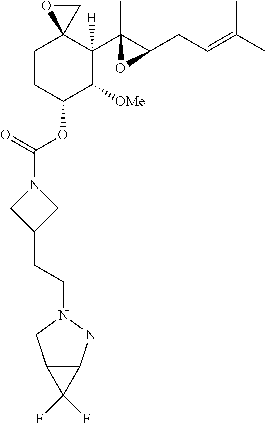 [SMILES: CO[C@@H]1[C@H](OC(=O)N2CC(CCN3CC4C(N3)C4(F)F)C2)CC[C@]2(CO2)[C@H]1[C@@]1(C)O[C@@H]1CC=C(C)C] |
| 88 | 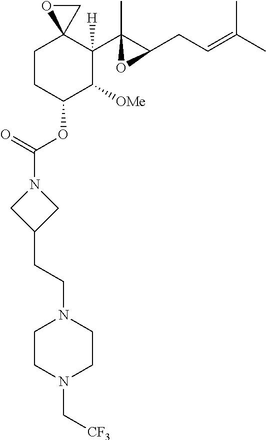 [SMILES: CO[C@@H]1[C@H](OC(=O)N2CC(CCN3CCN(CC(F)(F)F)CC3)C2)CC[C@]2(CO2)[C@H]1[C@@]1(C)O[C@@H]1CC=C(C)C] |
| 89 | 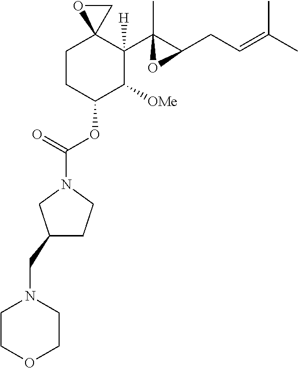 [SMILES: CO[C@@H]1[C@H](OC(=O)N2CC[C@@H](CN3CCOCC3)C2)CC[C@]2(CO2)[C@H]1[C@@]1(C)O[C@@H]1CC=C(C)C] |
| 90 | 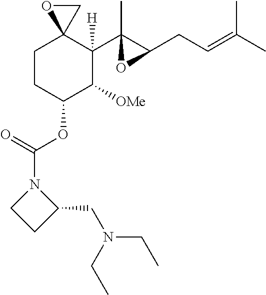 [SMILES: CCN(CC)C[C@@H]1CCN1C(=O)O[C@@H]1CC[C@]2(CO2)[C@@H]([C@@]2(C)O[C@@H]2CC=C(C)C)[C@@H]1OC] |
| 91 | 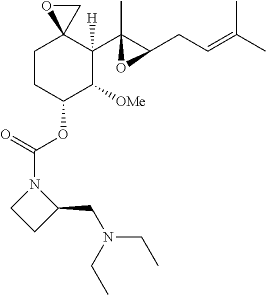 [SMILES: CCN(CC)C[C@H]1CCN1C(=O)O[C@@H]1CC[C@]2(CO2)[C@@H]([C@@]2(C)O[C@@H]2CC=C(C)C)[C@@H]1OC] |
| 92 | 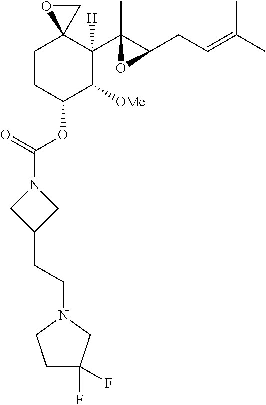 [SMILES: CO[C@@H]1[C@H](OC(=O)N2CC(CCN3CCC(F)(F)C3)C2)CC[C@]2(CO2)[C@H]1[C@@]1(C)O[C@@H]1CC=C(C)C] |
| 93 | 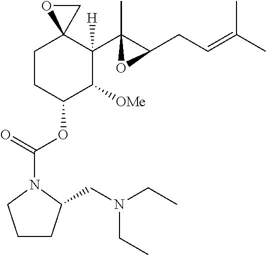 [SMILES: CCN(CC)C[C@@H]1CCCN1C(=O)O[C@@H]1CC[C@]2(CO2)[C@@H]([C@@]2(C)O[C@@H]2CC=C(C)C)[C@@H]1OC] |
| 94 | 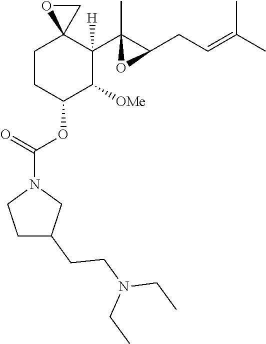 [SMILES: CCN(CC)CCC1CCN(C(=O)O[C@@H]2CC[C@]3(CO3)[C@@H]([C@@]3(C)O[C@@H]3CC=C(C)C)[C@@H]2OC)C1] |
| 95 | 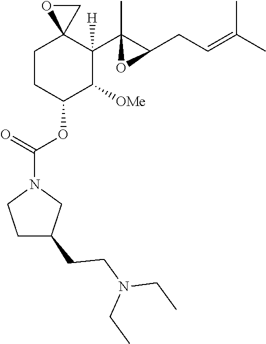 [SMILES: CCN(CC)CC[C@H]1CCN(C(=O)O[C@@H]2CC[C@]3(CO3)[C@@H]([C@@]3(C)O[C@@H]3CC=C(C)C)[C@@H]2OC)C1] |
| 96 | 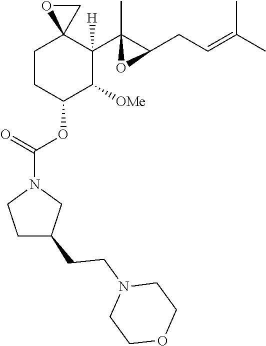 [SMILES: CO[C@@H]1[C@H](OC(=O)N2CC[C@H](CCN3CCOCC3)C2)CC[C@]2(CO2)[C@H]1[C@@]1(C)O[C@@H]1CC=C(C)C] |
| 97 | 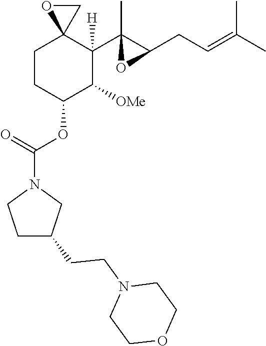 [SMILES: CO[C@@H]1[C@H](OC(=O)N2CC[C@@H](CCN3CCOCC3)C2)CC[C@]2(CO2)[C@H]1[C@@]1(C)O[C@@H]1CC=C(C)C] |
| 98 | 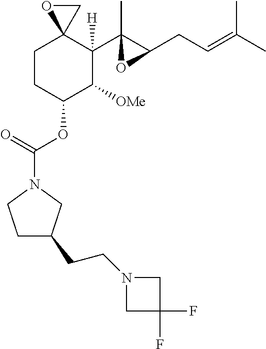 [SMILES: CO[C@@H]1[C@H](OC(=O)N2CC[C@H](CCN3CC(F)(F)C3)C2)CC[C@]2(CO2)[C@H]1[C@@]1(C)O[C@@H]1CC=C(C)C] |
| 99 | 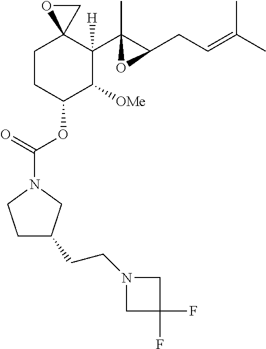 [SMILES: CO[C@@H]1[C@H](OC(=O)N2CC[C@@H](CCN3CC(F)(F)C3)C2)CC[C@]2(CO2)[C@H]1[C@@]1(C)O[C@@H]1CC=C(C)C] |
| 100 | 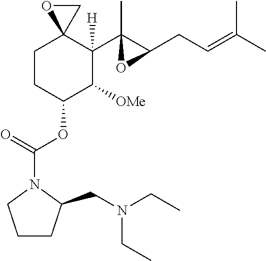 [SMILES: CCN(CC)C[C@H]1CCCN1C(=O)O[C@@H]1CC[C@]2(CO2)[C@@H]([C@@]2(C)O[C@@H]2CC=C(C)C)[C@@H]1OC] |
| 101 | 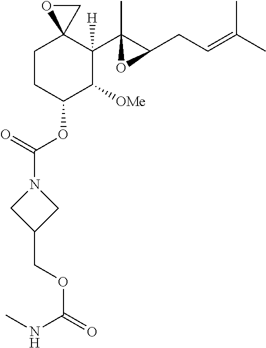 [SMILES: CNC(=O)OCC1CN(C(=O)O[C@@H]2CC[C@]3(CO3)[C@@H]([C@@]3(C)O[C@@H]3CC=C(C)C)[C@@H]2OC)C1] |
| 102 | 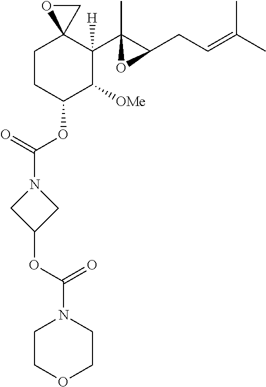 [SMILES: CO[C@@H]1[C@H](OC(=O)N2CC(OC(=O)N3CCOCC3)C2)CC[C@]2(CO2)[C@H]1[C@@]1(C)O[C@@H]1CC=C(C)C] |
| 103 | 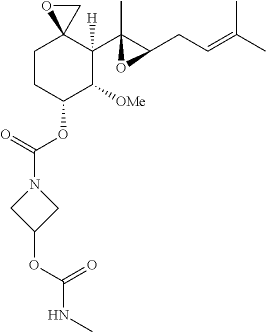 [SMILES: CNC(=O)OC1CN(C(=O)O[C@@H]2CC[C@]3(CO3)[C@@H]([C@@]3(C)O[C@@H]3CC=C(C)C)[C@@H]2OC)C1] |
| 104 | 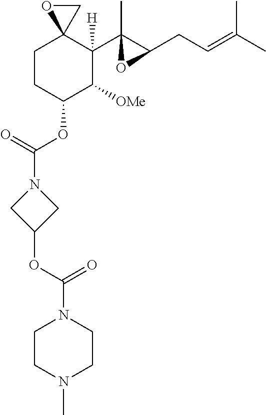 [SMILES: CO[C@@H]1[C@H](OC(=O)N2CC(OC(=O)N3CCN(C)CC3)C2)CC[C@]2(CO2)[C@H]1[C@@]1(C)O[C@@H]1CC=C(C)C] |
| 105 | 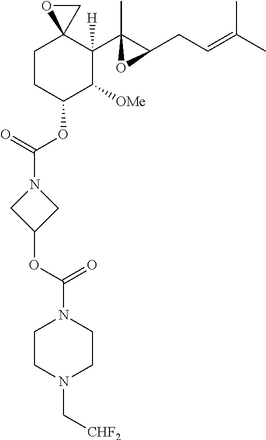 [SMILES: CO[C@@H]1[C@H](OC(=O)N2CC(OC(=O)N3CCN(CC(F)F)CC3)C2)CC[C@]2(CO2)[C@H]1[C@@]1(C)O[C@@H]1CC=C(C)C] |
| 106 | 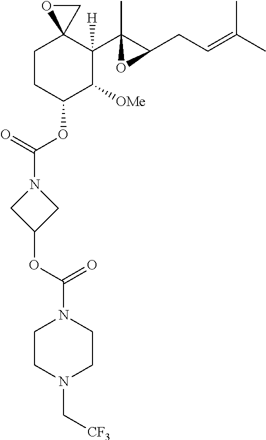 [SMILES: CO[C@@H]1[C@H](OC(=O)N2CC(OC(=O)N3CCN(CC(F)(F)F)CC3)C2)CC[C@]2(CO2)[C@H]1[C@@]1(C)O[C@@H]1CC=C(C)C] |
| 107 | 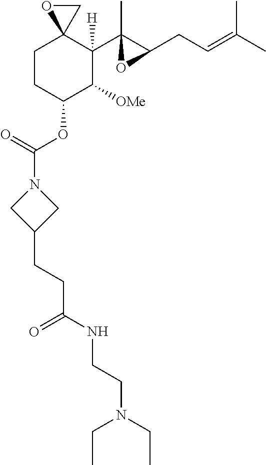 [SMILES: CCN(CC)CCNC(=O)CCC1CN(C(=O)O[C@@H]2CC[C@]3(CO3)[C@@H]([C@@]3(C)O[C@@H]3CC=C(C)C)[C@@H]2OC)C1] |
| 108 | 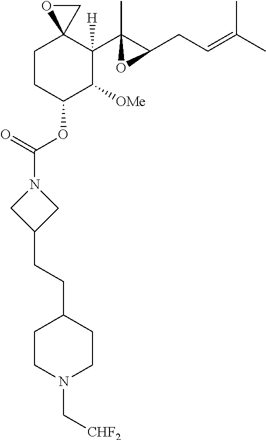 [SMILES: CO[C@@H]1[C@H](OC(=O)N2CC(CCC3CCN(CC(F)F)CC3)C2)CC[C@]2(CO2)[C@H]1[C@@]1(C)O[C@@H]1CC=C(C)C] |
| 109 | 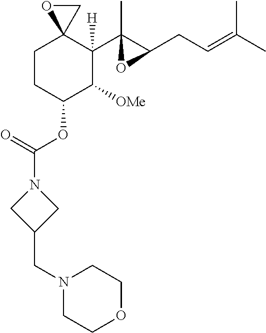 [SMILES: CO[C@@H]1[C@H](OC(=O)N2CC(CN3CCOCC3)C2)CC[C@]2(CO2)[C@H]1[C@@]1(C)O[C@@H]1CC=C(C)C] |
| 110 | 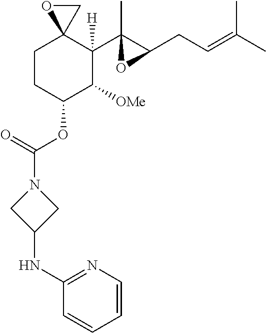 [SMILES: CO[C@@H]1[C@H](OC(=O)N2CC(Nc3ccccn3)C2)CC[C@]2(CO2)[C@H]1[C@@]1(C)O[C@@H]1CC=C(C)C] |
| 111 | 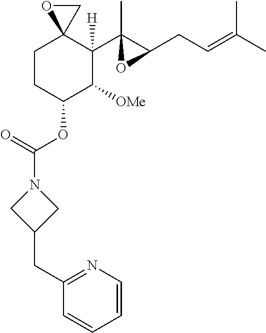 [SMILES: CO[C@@H]1[C@H](OC(=O)N2CC(Cc3ccccn3)C2)CC[C@]2(CO2)[C@H]1[C@@]1(C)O[C@@H]1CC=C(C)C] |
| 112 | 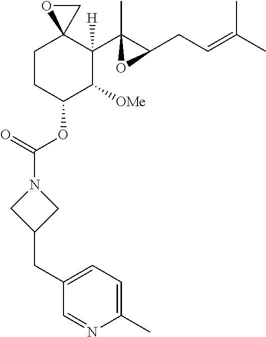 [SMILES: CO[C@@H]1[C@H](OC(=O)N2CC(Cc3ccc(C)nc3)C2)CC[C@]2(CO2)[C@H]1[C@@]1(C)O[C@@H]1CC=C(C)C] |
| 113 | 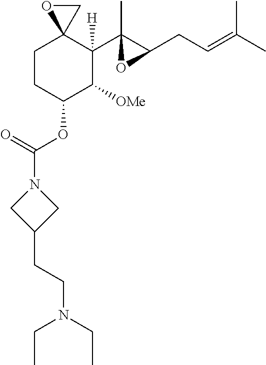 [SMILES: CCN(CC)CCC1CN(C(=O)O[C@@H]2CC[C@]3(CO3)[C@@H]([C@@]3(C)O[C@@H]3CC=C(C)C)[C@@H]2OC)C1] |
| 114 | 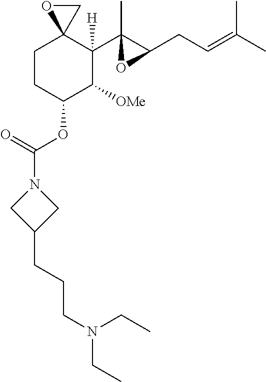 [SMILES: CCN(CC)CCCC1CN(C(=O)O[C@@H]2CC[C@]3(CO3)[C@@H]([C@@]3(C)O[C@@H]3CC=C(C)C)[C@@H]2OC)C1] |
| 115 | 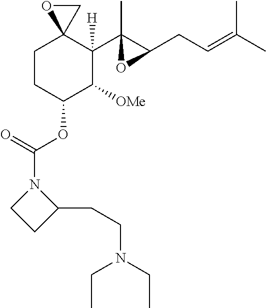 [SMILES: CCN(CC)CCC1CCN1C(=O)O[C@@H]1CC[C@]2(CO2)[C@@H]([C@@]2(C)O[C@@H]2CC=C(C)C)[C@@H]1OC] |
| 116 | 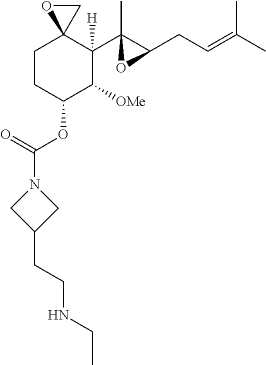 [SMILES: CCNCCC1CN(C(=O)O[C@@H]2CC[C@]3(CO3)[C@@H]([C@@]3(C)O[C@@H]3CC=C(C)C)[C@@H]2OC)C1] |
| 117 | 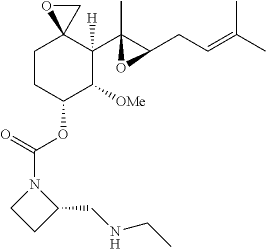 [SMILES: CCNC[C@@H]1CCN1C(=O)O[C@@H]1CC[C@]2(CO2)[C@@H]([C@@]2(C)O[C@@H]2CC=C(C)C)[C@@H]1OC] |
| 118 | 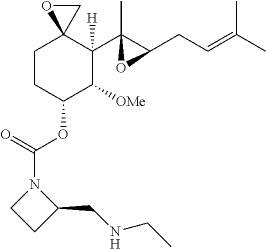 [SMILES: CCNC[C@H]1CCN1C(=O)O[C@@H]1CC[C@]2(CO2)[C@@H]([C@@]2(C)O[C@@H]2CC=C(C)C)[C@@H]1OC] |
| 119 | 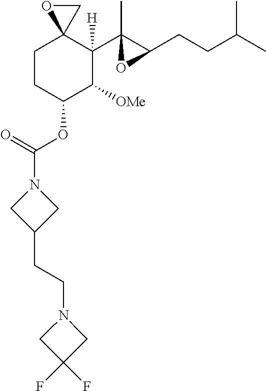 [SMILES: CO[C@@H]1[C@H](OC(=O)N2CC(CCN3CC(F)(F)C3)C2)CC[C@]2(CO2)[C@H]1[C@@]1(C)O[C@@H]1CCC(C)C] |
| 120 | 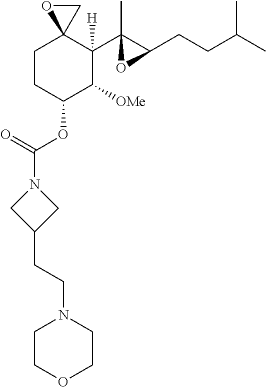 [SMILES: CO[C@@H]1[C@H](OC(=O)N2CC(CCN3CCOCC3)C2)CC[C@]2(CO2)[C@H]1[C@@]1(C)O[C@@H]1CCC(C)C] |
| 121 | 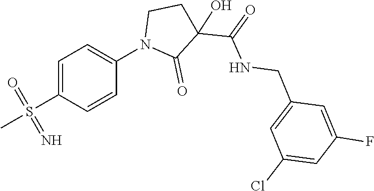 [SMILES: CS(=N)(=O)c1ccc(N2CCC(O)(C(=O)NCc3cc(F)cc(Cl)c3)C2=O)cc1] |
| 122 | 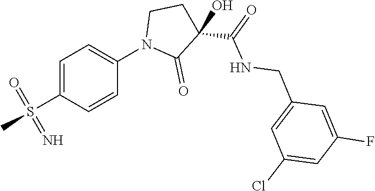 [SMILES: C[S@@](=N)(=O)c1ccc(N2CC[C@](O)(C(=O)NCc3cc(F)cc(Cl)c3)C2=O)cc1] |
| 123 | 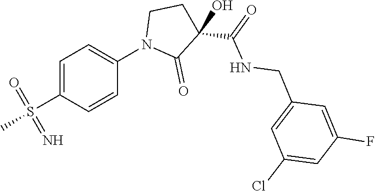 [SMILES: C[S@](=N)(=O)c1ccc(N2CC[C@](O)(C(=O)NCc3cc(F)cc(Cl)c3)C2=O)cc1] |
| 124 | 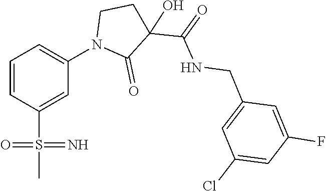 [SMILES: CS(=N)(=O)c1cccc(N2CCC(O)(C(=O)NCc3cc(F)cc(Cl)c3)C2=O)c1] |
| 125 | 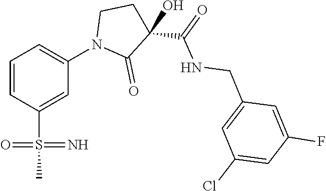 [SMILES: C[S@](=N)(=O)c1cccc(N2CC[C@](O)(C(=O)NCc3cc(F)cc(Cl)c3)C2=O)c1] |
| 126 | 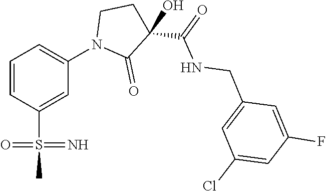 [SMILES: C[S@@](=N)(=O)c1cccc(N2CC[C@](O)(C(=O)NCc3cc(F)cc(Cl)c3)C2=O)c1] |
| 127 | 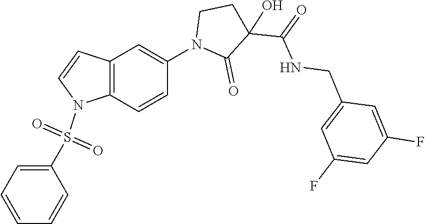 [SMILES: O=C(NCc1cc(F)cc(F)c1)C1(O)CCN(c2ccc3c(ccn3S(=O)(=O)c3ccccc3)c2)C1=O] |
| 128 | 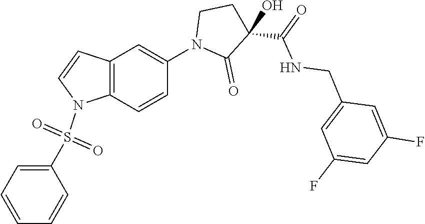 [SMILES: O=C(NCc1cc(F)cc(F)c1)[C@@]1(O)CCN(c2ccc3c(ccn3S(=O)(=O)c3ccccc3)c2)C1=O] |
| 129 | 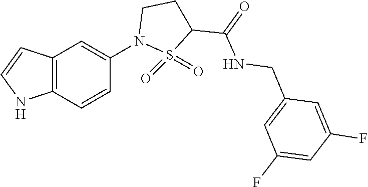 [SMILES: O=C(NCc1cc(F)cc(F)c1)C1CCN(c2ccc3[nH]ccc3c2)S1(=O)=O] |
| 130 | 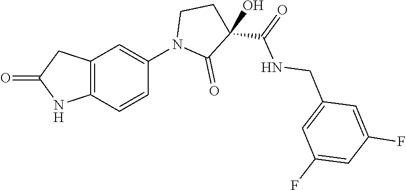 [SMILES: O=C1Cc2cc(N3CC[C@](O)(C(=O)NCc4cc(F)cc(F)c4)C3=O)ccc2N1] |
| 131 | 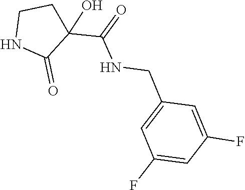 [SMILES: O=C1NCCC1(O)C(=O)NCc1cc(F)cc(F)c1] |
| 132 | 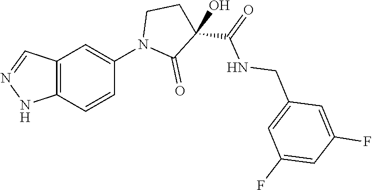 [SMILES: O=C(NCc1cc(F)cc(F)c1)[C@@]1(O)CCN(c2ccc3[nH]ncc3c2)C1=O] |
| 133 | 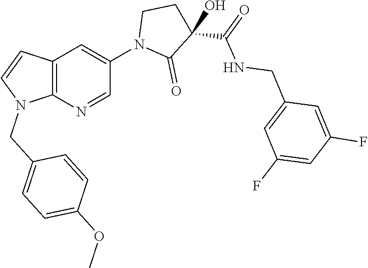 [SMILES: COc1ccc(Cn2ccc3cc(N4CC[C@](O)(C(=O)NCc5cc(F)cc(F)c5)C4=O)cnc32)cc1] |
| 134 | 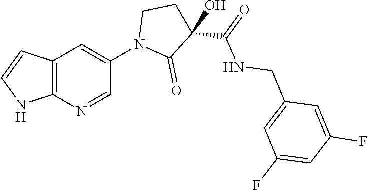 [SMILES: O=C(NCc1cc(F)cc(F)c1)[C@@]1(O)CCN(c2cnc3[nH]ccc3c2)C1=O] |
| 135 | 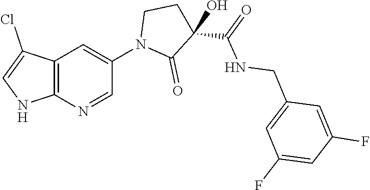 [SMILES: O=C(NCc1cc(F)cc(F)c1)[C@@]1(O)CCN(c2cnc3[nH]cc(Cl)c3c2)C1=O] |
| 136 | 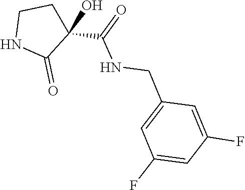 [SMILES: O=C1NCC[C@]1(O)C(=O)NCc1cc(F)cc(F)c1] |
| 137 | 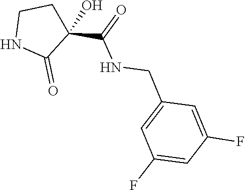 [SMILES: O=C1NCC[C@@]1(O)C(=O)NCc1cc(F)cc(F)c1] |
| 138 | 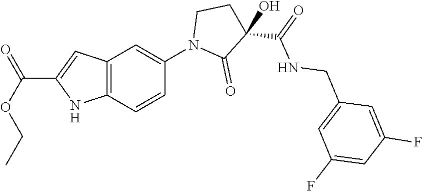 [SMILES: CCOC(=O)c1cc2cc(N3CC[C@](O)(C(=O)NCc4cc(F)cc(F)c4)C3=O)ccc2[nH]1] |

* wherein Polymer has the structure of:

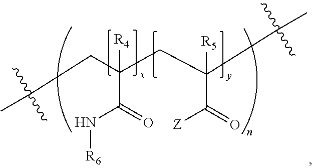

and preferably the structure of:

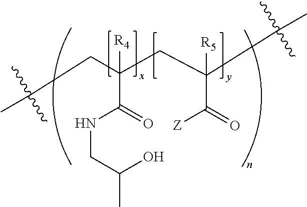

In some aspects, the MetAP2 inhibitor can be:

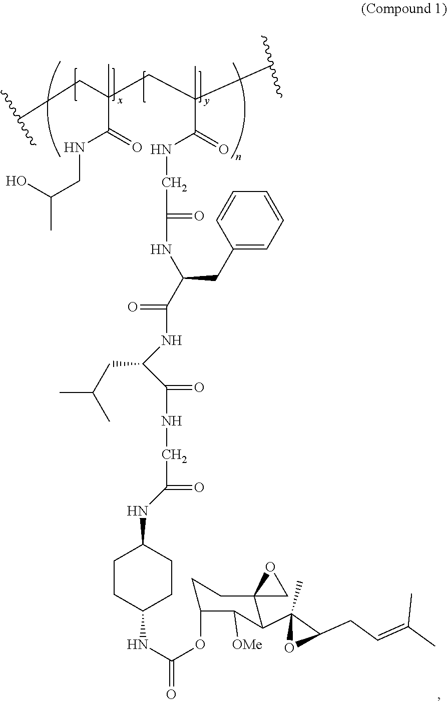

or a pharmaceutically acceptable salt, analog, derivative, salt or ester thereof.

In some aspects, the MetAP2 inhibitor can be:

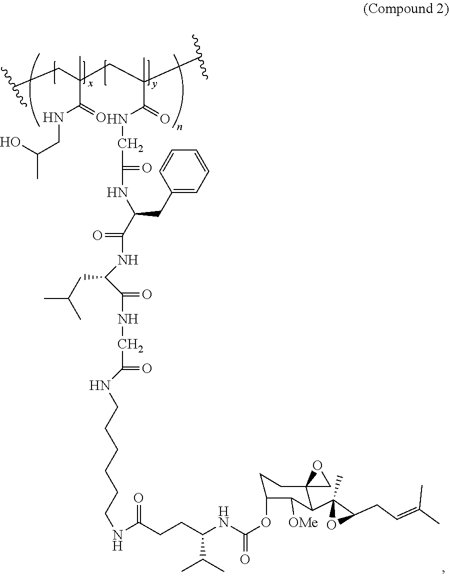

or a pharmaceutically acceptable salt, analog, derivative, salt or ester thereof.

In some aspects, the MetAP2 inhibitor can be:

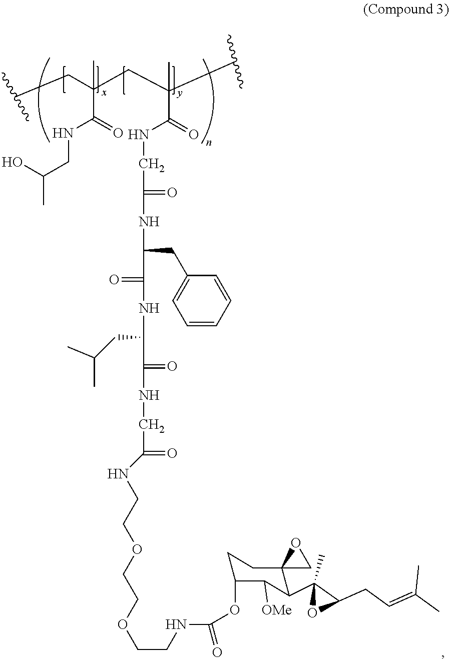

or a pharmaceutically acceptable salt, analog, derivative, salt or ester thereof.

In some aspects, the MetAP2 inhibitor can be:

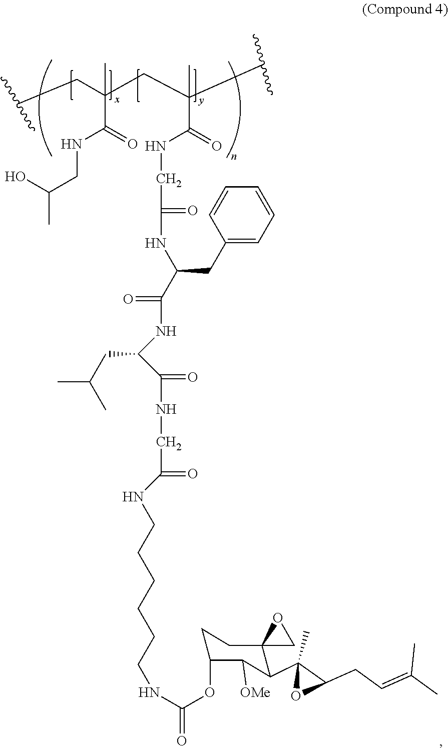

or a pharmaceutically acceptable salt, analog, derivative, salt or ester thereof.

In some aspects, the MetAP2 inhibitor can be:

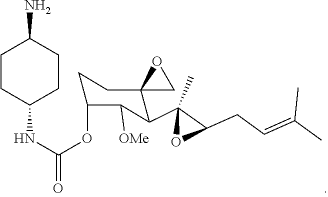

or a pharmaceutically acceptable salt, analog, derivative, salt or ester thereof.

In some aspects, the MetAP2 inhibitor can be:

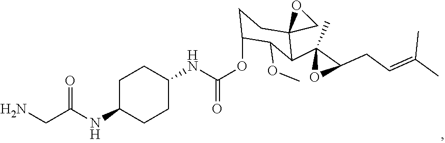

or a pharmaceutically acceptable salt, analog, derivative, salt or ester thereof.

In some aspects, the MetAP2 inhibitor can be:

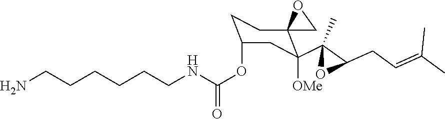

or a pharmaceutically acceptable salt, analog, derivative, salt or ester thereof.

In some aspects, the MetAP2 inhibitor can be:

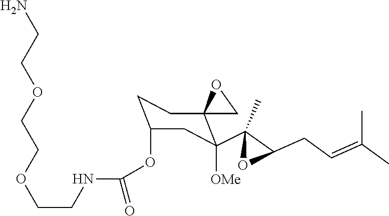

or a pharmaceutically acceptable salt, analog, derivative, salt or ester thereof.

In some aspects, the MetAP2 inhibitor can be:

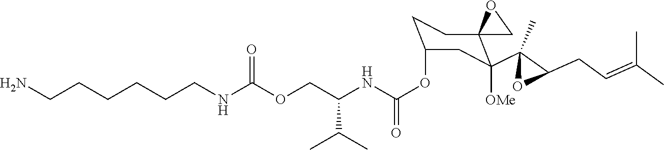

or a pharmaceutically acceptable salt, analog, derivative, salt or ester thereof.

In some aspects, the MetAP2 inhibitor can be selected from cis-(3aRS,9bRS)-7-(benzenesulfonylamino)-1,3a,4,9b-tetrahydro-H-furo[2,3-c]chromene-6-carboxylic acid; cis-(3 aRS,9bRS)-7-[2-(3-diethylaminopropyl)-4-fluorobenzenesulfonyl-amino]1,3a,4,9b-tetrahydro-2H-furo[2,3-c]chromene-6-carboxylic acid; cis-(3aRS,9bRS)-7-[2-(3-{pyrrolidin-1-yl}propyl)-4-fluorobenzenesulfonylamino]-1,3a,4,9b-tetrahydro-2H-furo[2,3-c]chromene-6-carboxylic acid; cis-(3aRS,9bRS)-7-[2-((Z)-3-diethylaminoprop-1-enyl)-4-fluorobenzenesulfonylamino]-1,3a,4,9b-tetrahydro-2H-furo[2,3-c]chromene-6-carboxylic acid, cis-(3aR,9bR)-7-[2-((Z)-3-diethylaminoprop-1-enyl)-4-fluoro-benzenesulfonylamino]-1,3a,4,9b-tetrahydro-2H-furo[2,3-c]chromene-6-carboxylic acid; cis-(3aS,9bS)-7-[2-((Z)-3-dimethylaminoprop-1-enyl)-4-fluorobenzenesulfonylamino]-1,3a,4,9b-tetrahydro-2H-furo[2,3-c]chromene-6-carboxylic acid; 7-[2-((Z)-3-diethylaminoprop-1-enyl)-4-fluorobenzenesulfonylamino]-1,2-dihydrofuro[2,3-c]quinoline-6-carboxylic acid formate salt; 7-(benzenesulfonylamino))-1,2-dihydrofuro[2,3-c]quinoline-6-carboxylic acid formate salt; cis-(3aRS,9bRS)-7-[2-((Z)-3-diethylaminoprop-1-enyl)-4-fluorobenzenesulfonylamino]-1,2,3a,4,5,9b-hexahydrofuro[2,3-c]quinoline-6-carboxylic acid; (1aRS,7bSR)-5-[2-((Z)-3-diethylaminoprop-1-enyl)-4-fluorobenzenesulfonylamino]-1,1a,2,7b-tetrahydrocyclopropa[c]chromene-4-carboxylic acid; (1aR,7bS)-5-[2-((Z)-3-diethylaminoprop-1-enyl)-4-fluorobenzenesulfonylamino]-1,1a,2,7b-tetrahydrocyclopropa[c]chromene-4-carboxylic acid; (1 aS,7bR)-5-[2-((Z)-3-diethylaminoprop-1-enyl)-4-fluorobenzenesulfonylamino]-1,1a,2,7b-tetrahydrocyclopropa[c]chromene-4-carboxylic acid; (1aRS,7bSR)-5-[2-((Z)-3-diethylaminoprop-1-enyl)-4-fluorobenzenesulfonylamino]-7b-methyl-1,1a,2,7b-tetrahydrocyclopropa[c]chromene-4-carboxylic acid; (1aRS,7bSR)-5-[2-((E)-3-diethylaminoprop-1-enyl)-4-fluorobenzenesulfonylamino]-7b-methyl-1,1a,2,7b-tetrahydrocyclopropa[c]chromene-4-carboxylic acid; cis-(3aRS,9bRS)-7-[2-(4-dimethylamino-butylamino)-benzenesulfonylamino]-1,3a,4,9b-tetrahydro-2H-furo[2,3-c]chromene-6-carboxylic acid; (1aR,7bS)-5-[2-(3-diethylaminopropyl)-4-fluorobenzenesulfonyl-amino]-1,1a,2,7b-tetrahydrocyclopropa[c]chromene-4-carboxylic acid; (1aRS,7bSR)-5-[2-((Z)-3-diethylaminoprop-1-enyl)-4-fluorobenzene-sulfonylamino]-1,1-difluoro-1,1a,2,7b-tetrahydrocyclopropa[c]chromene-4-carboxylic acid; (1aR,7bS)-5-[2-((Z)-3-diethylaminoprop-1-enyl)-4-fluorobenzene-sulfonylamino]-1,1-difluoro-1,1a,2,7b-tetrahydrocyclopropa[c]chromene-4-carboxylic acid; (1 aS,7bR)-5-[2-((Z)-3-diethylaminoprop-1-enyl)-4-fluorobenzene-sulfonylamino]-1,1-difluoro-1,1a,2,7b-tetrahydrocyclopropa[c]chromene-4-carboxylic acid; (1aRS,7bSR)-5-[2((Z)-3-ethylaminoprop-1-enyl)-4-fluoro-benzenesulfonylamino]-1,1a,2,7b-tetrahydrocyclopropa-[c]chromene-4-carboxylic acid; (1aR,7bS)-5-[2((Z)-3-ethylaminoprop-1-enyl)-4-fluorobenzenesulfonylamino]-1,1a,2,7b-tetrahydrocyclopropa[c]chromene-4-carboxylic acid; (1aS,7bR)-5-[2((Z)-3-ethylaminoprop-1-enyl)-4-fluorobenzene-sulfonylamino]-1,1a,2,7b-tetrahydro-cyclopropa[c]chromene-4-carboxylic acid; (1aRS,7bSR)-5-{2[(Z)-3-(pyrrolidin-1-yl)prop-1-enyl]-4-fluoro benzenesulfonylamino}-1,1a,2,7b-tetrahydro-cyclopropa[c]chromene-4-carboxylic acid; (1aR,7bS)-5-{2[(Z)-3-(pyrrolidin-1-yl)prop-1-enyl]-4-fluorobenzenesulfonyl-amino}-1,1a,2,7b-tetrahydro-cyclopropa[c]chromene-4-carboxylic acid; (I aS,7bR)-5-{2 [(Z)-3-(pyrrolidin-1-yl)prop-1-enyl]-4-fluorobenzenesulfonylamino}-1,1a,2,7b-tetrahydro-cyclopropa[c]chromene-4-carboxylic acid; (1aRS,7bSR)-5-[2-(3-dimethylaminopropylamino)-benzenesulfonylamino]-1,1a,2,7b-tetrahydrocyclopropa[c]chromene-4-carboxylic acid; (1aR,7bS)-5-[2-(3-dimethylaminopropylamino)benzene-sulfonylamino]-1,1a,2,7b-tetrahydrocyclopropa[c]chromene-4-carboxylic acid; (1aS,7bR)-5-[2-(3-dimethylaminopropyl-amino)benzenesulfonylamino]-1,1a,2,7b-tetrahydrocyclopropa[c]chromene-4-carboxylic acid; (1aRS,7bSR)-5-[2-(4-dimethylaminobutylamino)benzenesulfonylamino]-1,1a,2,7b-tetrahydrocyclopropa[c]chromene-4-carboxylic acid; (1aR,7bS)-5-[2-(4-dimethylamino-butylamino)benzenesulfonylamino]-1,1a,2,7b-tetrahydrocyclopropa[c]chromene-4-carboxylic acid; (1aS,7bR)-5-[2-(4-dimethylaminobutylamino]-1,1a,2,7b-tetrahydrocyclopropa[c]chromene-4-carboxylic acid; (1aRS,7bSR)-5-[2-(5-dimethylamino-pentylamino)benzene-sulfonylamino]-1,1a,2,7b-tetrahydrocyclopropa[c]chromene-4-carboxylic acid; (1aRS,7bSR)-5-{2[(Z)-3-(propan-2-yl)aminoprop-1-enyl]-4-fluorobenzenesulfonyl-amino}-1,1a,2,7b-tetrahydrocyclopropa[c]chromene-4-carboxylic acid; (1aRS,7bSR)-5-{2[(Z)-3-((S)-3-hydroxypyrrolidin-1-yl)aminoprop-1-enyl]-4-fluorobenzenesulfonylamino}-1,1a,2,7b-tetrahydrocyclopropa[c]chromene-4-carboxylic acid; (1aRS,7bSR)-5-{2[(Z)-3-((R)-3-hydroxypyrrolidin-1-yl)aminoprop-1-enyl]-4-fluorobenzene-sulfonylamino}-1,1a,2,7b-tetrahydro-cyclopropa[c]chromene-4-carboxylic acid; (1aRS,7bSR)-5-[2((Z)-4-diethylaminobut-1-enyl)-4-fluorobenzenesulfonyl-amino]-1,1a,2,7b-tetrahydrocyclopropa[c]chromene-4-carboxylic acid; (1aR,7bS)-5-[2((Z)-4-diethylaminobut-1-enyl)-4-fluorobenzenesulfonyl-amino]-1,1a,2,7b-tetrahydrocyclopropa[c]chromene-4-carboxylic acid; (1 aS,7bR)-5-[2((Z)-4-diethylaminobut-1-enyl)-4-fluorobenzenesulfonyl-amino]-1,1a,2,7b-tetrahydro-cyclopropa[c]chromene-4-carboxylic acid; (1aRS,7bSR)-5-{2-[2-(4-ethylpiperazin-1-yl)-ethyl]-4-fluorobenzenesulfonylamino}-1,1a,2,7b-tetrahydrocyclopropa[c]chromene-4-carboxylic acid; (1aRS,7bSR)-5-{2[(Z)-3-(azetidin-1-yl)prop-1-enyl]-4-fluorobenzene-sulfonylamino}-1,1a,2,7b-tetrahydro-cyclopropa[c]chromene-4-carboxylic acid; (1aRS,7bSR)-5-{2[(Z)-3-(3-hydroxy-azetidin-1-yl)prop-1-enyl]-4-fluorobenzene-sulfonylamino}-1,1a,2,7b-tetrahydrocyclopropa-[c]chromene-4-carboxylic acid; (1aRS,7bSR)-5-{2[(Z)-3-(azetidin-1-yl)propyl]-4-fluorobenzenesulfonylamino}-1,1a,2,7b-tetrahydrocyclopropa[c]chromene-4-carboxylic acid, (1 aRS,7bSR)-5-[2((Z)-4-diethylaminobutyl)-4-fluorobenzenesulfonylamino]-1,1a,2,7b-tetrahydrocyclopropa[c]chromene-4-carboxylic acid; (1aRS,7bSR)-5-{2-[N-(4-dimethylaminobutyl)-N-methylamino]-benzenesulfonyl-amino}-1,1a,2,7b-tetrahydrocyclopropa-[c]chromene-4-carboxylic acid, (1aRS,7bSR)-5-{2-[((S)-1-ethylpyrrolidin-3-ylcarbamoyl)-methyl]-4-fluoro-benzenesulfonyl-amino}-1,1a,2,7b-tetrahydrocyclopropa-[c]chromene-4-carboxylic acid; (1aRS,7bSR)-5-[2-(1-ethylazetidin-3-yl)-4-fluorobenzenesulfonylamino]-1,1a,2,7b-tetrahydro-cyclopropa[c]chromene-4-carboxylic acid; (1aRS,7bSR)-5-{2-[((R)-1-ethylpyrrolidin-3-ylcarbamoyl)methyl]-4-fluorobenzenesulfonyl-amino}-1,1a,2,7b-tetrahydro-cyclopropa[c]chromene-4-carboxylic acid; (1aRS,7bSR)-5-{2-[2-(pyrrolidin-1-yl)-ethyl]-4-fluorobenzenesulfonylamino}-1,1a,2,7b-tetrahydrocyclopropa[c]chromene-4-carboxylic acid; (1aRS,7bSR)-5-[2-((R)-1-ethylpyrrolidin-3-ylmethyl)-4-fluorobenzenesulfonyl-amino]-1,1a,2,7b-tetrahydro-cyclopropa[c]chromene-4-carboxylic acid; (1aS,7bR)-5-[2-((R)-1-ethylpyrrolidin-3-ylmethyl)-4-fluorobenzenesulfonyl-amino]-1,1a,2,7b-tetrahydro-cyclopropa[c]chromene-4-carboxylic acid; (1aR,7bS)-5-[2-((R)-1-ethylpyrrolidin-3-ylmethyl)-4-fluorobenzenesulfonyl-amino]-1,1a,2,7b-tetrahydro-cyclopropa[c]chromene-4-carboxylic acid; (1aRS,7bSR)-5-{2-[((S)-1-ethylpyrrolidin-2-yl)carbonyl-aminomethyl]-4-fluorobenzene-sulfonylamino}-1,1a,2,7b-tetrahydrocyclopropa[c]chromene-4-carboxylic acid; (1aRS,7bSR)-5-[2-(4-dimethylaminobutyrylamino)-4-fluorobenzenesulfonyl-amino]-1,1a,2,7b-tetrahydrocyclopropa-[c]chromene-4-carboxylic acid; (1aRS,7bSR)-5-[2-((S)-1-ethyl-pyrrolidin-3-ylmethyl)-4-fluorobenzenesulfonyl-amino]-1,1a,2,7b-tetrahydrocyclopropa[c]chromene-4-carboxylic acid; (1aRS,7bSR)-5-[2-(3-dimethylaminopropylcarbanoyl)benzene-sulfonylamino]-1,1a,2,7b-tetrahydrocyclopropa[c]chromene-4-carboxylic acid; (1aRS,7bSR)-5-(2-{[N—((S)-1-ethyl-pyrrolidin-3-yl)-N-methylcarbamoyl]methyl}-4-fluoro-benzenesulfonylamino)-1,1a,2,7b-tetrahydrocyclopropa-[c]chromene-4-carboxylic acid; (1aRS,7bSR)-5-(2-{[N—((R)-1-ethyl-pyrrolidin-3-yl)-N-methylcarbamoyl]methyl}-4-fluoro-benzenesulfonylamino)-1,1a,2,7b-tetrahydrocyclopropa-[c]chromene-4-carboxylic acid; (1aRS,7bSR)-5-{2-[2-((S)-1-ethylpyrrolidin-2-yl)ethylamino]-benzenesulfonyl-amino}-1,1a,2,7b-tetrahydrocyclopropa-[c]chromene-4-carboxylic acid; (1aRS,7bSR)-5-{2-[2-((R)-1-ethylpyrrolidin-2-yl)ethylamino]-benzenesulfonyl-amino}-1,1a,2,7b-tetrahydrocyclopropa-[c]chromene-4-carboxylic acid; (1aRS,7bSR)-5-[2-(3-N,N-diethylaminopropylamino)benzene-sulfonylamino]-1,1a,2,7b-tetrahydrocyclopropa[c]chromene-4-carboxylic acid; (1aRS,7bSR)-5-(2-{[((R)-1-ethylpyrrolidine-2-yl)carbonyl-amino]methyl}-4-fluorobenzenesulfonylamino)-1,1a,2,7b-tetrahydrocyclopropa[c]chromene-4-carboxylic acid; (1aRS,7bSR)-5-{2-[(1-ethylazetidin-3-ylmethyl)amino]benzene-sulfonylamino}-1,1a,2,7b-tetrahydrocyclopropa[c]chromene-4-carboxylic acid; (1 aS,7bR)-5-[2-((Z)-3-diethylaminoprop-1-enyl)benzenesulfonylamino]-1,1a,2,7b-tetrahydrocyclopropa[c]chromene-4-carboxylic acid; (1aR,7bS)-5-[2-((Z)-3-diethylaminoprop-1-enyl)benzenesulfonylamino]-1,1a,2,7b-tetrahydrocyclopropa[c]chromene-4-carboxylic acid; (1aRS,7bSR)-5-(2-{N—[((R)-1-ethylpyrrolidine-2-yl)carbonyl]-N-methyl-aminomethyl}-4-fluorobenzenesulfonylamino)-1,1a,2,7b-tetrahydrocyclopropa[c]chromene-4-carboxylic acid; (1aRS,7bSR)-5-(2-{N—[((S)-1-ethylpyrrolidine-2-yl)carbonyl]-N-methylamino-methyl}-4-fluorobenzenesulfonylamino)-1,1a,2,7b-tetrahydrocyclopropa[c]chromene-4-carboxylic acid; (1aRS,7bSR)-5-[2-(4-dimethylaminobutylamino)-4-fluorobenzenesulfonyl-amino]-1,1a,2,7b-tetrahydrocyclopropa[c]chromene-4-carboxylic acid; (1aRS,7bSR)-5-{2-[((R)-1-ethylpyrrolidin-3-ylmethyl)amino]-benzenesulfonylamino}-1,1a,2,71b-tetrahydrocyclopropa-[c]chromene-4-carboxylic acid; (1aRS,7bSR)-5-{2-[((S)-1-ethylpyrrolidin-3-ylmethyl)amino]-benzenesulfonylamino}-1,1a,2,7b-tetrahydrocyclopropa-[c]chromene-4-carboxylic acid; (1aRS,7bSR)-5-[2-(4-ethyl-2-oxopiperazin-1-ylmethyl)-4-fluorobenzene-sulfonylamino]-1,1a,2,7b-tetrahydrocyclopropa[c]chromene-4-carboxylic acid; (1aRS,7bSR)-5-[2-(1-ethylpiperidin-4-ylmethyl)-4-fluoro-benzenesulfonylamino]-1,1a,2,7b-tetrahydrocyclopropa-[c]chromene-4-carboxylic acid; (1aRS,7bSR)-5-{2-[2-(1-ethylazetidin-3-yl)ethyl]-4-fluoro-benzenesulfonyl-amino}-1,1a,2,7b-tetrahydrocyclopropa[c]chromene-4-carboxylic acid; (1 aRS,7bSR)-5-{2-[((S)-1-azabicyclo[2.2.2]oct-3-yl)amino]benzenesulfonyl-amino}-1,1a,2,7b-tetrahydrocyclopropa[c]chromene-4-carboxylic acid; (1aRS,7bSR)-5-{2-[((R)-1-azabicyclo-[2.2.2]oct-3-yl)amino]benzenesulfonyl-amino}-1,1a,2,7b-tetrahydrocyclopropa[c]chromene-4-carboxylic acid; (1aRS,7bSR)-5-(2-{[((S)-1-ethylpyrrolidine-3-carbonyl)amino]methyl}-4-fluoro-benzenesulfonylamino)-1,1a,2,7b-tetrahydrocyclopropa[c]chromene-4-carboxylic acid; (1 aRS,7bSR)-5-{2-[2-((R)-1-ethylpyrrolidin-3-ylamino)ethyl]-4-fluoro-benzenesulfonylamino}-1,1a,2,7b-tetrahydrocyclopropa-[c]chromene-4-carboxylic acid; (1aRS,7bSR)-5-{2-[((R)-1-ethylpyrrolidin-3-yl)amino]-benzenesulfonylamino}-1,1a,2,7b-tetrahydrocyclopropa-[c]chromene-4-carboxylic acid; (1aRS,7bSR)-5-{2-[((S)-1-ethylpyrrolidin-3-yl)amino]-benzenesulfonylamino}-1,1a,2,7b-tetrahydrocyclopropa-[c]chromene-4-carboxylic acid; (1 aRS,7bSR)-5-(2-{[((R)-1-ethylpyrrolidine-3-carbonyl)amino]-methyl)}-4-fluoro-benzenesulfonylamino)-1,1a,2,7b-tetrahydro-cyclopropa[c]chromene-4-carboxylic acid; (1aRS,7bSR)-5-[2-((Z)-3-diethylamino-2-methylprop-1-enyl)-4-fluorobenzene-sulfonylamino]-1,1a,2,7b-tetrahydrocyclopropa[c]chromene-4-carboxylic acid; (1aRS,7bSR)-5-(2-[2-((R)-1-ethylpyrrolidin-3-yl)ethylamino]-benzenesulfonylamino}-1,1a,2,7b-tetrahydrocyclopropa-[c]chromene-4-carboxylic acid; (1aRS,7bSR)-5-{2-[2-((S)-1-ethylpyrrolidin-3-yl)ethylamino]-benzenesulfonyl-amino}-1,1a,2,7b-tetrahydrocyclopropa-[c]chromene-4-carboxylic acid; (1aR,7bS)-5-[2-((S)-1-ethylpyrrolidin-3-yloxymethyl)-4-fluoro-benzenesulfonylamino]-1,1a,2,7b-tetrahydrocyclopropa-[c]chromene-4-carboxylic acid; (1aR,7bS)-5-[2-((R)-1-ethylpyrrolidin-3-yloxymethyl)-4-fluoro-benzenesulfonylamino]-1,1a,2,7b-tetrahydrocyclopropa-[c]chromene-4-carboxylic acid; (1aR,7bS)-5-[2-(1-ethylpiperidin-3-ylmethyl)-4-fluorobenzene-sulfonylamino]-1,1a,2,7b-tetrahydrocyclopropa[c]chromene-4-carboxylic acid; (1aR,7bS)-5-{2-[2-((R)-1-ethylpyrrolidin-2-yl)ethyl]-4-fluorobenzenesulfonyl-amino}-1,1a,2,7b-tetrahydrocyclopropa-[c]chromene-4-carboxylic acid; and pharmaceutically acceptable salts, stereoisomers, esters and prodrugs thereof.

In some aspects, the MetAP2 inhibitor can be selected from:

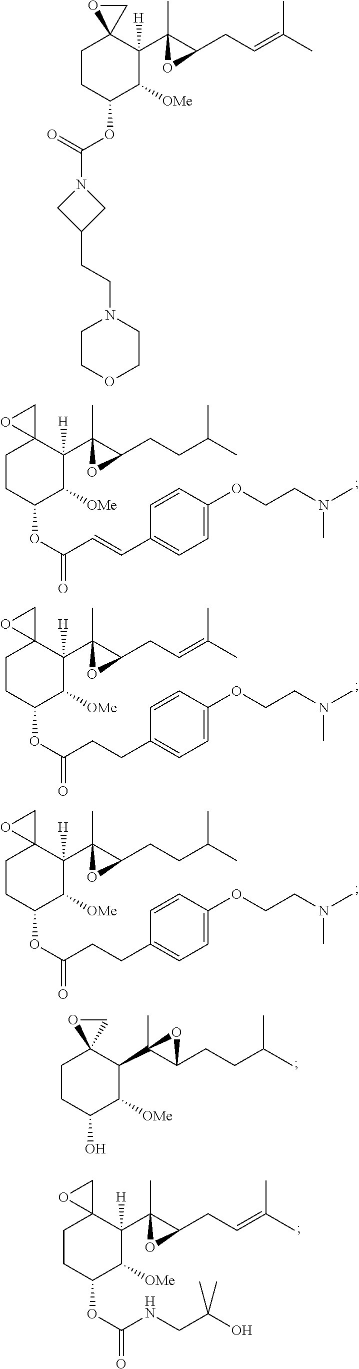

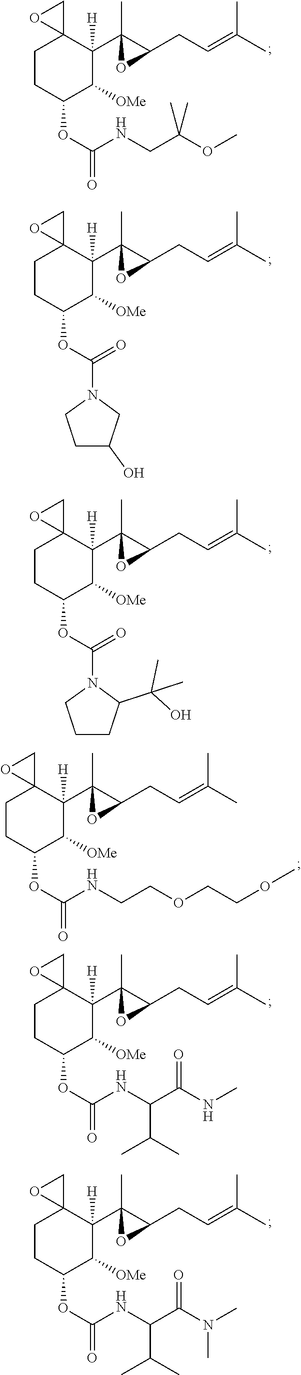

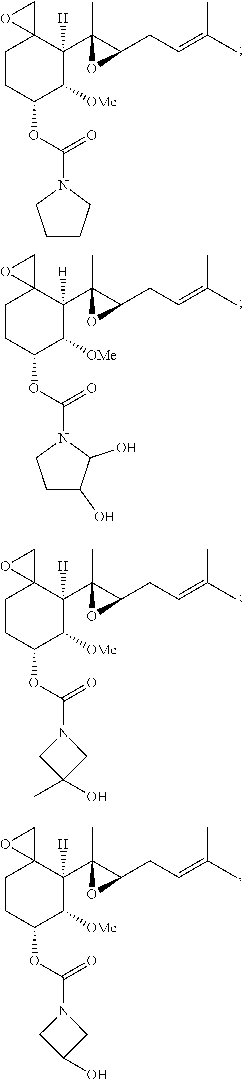

or a pharmaceutically acceptable salt, analog, derivative, salt or ester thereof.

For purposes of this disclosure, the chemical elements are identified in accordance with the Periodic Table of the Elements, CAS version, Handbook of Chemistry and Physics, 67th Ed., 1986-87, inside cover.

The term “alkyl” refers to a fully saturated branched or unbranched carbon chain radical having the number of carbon atoms specified, or up to 30 carbon atoms if no specification is made. For example, a “lower alkyl” refers to an alkyl having from 1 to 10 carbon atoms, such as methyl, ethyl, propyl, butyl, pentyl, hexyl, heptyl, and octyl, and those which are positional isomers of these alkyls. Alkyl of 10 to 30 carbon atoms includes decyl, undecyl, dodecyl, tridecyl, tetradecyl, pentadecyl, hexadecyl, heptadecyl, octadecyl, nonadecyl, eicosyl, heneicosyl, docosyl, tricosyl and tetracosyl. In certain aspects, a straight chain or branched chain alkyl has 30 or fewer carbon atoms in its backbone (e.g., C₁-C₃₀ for straight chains, C₃-C₃₀ for branched chains), and more preferably 20 or fewer. Likewise, certain cycloalkyls have from 3-10 carbon atoms in their ring structure, and may have 5, 6, or 7 carbons in the ring structure.

Unless the number of carbons is otherwise specified, “lower alkyl”, as used herein, means an alkyl group, as defined above, but having from one to ten carbons, or from one to six carbon atoms in its backbone structure such as methyl, ethyl, n-propyl, isopropyl, n-butyl, isobutyl, sec-butyl, and tert-butyl. Likewise, “lower alkenyl” and “lower alkynyl” have similar chain lengths. Throughout the application, certain alkyl groups are lower alkyls. In certain aspects, a substituent designated herein as alkyl is a lower alkyl.

The term “carbocycle”, as used herein, refers to an aromatic or non-aromatic ring in which each atom of the ring is carbon.

The term “aryl” as used herein includes 5-, 6- and 7-membered single-ring aromatic groups that may include from zero to four heteroatoms, for example, benzene, pyrrole, furan, thiophene, imidazole, oxazole, thiazole, triazole, pyrazole, pyridine, pyrazine, pyridazine and pyrimidine, and the like. Those aryl groups having heteroatoms in the ring structure may also be referred to as “aryl heterocycles” or “heteroaromatics”. The aromatic ring can be substituted at one or more ring positions with such substituents as described above, for example, halogen, azide, alkyl, aralkyl, alkenyl, alkynyl, cycloalkyl, hydroxyl, alkoxyl, amino, nitro, sulfhydryl, imino, amido, phosphonate, phosphinate, carbonyl, carboxyl, silyl, ether, alkylthio, sulfonyl, sulfonamido, ketone, aldehyde, ester, heterocyclyl, aromatic or heteroaromatic moieties, —CF₃, —CN, or the like. The term “aryl” also includes polycyclic ring systems having two or more cyclic rings in which two or more carbons are common to two adjoining rings (the rings are “fused rings”) wherein at least one of the rings is aromatic, e.g., the other cyclic rings can be cycloalkyls, cycloalkenyls, cycloalkynyls, aryls and/or heterocyclyls.

“Alkenyl” refers to any branched or unbranched unsaturated carbon chain radical having the number of carbon atoms specified, or up to 26 carbon atoms if no limitation on the number of carbon atoms is specified; and having 1 or more double bonds in the radical. Alkenyl of 6 to 26 carbon atoms is exemplified by hexenyl, heptenyl, octenyl, nonenyl, decenyl, undecenyl, dodenyl, tridecenyl, tetradecenyl, pentadecenyl, hexadecenyl, heptadecenyl, octadecenyl, nonadecenyl, eicosenyl, heneicosenyl, docosenyl, tricosenyl and tetracosenyl, in their various isomeric forms, where the unsaturated bond(s) can be located anywhere in the radical and can have either the (Z) or the (E) configuration about the double bond(s).

The term “alkynyl” refers to hydrocarbyl radicals of the scope of alkenyl, but having one or more triple bonds in the radical.

The terms “alkoxyl” or “alkoxy” as used herein refers to an alkyl group, as defined below, having an oxygen radical attached thereto. Representative alkoxy groups include methoxy, ethoxy, propoxy, tert-butoxy and the like. An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, —O-alkynyl, —(CH₂)ₘ—R₁, where m and R₁ are described below.

The terms “heterocyclyl” or “heterocyclic group” refer to 3- to 10-membered ring structures, more preferably 3- to 7-membered rings, whose ring structures include one to four heteroatoms. Heterocycles can also be polycycles. Heterocyclyl groups include, for example, thiophene, thianthrene, furan, pyran, isobenzofuran, chromene, xanthene, phenoxathiin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring can be substituted at one or more positions with such substituents as described above, as for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, sulfamoyl, sulfinyl, ether, alkylthio, sulfonyl, ketone, aldehyde, ester, a heterocyclyl, an aromatic or heteroaromatic moiety, —CF₃, —CN, or the like.

The term “alkylthio” refers to an alkyl group, as defined above, having a sulfur radical attached thereto. In certain aspects, the “alkylthio” moiety is represented by one of —(S)-alkyl, —(S)-alkenyl, —(S)-alkynyl, and —(S)—(CH₂)ₘ—Rr, wherein n and R₁ are defined below. Representative alkylthio groups include methylthio, ethylthio, and the like.

As used herein, the term “nitro” means —NO₂; the term “halogen” designates F, Cl, Br or t; the term “sulfhydryl” means —SH; the term “hydroxyl” means —OH; and the term “sulfonyl” means —SO₂—.

The terms “amine” and “amino” are art-recognized and refer to both unsubstituted and substituted amines, e.g., a moiety that can be represented by the general formulae:

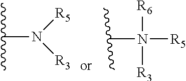

wherein R₃, R₅ and R₆ each independently represent a hydrogen, an alkyl, an alkenyl, —(CH₂)ₘ—R₁, or R₃ and R₅ taken together with the N atom to which they are attached complete a heterocycle having from 4 to 8 atoms in the ring structure, R₁ represents an alkenyl, aryl, cycloalkyl, a cycloalkenyl, a heterocyclyl or a polycyclyl; and m is zero or an integer in the range of 1 to 8. In certain aspects, only one of R₃ or R₅ can be a carbonyl, e.g., R₃, R₅ and the nitrogen together do not form an imide. In certain aspects, R₃ and R₅ (and optionally R₆) each independently represent a hydrogen, an alkyl, an alkenyl, or —(CH₂)ₘ—R₁. Thus, the term “alkylamine” as used herein means an amine group, as defined above, having a substituted or unsubstituted alkyl attached thereto, i.e., at least one of R₃ and R₅ is an alkyl group. In certain aspects, an amino group or an alkylamine is basic, meaning it has a pKₐ≥7.00. The protonated forms of these functional groups have pKₐs relative to water above 7.00.

The term “carbonyl” (C(O)) is art-recognized and includes such moieties as can be represented by the general formula:

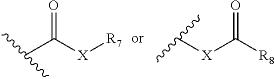

wherein X is a bond or represents an oxygen or a sulfur, and R₇ represents a hydrogen, an alkyl, an alkenyl, —(CH₂)ₘ—R₁ or a pharmaceutically acceptable salt, R₈ represents a hydrogen, an alkyl, an alkenyl or —(CH₂)ₘ—R₁, where m and R₁ are as defined above. Where X is an oxygen and R₇ or R₈ is not hydrogen, the formula represents an “ester”. Where X is an oxygen, and R₇ is as defined above, the moiety is referred to herein as a carboxyl group, and particularly when R₇ is a hydrogen, the formula represents a “carboxylic acid”. Where X is an oxygen, and R₈ is hydrogen, the formula represents a “formate”. In general, where the oxygen atom of the above formula is replaced by sulfur, the formula represents a “thiocarbonyl” group. Where X is a sulfur and R₇ or R₈ is not hydrogen, the formula represents a “thioester” group. Where X is a sulfur and R₇ is hydrogen, the formula represents a“thiocarboxylic acid” group. Where X is a sulfur and R₈ is hydrogen, the formula represents a “thioformate” group. On the other hand, where X is a bond, and R₇ is not hydrogen, the above formula represents a “ketone” group. Where X is a bond, and R₇ is hydrogen, the above formula represents an “aldehyde” group.

As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. Illustrative substituents include, for example, those described herein above. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. This disclosure is not intended to be limited in any manner by the permissible substituents of organic compounds. It will be understood that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc.

The term “sulfamoyl” is art-recognized and includes a moiety that can be represented by the general formula:

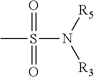

In which R₃ and R₅ are as defined above.

The term “sulfate” is art recognized and includes a moiety that can be represented by the general formula:

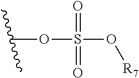

In which R₇ is as defined above.

The term “sulfamido” is art recognized and includes a moiety that can be represented by the general formula:

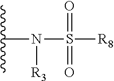

In which R₂ and R₄ are as defined above.

The term “sulfonate” is art-recognized and includes a moiety that can be represented by the general formula:

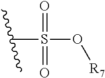

In which R₇ is an electron pair, hydrogen, alkyl, cycloalkyl, or aryl.

The terms “sulfoxido” or “sulfinyl”, as used herein, refers to a moiety that can be represented by the general formula:

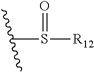

In which R₁₂ is selected from the group consisting of hydrogen, alkyl, alkenyl, alkynyl, cycloalkyl, heterocyclyl, aralkyl, or aryl.

Analogous substitutions can be made to alkenyl and alkynyl groups to produce, for example, aminoalkenyls, aminoalkynyls, amidoalkenyls, amidoalkynyls, iminoalkenyls, iminoalkynyls, thioalkenyls, thioalkynyls, carbonyl-substituted alkenyls or alkynyls.

As used herein, the definition of each expression, e.g., alkyl, in, n, etc., when it occurs more than once in any structure, is intended to be independent of its definition elsewhere in the same structure.

The term “amino acid” is intended to embrace all compounds, whether natural or synthetic, which include both an amino functionality and an acid functionality, including amino acid analogs and derivatives. In certain aspects, the amino acids contemplated in the present disclosure are those naturally occurring amino acids found in proteins, or the naturally occurring anabolic or catabolic products of such amino acids, which contain amino and carboxyl groups. Naturally occurring amino acids are identified throughout by the conventional three-letter and/or one-letter abbreviations, corresponding to the trivial name of the amino acid, in accordance with the following list. The abbreviations are accepted in the peptide art and are recommended by the IUPAC-IUB commission in biochemical nomenclature.

By the term “amino acid residue” is meant an amino acid. In general the abbreviations used herein for designating the naturally occurring amino acids are based on recommendations of the IUPAC-IUB Commission on Biochemical Nomenclature (see Biochemistry (1972) 11:1726-1732). For instance Met, Ile, Leu, Ala and Gly represent “residues” of methionine, isoleucine, leucine, alanine and glycine, respectively. By the residue is meant a radical derived from the corresponding α-amino acid by eliminating the OH portion of the carboxyl group and the 11 portion of the α-amino group.

The term “amino acid side chain” is that part of an amino acid residue exclusive of the backbone, as defined by K. D. Kopple, “Peptides and Amino Acids”, W. A. Benjamin Inc., New York and Amsterdam, 1966, pages 2 and 33; examples of such side chains of the common amino acids are —CH₂CH₂SCH₃ (the side chain of methionine), —CH₂(CH₃)—CH₂CH₃ (the side chain of isoleucine), —CH₂CH(CH₃)₂ (the side chain of leucine) or H-(the side chain of glycine). These side chains are pendant from the backbone Ca carbon.

The term “peptide,” as used herein, refers to a sequence of amino acid residues linked together by peptide bonds or by modified peptide bonds. The term “peptide” is intended to encompass peptide analogs, peptide derivatives, peptidomimetics and peptide variants. The term “peptide” is understood to include peptides of any length. Peptide sequences set out herein are written according to the generally accepted convention whereby the N-terminal amino acid is on the left, and the C-terminal amino acid is on the right (e.g., H₂N-AA₁-AA₂-AA₃-AA₄-AA₅-AA₆-CO₂H).

Certain compounds of the present disclosure may exist in particular geometric or stereoisomeric forms. The present disclosure contemplates all such compounds, including cis- and trans-isomers, R- and S-enantiomers, diastereomers, (D)-isomers, (L)-isomers, the racemic mixtures thereof, and other mixtures thereof, as falling within the scope of the disclosure.

Additional asymmetric carbon atoms may be present in a substituent such as an alkyl group. All such isomers, as well as mixtures thereof, are intended to be included in this disclosure. Any representation of a particular isomer is merely exemplary (e.g., the exemplification of a trans-isomer, also encompasses a cis-isomer).

If, for instance, a particular enantiomer of a compound of the present disclosure is desired, it may be prepared by asymmetric synthesis or by derivation with a chiral auxiliary, where the resulting diastereomeric mixture is separated and the auxiliary group cleaved to provide the pure desired enantiomer. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomer.

The term “substituted”, as used herein, means that any one or more hydrogen atoms on the designated atom is replaced with a selection from the indicated groups, provided that the designated atom's normal valency is not exceeded, and that the substitution results in a stable compound. When a substituent is keto (i.e., ═O), then 2 hydrogen atoms on the atom are replaced. Keto substituents are not present on aromatic moieties. Ring double bonds, as used herein, are double bonds that are formed between two adjacent ring atoms (e.g., C═C, C═N or N═N). “Stable compound” and “stable structure” are meant to indicate a compound that is sufficiently robust to survive isolation to a useful degree of purity from a reaction mixture, and formulation into an efficacious therapeutic agent.

When a bond to a substituent is shown to cross a bond connecting two atoms in a ring, then such substituent may be bonded to any atom in the ring. When a substituent is listed without indicating the atom via which such substituent is bonded to the rest of the compound of a given formula, then such substituent may be bonded via any atom in such formula. Combinations of substituents and/or variables are permissible, but only if such combinations result in stable compounds.

When any variable (e.g., R₁) occurs more than one time in any constituent or formula for a compound, its definition at each occurrence is independent of its definition at every other occurrence. Thus, for example, if a group is shown to be substituted with 0-2 R₁ moieties, then the group may optionally be substituted with up to two R₁ moieties and R₁ at each occurrence is selected independently from the definition of R₁. Also, combinations of substituents and/or variables are permissible, but only if such combinations result in stable compounds.

In the present specification, the structural formula of the compound represents a certain isomer for convenience in some cases, but the present disclosure includes all isomers, such as geometrical isomers, optical isomers based on an asymmetrical carbon, stereoisomers, tautomers, and the like. In addition, a crystal polymorphism may be present for the compounds represented by the formula. It is noted that any crystal form, crystal form mixture, or anhydride or hydrate thereof is included in the scope of the present disclosure. Furthermore, so-called metabolite which is produced by degradation of the present compound in vivo is included in the scope of the present disclosure.

“Isomerism” means compounds that have identical molecular formulae but differ in the sequence of bonding of their atoms or in the arrangement of their atoms in space. Isomers that differ in the arrangement of their atoms in space are termed “stereoisomers”. Stereoisomers that are not mirror images of one another are termed “diastereoisomers”, and stereoisomers that are non-superimposable mirror images of each other are termed “enantiomers” or sometimes optical isomers. A mixture containing equal amounts of individual enantiomeric forms of opposite chirality is termed a “racemic mixture”.

A carbon atom bonded to four nonidentical substituents is termed a “chiral center”.

“Chiral isomer” means a compound with at least one chiral center. Compounds with more than one chiral center may exist either as an individual diastereomer or as a mixture of diastereomers, termed “diastereomeric mixture”. When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116).

“Geometric isomer” means the diastereomers that owe their existence to hindered rotation about double bonds. These configurations are differentiated in their names by the prefixes cis and trans, or Z and E, which indicate that the groups are on the same or opposite side of the double bond in the molecule according to the Cahn-Ingold-Prelog rules.

Furthermore, the structures and other compounds discussed in this disclosure include all atropic isomers thereof. “Atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques; it has been possible to separate mixtures of two atropic isomers in select cases.

“Tautomer” is one of two or more structural isomers that exist in equilibrium and is readily converted from one isomeric form to another. This conversion results in the formal migration of a hydrogen atom accompanied by a switch of adjacent conjugated double bonds. Tautomers exist as a mixture of a tautomeric set in solution. In solid form, usually one tautomer predominates. In solutions where tautomerization is possible, a chemical equilibrium of the tautomers will be reached. The exact ratio of the tautomers depends on several factors, including temperature, solvent and pH. The concept of tautomers that are interconvertible by tautomerizations is called tautomerism.

Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose.

Common tautomeric pairs are: ketone-enol, amide-nitrile, lactam-lactim, amide-imidic acid tautomerism in heterocyclic rings (e.g., in nucleobases such as guanine, thymine and cytosine), amine-enamine and enamine-enamine.

It is to be understood that the compounds of the present disclosure may be depicted as different tautomers. It should also be understood that when compounds have tautomeric forms, all tautomeric forms are intended to be included in the scope of the present disclosure, and the naming of the compounds does not exclude any tautomer form.

The term “crystal polymorphs”, “polymorphs” or “crystal forms” means crystal structures in which a compound (or a salt or solvate thereof) can crystallize in different crystal packing arrangements, all of which have the same elemental composition. Different crystal forms usually have different X-ray diffraction patterns, infrared spectral, melting points, density hardness, crystal shape, optical and electrical properties, stability and solubility. Recrystallization solvent, rate of crystallization, storage temperature, and other factors may cause one crystal form to dominate. Crystal polymorphs of the compounds can be prepared by crystallization under different conditions.

Additionally, the compounds of the present disclosure, for example, the salts of the compounds, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

“Solvate” means solvent addition forms that contain either stoichiometric or non stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as 1120.

As used herein, the term “analog” refers to a chemical compound that is structurally similar to another but differs slightly in composition (as in the replacement of one atom by an atom of a different element or in the presence of a particular functional group, or the replacement of one functional group by another functional group). Thus, an analog is a compound that is similar or comparable in function and appearance, but not in structure or origin to the reference compound.

As defined herein, the term “derivative” refers to compounds that have, a common core structure, and are substituted with various groups as described herein.

The term “bioisostere” refers to a compound resulting from the exchange of an atom or of a group of atoms with another, broadly similar, atom or group of atoms. The objective of a bioisosteric replacement is to create a new compound with similar biological properties to the parent compound. The bioisosteric replacement may be physicochemically or topologically based. Examples of carboxylic acid bioisosteres include, but are not limited to, acyl sulfonimides, tetrazoles, sulfonates and phosphonates. See, e.g., Patani and LaVoie, Chem. Rev. 96, 3147-3176, 1996.

In some aspects, a MetAP2 inhibitor can be administered by subcutaneous injection (SC).

In some aspects, a MetAP2 inhibitor can be administered about every four days (Q4D).

In some aspects, a MetAP2 inhibitor can be administered about once every day (QD), about once every two days (Q2D), about once every three days (Q3D), about once every four days (Q4D), about once every 5 days (Q5D), about once every 6 days (Q6D), about once every 7 days (Q7D), about once every 8 days (Q8D), about once every 9 days (Q9D), about once every 10 days (Q10D), about once every 11 days (Q11D), about once every 12 days (Q12D), about once every 13 days (Q13D), about once every 14 days (Q14D), or about once every 15 days (Q15D). In some aspects, a MetAP2 inhibitor can be administered about once every 7 days (Q7D). In some aspects, a MetAP2 inhibitor can be administered about once every 14 days (Q14D).

In some aspects, a MetAP2 inhibitor can be administered in an amount of about 1 mg/m², or about 2 mg/m², or about 3 mg/m², or about 4 mg/m², or about 5 mg/m², or about 6 mg/m², or about 7 mg/m², or about 8 mg/m², or about 9 mg/m², or about 10 mg/m², or about 11 mg/m², or about 12 mg/m², or about 13 mg/m², or about 14 mg/m², or about 15 mg/m², or about 16 mg/m² or about 17 mg/m², or about 18 mg/m, or about 19 mg/m², or about 20 mg/m, or about 21 mg/m², or about 22 mg/m², or about 23 mg/m², or about 24 mg/m², or about 25 mg/m², or about 26 mg/m², or about 27 mg/m², or about 28 mg/m², or about 29 mg/m², or about 30 mg/m², or about 31 mg/m² or about 32 mg/m², or about 33 mg/m², or about 34 mg/m², or about 35 mg/m², or about 36 mg/m², or about 37 mg/m², or about 38 mg/m², or about 39 mg/m², or about 40 mg/m², or about 41 mg/m² or about 42 mg/m², or about 43 mg/m², or about 44 mg/m², or about 45 mg/m², or about 46 mg/m², or about 47 mg/m², or about 48 mg/n, or about 49 mg/m², or about 50 mg/m² or about 51 mg/m², or about 52 mg/n, or about 53 mg/m², or about 54 mg/m², or about 55 mg/m², or about 56 mg/m² or about 57 mg/m², or about 58 mg/m², or about 59 mg/m², or about 60 mg/ml, or about mg/m², or about 61 mg/nm, or about 62 mg/m², or about 63 mg/m², or about 64 mg/n, or about 65 mg/m² or about 66 mg/m², or about 67 mg/m, or about 68 mg/m², or about 69 mg/m², or about 70 mg/m² or about 81 mg/n, or about 82 mg/m², or about 83 mg/m² or about 84 mg/m², or about 85 mg/m² or about 86 mg/m², or about 87 mg/m², or about 88 mg/m², or about 89 mg/m², or about 90 mg/m², or about 91 mg/m², or about 92 mg/m², or about 93 mg/nm, or about 94 mg/m² or about 95 mg/m², or about 96 mg/m², or about 97 mg/m, or about 98 mg/m, or about 99 mg/2, or about 100 mg/m².

In some aspects, a MetAP2 inhibitor can be administered in an amount of about 49 mg/m². In some aspects, a MetAP2 inhibitor can be administered in an amount of about 39 mg/m² to about 59 mg/m². In some aspects, a MetAP2 inhibitor can be administered in an amount of about 44 mg/m² to about 54 mg/m².

In some aspects, a MetAP2 inhibitor can be administered in an amount of about 36 mg/m², In some aspects, a MetAP2 inhibitor can be administered in an amount of about 26 mg/m² to about 49 mg/m. In some aspects, a MetAP2 inhibitor can be administered in an amount of about 31 mg/m² to about 65 mg/m².

In some aspects, a MetAP2 inhibitor can be administered in an amount of about 65 mg/m². In some aspects, a MetAP2 inhibitor can be administered in an amount of about 55 mg/m² to about 75 mg/m. In some aspects, a MetAP2 inhibitor can be administered in an amount of about 60 mg/m² to about 70 mg/m².

In some aspects, a therapeutically effective amount of a MetAP2 inhibitor can be about 1 mg/m², or about 2 mg/m², or about 3 mg/m², or about 4 mg/m², or about 5 mg/m², or about 6 mg/m², or about 7 mg/m², or about 8 mg/m², or about 9 mg/m², or about 10 mg/m², or about 11 mg/m², or about 12 mg/m², or about 13 mg/m², or about 14 mg/m², or about 15 mg/m², or about 16 m g/m², or about 17 mg/m², or about 18 mg/m², or about 19 ng/m², or about 20 mg/m², or about 21 mg/n, or about 22 mg/m², or about 2.3 mg/m², or about 24 mg/nm, or about 25 mg/m², or about 26 mg/m², or about 27 mg/m², or about 28 mg/m², or about 29 mg/m², or about 30 mg/m², or about 31 mg/nm, or about 32 mg/m², or about 33 mg/m², or about 34 mg/nm, or about 35 mg/m², or about 36 ng/m², or about 37 mg/m², or about 38 mg/m², or about 39 mg/m², or about 40 mg/m², or about 41 mg/m², or about 42 mg/m², or about 43 mg/m², or about 44 mg/m², or about 45 mg/m², or about 46 mg/m², or about 47 mg/m², or about 48 mg/m², or about 49 mg/m², or about 50 mg/m², or about 51 ng/n, or about 52 mg/m², or about 53 mg/m², or about 54 ng/n, or about 55 mg/m², or about 56 mg/m², or about 57 mg/m², or about 58 mg/m², or about 59 mg/m², or about 60 mg/m², or about mg/m², or about 61 mg/m², or about 62 mg/m², or about 63 mg/m², or about 64 mg/m², or about 65 mg/m², or about 66 mg/m², or about 67 mg/m², or about 68 mg/m², or about 69 mg/m², or about 70 mg/m², or about 81 mg/m², or about 82 mg/m² or about 83 mg/m², or about 84 mg/m², or about 85 mg/m², or about 86 mg/m², or about 87 mg/m², or about 88 mg/m², or about 89 mg/m², or about 90 mg/m², or about 91 mg/m², or about 92 mg/m², or about 93 mg/m², or about 94 mg/m², or about 95 mg/m², or about 96 mg/m, or about 97 mg/m², or about 98 mg/m², or about 99 mg/nm, or about 100 mg/m².

In some aspects, a therapeutically effective amount of a MetAP2 inhibitor can be about 49 mg/m². In some aspects, a therapeutically effective amount of a MetAP2 inhibitor can be about 39 mg/m² to about 59 mg/m². In some aspects, a therapeutically effective amount of a MetAP2 inhibitor can be about 44 mg/m² to about 54 mg/m².

In some aspects, a therapeutically effective amount of a MetAP2 inhibitor can be about 36 mg/m². In some aspects, a therapeutically effective amount of a MetAP2 inhibitor can be about 26 mg/m² to about 49 mg/m². In some aspects, a therapeutically effective amount of a MetAP2 inhibitor can be about 31 mg/m² to about 49 mg/m².

In some aspects, a therapeutically effective amount of a MetAP2 inhibitor can be about 65 mg/m². In some aspects, a therapeutically effective amount of a MetAP2 inhibitor can be about 55 mg/m² to about 75 mg/m². In some aspects, a therapeutically effective amount of a MetAP2 inhibitor can be about 60 ng/m² to about 70 mg/m².

In some aspects, a MetAP2 inhibitor can be administered in an amount of about 10 mg, or about 20 mg, or about 30 mg, or about 40 mg, or about 50 mg, or about 60 mg, or about 70 mg, or about 80 mg, or about 90 mg, or about 100 mg or about 110 mg, or about 120 mg, or about 130 mg, or about 140 mg, or about 150 mg, or about 160 mg, or about 170 mg, or about 180 mg, or about 190 mg, or about 200 mg. In some aspects a MetAP2 inhibitor can be administered in an amount of about 80 mg. In some aspects, a MetAP2 inhibitor can be administered in an amount of about of about 70 mg to about 90 mg. In some aspects, a MetAP2 inhibitor can be administered in an amount of about 75 mg to about 85 mg.

In some aspects, a therapeutically effective amount of a MetAP2 inhibitor can be about 10 mg, or about 20 mg, or about 30 mg, or about 40 mg, or about 50 mg, or about 60 mg, or about 70 mg, or about 80 mg, or about 90 mg, or about 100 mg. In some aspects a therapeutically effective amount of a MetAP2 inhibitor can be about 80 mg. In some aspects, a therapeutically effective amount of a MetAP2 inhibitor can be about of about 70 mg to about 90 mg. In some aspects, a therapeutically effective amount of a MetAP2 inhibitor can be about 75 mg to about 85 mg.

CDK4/6 Inhibitors

As used herein, the term “CDK4/6 inhibitor” is used to refer to a compound that inhibits cyclin-dependent kinase CDK4 and/or cyclin-dependent kinase CDK6.

As would be appreciated by the skilled artisan, CDK4/6 inhibitors have also been shown in some cases to inhibit cyclin-dependent kinase CDK2. Thus, as used herein, the term CDK4/6 inhibitor can also refer to a compound that inhibit cyclin-dependent kinase CDK4 and/or cyclin-dependent kinase CDK6 and/or cyclin-dependent kinase CDK2.

In some aspects, a CDK4/6 inhibitor can be selected from palbociclib, abemaciclib, ribociclib, trilaciclib, SHR-6390, FCN-437c, lerociclib, milciclib, PF-06873600, XZP-3287, zotiraciclib, BEBT-209, BPI-16350, CS-3′002, fadraciclib, HS-10342, ON-123300, PF-06842874, TQ-05510, BPI-1178, JS-101, NUV-122, AU-294, CCT-68127, ETH-155008, HEC-80797, JRP-890, JS-104, NEOS-518, PF-07104091, PF-07220060, RMC-4550, SRX-3177, VS-2370, VS-2370. In some aspects, a CDK4/6 inhibitor can be selected from any of the pharmaceutically acceptable salts of the aforementioned compounds.

In some aspects, a CDK4/6 inhibitor can be palbociclib, or a pharmaceutically acceptable salt thereof.

In some aspects, palbociclib can be administered orally. In some aspects, palbociclib can be administered once daily. In some aspects, palbociclib can be administered at an amount of about 75 mg/day, or about 100 mg/day, or about 125 mg/day, or about 150 mg/day, or about 175 mg/day, or about 200 mg/day. In some aspects, palbociclib can be administered in an amount of about 125 mg/day. In some aspects, palbociclib can be administered in an amount of about 50 mg/day to about 150 mg/day, or about 75 mg/day to about 175 mg/day, or about 100 mg/day to about 200 mg/day, or about 125 mg/day to about 225 mg/day, or about 150 to about 250 mg/day. In some aspects, palbociclib can be administered one daily for about 21 days, followed by about 7 days of no administration.

In some aspects, a therapeutically effective amount of palbociclib can be about 75 mg/day, or about 100 mg/day, or about 125 mg/day, or about 150 mg/day, or about 175 mg/day, or about 200 mg/day. In some aspects, a therapeutically effective amount of palbociclib can be about 125 mg/day. In some aspects, a therapeutically effective amount of palbociclib can be about 50 mg/day to about 150 mg/day, or about 75 mg/day to about 175 mg/day, or about 100 mg/day to about 200 mg/day, or about 125 mg/day to about 225 mg/day, or about 150 to about 250 mg/day.

In some aspects, a therapeutically effective amount of palbociclib can be about 75 mg, or about 100 mg, or about 125 mg, or about 150 mg, or about 175 mg, or about 200 mg. In some aspects, a therapeutically effective amount of palbociclib can be about 125 mg. In some aspects, a therapeutically effective amount of palbociclib can be about 50 ng to about 150 mg, or about 75 mg to about 175 mg, or about 100 mg to about 200 mg, or about 125 mg to about 225 mg, or about 150 to about 250 mg.

In some aspects, a CDK4/6 inhibitor can be abemaciclib, or a pharmaceutically acceptable salt thereof.

In some aspects, abemaciclib can be administered orally. In some aspects, abemaciclib can be administered twice daily. In some aspects, abemaciclib can be administered in an amount of about 75 mg administered twice daily (total about 150 mg/day), or about 100 mg administered twice daily (total about 200 mg/day), or about 125 mag administered twice daily (total about 250 mg/day), or about 150 mg administered twice daily (total about 300 mg/day), or about 175 mg administered twice daily (total about 350 mg/day), or about 200 mg administered twice daily (total about 400 mg/day), or about 225 mg administered twice daily (total about 450 mg/day). In some aspects, abemaciclib can be administered in an amount of about 50 mg/day to about 150 mg/day, or about 75 mg/day to about 175 mg/day, or about 100 mg/day to about 200 mg/day, or about 125 mg/day to about 225 mg/day, or about 150 to about 250 mg/day, or about 175 trig/day to about 275 mg/day, or about 200 mg/day to about 300 mg/day, or about 225 mg/day to about 325 mg/day, or about 250 mg/day or about 350 mg/day, or about 275 mg/day or about 375 mg/day, or about 300 mg/day to about 400 mg/day, or about 325 mg/day to about 425 mg/day, or about 350 mg/day to about 450 mg/day, or about 375 mg/day to about 475 mg/day, or about 400 mg/day to about 500 mg/day, or about 425 mg/day to about 525 mg/day, or about 450 mg/day to about 550 mg/day, or about 475 mg/day to about 575 mg/day, or about 500 mg/day to about 600 mg/day, or about 525 mg/day to about 625 mg/day, or about 550 mg/day to about 650 mg/day, or about 575 mg/day to about 675 mg/day, or about 600 mg/day to about 700 mg/day.

In some aspects, a therapeutically effective amount of abemaciclib can be about 75 mg/day, or about 100 mg/day, or about 125 mg/day, or about 150 mg/day, or about 175 mg/day, or about 200 mg/day, or about 225 mg/day, or about 250 mg/day, or about 275 mg/day, or about 300 mg/day, or about 325 mg/day, or about 350 mg/day, or about 375 mg/day, or about 400 mg/day, or about 425 mg/day, or about 450 mg/day, or about 475 mg/day or about 500 mg/day. In some aspects, a therapeutically effective amount of abemaciclib can be about 50 mg/day to about 150 mg/day, or about 75 mg/day to about 175 mg/day, or about 100 mg/day to about 200 mg/day, or about 125 mg/day to about 225 mg/day, or about 150 to about 250 mg/day, or about 175 mg/day to about 275 mg/day, or about 200 mg/day to about 300 mg/day, or about 225 mg/day to about 325 mg/day, or about 250 mg/day or about 350 mg/day, or about 275 mg/day or about 375 mg/day, or about 300 mg/day to about 400 mg/day, or about 325 mg/day to about 425 mg/day, or about 350 mg/day to about 450 mg/day, or about 375 mg/day to about 475 mg/day, or about 400 mg/day to about 500 mg/day, or about 425 mg/day to about 525 mg/day, or about 450 mg/day to about 550 mg/day, or about 475 mg/day to about 575 mg/day, or about 500 mg/day to about 600 mg/day, or about 525 mg/day to about 625 mg/day, or about 550 mg/day to about 650 mg/day, or about 575 mg/day to about 675 mg/day, or about 600 mg/day to about 700 mg/day.

In some aspects, a therapeutically effective amount of abemaciclib can be about 75 mg, or about 100 mg, or about 125 mg, or about 150 mg, or about 175 mg, or about 200 mg, or about 225 tug, or about 250 mg, or about 275 mg, or about 300 mg, or about 325 mg, or about 350 mg, or about 375 mg, or about 400 mg, or about 425 mg, or about 450 mg, or about 475 mg or about 500 mg. In some aspects, a therapeutically effective amount of abemaciclib can be about 50 mg to about 150 mg, or about 75 mg to about 175 mg, or about 100 mg to about 200 mg, or about 125 tug to about 225 mg, or about 150 to about 250 mg, or about 175 mg to about 275 mg, or about 200 mg to about 300 mg, or about 225 mg to about 325 mg, or about 250 mg or about 350 rug, or about 275 mg or about 375 mg, or about 300 mg to about 400 mg, or about 325 mg to about 425 mg, or about 350 mg to about 450 mg, or about 375 mg to about 475 mg, or about 400 mg to about 500 mg, or about 425 mg to about 525 mg, or about 450 mg to about 550 mg, or about 475 mg to about 575 ng, or about 500 mg to about 600 mg, or about 525 ng to about 625 mg, or about 550 mg to about 650 mg, or about 575 mg to about 675 mg, or about 600 mg to about 700 mg.

In some aspects, a CDK4/6 inhibitor can be ribociclib, or a pharmaceutically acceptable salt thereof. In some aspects, the pharmaceutically acceptable salt can be ribociclib succinate.

In some aspects, ribociclib can be administered orally. In some aspects, ribociclib can be administered one daily. In some aspects, ribociclib can be administered in an amount of about 100 mg/day, or about 200 mg/day, or about 300 mg/day, or about 400 mg/day, or about 500 mg/day, or about 600 mg/day, or about 700 mg/day. In some aspects, ribociclib can be administered in an amount of about 600 mg/day. In some aspects, ribociclib can be administered in an amount of about 50 mg/day to about 150 mg/day, or about 75 mg/day to about 175 mg/day, or about 100 mg/day to about 200 mg/day, or about 125 mg/day to about 225 mg/day, or about 150 to about 250 mg/day, or about 175 mg/day to about 275 mg/day, or about 200 mg/day to about 300 mg/day, or about 225 mg/day to about 325 mg/day, or about 250 mg/day or about 350 mg/day, or about 275 mg/day or about 375 mg/day, or about 300 mg/day to about 400 mg/day, or about 325 mg/day to about 425 mg/day, or about 350 mg/day to about 450 mg/day, or about 375 mg/day to about 475 mg/day, or about 400 mg/day to about 500 mg/day, or about 425 mg/day to about 525 mg/day, or about 450 mg/day to about 550 mg/day, or about 475 mg/day to about 575 mg/day, or about 500 mg/day to about 600 mg/day, or about 525 mg/day to about 625 mg/day, or about 550 mg/day to about 650 mg/day, or about 575 mg/day to about 675 mg/day, or about 600 mg/day to about 700 mg/day. In some aspects, palbociclib can be administered one daily for about 21 days, followed by about 7 days of no administration.

In some aspects, a therapeutically effective amount of ribociclib can be about 100 mg/day, or about 200 mg/day, or about 300 mg/day, or about 400 mg/day, or about 500 mg/day, or about 600 mg/day, or about 700 mg/day. In some aspects, a therapeutically effective amount of ribociclib can be about 600 mg/day. In some aspects, a therapeutically effective amount of ribociclib can be about 50 mg/day to about 150 mg/day, or about 75 mg/day to about 175 mg/day, or about 100 mg/day to about 200 mg/day, or about 125 mg/day to about 225 mg/day, or about 150 to about 250 mg/day, or about 175 mg/day to about 275 mg/day, or about 200 mg/day to about 300 mg/day, or about 225 mg/day to about 325 mg/day, or about 250 mg/day or about 350 mg/day, or about 275 mg/day or about 375 mg/day, or about 300 mg/day to about 400 mg/day, or about 325 mg/day to about 425 mg/day, or about 350 mg/day to about 450 mg/day, or about 375 mg/day to about 475 mg/day, or about 400 mg/day to about 500 mg/day, or about 425 mg/day to about 525 mg/day, or about 450 mg/day to about 550 mg/day, or about 475 mg/day to about 575 mg/day, or about 500 mg/day to about 600 mg/day, or about 525 mg/day to about 625 rug/day, or about 550 rug/day to about 650 rug/day, or about 575 mg/day to about 675 mg/day, or about 600 mg/day to about 700 mg/day.

In some aspects, a therapeutically effective amount of ribociclib can be about 100 rug, or about 200 rug, or about 300 mg, or about 400 rug, or about 500 mg, or about 600 mg, or about 700 mg. In some aspects, a therapeutically effective amount of ribociclib can be about 600 rug. In some aspects, a therapeutically effective amount of ribociclib can be about 50 mg to about 150 mg, or about 75 mg to about 175 mg, or about 100 mg to about 200 mg, or about 125 mg to about 225 mg, or about 150 to about 250 mg, or about 175 mg to about 275 mg, or about 200 mg to about 300 mg, or about 225 mg to about 325 mg, or about 250 mg or about 350 mg, or about 275 mg or about 375 mg, or about 300 mg to about 400 mg, or about 325 mg to about 425 mg, or about 350 mg to about 450 mg, or about 375 mg to about 475 mg, or about 400 mg to about 500 mg, or about 425 mg to about 525 mg, or about 450 mg to about 550 mg, or about 475 mg to about 575 mg, or about 500 mg to about 600 rug, or about 525 mg to about 625 mg, or about 550 mg to about 650 mg, or about 575 rug to about 675 rug, or about 600 mg to about 700 mg.

CDK4/6 inhibitors can be further administered with at least one additional therapeutic. Accordingly, the compositions of the present disclosure can comprise a combination of at least one MetAP2 inhibitor of the present disclosure, at least one CDK4/6 inhibitor and at least one additional therapeutic.

In some aspects, the at least one additional therapeutic can comprise a hormone therapy.

In some aspects, the at least one additional therapeutic can comprise an aromatase inhibitor.

In some aspects, the aromatase inhibitor can comprise a non-steroidal aromatase inhibitor.

In some aspects, an aromatase inhibitor can comprise anastrozole, exemestane, letrozole or any combination thereof.

In some aspects, the at least one additional therapeutic can comprise a selective estrogen receptor degrader (SERD). In some aspects, a SERD can comprise fulvestrant.

In some aspects, the at least one additional therapeutic can comprise a gonadotropin releasing hormone agonist. In some aspects, a gonadotropin releasing hormone agonist can comprise goserelin.

In some aspects, the at least one additional therapeutic can comprise a PI3K inhibitor, an AKT inhibitor, an mTOR inhibitor or a PI3K/Akt/mTOR pathway inhibitor.

In some aspects, the at least one additional therapeutic can comprise Serabelisib (TAK-117), BYL-719, AZD5363 (capavasertib), ipaseratib (GDC0068), (paclitaxel+sirolimus+tanespimycin), (paelitaxel+sirolimus+tanespimycin), A-443654, AB-610, ACP-2127, ADC-0008830, AE-116, AEZS-126, AEZS-127, afuresertib+trametinib, AL-58203, AL-58805, AL-58922, ALM-301, AP-185, AP-23675, AP-23841, apitolisib, ARQ-751, ASP-7486, AST-0669, AT-104, AT-13148, AUM-302, AZD-3147, AZD-8055, AZD-8154, BAY-1001931, BAY-1125976, BAY-125976, BGT-226, bimiralisib, BN-107, BN-108, borussertib, buformin, BVD-723, capivasertib, CC-115, CC-2141, CC-2142, Certican ODT, CL-27, COTI-2, CT-365, dactolisib tosylate, DC-120, DHM-25, dihydroarterisinin, DS-3078, DS-7423, duvelisib, EM-101, everolimus, FP-208, FT-1518, FXY-1, galarmin, GDC-0349, gedatolisib, GM-6, GNE-317, GNE-555, GSK-690693, GT-0486, HD-148 series, NEC-68498, HM-032, HM-5016699, HMPL-518, ipatasertib, IPI-549, ISC-4, J-9, JRP-890, KIT-2014, KS-99, LD-101, lithium carbonate, LY-2503029, LY-2780301, M-2698, ME-344, miransertib mesylate, MK-2206, MKC-1, monepantel, NISC-6, nPT-mTOR, NSC-765844, NV-128, onatasertib, ONC-201, ONC-222, ONC-235, OSU-53, OT-043, OT-043, P-7170, P-7170, PBD-1226, perifosine, PF-04691502, pimasertib hydrochloride+voxtalisib, PK-179, PQR-311, PQR-316, PQR-401, PQR-4XX, PQR-514, PQR-530, PQR-620, PWT-33597, PX-316, recilisib sodium, RES-529, ridaforolirnus, RMC-5552, RP-6503, RV-1729, RX-0183, RX-0201, RX-0201N, RX-0301, RX-1792, RX-8243, samotolisib, sapanisertib, SB-2602, SCC-31, SF-1126, SF-2523, SN-202, SPR-965, SR-13668, STP-503, SX-MTRI, TAFA-93, TAM-01, TAM-03, TAS-117, TASP-0415914, TE-7105, ternsiroiimus, tenalisib, TOP-216, trarnerinib dimethyl sulfoxide+uprosertib, triciribine phosphate, UB-1201, uprosertib, VCC-405567, VCC-668662, vistusertib, VLI-27, voxtalisib, VS-5584, WX-008, WXFL-10030390, X-387, X-414, X-480, XL-388, XL-418, XP-105, Y-31, Zortress or any combination thereof.

Treated Subjects and Cancers

In some aspects, the subject in need thereof is an animal. In some aspects, the animal can be a mammal. In some aspects, the subject in need thereof is a human.

In some aspects, the subject in need thereof is a human of 18 years or older. In some aspects, the subject in need thereof is a human younger than 18 years.

In some aspects, the subject in need thereof has a cancer. In some aspects, the cancer is characterized by at least one tumor present in the subject.

The terms “cancer” and “cancerous” refer to or describe the physiological condition in mammals that is typically characterized by unregulated cell growth. Included in this definition are benign and malignant cancers. Examples of cancer include but are not limited to, carcinoma, lymphoma, blastoma, sarcoma, leukemia and germ cell tumors. More particular examples of such cancers include adrenocortical carcinoma, bladder urothelial carcinoma, breast invasive carcinoma, cervical squamous cell carcinoma, endocervical adenocarcinoma, cholangiocarcinoma, colon adenocarcinoma, lymphoid neoplasm diffuse large B-cell lymphoma, esophageal carcinoma, glioblastoma multiforme, head and neck squamous cell carcinoma, kidney chromophobe, kidney renal clear cell carcinoma, kidney renal papillary cell carcinoma, acute myeloid leukemia, brain lower grade glioma, liver hepatocellular carcinoma, lung adenocarcinoma, lung squamous cell carcinoma, mesothelioma, ovarian serous cystadenocarcinoma, pancreatic adenocarcinoma, pheochromocytoma, paraganglioma, prostate adenocarcinoma, rectum adenocarcinoma, sarcoma, skin cutaneous melanoma, stomach adenocarcinoma, testicular germ cell tumors, thyroid carcinoma, thymoma, uterine carcinosarcoma, uveal melanoma Other examples include breast cancer, lung cancer, lymphoma, melanoma, liver cancer, colorectal cancer, ovarian cancer, bladder cancer, renal cancer or gastric cancer. Further examples of cancer include neuroendocrine cancer, non-small cell lung cancer (NSCLC), small cell lung cancer, thyroid cancer, endometrial cancer, biliary cancer, esophageal cancer, anal cancer, salivary, cancer, vulvar cancer, cervical cancer, Acute lymphoblastic leukemia (ALL). Acute myeloid leukemia (AML). Adrenal gland tumors, Anal cancer, Bile duct cancer, Bladder cancer, Bone cancer, Bowel cancer, Brain tumors, Breast cancer, Cancer of unknown primary (CUP), Cancer spread to bone, Cancer spread to brain, Cancer spread to liver, Cancer spread to lung, Carcinoid, Cervical cancer, Children's cancers, Chronic lymphocytic leukemia (CLL), Chrome myeloid leukemia (CML), Colorectal cancer, Ear cancer, Endometrial cancer, Eye cancer, Follicular dendritic cell sarcoma, Gallbladder cancer, Gastric cancer, Gastro esophageal junction cancers, Germ cell tumors, Gestational trophoblastic disease (GIT)), Hairy cell leukemia, Head and neck cancer, Hodgkin lymphoma, Kaposi's sarcoma, Kidney cancer, Laryngeal cancer, Leukemia, Gastric linitis plastica, Liver cancer, Lung cancer, Lymphoma, Malignant schwannoma, Mediastinal germ cell tumors, Melanoma skin cancer, Men's cancer, Merkel cell skin cancer, Mesothelioma, Molar pregnancy, Mouth and oropharyngeal cancer, Myeloma, Nasal and paranasal sinus cancer, Nasopharvngeal cancer, Neuroblastoma, Neuroendocrine tumors, Non-Hodgkin lymphoma (NHL), Esophageal cancer, Ovarian cancer, Pancreatic cancer, Penile cancer, Persistent trophoblastic disease and choriocarcinoma, Pheochromocytoma, Prostate cancer, Pseudomyxoma peritonei, Rectal cancer. Retinoblastoma, Salivary gland cancer, Secondary' cancer, Signet cell cancer, Skin cancer, Small bowel cancer, Soft tissue sarcoma, Stomach cancer, T cell childhood non Hodgkin lymphoma (NHL). Testicular cancer, Thymus gland cancer, Thyroid cancer, Tongue cancer, Tonsil cancer, Tumors of the adrenal gland, Uterine cancer. Vaginal cancer, Vulval cancer, Wilms' tumor, Womb cancer and Gynaecological cancer. Examples of cancer also include, but are not limited to, Hematologic malignancies, Lymphoma, Cutaneous T-cell lymphoma, Peripheral T-cell lymphoma, Hodgkin's lymphoma, Non-Hodgkin's lymphoma, Multiple myeloma, Chrome lymphocytic leukemia, chronic myeloid leukemia, acute myeloid leukemia, Myelodysplastic syndromes, Myelofibrosis, Biliary tract cancer, Hepatocellular cancer, Colorectal cancer, Breast cancer, Lung cancer, Non-small cell lung cancer, Ovarian cancer, Thyroid Carcinoma, Renal Cell Carcinoma, Pancreatic cancer, Bladder cancer, skin cancer, malignant melanoma, merkel cell carcinoma, Uveal Melanoma or Glioblastoma multiforme.

In some aspects, the cancer is a carcinoma, a lymphoma, a blastoma, a sarcoma, a leukemia, a brain cancer, a breast cancer, a blood cancer, a bone cancer, a lung cancer, a skin cancer, a liver cancer, an ovarian cancer, a bladder cancer, a renal cancer, a kidney cancer, a gastric cancer, a thyroid cancer, a pancreatic cancer, an esophageal cancer, a prostate cancer, a cervical cancer, a uterine cancer, a stomach cancer, a soft tissue cancer, a laryngeal cancer, a small intestine cancer, a testicular cancer, an anal cancer, a vulvar cancer, a joint cancer, an oral cancer, a pharynx cancer or a colorectal cancer.

In some aspects, the cancer is breast cancer.

In some aspects, the breast cancer is metastatic breast cancer. As used herein, metastatic breast cancer is stage III or IV breast cancer that has spread to another part of the body, including, but not limited to, the liver, brain, bones, etc.

In some aspects, the breast cancer is human epidermal growth factor 2 (HER2)-negative breast cancer.

In some aspects, the breast cancer is HR+HER2− breast cancer.

In some aspects, the breast cancer can be a Luminal A breast cancer. In some aspects, the breast cancer can be a Luminal B breast cancer. In some aspects, the breast cancer can be a triple negative or basal-like breast cancer. In some aspects the breast cancer can be a HER2-enriched breast cancer.

In some aspects, the cancer is a head and neck cancer.

In some aspects, the cancer is a non-small cell lung cancer.

In some aspects, the cancer is a brain cancer. In some aspects, the brain cancer can be a recurring brain metastasis.

In some aspects, the cancer is a squamous cell carcinoma.

In some aspects, the cancer is a central nervous system tumor.

In some aspects, the cancer is liposarcoma.

In some aspects, the cancer is endometrial carcinoma.

In some aspects, the cancer is a neuroendocrine tumor.

In some aspects, the cancer is a small cell lung cancer (SCLC).

### General Definitions

It is to be understood that the compounds of the present disclosure may be depicted as different tautomers. It should also be understood that when compounds have tautomeric forms, all tautomeric forms are intended to be included in the scope of the present disclosure, and the naming of the compounds does not exclude any tautomer form. It will be understood that certain tautomers may have a higher level of activity than others.

As used herein, the term “crystal polymorphs”, “polymorphs” or “crystal forms” means crystal structures in which a compound (or a salt or solvate thereof) can crystallize in different crystal packing arrangements, all of which have the same elemental composition. Different crystal forms usually have different X-ray diffraction patterns, infrared spectral, melting points, density hardness, crystal shape, optical and electrical properties, stability and solubility. Recrystallization solvent, rate of crystallization, storage temperature, and other factors may cause one crystal form to dominate. Crystal polymorphs of the compounds can be prepared by crystallization under different conditions.

It is to be understood that the compounds of any Formula described herein include the compounds themselves, as well as their salts, and their solvates, if applicable. A salt, for example, can be formed between an anion and a positively charged group (e.g., amino) on a substituted benzene compound. Suitable anions include chloride, bromide, iodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate (e.g., trifluoroacetate).

As used herein, the term “pharmaceutically acceptable anion” refers to an anion suitable for forming a pharmaceutically acceptable salt. Likewise, a salt can also be formed between a cation and a negatively charged group (e.g., carboxylate) on a substituted benzene compound. Suitable cations include sodium ion, potassium ion, magnesium ion, calcium ion, and an ammonium cation such as tetramethylammonium ion. The substituted benzene compounds also include those salts containing quaternary nitrogen atoms.

It is to be understood that the compounds of the present disclosure, for example, the salts of the compounds, can exist in either hydrated or unhydrated (the anhydrous) form or as solvates with other solvent molecules. Nonlimiting examples of hydrates include monohydrates, dihydrates, etc. Nonlimiting examples of solvates include ethanol solvates, acetone solvates, etc.

As used herein, the term “solvate” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

As used herein, the term “analog” refers to a chemical compound that is structurally similar to another but differs slightly in composition (as in the replacement of one atom by an atom of a different element or in the presence of a particular functional group, or the replacement of one functional group by another functional group). Thus, an analog is a compound that is similar or comparable in function and appearance, but not in structure or origin to the reference compound.

As used herein, the term “derivative” refers to compounds that have a common core structure, and are substituted with various groups as described herein.

As used herein, the term “bioisostere” refers to a compound resulting from the exchange of an atom or of a group of atoms with another, broadly similar, atom or group of atoms. The objective of a bioisosteric replacement is to create a new compound with similar biological properties to the parent compound. The bioisosteric replacement may be physicochemically or topologically based. Examples of carboxylic acid bioisosteres include, but are not limited to, acyl sulfonimides, tetrazoles, sulfonates and phosphonates. See, e.g., Patani and LaVoie, Chem. Rev. 96, 3147-3176, 1996.

It is to be understood that the present disclosure is intended to include all isotopes of atoms occurring in the present compounds. Isotopes include those atoms having the same atomic number but different mass numbers. By way of general example and without limitation, isotopes of hydrogen include tritium and deuterium, and isotopes of carbon include C-13 and C-14.

used herein, the expressions “one or more of A, B, or C,” “one or more A, B, or C,” “one or more of A, B, and C,” “one or more A, B, and C,” “selected from the group consisting of A, B, and C”, “selected from A, B, and C”, and the like are used interchangeably and all refer to a selection from a group consisting of A, B, and/or C, i.e., one or more As, one or more Bs, one or more Cs, or any combination thereof, unless indicated otherwise.

It is to be understood that the present disclosure provides methods for the synthesis of the compounds of any of the Formulae described herein. The present disclosure also provides detailed methods for the synthesis of various disclosed compounds of the present disclosure according to the following schemes as well as those shown in the Examples.

It is to be understood that, throughout the description, where compositions are described as having, including, or comprising specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the invention remains operable. Moreover, two or more steps or actions can be conducted simultaneously.

It is to be understood that the synthetic processes of the disclosure can tolerate a wide variety of functional groups, therefore various substituted starting materials can be used. The processes generally provide the desired final compound at or near the end of the overall process, although it may be desirable in certain instances to further convert the compound to a pharmaceutically acceptable salt thereof.

It is to be understood that compounds of the present disclosure can be prepared in a variety of ways using commercially available starting materials, compounds known in the literature, or from readily prepared intermediates, by employing standard synthetic methods and procedures either known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March's Advanced Organic Chemistry: Reactions, Mechanisms, and Structure, 5^{th} edition, John Wiley & Sons: New York, 2001; Greene, T. W., Wuts, P. G. M., Protective Groups in Organic Synthesis, 3^{rd} edition, John Wiley & Sons: New York, 1999; R. Larock, Comprehensive Organic Transformations, VCH Publishers (1989); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), incorporated by reference herein, are useful and recognized reference textbooks of organic synthesis known to those in the art

It is to be understood that, unless otherwise stated, any description of a method of treatment includes use of the compounds to provide such treatment or prophylaxis as is described herein, as well as use of the compounds to prepare a medicament to treat or prevent such condition. The treatment includes treatment of human or non-human animals including rodents and other disease models.

As used herein, the term “subject” is interchangeable with the term “subject in need thereof”, both of which refer to a subject having a disease or having an increased risk of developing the disease. A “subject” includes a mammal. The mammal can be e.g., a human or appropriate non-human mammal, such as primate, mouse, rat, dog, cat, cow, horse, goat, camel, sheep or a pig. The subject can also be a bird or fowl. In one embodiment, the mammal is a human.

As used herein, the term “treating” or “treat” describes the management and care of a patient for the purpose of combating a disease, condition, or disorder and includes the administration of a compound of the present disclosure, or a pharmaceutically acceptable salt, polymorph or solvate thereof, to alleviate the symptoms or complications of a disease, condition or disorder, or to eliminate the disease, condition or disorder. The term “treat” can also include treatment of a cell in vitro or an animal model.

It is to be understood that a compound of the present disclosure, or a pharmaceutically acceptable salt, polymorph or solvate thereof, can or may also be used to prevent a relevant disease, condition or disorder, or used to identify suitable candidates for such purposes.

As used herein, the term “preventing,” “prevent,” or “protecting against” describes reducing or eliminating the onset of the symptoms or complications of such disease, condition or disorder.

It is to be understood that one skilled in the art may refer to general reference texts for detailed descriptions of known techniques discussed herein or equivalent techniques. These texts include Ausubel et al., Current Protocols in Molecular Biology, John Wiley and Sons, Inc. (2005); Sambrook et al., Molecular Cloning, A Laboratory Manual (3^{th} edition), Cold Spring Harbor Press, Cold Spring Harbor, New York (2000); Coligan et al., Current Protocols in Immunology, John Wiley & Sons, N.Y.; Enna et al., Current Protocols in Pharmacology, John Wiley & Sons, N.Y.; Fingl et al., The Pharmacological Basis of Therapeutics (1975), Remington's Pharmaceutical Sciences, Mack Publishing Co., Easton, PA, 18^{th} edition (1990), Mandell, et al., Principles and Practice of Infectious Diseases, Saunders Publishing (8th edition, 2014). These texts can, of course, also be referred to in making or using an aspect of the disclosure.

As used herein, the term “combination therapy” or “co-therapy” includes the administration of a compound of the present disclosure, or a pharmaceutically acceptable salt, polymorph or solvate thereof, and at least a second agent as part of a specific treatment regimen intended to provide the beneficial effect from the co-action of these therapeutic agents. The beneficial effect of the combination includes, but is not limited to, pharmacokinetic or pharmacodynamic co-action resulting from the combination of therapeutic agents.

It is to be understood that the present disclosure also provides pharmaceutical compositions comprising any compound described herein in combination with at least one pharmaceutically acceptable excipient or carrier.

As used herein, the term “pharmaceutical composition” is a formulation containing the compounds of the present disclosure in a form suitable for administration to a subject. In one embodiment, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this disclosure include powders, sprays, ointments, pastes, creams, lotions, gels, solutions, patches and inhalants. In one embodiment, the active compound is mixed under sterile conditions with a pharmaceutically acceptable carrier, and with any preservatives, buffers, or propellants that are required.

The terms “effective amount” and “therapeutically effective amount” of an agent or compound are used in the broadest sense to refer to a nontoxic but sufficient amount of an active agent or compound to provide the desired effect or benefit.

The term “benefit” is used in the broadest sense and refers to any desirable effect and specifically includes clinical benefit as defined herein. Clinical benefit can be measured by assessing various endpoints, e.g., inhibition, to some extent, of disease progression, including slowing down and complete arrest; reduction in the number of disease episodes and/or symptoms; reduction in lesion size; inhibition (i.e., reduction, slowing down or complete stopping) of disease cell infiltration into adjacent peripheral organs and/or tissues; inhibition (i.e. reduction, slowing down or complete stopping) of disease spread; decrease of auto-immune response, which may, but does not have to, result in the regression or ablation of the disease lesion; relief, to some extent, of one or more symptoms associated with the disorder; increase in the length of disease-free presentation following treatment, e.g., progression-free survival; increased overall survival; higher response rate; and/or decreased mortality at a given point of time following treatment.

As used herein, the term “pharmaceutically acceptable” refers to those compounds, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

It is to be understood that a pharmaceutical composition of the disclosure is formulated to be compatible with its intended route of administration. Examples of routes of administration include parenteral, e.g., intravenous, intradermal, subcutaneous, oral (e.g., inhalation), transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

It is to be understood that a compound or pharmaceutical composition of the disclosure can be administered to a subject in many of the well-known methods currently used for chemotherapeutic treatment. For example, a compound of the disclosure may be injected into the blood stream or body cavities or taken orally or applied through the skin with patches. The dose chosen should be sufficient to constitute effective treatment but not so high as to cause unacceptable side effects. The state of the disease condition and the health of the patient should preferably be closely monitored during and for a reasonable period after treatment.

As used herein, the term “therapeutically effective amount”, refers to an amount of a pharmaceutical agent to treat, ameliorate, or prevent an identified disease or condition, or to exhibit a detectable therapeutic or inhibitory effect. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

It is to be understood that, for any compound, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. Such information can then be used to determine useful doses and routes for administration in humans. Therapeutic/prophylactic efficacy and toxicity may be determined by standard pharmaceutical procedures in cell cultures or experimental animals, e.g., ED₅₀ (the dose therapeutically effective in 50% of the population) and LD₅₀ (the dose lethal to 50% of the population). The dose ratio between toxic and therapeutic effects is the therapeutic index, and it can be expressed as the ratio, LD₅₀/ED₅₀. Pharmaceutical compositions that exhibit large therapeutic indices are preferred. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

The pharmaceutical compositions containing active compounds of the present disclosure may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of the active compounds into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol and sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin, or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The active compounds can be prepared with pharmaceutically acceptable carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the disclosure are dictated by and directly dependent on the unique characteristics of the active compound and the particular therapeutic effect to be achieved.

In therapeutic applications, the dosages of the pharmaceutical compositions used in accordance with the disclosure vary depending on the agent, the age, weight, and clinical condition of the recipient patient, and the experience and judgment of the clinician or practitioner administering the therapy, among other factors affecting the selected dosage. Generally, the dose should be sufficient to result in slowing, and preferably regressing, the symptoms of the disease and also preferably causing complete regression of the disease. An effective amount of a pharmaceutical agent is that which provides an objectively identifiable improvement as noted by the clinician or other qualified observer. Improvement in survival and growth indicates regression. As used herein, the term “dosage effective manner” refers to amount of an active compound to produce the desired biological effect in a subject or cell.

It is to be understood that the pharmaceutical compositions can be included in a container, pack, or dispenser together with instructions for administration.

It is to be understood that, for the compounds of the present disclosure being capable of further forming salts, all of these forms are also contemplated within the scope of the claimed disclosure.

As used herein, the term “pharmaceutically acceptable salts” refer to derivatives of the compounds of the present disclosure wherein the parent compound is modified by making acid or base salts thereof. In some embodiments, the pharmaceutically acceptable salt of a compound (e.g., a β-lactam compound or probenecid described herein) is also a prodrug of the compound. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids. For example, such conventional non-toxic salts include, but are not limited to, those derived from inorganic and organic acids selected from 2-acetoxybenzoic, 2-hydroxyethane sulfonic, acetic, ascorbic, benzene sulfonic, benzoic, bicarbonic, carbonic, citric, edetic, ethane disulfonic, 1,2-ethane sulfonic, fumaric, glucoheptonic, gluconic, glutamic, glycolic, glycollyarsanilic, hexylresorcinic, hydrabamic, hydrobromic, hydrochloric, hydroiodic, hydroxymaleic, hydroxynaphthoic, isethionic, lactic, lactobionic, lauryl sulfonic, maleic, malic, mandelic, methane sulfonic, napsylic, nitric, oxalic, pamoic, pantothenic, phenylacetic, phosphoric, polygalacturonic, propionic, salicylic, stearic, subacetic, succinic, sulfamic, sulfanilic, sulfuric, tannic, tartaric, toluene sulfonic, and the commonly occurring amine acids, e.g., glycine, alanine, phenylalanine, arginine, etc.

As used herein, the term “metabolite” means a product of metabolism of the compound of present disclosure, or pharmaceutically acceptable salts, solvates, diastereomers, and polymorphs thereof, that exhibits a similar activity in vivo to the compound of present disclosure, or pharmaceutically acceptable salts, solvates, diastereomers, and polymorphs thereof.

Other examples of pharmaceutically acceptable salts include hexanoic acid, cyclopentane propionic acid, pyruvic acid, malonic acid, 3-(4-hydroxybenzoyl)benzoic acid, cinnamic acid, 4-chlorobenzenesulfonic acid, 2-naphthalenesulfonic acid, 4-toluenesulfonic acid, camphorsulfonic acid, 4-methylbicyclo-[2.2.2]-oct-2-ene-1-carboxylic acid, 3-phenylpropionic acid, trimethylacetic acid, tertiary butylacetic acid, muconic acid, and the like. The present disclosure also encompasses salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like. In the salt form, it is understood that the ratio of the compound to the cation or anion of the salt can be 1:1, or any ration other than 1:1, e.g., 3:1, 2:1, 1:2, or 1:3.

It is to be understood that all references to pharmaceutically acceptable salts include solvent addition forms (solvates) or crystal forms (polymorphs) as defined herein, of the same salt.

As used herein, the term “prodrug” refers to any agent which, when administered to a mammal, is converted in whole or in part to a targeted compound (e.g., a any of the fumagillol derivatives described herein). In some embodiments, the prodrug of a compound (e.g., any of the fumagillol derivatives described herein) is also a pharmaceutically acceptable salt of the compound.

It is to be understood that the compounds of the present disclosure can also be prepared as esters, for example, pharmaceutically acceptable esters. For example, a carboxylic acid function group in a compound can be converted to its corresponding ester, e.g., a methyl, ethyl or other ester. Also, an alcohol group in a compound can be converted to its corresponding ester, e.g., acetate, propionate or other ester.

The compounds, or pharmaceutically acceptable salts thereof, are administered orally, nasally, transdermally, pulmonary, inhalationally, buccally, sublingually, intraperitoneally, subcutaneously, intramuscularly, intravenously, rectally, intrapleurally, intrathecally and parenterally. In one embodiment, the compound is administered orally. One skilled in the art will recognize the advantages of certain routes of administration.

The dosage regimen utilizing the compounds is selected in accordance with a variety of factors including type, species, age, weight, body surface area (BSA), sex and medical condition of the patient, the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter, or arrest the progress of the condition.

Techniques for formulation and administration of the disclosed compounds of the disclosure can be found in Remington: the Science and Practice of Pharmacy, 19^{th} edition, Mack Publishing Co., Easton, PA (1995). In an embodiment, the compounds described herein, and the pharmaceutically acceptable salts thereof, are used in pharmaceutical preparations in combination with a pharmaceutically acceptable carrier or diluent. Suitable pharmaceutically acceptable carriers include inert solid fillers or diluents and sterile aqueous or organic solutions. The compounds will be present in such pharmaceutical compositions in amounts sufficient to provide the desired dosage amount in the range described herein.

Other features and advantages of the present disclosure are apparent from the different examples. The provided examples illustrate different components and methodology useful in practicing the present disclosure. The examples do not limit the claimed disclosure. Based on the present disclosure the skilled artisan can identify and employ other components and methodology useful for practicing the present disclosure.

In the synthetic schemes described herein, compounds may be drawn with one particular configuration for simplicity. Such particular configurations are not to be construed as limiting the disclosure to one or another isomer, tautomer, regioisomer or stereoisomer, nor does it exclude mixtures of isomers, tautomers, regioisomers or stereoisomers; however, it will be understood that a given isomer, tautomer, regioisomer or stereoisomer may have a higher level of activity than another isomer, tautomer, regioisomer or stereoisomer.

Compounds designed, selected and/or optimized by methods described above, once produced, can be characterized using a variety of assays known to those skilled in the art to determine whether the compounds have biological activity. For example, the molecules can be characterized by conventional assays, including but not limited to those assays described herein, to determine whether they have a predicted or unexpected activity, target binding activity and/or binding specificity.

Furthermore, high-throughput screening can be used to speed up analysis using such assays. As a result, it can be possible to rapidly screen the molecules described herein for activity, using techniques known in the art. General methodologies for performing high-throughput screening are described, for example, in Devlin (1998) High Throughput Screening, Marcel Dekker; and U.S. Pat. No. 5,763,263. High-throughput assays can use one or more different assay techniques including, but not limited to, those described below.

### EXAMPLES

### Example 1

The following is a non-limiting example demonstrating that the MetAP2 inhibitors of the present disclosure and CDK4/6 inhibitors can be used in combination to treat cancer. Moreover, the following non-limiting example demonstrates that the combination of the MetAP2 inhibitors of the present disclosure and CDK4/6 inhibitors exhibits unexpectedly superior anti-tumor activity as compared to the use of either agent alone.

In the following experiment, mice bearing MCF-7 tumors were treated with either a vehicle control, Compound 1 alone, palbociclib alone, or with a combination of Compound 1 and palbociclib. Female nude mice (Charles River) were first implanted with one 90-day (0.72 mg) β-estradiol pellet 3-days prior to cell inoculation. MCF-7 cells (5×10⁶ in 1:1 PBS/Matrigel) were injected into the fourth mammary gland of the mice. Dosing of the various therapeutics began when group mean tumor volume was 125-175 mm³ with no tumors <100 mm³. During the course of the experiment, tumor volume and body weight were measured twice weekly and gross observations were made daily. At the conclusion of the study, whole blood was collected from the treated mice by cardiac puncture for complete blood count (CBC). Moreover, tumors from the mice were dissected and analyzed.

The experimental design is summarized in Table 2.

TABLE 2

| | | | Vehicle | | |
|---|---|---|---|---|---|
| | | | (QD to | Compound 1 | Palbociclib |
| | | | End of | (Q4D to End | (QD to End |
| | | | Study | of Study | of Study |
| Experimental | | Number | Starting | Starting | Starting |
| Group # | Group Description | of Mice | Day 2) | Day 1) | Day 2) |
| 1 | Vehicle (PO) | 10 | X | | |
| 2 | Compound 1 8 mg/kg (SC) | 10 | | X | |
| 3 | Compound 1 8 mg/kg (SC) + | 10 | | X | X |
| | Palbociclib 20 mg/kg (PO) | | | | |
| 4 | Compound 1 8 mg/kg (SC) + | 10 | | X | X |
| | Palbo 40 mg/kg (PO) | | | | |
| 5 | Palbociclib 20 mg/kg (PO) | 10 | | | X |
| 6 | Palbociclib 40 mg/kg (PO) | 10 | | | X |

The first group of mice (Group #1) were administered the vehicle control by oral gavage once a day (QD) starting day 2 of the study to the end of the study.

The second group of mice (Group #2) were administered Compound 1 at a dose of 8 mg/kg by subcutaneous injection every four days (Q4D) starting day 1 of the study to the end of the study.

The third group of mice (Group #3) were administered Compound 1 at a dose of 8 mg/kg by subcutaneous injection every four days starting day 1 of the study to the end of the study and Palbociclib at a dose of 20 mg/kg by oral gavage once a day (QD) starting day 2 of the study to the end of the study.

The fourth group of mice (Group #4) were administered Compound 1 at a dose of 8 mg/kg by subcutaneous injection every four days starting day 1 of the study to the end of the study and Palbociclib at a dose of 40 mg/kg by oral gavage once a day (QD) starting day 2 of the study to the end of the study.

The fifth group of mice (Group #5) were administered Palbociclib at a dose of 20 mg/kg by oral gavage once a day (QD) starting day 2 of the study to the end of the study.

The sixth group of mice (Group #6) were administered Palbociclib at a dose of 40 mg/kg by oral gavage once a day (QD) starting day 2 of the study to the end of the study.

Compound 1 was dissolved in 5% aqueous mannitol (w/v) and sterile filtered prior to administration to the mice by subcutaneous injection.

Palbociclib was dissolved in lactic acid buffer (50 mM, pH 4.0) and sterile filtered prior to administration to the mice by oral gavage.

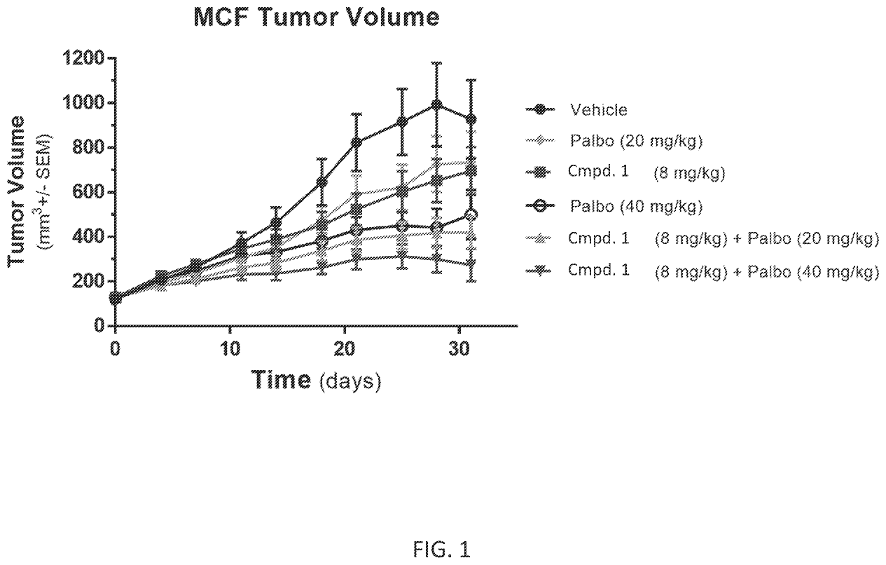

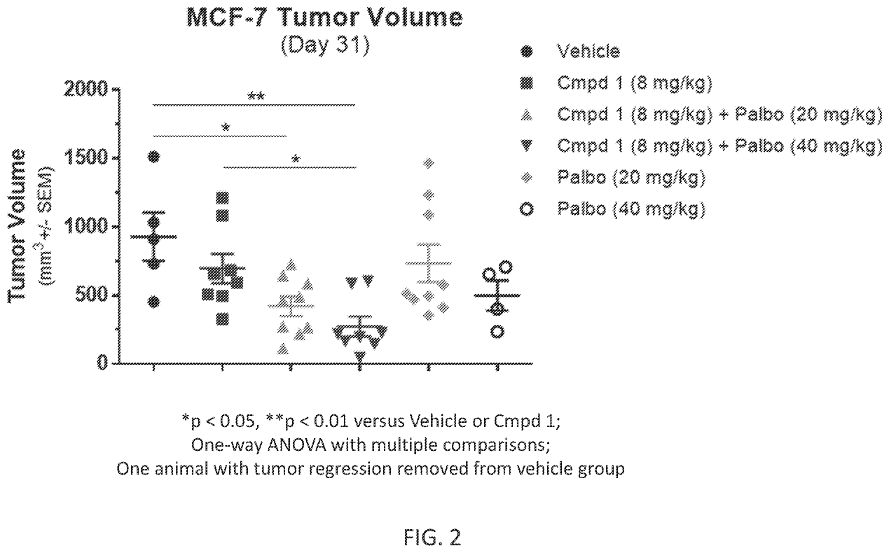

FIG. 1 shows the analysis of MCF tumor volume over the course of the study. FIG. 2 shows the MCF-tumor volume at the conclusion (day 31) of the study. As shown in FIG. 1 and FIG. 2, the combination of Compound 1 (Cmpd. 1) and palbociclib (palbo) resulted in greater reductions in tumor volume as compared to either Compound 1 alone or palbociclib alone.

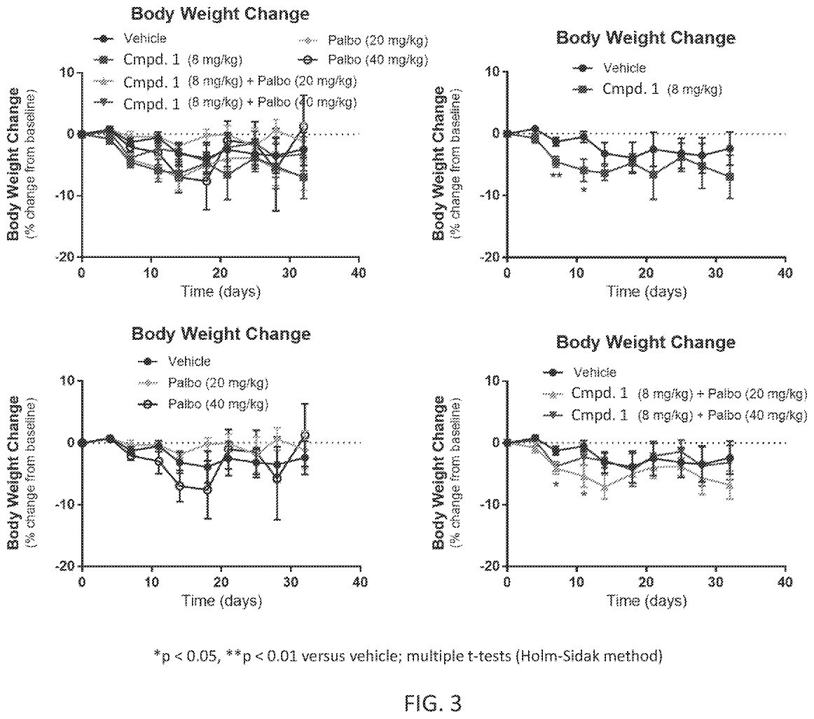

FIG. 3 shows the body weight changes in the various experimental groups.

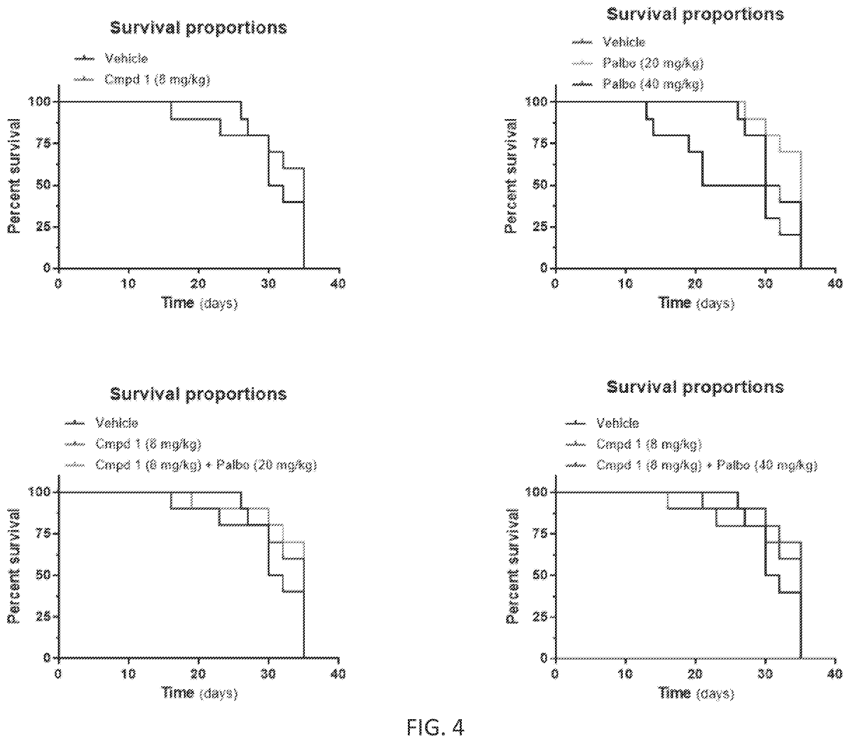

FIG. 4 shows the percent survival in each of the experimental groups over the course of the study.

As would be appreciated by the skilled artisan, several genes and cellular pathways have been implicated in acquired resistance to CDK4/6 inhibitors, such as palbociclib and ribociclib, including, but not limited to, Cyclin E1, CDK2, CDK4, Akt signaling, Autophagy pathways and cancer stem cell initiation pathways. To that end, expression levels of various proteins were analyzed in tumor samples collected at the conclusion of the study to determine how the administration of a combination of MetAP2 inhibitors of the present disclosure and CDK4/6 inhibitors may impact the expression of various proteins implicated in CDK4/6 inhibitor resistance.

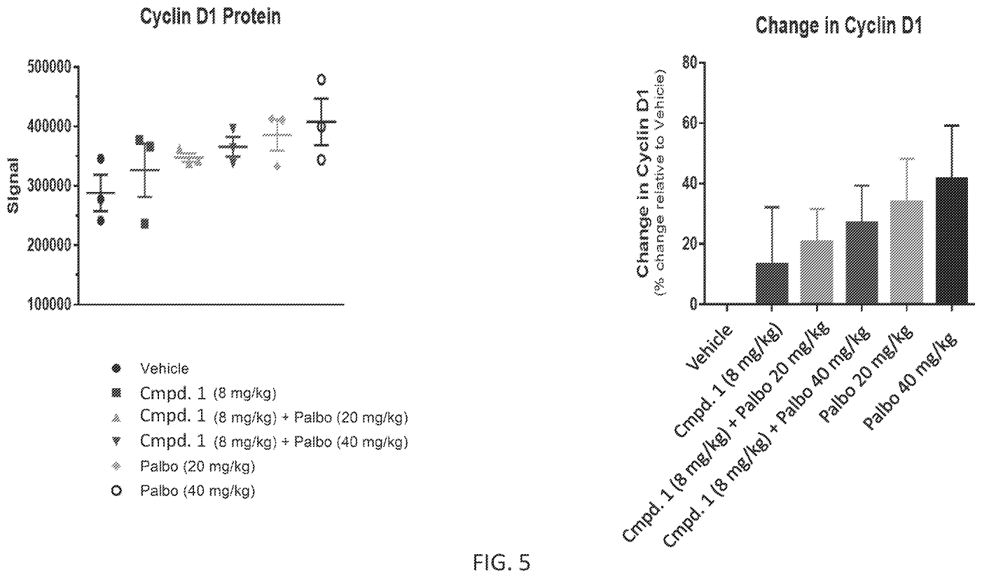

FIG. 5 shows the expression levels of Cyclin D1 protein in tumor samples collected at the conclusion of the study in each of the experimental groups.

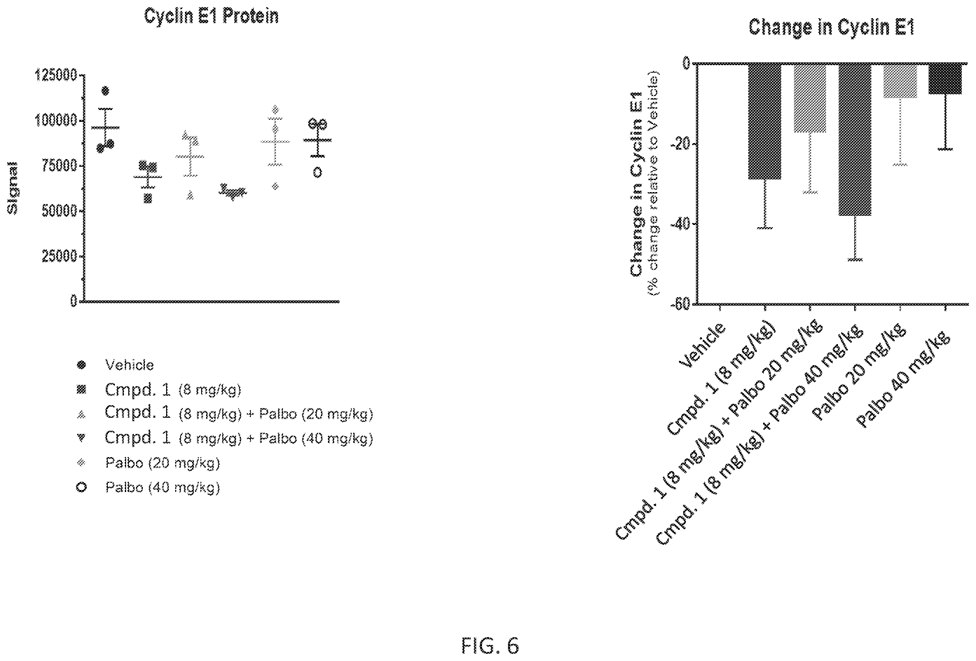

FIG. 6 shows the expression levels of Cyclin E1 protein in tumor samples collected at the conclusion of the study in each of the experimental groups. As would be appreciated by the skilled artisan, the level of cyclin E1 expression in metastatic breast cancer has been associated with response to Palbociclib. Specifically, in a clinical trial of women with ER+/Her2− metastatic breast cancer, Palbociclib plus fulvestrant was significantly less effective (shorter progression-free survival) if the tumor expressed high levels of cyclin E1 compared to tumors with low cyclin E1 expression (Turner, DOI: 10.1200/JCO.18.00925 Journal of Clinical Oncology 37, no. 14 (May 10, 2019) 1169-1178).

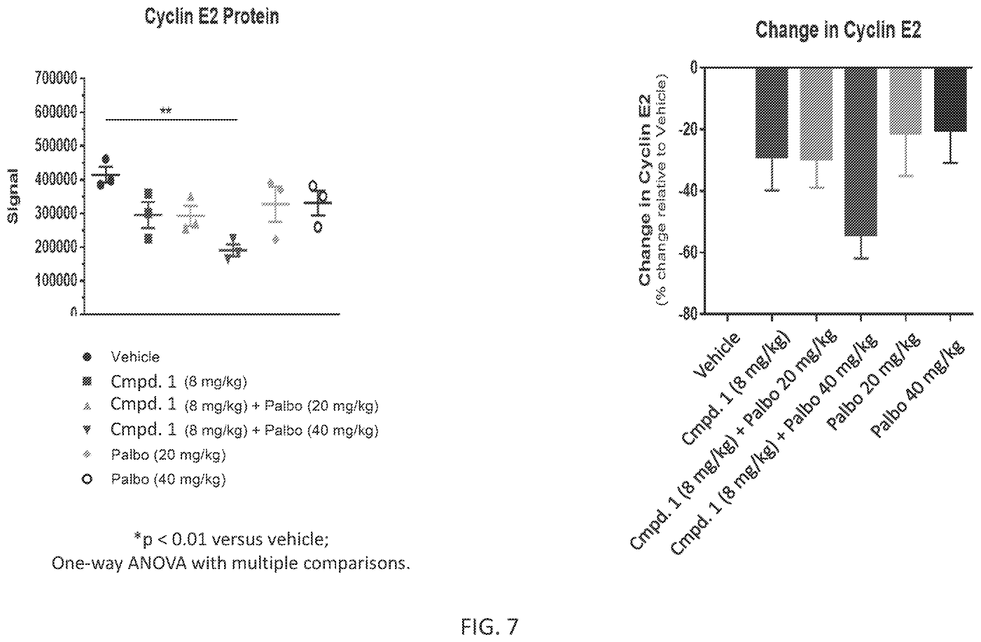

FIG. 7 shows the expression levels of Cyclin E2 protein in tumor samples collected at the conclusion of the study in each of the experimental groups. As shown in FIG. 7, treatment with the combination of Compound 1 and palbociclib resulted in lower levels of Cyclin E2 protein in the tumor samples, as compared to both the vehicle controls, treatment with compound 1 alone, or palbociclib alone. As would be appreciated by the skilled artisan, expression level of cyclin E2 is associated with tumor progression in ER+ breast cancer, with shorter overall survival or recurrence-free survival observed in patients with high cyclin E2 expression (Sieuwerts, 2006, Clinical Cancer Research 12 3319-3328. (https://doi.org/10.1158/1078-0432. CCR-06-0225; Millioli, Endocrine-Related Cancer (2020) 27, R93-R112)

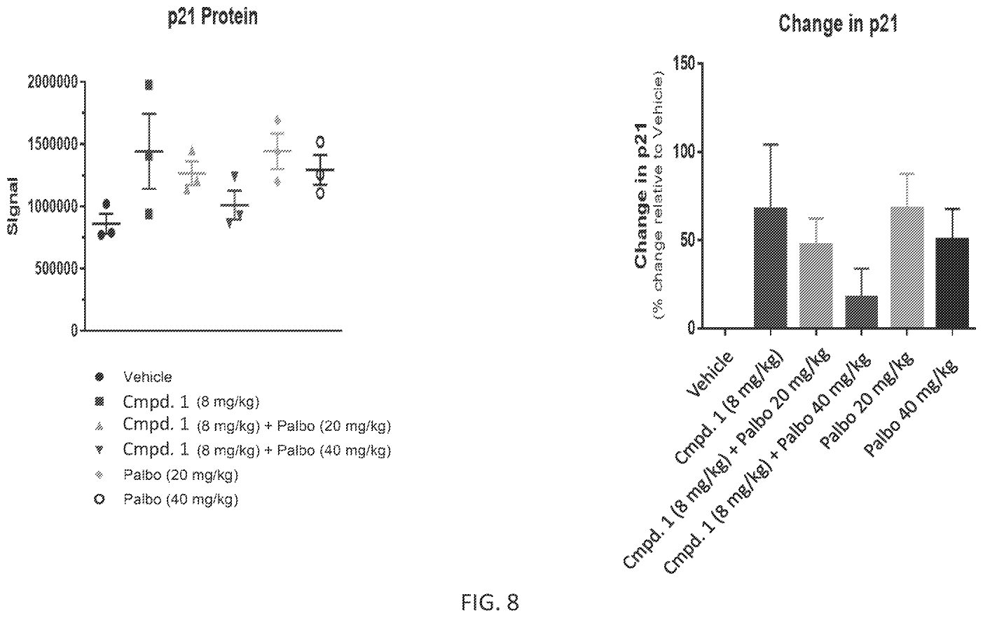

FIG. 8 shows the expression levels of p21 protein in tumor samples collected at the conclusion of the study in each of the experimental groups.

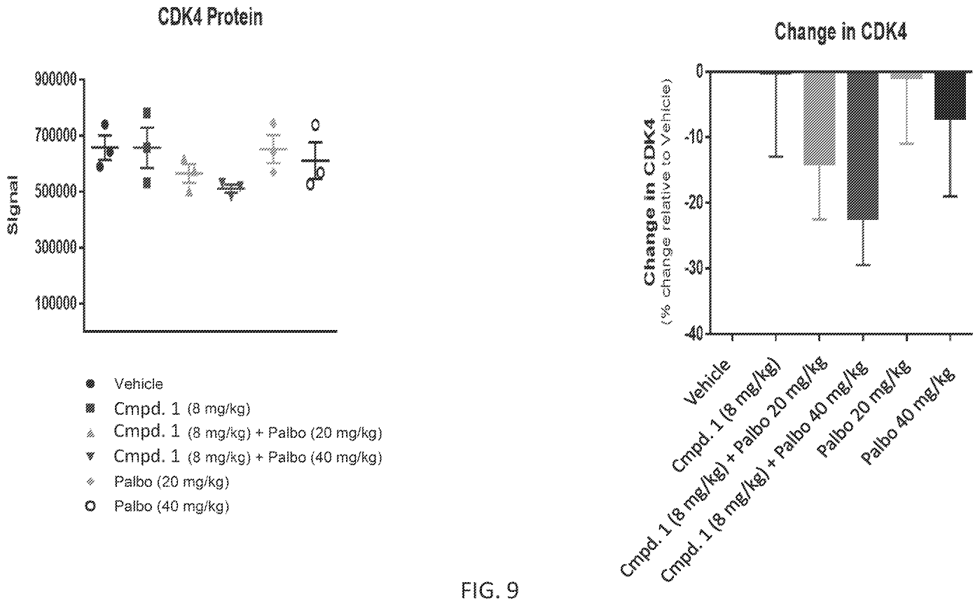

FIG. 9 shows the expression levels of CDK4 protein in tumor samples collected at the conclusion of the study in each of the experimental groups. As shown in FIG. 9, treatment with the combination of Compound 1 and palbociclib resulted in lower levels of CDK4 protein in the tumor samples, as compared to both the vehicle controls, treatment with compound 1 alone, or palbociclib alone.

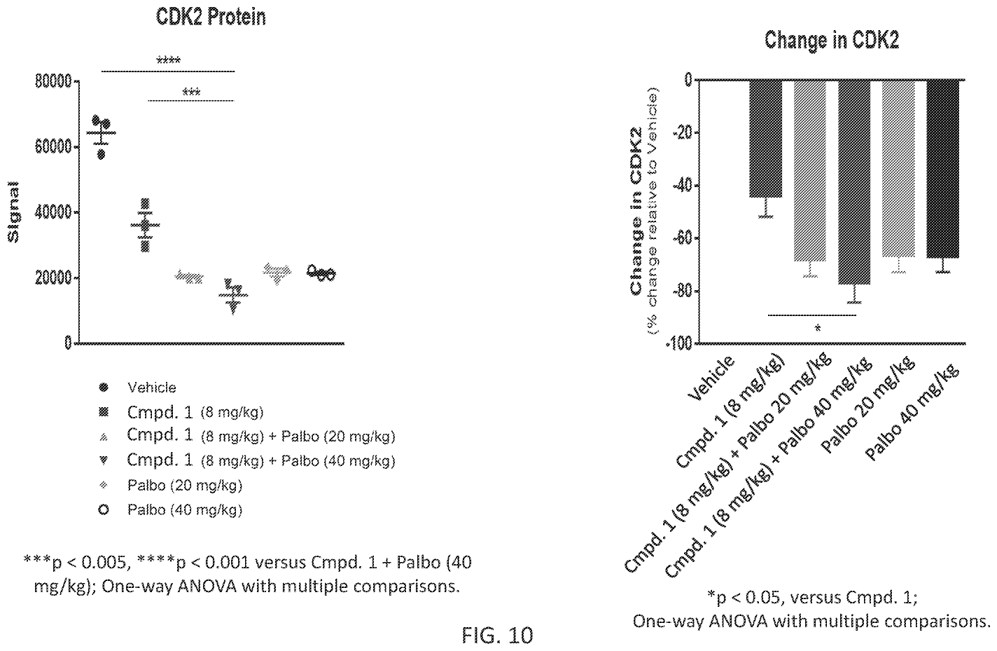

FIG. 10 shows the expression levels of CDK2 protein in tumor samples collected at the conclusion of the study in each of the experimental groups. As shown in FIG. 10, treatment with the combination of Compound 1 and palbociclib resulted in lower levels of CDK2 protein in the tumor samples, as compared to both the vehicle controls, treatment with compound 1 alone, or palbociclib alone.

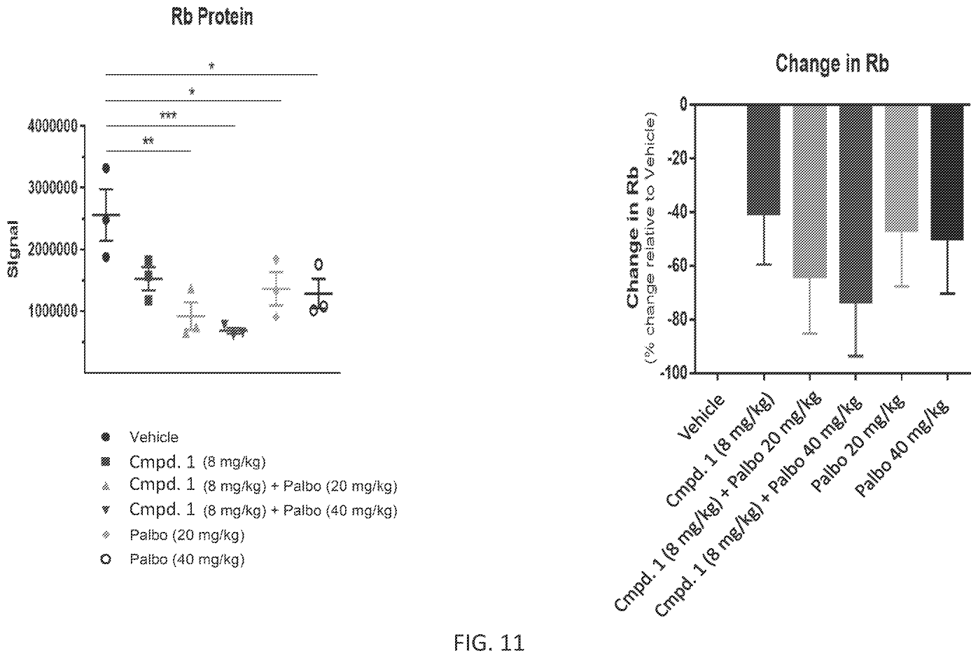

FIG. 11 shows the expression levels of Rb protein in tumor samples collected at the conclusion of the study in each of the experimental groups. As shown in FIG. 11, treatment with the combination of Compound 1 and palbociclib resulted in lower levels of Rb protein in the tumor samples, as compared to both the vehicle controls, treatment with compound 1 alone, or palbociclib alone. As would be appreciated by the skilled artisan, the level of Rb protein expression in metastatic breast cancer has been associated with response to Palbociclib.

Specifically, in a clinical trial of women with ER+/Her2− metastatic breast cancer, Palbociclib plus fulvestrant was significantly less effective (shorter progression-free survival) if the tumor expressed high levels of Rb protein compared to tumors with low Rb protein expression (Turner, DOI: 10.1200/JCO.18.00925 Journal of Clinical Oncology 37, no. 14 (May 10, 2019) 1169-1178).

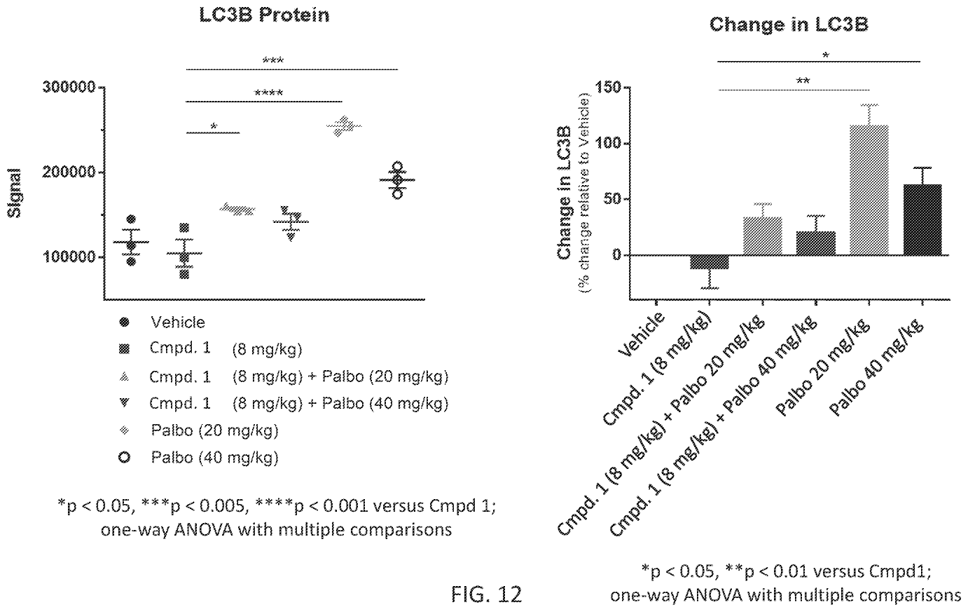

FIG. 12 shows the changes to the autophagy protein LC3B measured in tumor tissue at the conclusion of the study. As would be appreciated by the skilled artisan, LC3B is a marker for autophagy. As would be appreciated by the skilled artisan, CDK4/6 inhibitors can induce autophagy (Vijayaraghavan, S. et al. CDK4/6 and autophagy inhibitors synergistically induce senescence in Rb positive cytoplasmic cyclin E negative cancers: Nat. Commun. 8, 15916 doi: 10.1038/ncomms15916 (2017)). As shown in FIG. 12, combinations of Palbociclib and Compound 1 reduce the induction of LC3B autophagy protein.

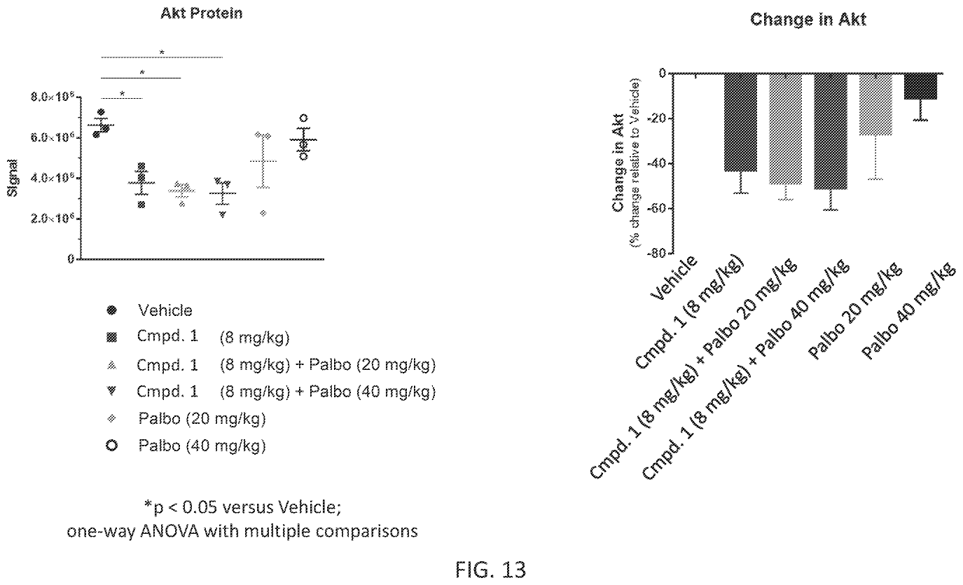

FIG. 13 shows the expression levels of Akt protein in tumor samples collected at the conclusion of the study in each of the experimental groups. As shown in FIG. 13, treatment with a combination of Compound 1 and palbociclib resulted in lower levels of Akt protein in the tumor samples, as compared to both the vehicle control, treatment with Compound 1 alone, or palbociclib alone.

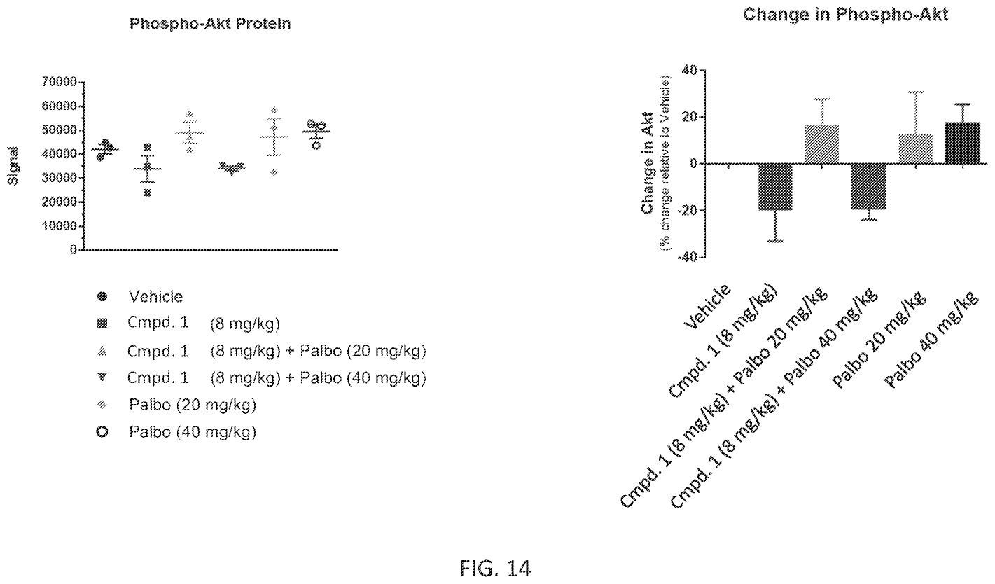

FIG. 14 shows the expression level of Phospho-Akt protein in tumor samples collected at the conclusion of the study in each of the experimental groups. As shown in FIG. 14, treatment with Compound 1 alone and Compound 1 in combination with the high dose of palbociclib resulted in lower levels of Phospho-Akt protein as compared to Compound 1 in combination with the low dose of palbociclib and palbociclib alone.

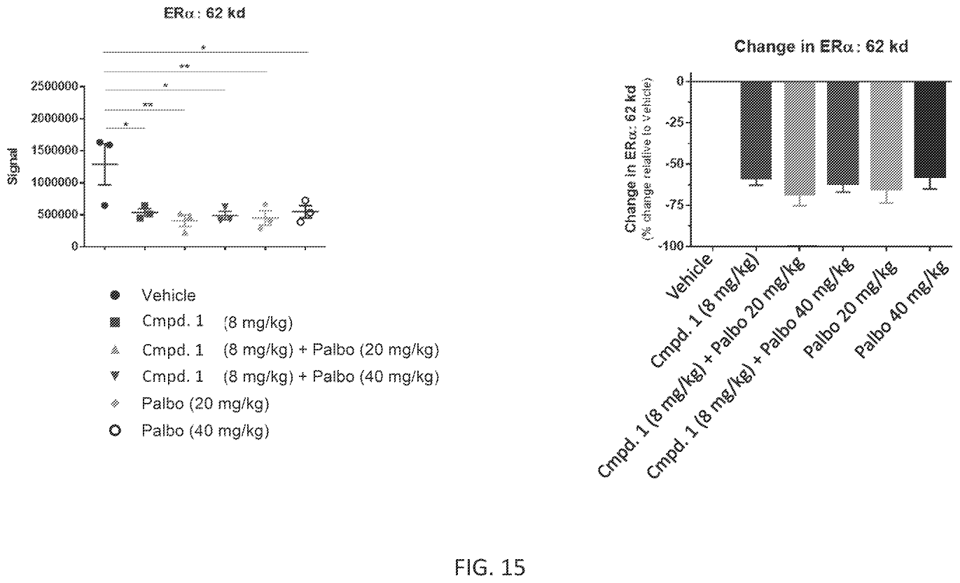

FIG. 15 shows the expression levels of estrogen receptor alpha (ERα)-62 kDa protein in tumor samples collected at the conclusion of the study in each of the experimental groups. As shown in FIG. 15, treatment with a combination of Compound 1 and palbociclib resulted in a decrease in the level of ERα-62 kDa protein.

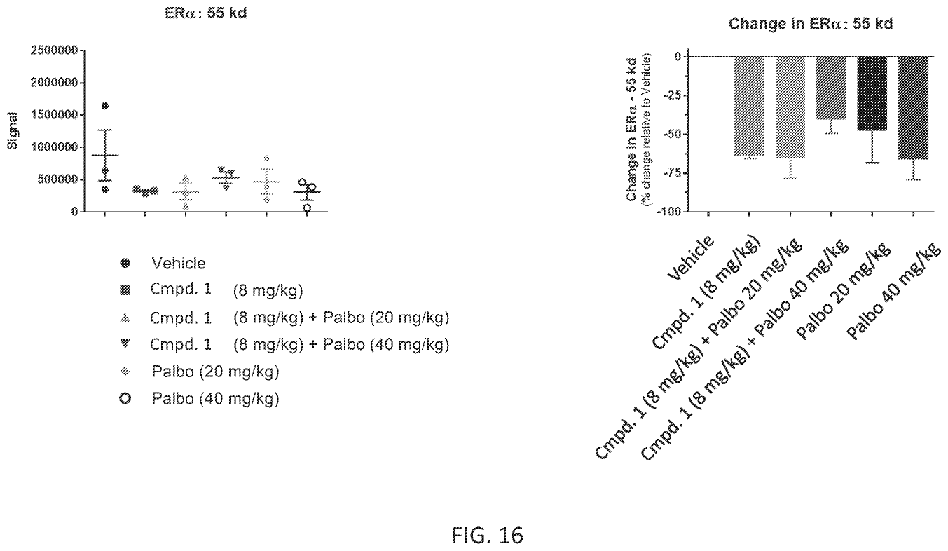

FIG. 16 shows the expression levels of ERα-55 kDa protein in tumor samples collected at the conclusion of the study in each of the experimental groups. As shown in FIG. 16, treatment with a combination of Compound 1 and palbociclib at 20 mg/kg resulted in a decrease in the level of ERα-55 kDa protein that was greater than the decrease in ERα-55 kDa protein upon treatment with 20 mg/kg palbociclib alone.

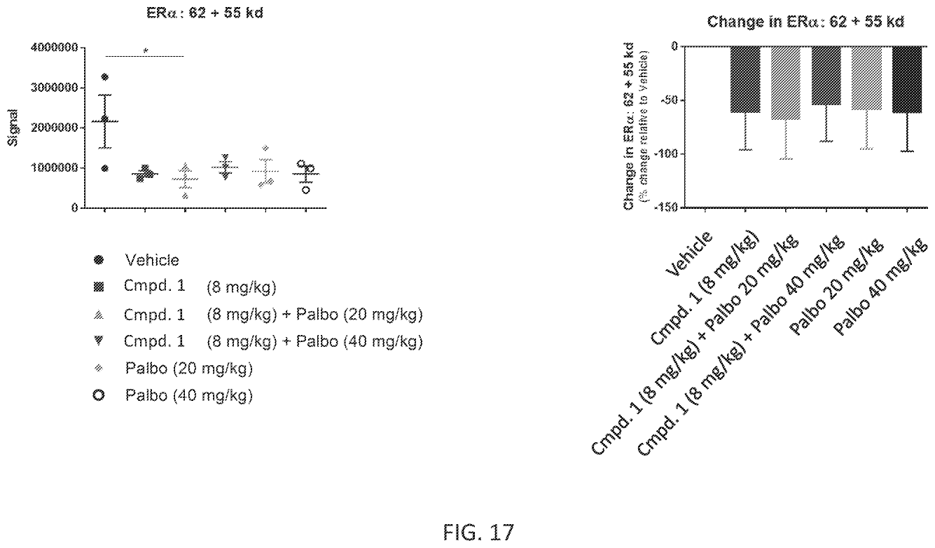

FIG. 17 shows the sum of the expression levels of ERα-55 kDa protein and ERα-62 kDa protein in tumor samples collected at the conclusion of the study in each of the experimental groups.

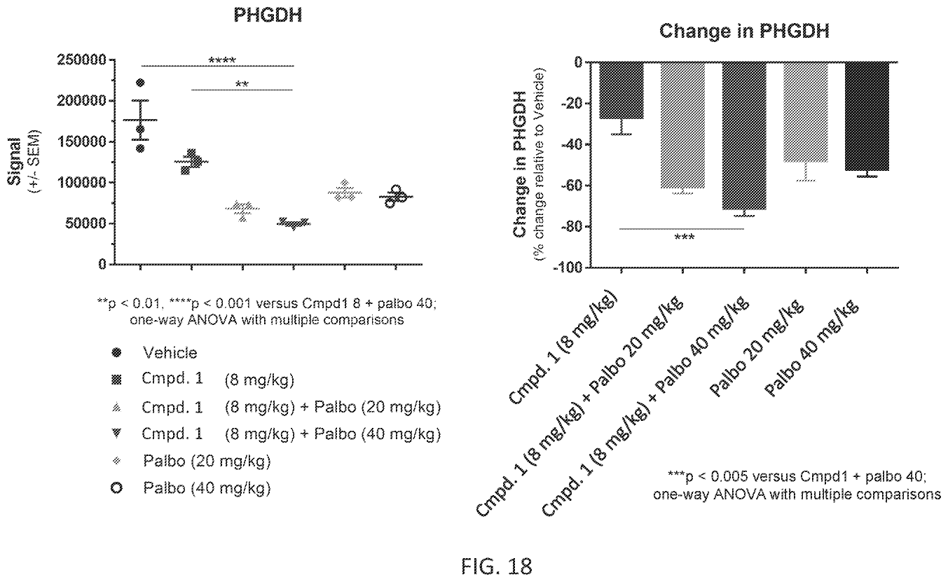

FIG. 18 shows the expression levels of PHGDH protein in tumor samples collected at the conclusion of the study in each of the experimental groups. As shown in FIG. 18, treatment with a combination of Compound 1 and palbociclib resulted in a decrease in the level PHGDH protein that was greater than the decrease PHGDH protein upon treatment with compound 1 alone or palbociclib alone.

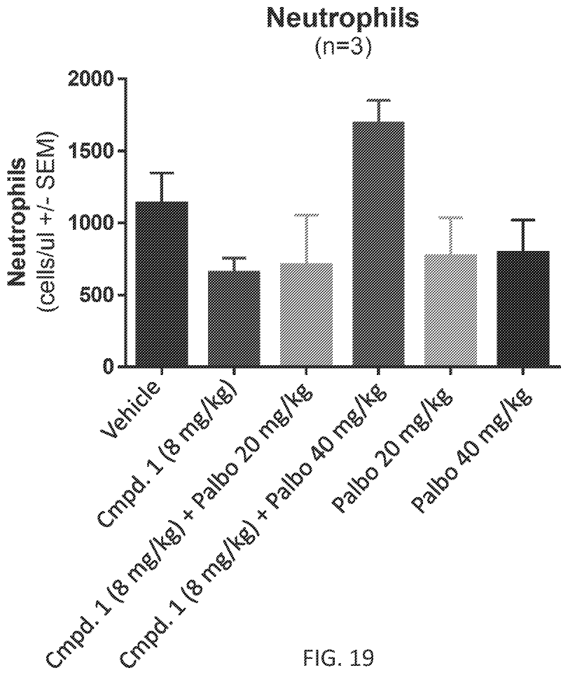

FIG. 19 shows the number of neutrophils in whole blood collected at the conclusion of the study in each of the experimental groups. As shown in FIG. 19, both Palbociclib and SDX-7320 alone suppressed levels of neutrophils by 30-40% relative to Vehicle-treated mice, while the combination of SDX-7320 and Palbociclib (40 mg/kg) increased neutrophils 49% relative to Vehicle-treated mice. As would be appreciated by the skilled artisan, neutropenia is a major side effect of Palbociclib. Accordingly, without wishing to be bound by theory, these results indicate that the combination of Compound 1 and palbociclib can attenuate palbociclib-induced neutropenia, thereby providing an improved hematologic safety profile.

The results presented in this example demonstrate that the combination of the MetAP2 inhibitors of the present disclosure and CDK4/6 inhibitors, more specifically palbociclib, can be used to treat cancer and to prevent CDK4/6 treatment resistance. Additionally, the results presented in this example demonstrate that the combination of MetAP2 inhibitors of the present disclosure and CDK4/6 inhibitors induce changes in gene expression that are consistent with increased rates of survival in patients and decreased levels of CDK4/6 inhibitor resistance. Without wishing to be bound by theory, these changes in gene expression demonstrate that the administration of a combination of MetAP2 inhibitors of the present disclosure and CDK4/6 inhibitors can overcome existing limitations of CDK4/6 inhibitor therapy.

### Example 2

The following is a non-limiting example demonstrating that the MetAP2 inhibitors of the present disclosure and CDK4/6 inhibitors can be used in combination to treat cancer. Moreover, the following non-limiting example demonstrates that the combination of the MetAP2 inhibitors of the present disclosure and CDK4/6 inhibitors exhibits unexpectedly superior anti-tumor activity as compared to the use of either agent alone. As would be appreciated by the skilled artisan, several genes and cellular pathways have been implicated in acquired resistance to CDK4/6 inhibitors such as palbociclib and ribociclib, including, but not limited to, Cyclin E1, CDK2, CDK4, Akt signaling, Autophagy pathways and cancer stem cell initiation pathways. To that end, expression levels of various genes were analyzed in tumor samples collected at the conclusion of the study to determine how the administration of a combination of MetAP2 inhibitors of the present disclosure and CDK4/6 inhibitors may impact the expression of various genes implicated in CDK4/6 inhibitor resistance.

Tumor samples from the mice treated in Example 1 were subjected to further analysis. Briefly, tumors isolated form the mice were placed into a solution of RNALater then frozen at −70° C. PolyA+ RNA was then isolated and reverse transcribed into cDNA. The cDNA was then analyzed by sequencing. The sequencing data was aligned against the human genome (GRCh38) to provide relative RNA expression levels for each animal and for each gene mentioned in GRCh38. Statistical analysis was then performed to identify genes and pathways that were regulated uniquely when mice were treated with a combination of Compound 1 and palbociclib as opposed to when mice were treated with monotherapies.

Gene set enrichment analysis (GSEA) was performed using methods standard in the art, as would be appreciated by the skilled artisan. Specifically, the gene set KEGG_ONE_CARBON_POOL_BY_FOLATE was analyzed in tumors from two treatment groups: mice that received a combination of Compound 1 at 8 mg/kg (SC) and palbociclib at 40 mg/kg (PO), and mice that were treated only with palbociclib at 40 mg/kg (PO). The GSEA analysis demonstrated the genes MTHFD1L, TYMS, ALDH11, MTHFD1, MTHFD2, GART, SHMT1, DHFR, MTR, SHMT2 and MTFMT had lower expression in the tumors from mice treated with the combination of Compound 1 and palbociclib as compared to the tumors from mice treated with palbociclib alone. As would be appreciated by the skilled artisan, the preceding genes are biologically related to metabolism.

Based on the preceding analysis, the expression levels of the genes PHGDH, PSPH, TYMS, MTHFDIL, MTHFD1, MTHFD2, SHMT1, SHMT2 and DHFR were analyzed in tumor samples isolated from each of the treatment groups put forth in Example 1.

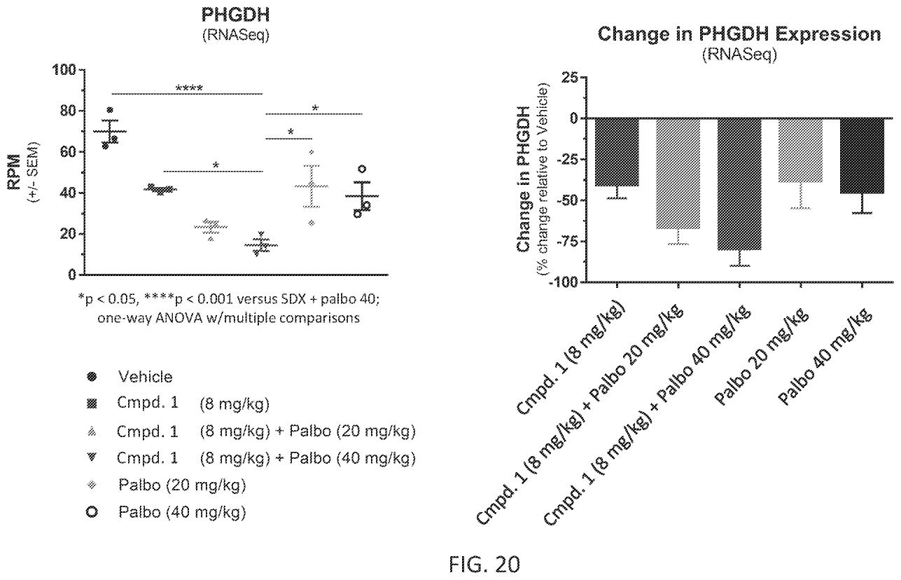

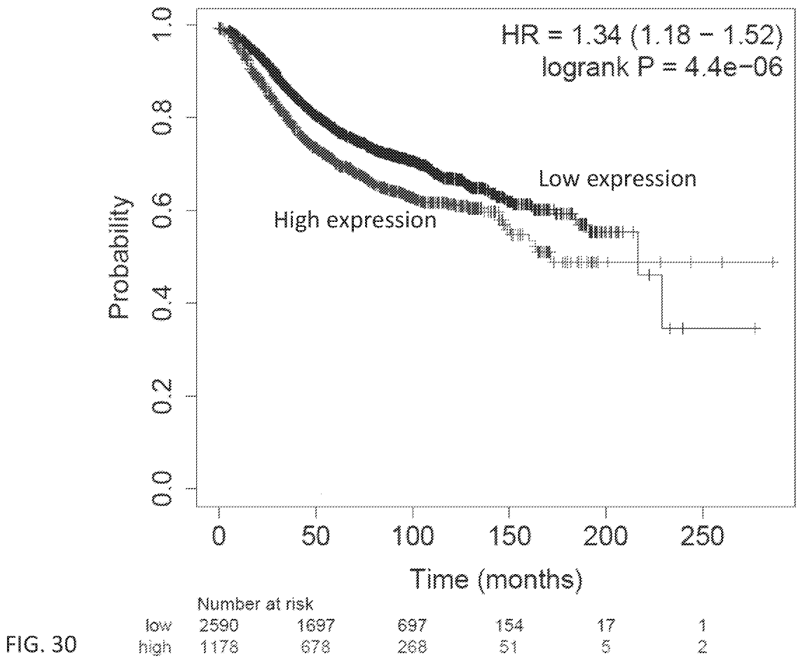

FIG. 20 shows the expression levels of PHGDH (phosphoglycerate dehydrogenase) in tumor samples collected at the conclusion of the study in each of the experimental groups put forth in Example 1. As shown in FIG. 20, treatment with a combination of Compound 1 and palbociclib resulted in a greater decrease in the level of PHGDH as compared to treatment with either Compound 1 alone or palbociclib alone. As would be appreciated by the skilled artisan, decrease PHDGH reduces de novo synthesis of serine and glycine, which cancer cells have been shown to rely upon for multiple cellular processes (see Zhao X, Fu J, Du J, Xu W. Int. Biol Sci. 2020;16(9):1495-1506). Moreover, as shown in FIG. 30, there is an increased risk of recurrence in ER+ Breast Cancer in subjects with high expression levels of PHGDH. Accordingly, without wishing to be bound by theory, the decrease in expression of PHGDH induced by treatment with a combination of Compound 1 and a CDK4/6 inhibitor may help to decrease the risk of recurrence and prolong survival in subjects with cancer, including ER+ breast cancer.

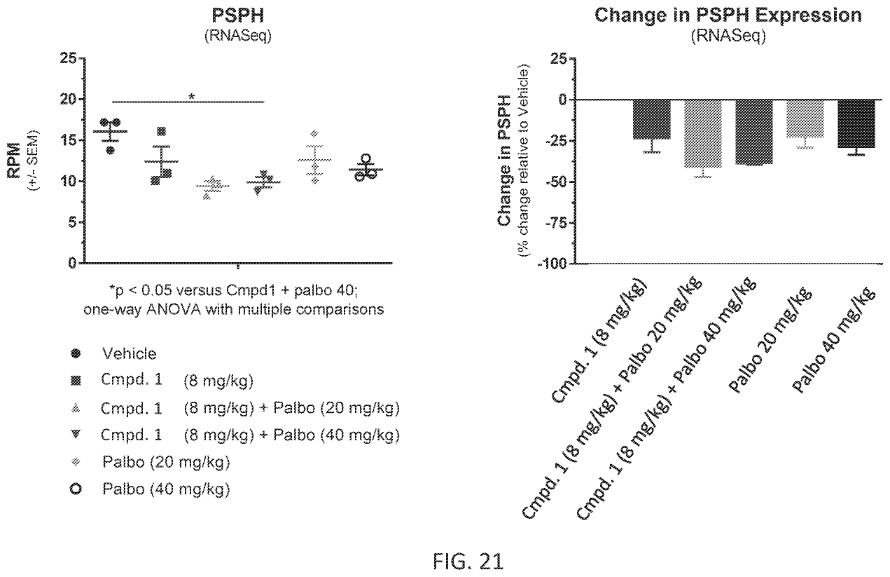

FIG. 21 shows the expression levels of PSPH (phosphoserine phosphatase) in tumor samples collected at the conclusion of the study in each of the experimental groups put forth in Example 1. As shown in FIG. 21, treatment with a combination of Compound 1 and palbociclib resulted in a greater decrease in the level of PSPH as compared to treatment with either Compound 1 alone or palbociclib alone.

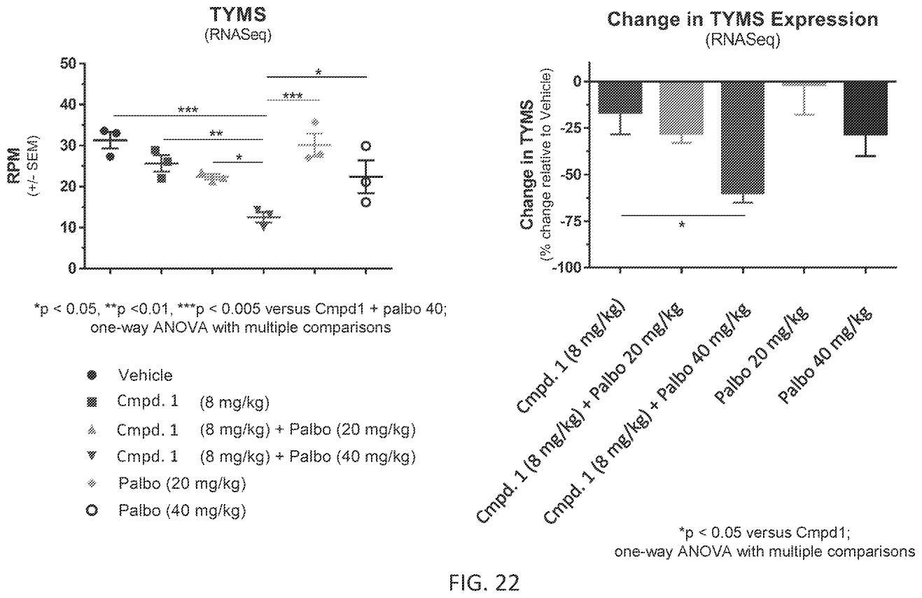

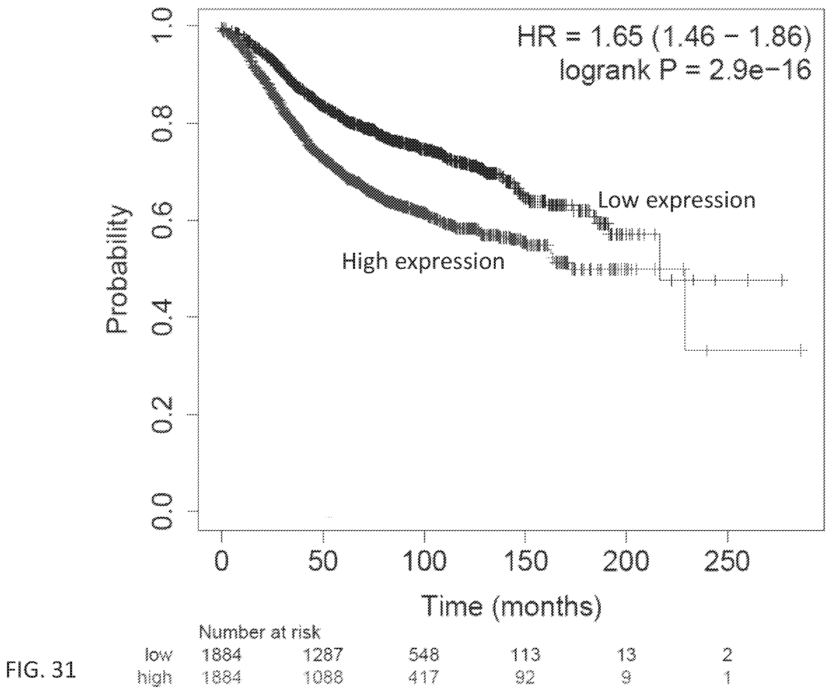

FIG. 22 shows the expression levels of TYMS (thymidylate synthetase) in tumor samples collected at the conclusion of the study in each of the experimental groups put forth in Example 1. As shown in FIG. 22, treatment with a combination of Compound 1 and palbociclib resulted in a greater decrease in the level of TYMS as compared to treatment with either Compound 1 alone or palbociclib alone. As would be appreciated by the skilled artisan, decreased expression of TYMS1 slows the production of pyrimidine nucleotides, which are essential for DNA synthesis and cell growth, specifically in the context of cancer cells. Moreover, as shown in FIG. 31, there is an increased risk of recurrence in ER+ Breast Cancer in subjects with high expression levels of TYMS. Accordingly, without wishing to be bound by theory, the decrease in expression of TYMS induced by treatment with a combination of Compound 1 and a CDK4/6 inhibitor may help to decrease the risk of recurrence and prolong survival in subjects with cancer, including ER+ breast cancer.

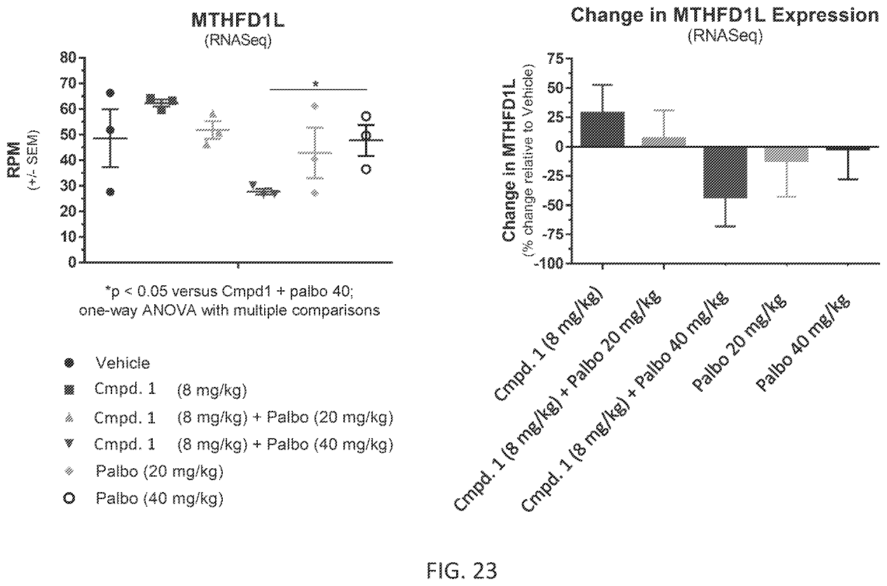

FIG. 23 shows the expression levels of MTHFD1L (methylenetetrahydrofolate dehydrogenase-like 1) in tumor samples collected at the conclusion of the study in each of the experimental groups put forth in Example 1. As shown in FIG. 23, treatment with a combination of Compound 1 and the higher dose of palbociclib resulted in a greater decrease in the level of MTHFD1L as compared to treatment with palbociclib at a high or low dose. Treatment with Compound 1 alone resulted in an increase in the expression of MTHFD1L.

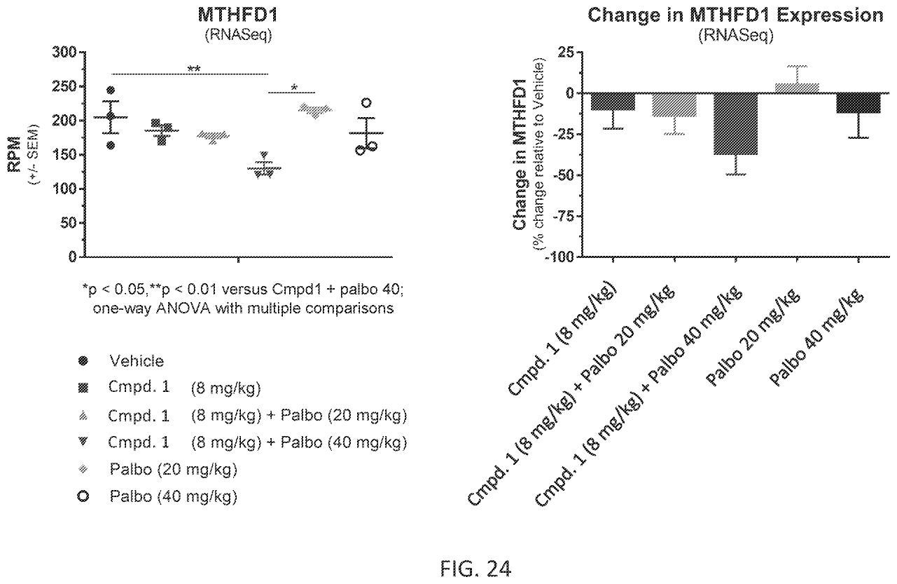

FIG. 24 shows the expression levels of MTHFD1 (methylenetetrahydrofolate dehydrogenase) in tumor samples collected at the conclusion of the study in each of the experimental groups put forth in Example 1. As shown in FIG. 24, treatment with a combination of Compound 1 and the higher dose of palbociclib resulted in a greater decrease in the level of MTHFD1 as compared to treatment with either Compound 1 alone or palbociclib alone.

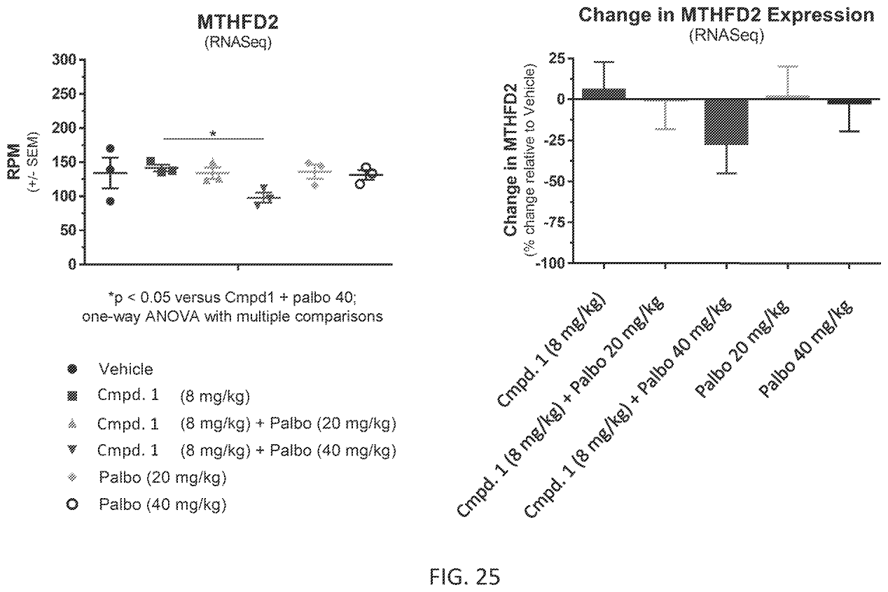

FIG. 25 shows the expression levels of MTHFD2 (methylenetetrahydrofolate dehydrogenase (NADP+ dependent) 2) in tumor samples collected at the conclusion of the study in each of the experimental groups put forth in Example 1. As shown in FIG. 25, treatment with a combination of Compound 1 and the higher dose of palbociclib resulted in a greater decrease in the level of MTHFD2 as compared to treatment with the higher dose of palbociclib alone. Treatment with Compound 1 alone and the lower dose of palbociclib resulted in an increase in the expression of MTHFD2.

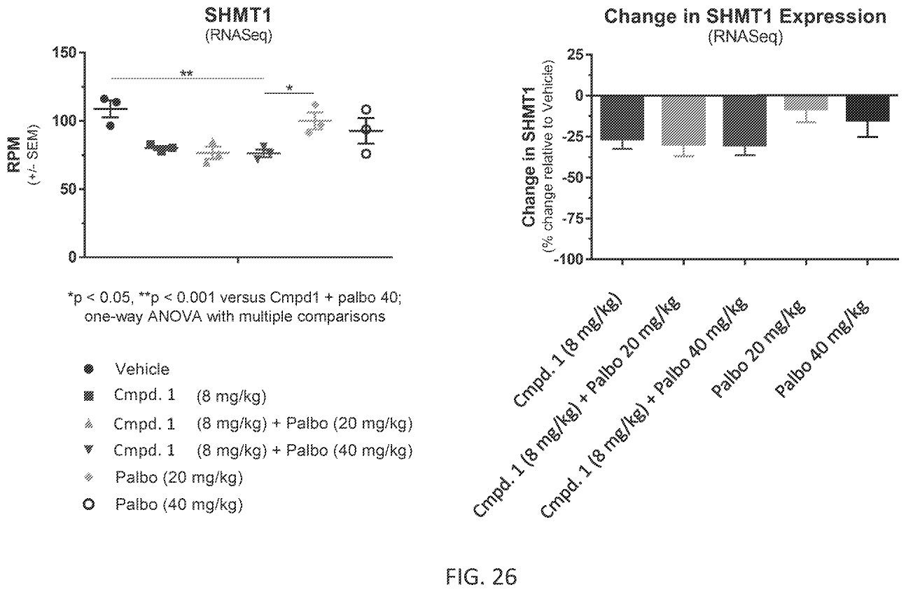

FIG. 26 shows the expression levels of SHMT1 (serine hydroxymethyltransferase 1) in tumor samples collected at the conclusion of the study in each of the experimental groups put forth in Example 1. As shown in FIG. 26, treatment with a combination of Compound 1 and palbociclib resulted in a greater decrease in the level of SHMT1 as compared to treatment with palbociclib alone.

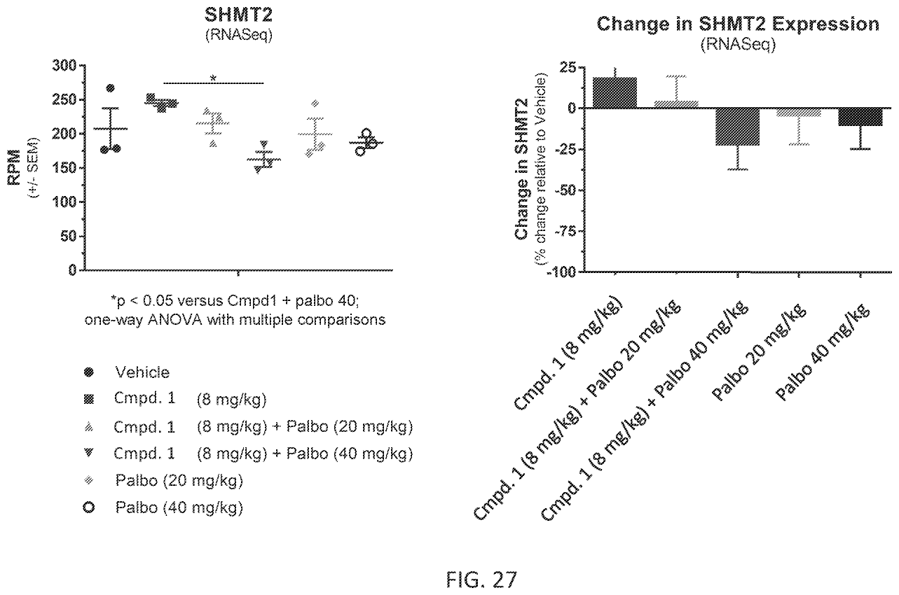

FIG. 27 shows the expression levels of SHMT2 (serine hydroxymethyltransferase 2) in tumor samples collected at the conclusion of the study in each of the experimental groups put forth in Example 1. As shown in FIG. 27, treatment with a combination of Compound 1 and the higher dose of palbociclib resulted in a greater decrease in the level of SHMT2 as compared to treatment with palbociclib alone. Treatment with Compound 1 alone resulted in an increase in SHMT2 expression.

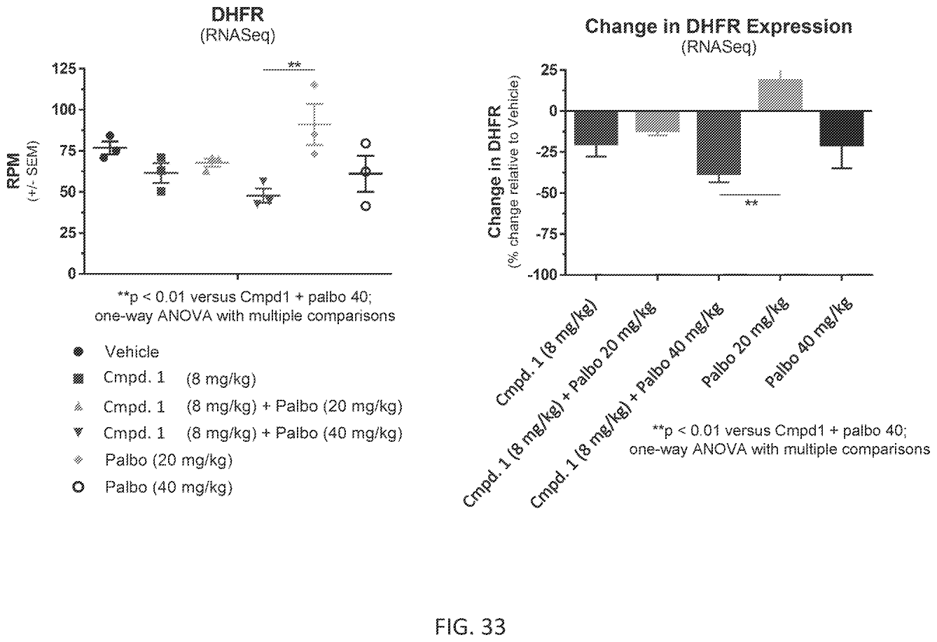

FIG. 33 shows the expression levels of DHFR (dihydrofolate reductase) in tumor samples collected at the conclusion of the study in each of the experimental groups put forth in Example 1. As shown in FIG. 33, treatment with a combination of Compound 1 and the higher dose of palbociclib resulted in a greater decrease in the level of DHFR as compared to treatment with the higher dose of palbociclib alone and Compound 1 alone.

Moreover, sequencing analysis of the tumor samples showed that the enzymes 3-phosphoglycerate dehydrogenase (PHGDH) and thymidylate synthase (TYMS) were downregulated in tumors from mice treated with a combination of Compound 1 and palbociclib as compared to tumors from mice treated with Compound 1 alone. Without wishing to be bound by theory, and as would be appreciated by the skilled artisan, decreased PHGDH reduces de novo synthesis of serine and glycine, which cancer cells are known to rely upon for multiple cellular processes, and decreased expression of the enzyme TS slows the production of purine nucleotides, which are essential for DNA synthesis and cell growth.

The expression levels of genes in the PI3K pathway were also analyzed in tumor samples isolated from each of the treatment groups put forth in Example 1.

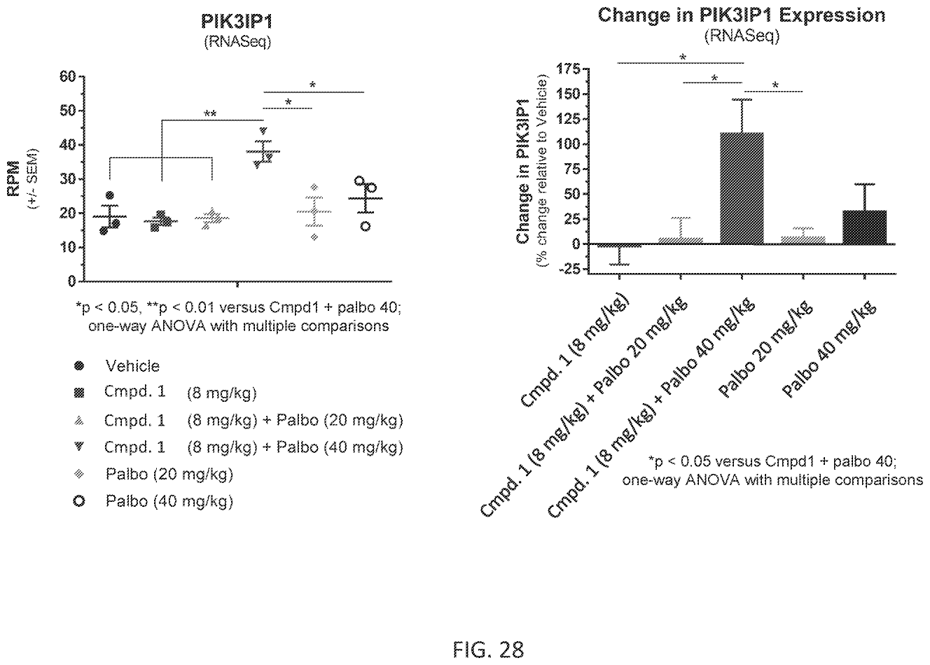

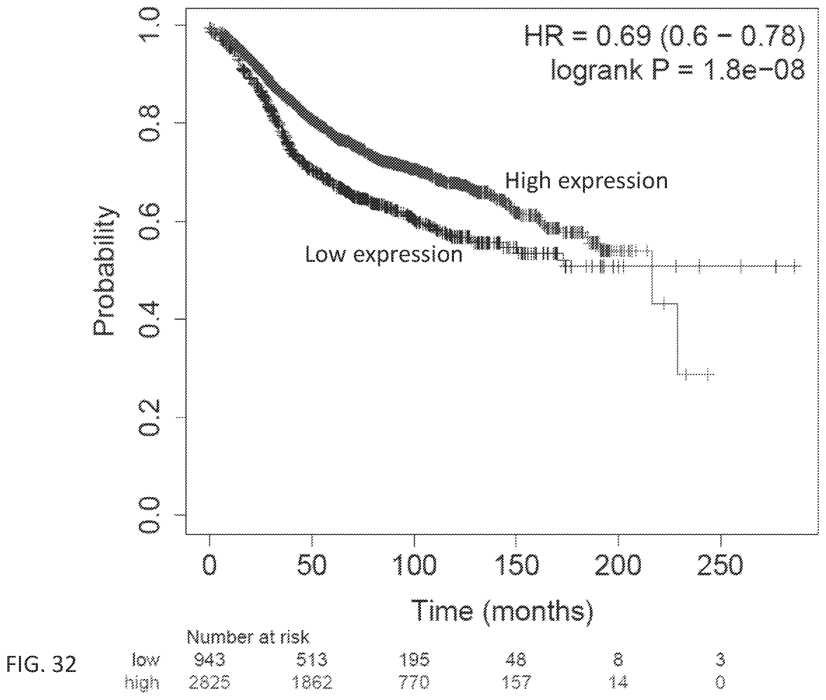

FIG. 28 shows the expression levels of PIK3IP1 in tumor samples collected at the conclusion of the study in each of the experimental groups put forth in Example 1. As shown in FIG. 28, treatment with a combination of Compound 1 and the higher dose of palbociclib resulted in a dramatic increase in PIK31P1 expression compared to treatment with either Compound 1 alone or palbociclib alone. As would be appreciated by the skilled artisan, PIK3IP1 is an endogenous PI3K inhibitor and suppressor of tumor development (see He X, Zhu Z, Johnson C, et al. Cancer Res. 2008; 68(14):5591-5598). Accordingly, treatment with the combination of Compound 1 and palbociclib unexpectedly results in increased expression of a tumor suppressor. As shown in FIG. 32, there is an increased risk of recurrence in ER+ Breast Cancer in subjects with low expression levels of PIK3IP1. Accordingly, without wishing to be bound by theory, the increase in expression of PIK3IP1 induced by treatment with a combination of Compound 1 and a CDK4/6 inhibitor may help to decrease the risk of recurrence and prolong survival in subjects with ER+ breast cancer.

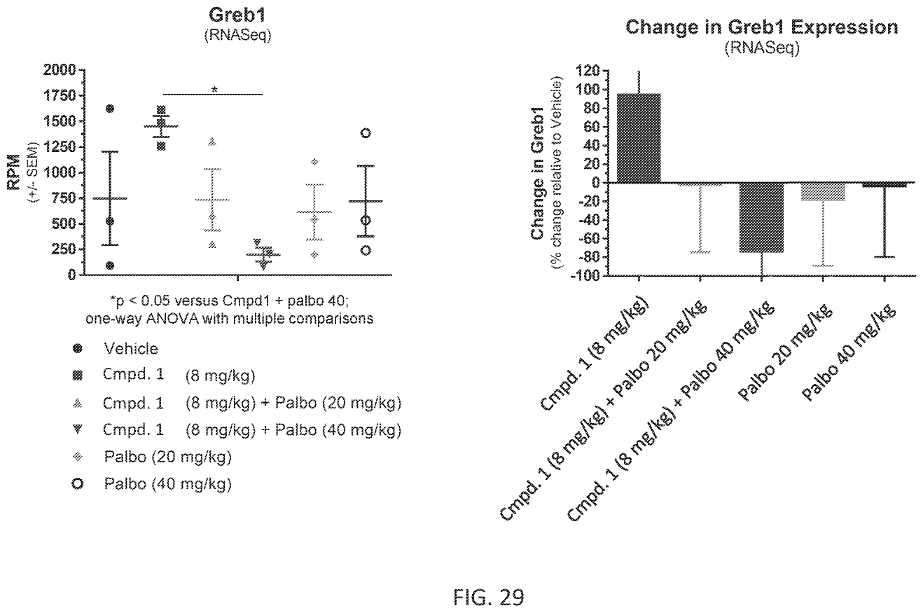

FIG. 29 shows the expression levels of Greb1 in tumor samples collected at the conclusion of the study in each of the experimental groups put forth in Example 1. As shown in FIG. 29, treatment with a combination of Compound 1 and the higher dose of palbociclib resulted in a greater decrease in the level of Greb1 as compared to treatment with palbociclib alone. Treatment with Compound 1 alone resulted in an increase in Greb1 expression. As would be appreciated by the skilled artisan, Greb1 is known to be an estrogen-regulated growth promoter in Breast Cancer and has been shown in vitro to be a PI3K activator (see Haines et al, Carcinogenesis, 2020, Vol. 41, No. 12, 1660-1670). Accordingly, treatment with the combination of Compound 1 and palbociclib unexpectedly results in decreased expression of a tumor promoter.

In addition to the genes described above, genes in the OnctoType 21-gene panel for recurrence in Breast Cancer (see Paik, 2004, NE/M, 351(27): 2817-26) were also analyzed. These genes included MybL2, Ki-67, BIRC5/Survivin, CCNB1/Cyclin B1 and SCUBE2.

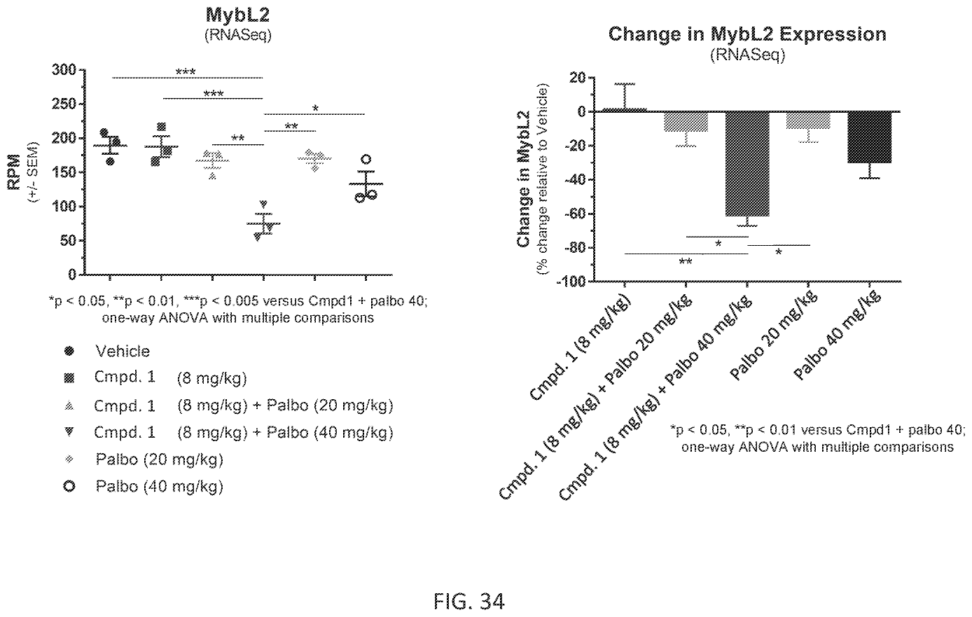

FIG. 34 shows the expression levels of MybL2 (MYB proto-oncogene like 2) in tumor samples collected at the conclusion of the study in each of the experimental groups put forth in Example 1. As shown in FIG. 34, treatment with a combination of Compound 1 and the higher dose of palbociclib resulted in a greater decrease in the level of MybL2 as compared to treatment with palbociclib alone.

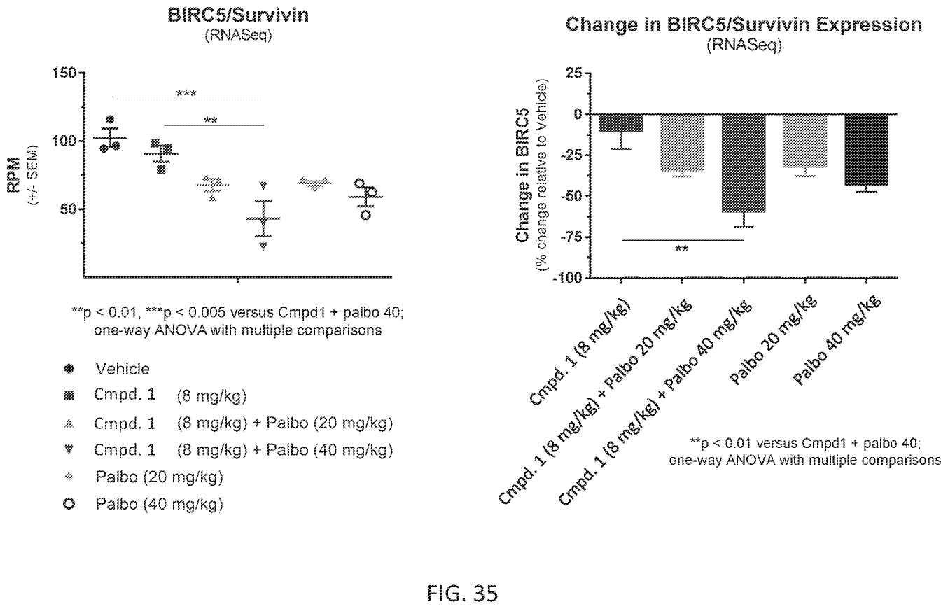

FIG. 35 shows the expression levels of BIRC5/Survivin (baculoviral IAP repeat containing 5) in tumor samples collected at the conclusion of the study in each of the experimental groups put forth in Example 1. As shown in FIG. 35, treatment with a combination of Compound 1 and the higher dose of palbociclib resulted in a greater decrease in the level of BIRC5/Survivin as compared to treatment with palbociclib alone and Compound 1 alone.

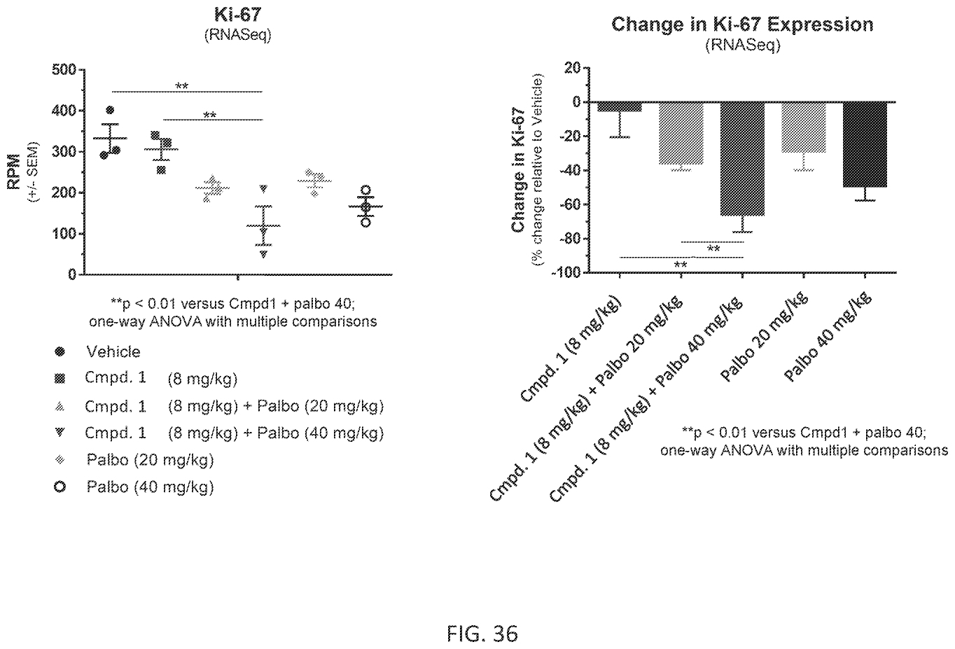

FIG. 36 shows the expression levels of Ki-67 in tumor samples collected at the conclusion of the study in each of the experimental groups put forth in Example 1. As shown in FIG. 36, treatment with a combination of Compound 1 and the higher dose of palbociclib resulted in a greater decrease in the level of Ki-67 as compared to treatment with palbociclib alone and Compound 1 alone.

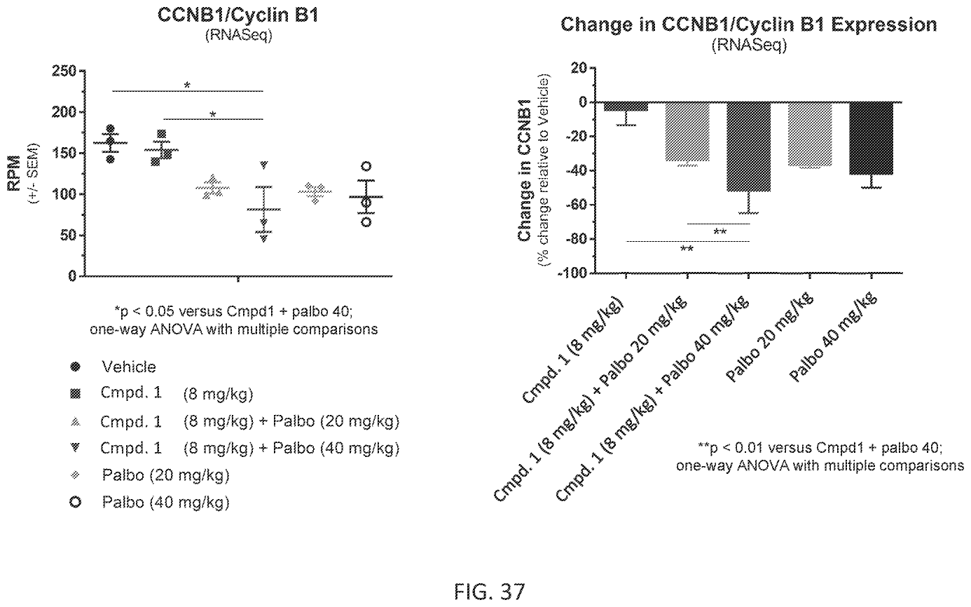

FIG. 37 shows the expression levels of CCNB1/cyclin B1 in tumor samples collected at the conclusion of the study in each of the experimental groups put forth in Example 1. As shown in FIG. 37, treatment with a combination of Compound 1 and the higher dose of palbociclib resulted in a greater decrease in the level of CCNB1/cyclin B1 as compared to treatment with palbociclib alone and Compound 1 alone.

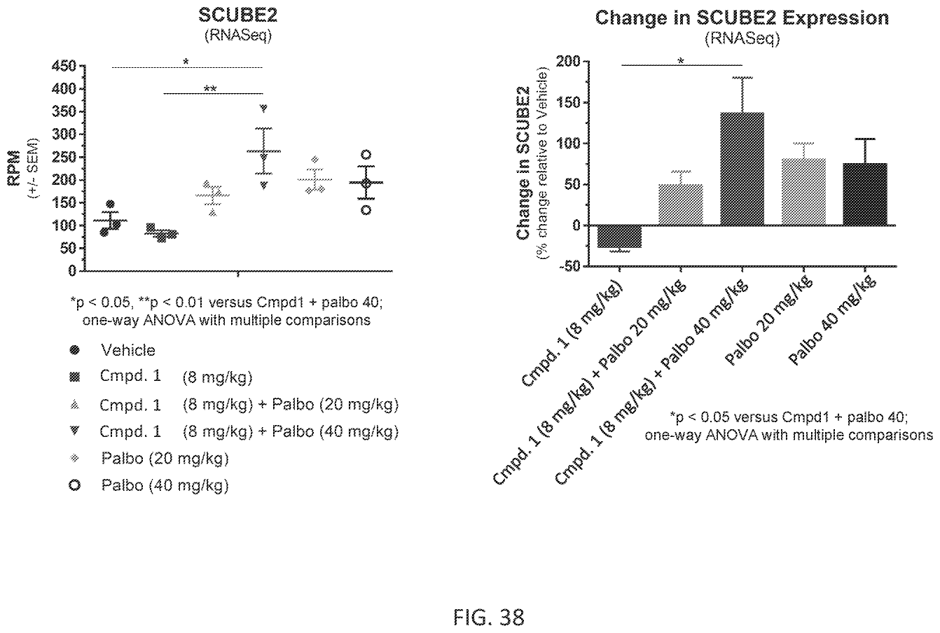

FIG. 38 shows the expression levels of SCUBE2 in tumor samples collected at the conclusion of the study in each of the experimental groups put forth in Example 1. As shown in FIG. 38, treatment with a combination of Compound 1 and the higher dose of palbociclib resulted in a greater increase in the level of SCUBE2 as compared to treatment with palbociclib alone and Compound 1 alone. As would be appreciated by the skilled artisan, SCUBE2 is a tumor suppressor that acts through the coordinated suppression of the BMP and (i-catenin signaling pathways in breast cancer (see Cheng, Cancer Res Apr. 15 2009 (69) (8) 3634-3641).

In addition to the genes described above, several other genes that regulate cell proliferation, cell metabolism, cell metastasis and/or cell viability were analyzed.

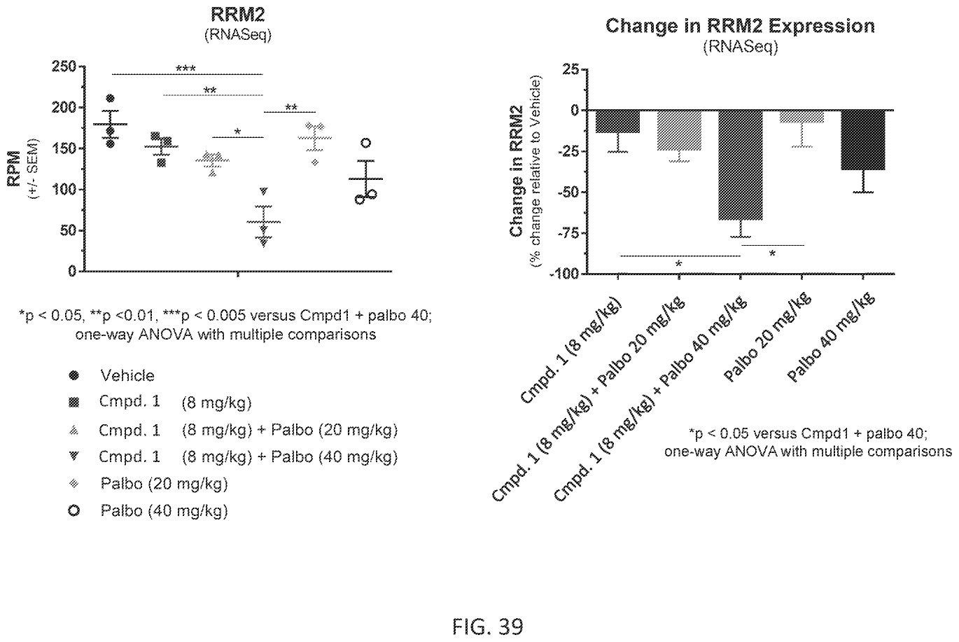

FIG. 39 shows the expression levels of RRM2, a subunit of ribonucleotide reductase (RNR) in tumor samples collected at the conclusion of the study in each of the experimental groups put forth in Example 1. As shown in FIG. 39, treatment with a combination of Compound 1 and palbociclib resulted in a greater decrease in the level of RRM2 as compared to treatment with palbociclib alone and Compound 1 alone. As would be appreciated by the skilled artisan, several chemotherapeutic agents inhibit the activity of RNR, establishing it as a drug target in the treatment of cancer.

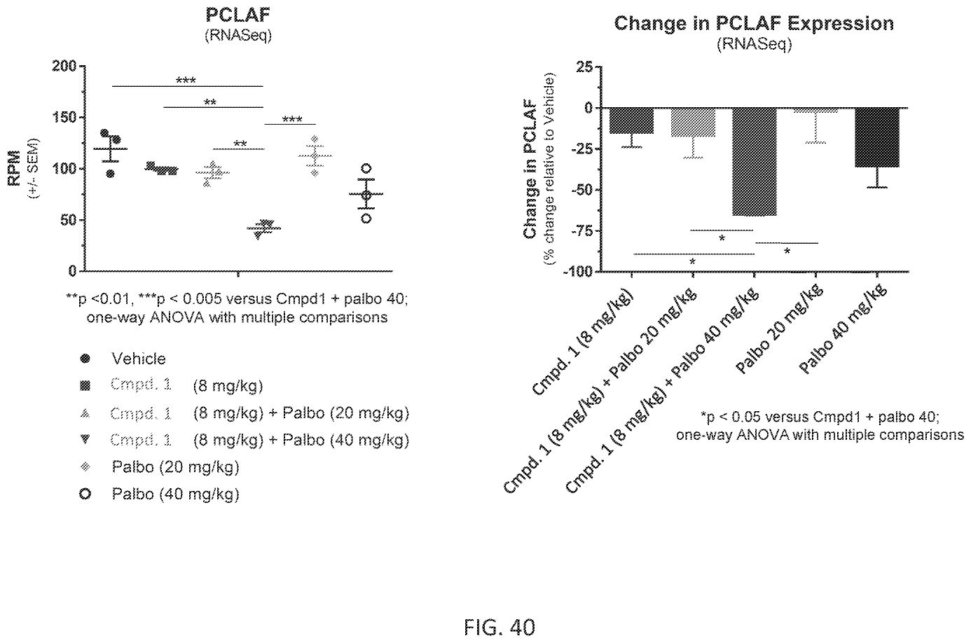

FIG. 40 shows the expression levels of PCLAF (PCNA-associated factor) in tumor samples collected at the conclusion of the study in each of the experimental groups put forth in Example 1. As shown in FIG. 40, treatment with a combination of Compound 1 and the higher dose of palbociclib resulted in a greater decrease in the level of PCLAF as compared to treatment with palbociclib alone and Compound 1 alone. As would be appreciated by the skilled artisan, the expression of PCLAF is elevated in breast cancer, and is associated with cancer stem-cell (CSC) characteristics (CSCs being associated with early recurrence of diseases) and with worse patient outcomes (see Wang, Nat Commun. 2016; 7:10633).

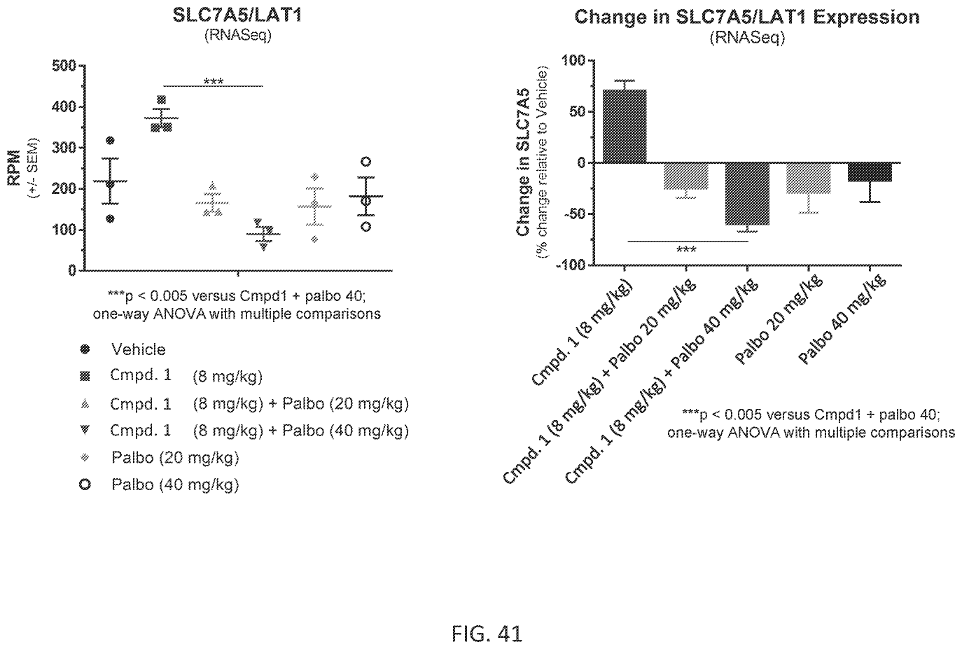

FIG. 41 shows the expression levels of SLC7A5/LAT1 in tumor samples collected at the conclusion of the study in each of the experimental groups put forth in Example 1. As shown in FIG. 41, treatment with a combination of Compound 1 and the higher dose of palbociclib resulted in a greater decrease in the level of SLC7A5/LAT1 as compared to treatment with palbociclib alone. Moreover, treatment with Compound 1 alone resulted in an increase in SLC7A5/LAT1 expression. and Compound 1 alone. As would be appreciated by the skilled artisan, SLC7A5/LAT1 is an amino acid transporter that is upregulated in ER+ breast cancer. Higher expression is associated with development of resistance to endocrine therapy as well as with a significantly higher risk of breast cancer recurrence (see Mihaly, Breast Cancer Res Treat. 2013; 140:219-232 and El Ansari et al. Breast Cancer Research (2018) 20(1):21).

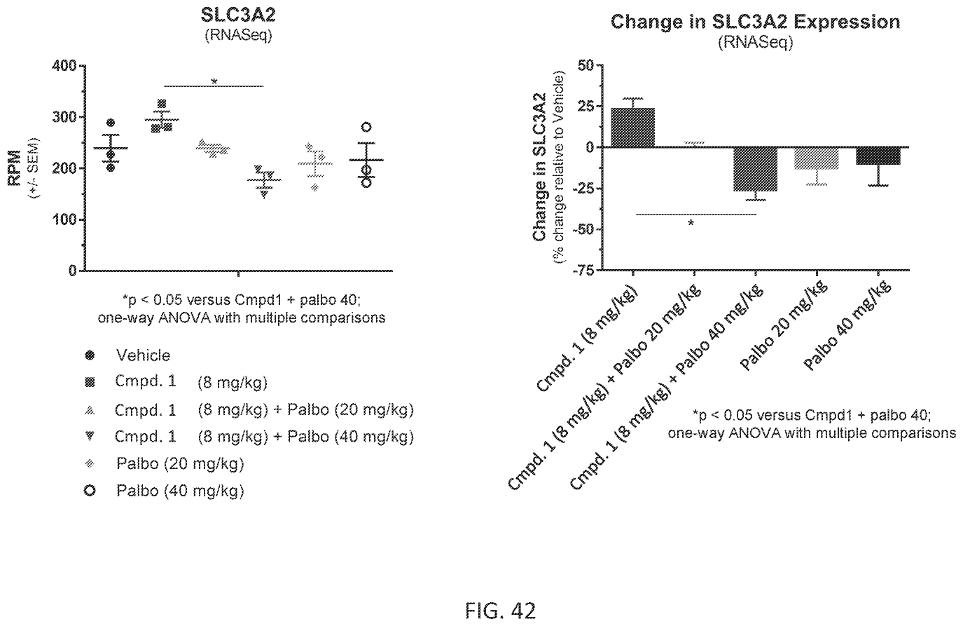

FIG. 42 shows the expression levels of SLC3A2 in tumor samples collected at the conclusion of the study in each of the experimental groups put forth in Example 1. As shown in FIG. 42, treatment with a combination of Compound 1 and the higher dose of palbociclib resulted in a greater decrease in the level of SLC3A2 as compared to treatment with palbociclib alone. Moreover, treatment with Compound 1 alone resulted in an increase in SLC3A2 expression. As would be appreciated by the skilled artisan, SLC3A2 binds to SLC7A5, to form a functional amino acid transporter complex.

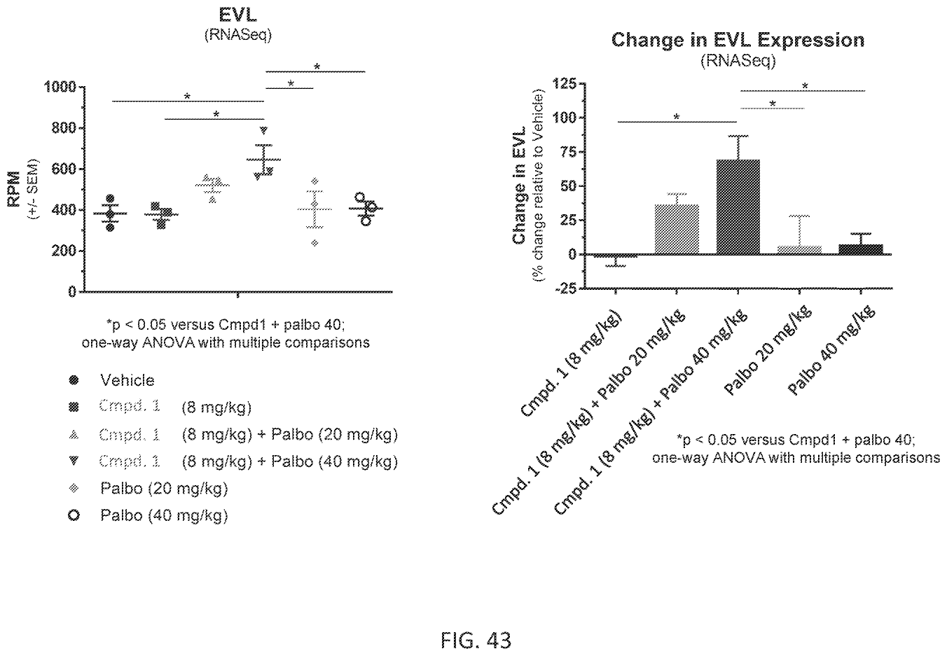

FIG. 43 shows the expression levels of EVL (Ena-VASP-like) in tumor samples collected at the conclusion of the study in each of the experimental groups put forth in Example 1. As shown in FIG. 43, treatment with a combination of Compound 1 and palbociclib resulted in a greater increase in the level of EVL as compared to treatment with palbociclib alone. As would be appreciated by the skilled artisan, EVL is an actin-binding protein and regulates the cytoskeleton of cells. Together with profilin-2, EVL suppresses metastatic behavior in breast cancer and patients with the lowest EVL expression have a significantly higher risk of poor outcomes (see Padilla-Rodriguez, Nat Commun. 2018;9(1):2980 and Mouneimne, Cancer Cell. 2012; 22(5):615-630).

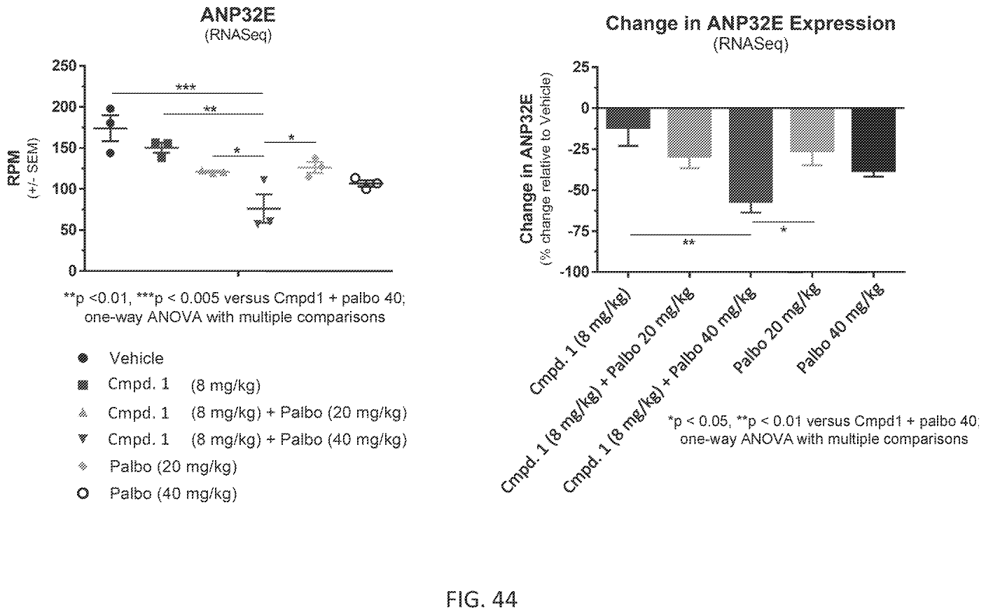

FIG. 44 shows the expression levels of ANP32E (acidic leucine rich nuclear phosphoprotein 32) in tumor samples collected at the conclusion of the study in each of the experimental groups put forth in Example 1. As shown in FIG. 44, treatment with a combination of Compound 1 and the higher dose of palbociclib resulted in a greater decrease in the level of ANP32E as compared to treatment with palbociclib alone and Compound 1 alone. As would be appreciated by the skilled artisan, ANP32E is a specific histone chaperone for the variant histone protein H2AZ1, and removes H2AZ1 from DNA. Elevated expression of ANP32E in breast cancer is associated with metastasis and worse outcomes relative to tumors with lower expression (see Obri, Nature, 2014, 505:648-653; Xiong, Molecular Oncology, 2018, 12: 896-912).

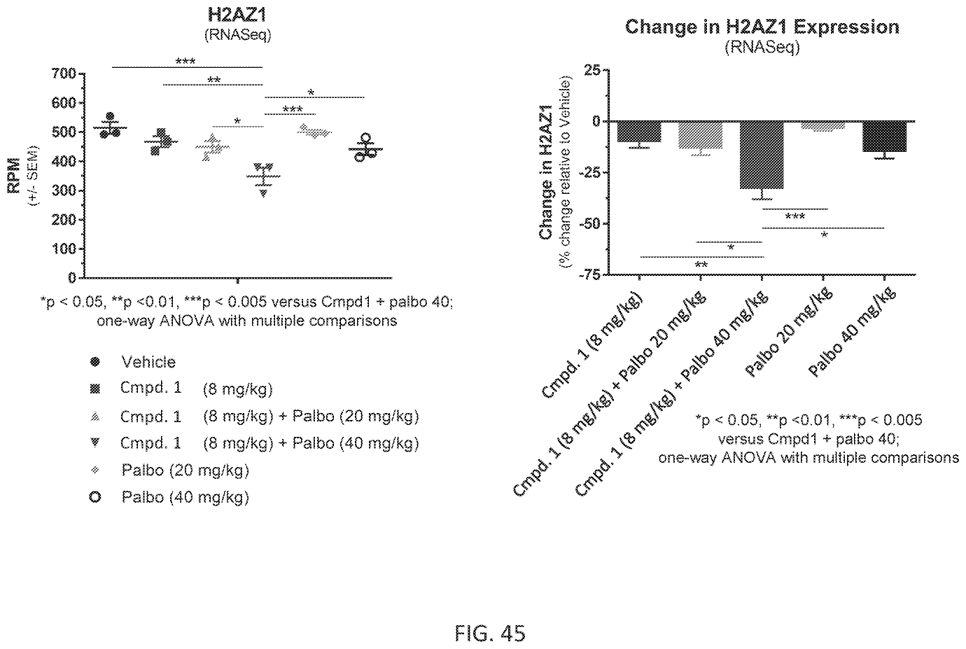

FIG. 45 shows the expression levels of H2AZ1 in tumor samples collected at the conclusion of the study in each of the experimental groups put forth in Example 1. As shown in FIG. 45, treatment with a combination of Compound 1 and the higher dose of palbociclib resulted in a greater decrease in the level of ANP32E as compared to treatment with palbociclib alone and Compound 1 alone. As would be appreciated by the skilled artisan, H2AZ1 promotes cell proliferation by regulating transcription of cell cycle proteins and also modulates the epithelial-mesenchymal transition (EMT), a cellular mechanism that initiates metastasis. Expression of H2AZ1 is upregulated in many cancers including breast cancer and elevated expression is associated with poor outcomes in breast cancer (see Qudnet D., Int Rev Cell Mol Biol. 2018; 335:1-39).

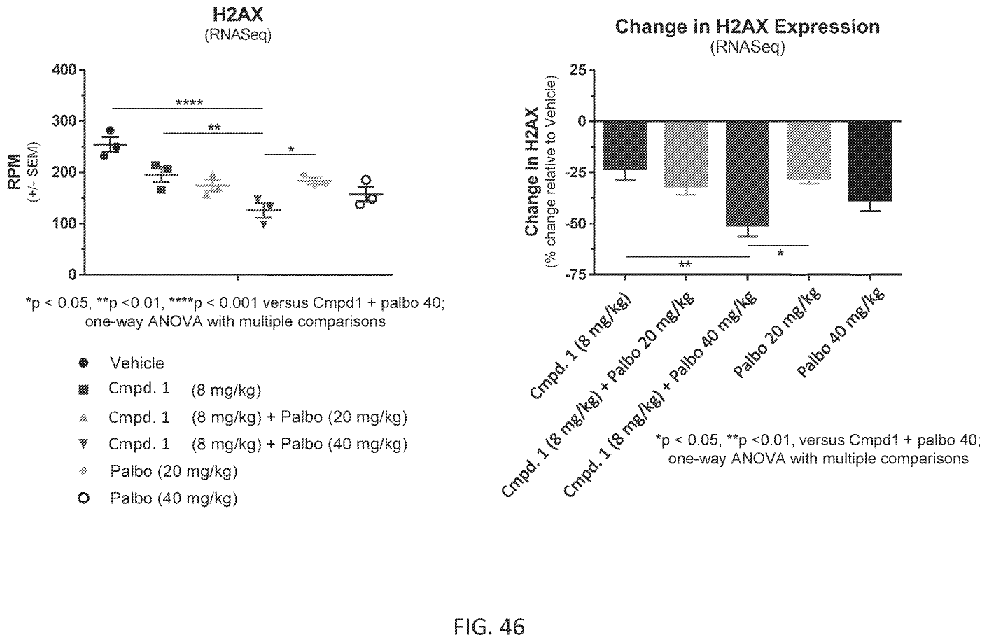

FIG. 46 shows the expression levels of H2AX in tumor samples collected at the conclusion of the study in each of the experimental groups put forth in Example 1. As shown in FIG. 46, treatment with a combination of Compound 1 and the higher dose of palbociclib resulted in a greater decrease in the level of H2AX as compared to treatment with palbociclib alone and Compound 1 alone. As would be appreciated by the skilled artisan, H2AX is known for its role in the DNA damage response as well as in the formation of the mitotic spindle assembly, which regulates mitotic progression during cell division. Lower levels of H2AX lead to chromosomal aberrations, increased sensitivity to radiation and impaired response to double-stranded breaks (DSBs) in DNA (see Ferrand, Cells, 2020;9(11):2424).

The results presented in this example demonstrate that the combination of the MetAP2 inhibitors of the present disclosure and CDK4/6 inhibitors can be used to treat cancer, as the treatment with the combination of a MetAP2 inhibitor and a CDK4/6 inhibitor result in gene expression changes that are associated with tumor reduction, reduced metastases and increased patient survival. Without wishing to be bound by theory, these changes in gene expression demonstrate that the administration of a combination of MetAP2 inhibitors of the present disclosure and CDK4/6 inhibitors can overcome existing limitation of CDK4/6 inhibitor therapy.

More specifically, the results described above demonstrate that the combination of Compound 1 and a CDK4/6 inhibitor results in an unexpectedly greater decrease in expression of genes relied on by tumor cells for survival and metastasis that is not observed upon treatment with either compound alone. As would be appreciated by the skilled artisan, this effect on gene expression can contribute to increased tumor reduction upon the administration of both Compound 1 and a CDK4/6 inhibitor.

### Example 3

The following is a non-limiting example demonstrating that the MetAP2 inhibitors of the present disclosure and CDK4/6 inhibitors can be used in combination to treat cancer. Moreover, the following non-limiting example demonstrates that the combination of the MetAP2 inhibitors of the present disclosure and CDK4/6 inhibitors exhibits unexpectedly superior anti-tumor activity as compared to the use the MetAP2 inhibitors alone.

In the following experiment, mice bearing MCF-7 tumors were treated with either a vehicle control, Compound 1 alone, ribociclib alone, or with a combination of Compound 1 and ribociclib.

MCF-7 were cultured in DMEM supplemented with 10% FBS. On the day of implantation, cells were washed 1× with phosphate-buffered saline (PBS). Following washing, cells were pelleted (5 min @ 1000 rpm, RT) and then counted with a hemocytometer. A cell concentration of 5×10⁶ per mouse was re-suspended in an appropriate amount of PBS and 1:1 Matrigel. The suspension was kept on ice until implantation. Forty-eight hours prior to cell implantation, a 17-β estradiol pellet (0.36 mg 60-day slow release pellet) was be implanted subcutaneously between the scapulae of each Female Nu/j mouse.

The mice were anesthetized with a combination of 4% Isoflurane and 2.5 L/min 02 in an induction chamber. Once anesthetized, the mice were positioned ventral side up and anesthesia maintained through a fitted nose cone. The MCF-7 cell suspension was injected into the mammary fat pad at a volume of 100 μl containing 5×10⁶ cells per mouse.

Beginning 5 days post cell implantation, tumors were measured two times weekly (length×width) using a wireless digital calipers in conjunction with UWAVE-R to record measurements. Once the average tumor volume had reached approximately 50 mm³ ((length×width²) π/6), animals were randomized by average tumor volume and placed into 6 treatment groups of 10 mice each.

The first day of treatment was designated “Day 1”. The mice were dosed either subcutaneously (SC), orally (PO), or by both dose routes, as put forth in the experimental designed summarized in Table 3. Doses were calculated by individual body weights.

TABLE 3

| Experimental | | Number | Dose |
|---|---|---|---|
| Group # | Group Description | of Mice | Frequency |
| 1 | Vehicle (PO) | 10 | QD |
| 2 | Compound 1 8 mg/kg (SC) | 10 | Q4D |
| 3 | Compound 1 8 mg/kg (SC) + | 10 | Q4D |
| | Ribociclib 35 mg/kg (PO) | | QD |
| 4 | Compound 1 8 mg/kg (SC) + | 10 | Q4D |
| | Ribociclib 70 mg/kg (PO) | | QD |
| 5 | Ribociclib 35 mg/kg (PO) | 10 | QD |
| 6 | Ribociclib 70 mg/kg (PO) | 10 | QD |

The first group of mice (Group #1) were administered the vehicle control by oral administration once a day (QD).

The second group of mice (Group #2) were administered Compound 1 at a dose of 8 mg/kg by subcutaneous injection every four days (Q4D).

The third group of mice (Group #3) were administered Compound 1 at a dose of 8 mg/kg by subcutaneous injection every four days and Ribociclib at a dose of 35 mg/kg by oral administration once a day (QD).

The fourth group of mice (Group #4) were administered Compound 1 at a dose of 8 mg/kg by subcutaneous injection every four days and Ribociclib at a dose of 70 mg/kg by oral administration once a day (QD).

The fifth group of mice (Group #5) were administered Ribociclib at a dose of 35 mg/kg by oral administration once a day (QD).

The sixth group of mice (Group #6) were administered Ribociclib at a dose of 70 mg/kg by oral administration once a day (QD).

Twice weekly body weights and tumor measurements were recorded for the duration of the study. Dosing and measurements were performed until the tumors reached a maximum volume of 1000 mm³ or adverse health events were observed (e.g. >20% weight loss, extreme lethargy, tumor necrosis, etc.), at which point the mouse was euthanized. Upon euthanasia, a maximum volume terminal blood sample was obtained via cardiac puncture and split 200 μL in a K₂EDTA MiniCollect tube for plasma separation and 420 μL in to a MiniCollect serum separator tube for half the mice for Clinical Chemistry analysis. For the other half of the mice, >200 μL of blood was collected in to K₂EDTA MiniCollect tubes (LTT) for CBC analysis. The MiniCollect blood tubes for both plasma and serum were centrifuged at 8000 rpm for 5 minutes. The plasma was retained for biomarker analysis at −80° C., the serum and whole blood (LTT) were further analyzed. Additionally, tumor from the mice were dissected, weighed, and split into two pieces. Half of the tumor was placed into buffered formalin and stored at room temperature. Half of the tumor was snap-frozen in liquid N2, and stored at −80° C. Adipose tissue (abdominopelvic, retroperitoneal, and inguinal) was also dissected and weighed. Finally, gross necropsies were performed to check for tumor metastasis (lung, liver, lymph nodes).

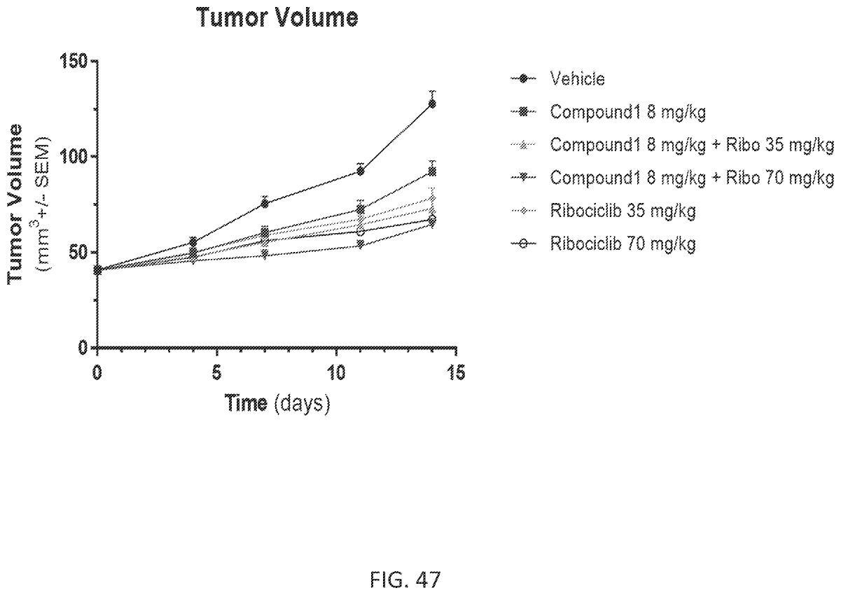

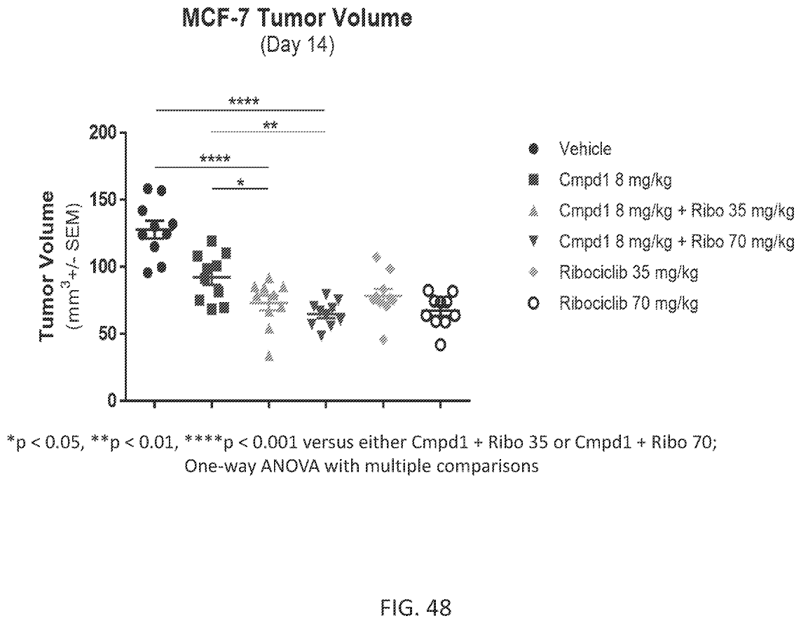

Table 4 shows the Tumor Growth Inhibition (TGI %) as measured on day 14 of the study in each of the treatment groups. As shown in Table 4, treatment with a combination of Compound 1 and the low dose of Ribociclib resulted in a tumor growth inhibition of 63% and treatment with a combination of Compound 1 and the high dose of Ribociclib result in tumor growth inhibition of 72%. FIG. 47 shows the analysis of MCF tumor volume over the course of the first 14 days of the study. FIG. 48 shows the MCF-tumor volume at day 14 of the study.

TABLE 4

| Experimental | |
|---|---|
| Group # | TGI % |
| 1 | — |
| 2 | 41 |
| 3 | 63 |
| 4 | 72 |
| 5 | 57 |
| 6 | 70 |

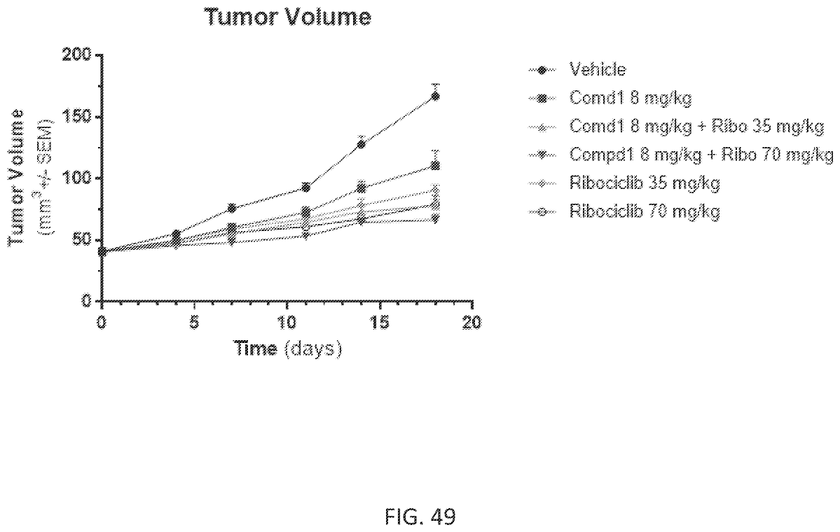

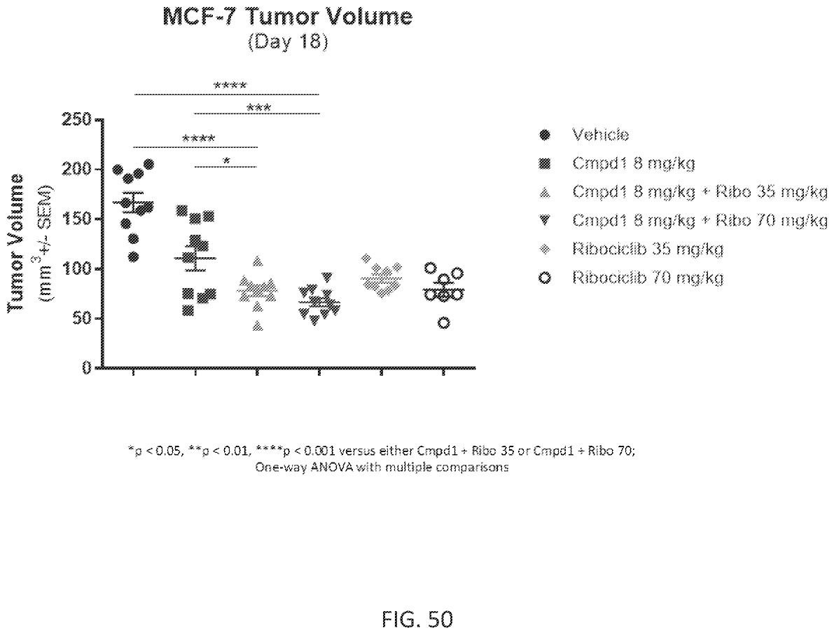

On day 16 of the study, the mice in experimental groups 3, 4, 5 and 6 received a dosage of ribociclib that was 10 times the amount indicated in Table 3. That is, the mice in experimental groups 2 and 5 received 350 mg/kg of ribociclib and the mice in experimental groups 4 and 6 received 700 mg/kg of ribociclib. On day 17, the mice in experimental groups 3, 4, 5 and 6 received a dosage of ribociclib as indicated in Table 3. On day 18, measurements of the tumors in the mice were performed. Table 5 shows the Tumor Growth Inhibition (TGI %) as measured on day 18 of the study in each of the treatment groups. As shown in Table 5, treatment with a combination of Compound 1 and the low dose of Ribociclib resulted in a tumor growth inhibition of 71% and treatment with a combination of Compound 1 and the high dose of Ribociclib result in tumor growth inhibition of 79%. FIG. 49 shows the analysis of MCF tumor volume over the course of the first 18 days of the study. FIG. 50 shows the MCF-tumor volume at day 18 of the study.

TABLE 5

| Experimental | |
|---|---|
| Group # | TGI % |
| 1 | — |
| 2 | 44 |
| 3 | 71 |
| 4 | 79 |
| 5 | 60 |
| 6 | 70 |

The results presented in this example demonstrate that the combination of the MetAP2 inhibitors of the present disclosure and CDK4/6 inhibitors, more specifically ribociclib, can be used to treat cancer and to prevent CDK4/6 treatment resistance.

What is claimed is:

1. A combination comprising at least one MetAP2 inhibitor, or a pharmaceutically acceptable salt thereof, and at least one CDK 4/6 inhibitor, or a pharmaceutically acceptable salt thereof, for use in treating a cancer.

2. A method of treating cancer in a subject in need thereof, the method comprising administering to the subject at least one therapeutically effective amount of at least one MetAP2 inhibitor, or a pharmaceutically acceptable salt thereof, and at least one therapeutically effective amount of at least one CDK4/6 inhibitor, or a pharmaceutically acceptable salt thereof.

3. A MetAP2 inhibitor, or a pharmaceutically acceptable salt thereof, for use in a method of treating a cancer, wherein the method further comprises administration of at least one CDK4/6 inhibitor or a pharmaceutically acceptable salt thereof.

4. A CDK4/6 inhibitor, or a pharmaceutically acceptable salt thereof, for use in a method of treating a cancer, wherein the method further comprises administration of at least one MetAP2 inhibitor or a pharmaceutically acceptable salt thereof.

5. The combination for use of claim 1, the method of claim 2, the MetAP2 inhibitor for use of claim 3, or the CDK4/6 inhibitor for use of claim 4, wherein the at least one MetAP2 inhibitor, or pharmaceutically acceptable salt thereof, and the at least one CDK4/6 inhibitor, or pharmaceutically acceptable salt thereof, are administered concurrently or in temporal proximity.

6. A pharmaceutical composition comprising at least one therapeutically effective amount of at least one MetAP2 inhibitor, or a pharmaceutically acceptable salt thereof and at least one therapeutically effective amount of at least one CDK4/6 inhibitor, or a pharmaceutically acceptable salt thereof.

7. A kit comprising at least one therapeutically effective amount of at least one MetAP2 inhibitor, or a pharmaceutically acceptable salt thereof and at least one therapeutically effective amount of at least one CDK4/6 inhibitor, or a pharmaceutically acceptable salt thereof.

8. The combination for use, the method, the MetAP2 inhibitor for use, the CDK4/6 inhibitor for use, the pharmaceutical composition or the kit of any of the preceding claims, wherein the MetAP2 inhibitor is a compound represented by Formula (I): 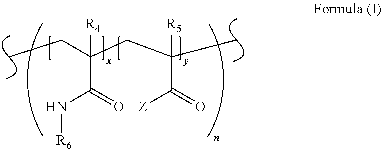
wherein, independently for each occurrence,
R₄ is H or C₁-C₆ alkyl;
R₅ is H or C₁-C₆ alkyl;
R₆ is C₂-C₆ hydroxyalkyl;
Z is —NH-AA₁-AA₂-AA₃-AA₄-AA₅-AA₆-C(O)-L or —NH-AA₁-AA₂-AA₃-AA₄-AA₅-AA₆-C(O)-Q-X—Y—C(O)—W;
AA₁ is glycine, alanine, or H₂N(CH₂)ₘCO₂H, wherein m is 2, 3, 4 or 5;
AA₂ is a bond, or alanine, cysteine, aspartic acid, glutamic acid, phenylalanine, glycine, histidine, isoleucine, lysine, leucine, methionine, asparagine, proline, glutamine, arginine, serine, threonine, valine, tryptophan, or tyrosine;
AA₃ is a bond, or alanine, cysteine, aspartic acid, glutamic acid, phenylalanine, glycine, histidine, isoleucine, lysine, leucine, methionine, asparagine, proline, glutamine, arginine, serine, threonine, valine, tryptophan, or tyrosine;
AA₄ is a bond, or alanine, cysteine, aspartic acid, glutamic acid, phenylalanine, glycine, histidine, isoleucine, lysine, leucine, methionine, asparagine, proline, glutamine, arginine, serine, threonine, valine, tryptophan, or tyrosine;
AA₅ is a bond, or glycine, valine, tyrosine, tryptophan, phenylalanine, methionine, leucine, isoleucine, or asparagine;
AA₆ is a bond, or alanine, asparagine, citrulline, glutamine, glycine, leucine, methionine, phenylalanine, serine, threonine, tryptophan, tyrosine, valine, or H₂N(CH₂)ₘCO₂H, wherein m is 2, 3, 4 or 5;
L is —OH, —O-succinimide, —O-sulfosuccinimide, alkoxy, aryloxy, acyloxy, aroyloxy, alkoxycarbonyloxy, aryloxycarbonyloxy, —NH₂, —NH(C₂-C₆ hydroxyalkyl), halide or perfluoroalkyloxy;
Q is NR, O, or S;
X is M-(C(R)₂)ₚ-M-J-M-(C(R)₂)ₚ-M-V;
M is a bond, or C(O);
J is a bond, or ((CH₂)_{q}Q)ᵣ, C₅-C₈ cycloalkyl, aryl, heteroaryl, NR, O, or S;
Y is NR, O, or S;
R is H or alkyl;
V is a bond or 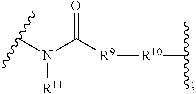
R⁹ is alkyl, aryl, aralkyl, or a bond; or R⁹ taken together with Y forms a heterocyclic ring;
R¹⁰ is amido or a bond;
R¹¹ is H or alkyl;
W is a MetAP2 inhibitor moiety or alkyl;
x is in the range of 1 to about 450;
y is in the range of 1 to about 30;
n is in the range of 1 to about 100;
p is 0 to 20;
q is 2 or 3;
r is 1, 2, 3, 4, 5, or 6;
or a pharmaceutically acceptable salt, prodrug, metabolite, analog or derivative thereof.

9. The combination for use, the method, the MetAP2 inhibitor for use, the CDK4/6 inhibitor for use, the pharmaceutical composition or the kit of claim 8, wherein -Q-X—Y is

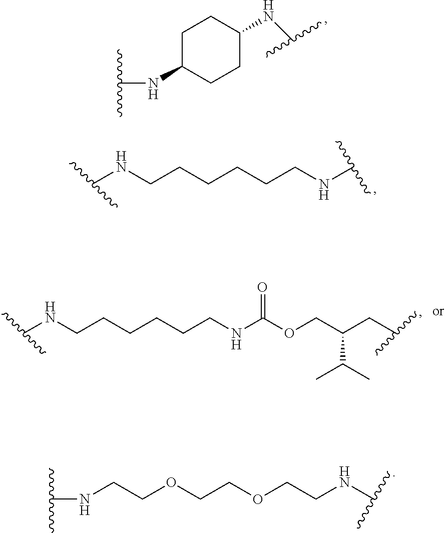 [SMILES: *N[C@H]1CC[C@H](N*)CC1 ; *NCCCCCCN* ; *C[C@@H](COC(=O)NCCCCCCN*)C(C)C ; *NCCOCCOCCN*]

10. The combination for use, the method, the MetAP2 inhibitor for use, the CDK4/6 inhibitor for use, the pharmaceutical composition or the kit of claim 8, wherein W is

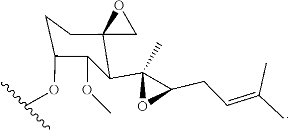

11. The combination for use, the method, the MetAP2 inhibitor for use, the CDK4/6 inhibitor for use, the pharmaceutical composition or the kit of any of the preceding claims, wherein the MetAP2 inhibitor is 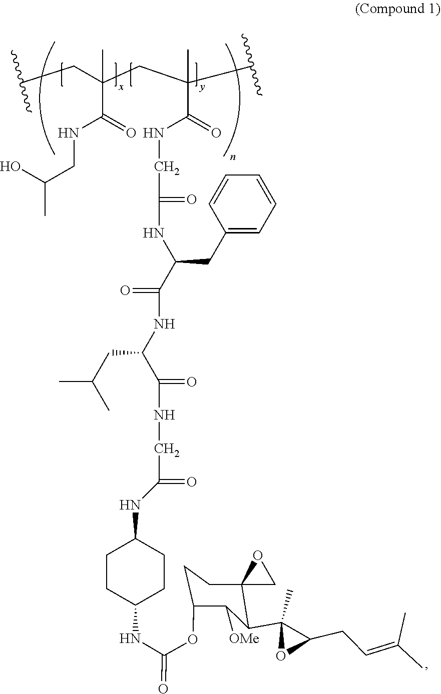
or a pharmaceutically acceptable salt, prodrug, metabolite, analog or derivative thereof.

12. The combination for use, the method, the MetAP2 inhibitor for use, the CDK4/6 inhibitor for use, the pharmaceutical composition or the kit of any of the preceding claims, wherein the MetAP2 inhibitor is 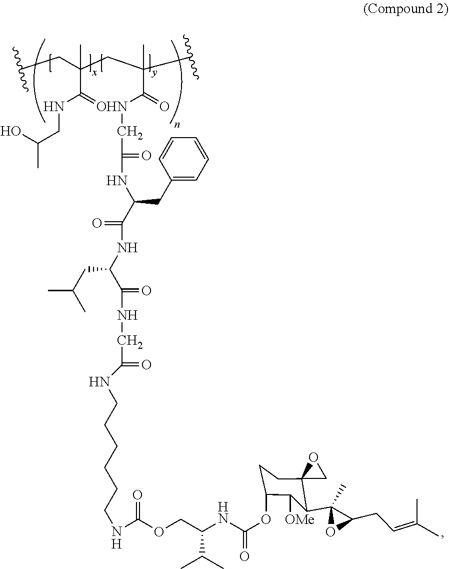
or a pharmaceutically acceptable salt, prodrug, metabolite, analog or derivative thereof.

13. The combination for use, the method, the MetAP2 inhibitor for use, the CDK4/6 inhibitor for use, the pharmaceutical composition or the kit of any of the preceding claims, wherein the MetAP2 inhibitor is 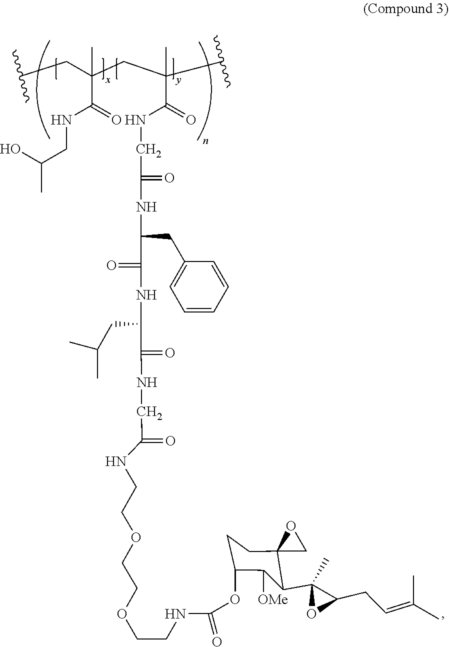
or a pharmaceutically acceptable salt, prodrug, metabolite, analog or derivative thereof.

14. The combination for use, the method, the MetAP2 inhibitor for use, the CDK4/6 inhibitor for use, the pharmaceutical composition or the kit of any of the preceding claims, wherein the MetAP2 inhibitor is 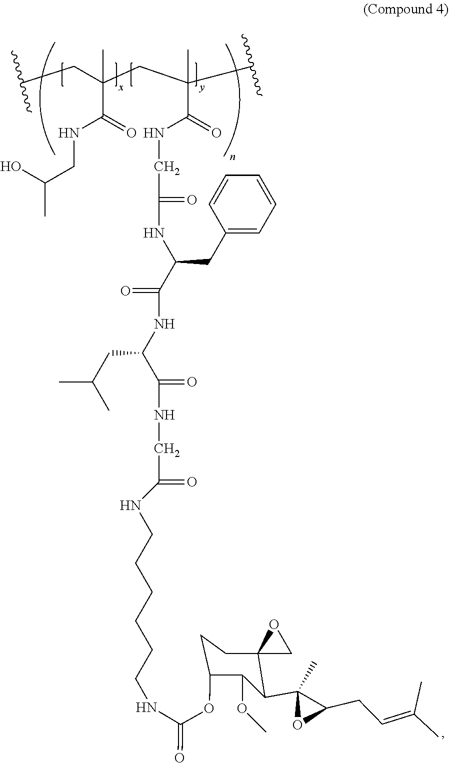
or a pharmaceutically acceptable salt, analog, derivative, salt or ester thereof.

15. The combination for use, the method, the MetAP2 inhibitor for use, the CDK4/6 inhibitor for use, the pharmaceutical composition or the kit of any of the preceding claims, wherein x is in the range of 1 to about 450, y is in the range of 1 to about 30, and n is in the range of 1 to about 100.

16. The combination for use, the method, the MetAP2 inhibitor for use, the CDK4/6 inhibitor for use, the pharmaceutical composition or the kit of any of the preceding claims, wherein the ratio of x to y is in the range of about 30:1 to about 3:1.

17. The combination for use, the method, the MetAP2 inhibitor for use, the CDK4/6 inhibitor for use, the pharmaceutical composition or the kit of claim 13, wherein the ratio of x to y is about 11:1.

18. The combination for use, the method, the MetAP2 inhibitor for use, the CDK4/6 inhibitor for use, the pharmaceutical composition or the kit of any of claims 1-7, wherein the MetAP2 inhibitor is

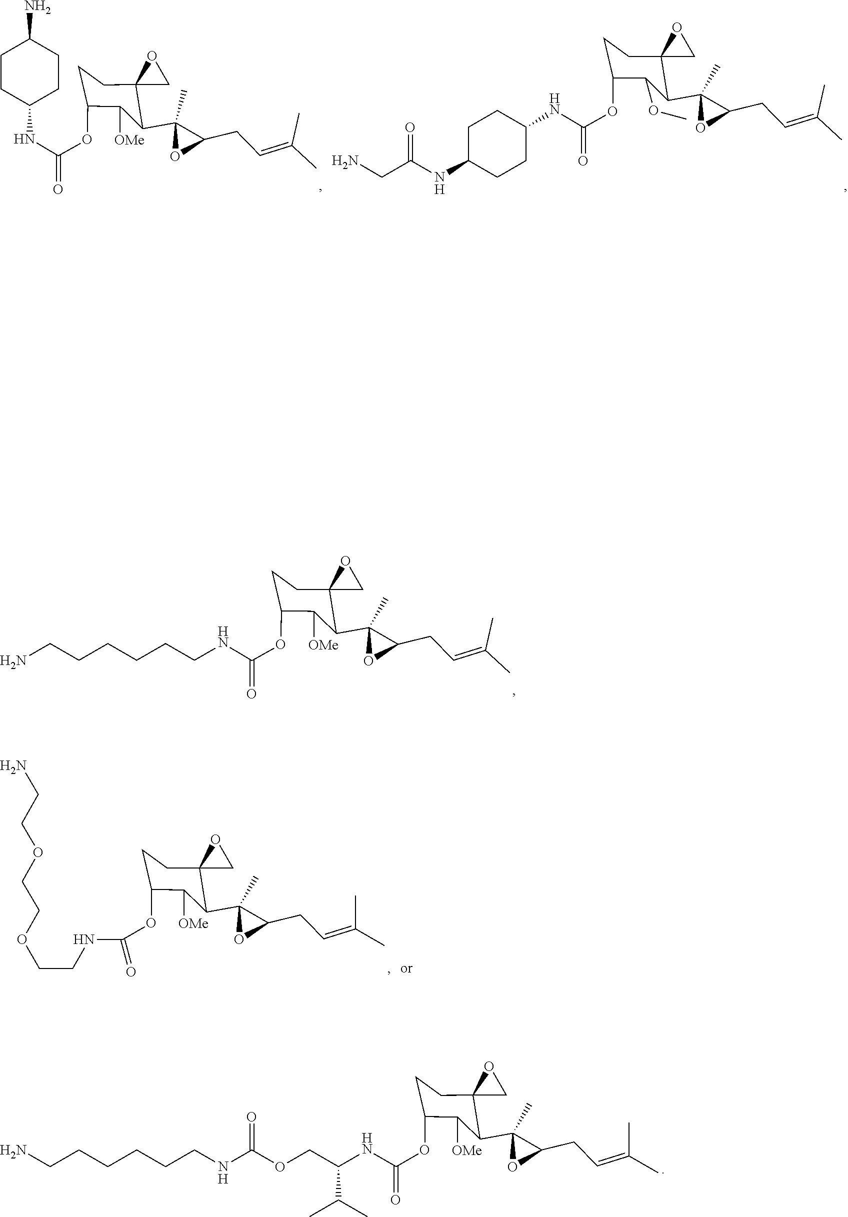

19. The combination for use, the method, the MetAP2 inhibitor for use, the CDK4/6 inhibitor for use, the pharmaceutical composition or the kit of any of claims 1-7, wherein the MetAP2 inhibitor is

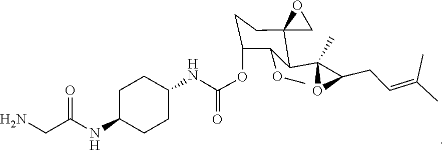

20. The combination for use, the method, the MetAP2 inhibitor for use, the CDK4/6 inhibitor for use, the pharmaceutical composition or the kit of any of the preceding claims, wherein the CDK4/6 inhibitor is selected from palbociclib, abemaciclib, ribociclib, trilaciclib, SHR-6390, FCN-437c, lerociclib, milciclib, PF-06873600, XZP-3287, zotiraciclib, BEBT-209, BPI-16350, CS-3002, fadraciclib, HS-10342, ON-123300, PF-06842874, TQ-05510, BPI-1178, JS-101, NUV-422, AU-294, CCT-68127, ETH-155008, HEC-80797, JRP-890, JS-104, NEOS-518, PF-07104091, PF-07220060, RMC-4550, SRX-3177, VS-2370, VS-2370, or a pharmaceutically acceptable salt thereof.

21. The combination for use, the method, the MetAP2 inhibitor for use, the CDK4/6 inhibitor for use, the pharmaceutical composition or the kit of any of the preceding claims, wherein the CDK4/6 inhibitor is palbociclib, or a pharmaceutically acceptable salt thereof.

22. The combination for use, the method, the MetAP2 inhibitor for use, the CDK4/6 inhibitor for use, the pharmaceutical composition or the kit of any of the preceding claims, wherein the CDK4/6 inhibitor is abemaciclib, or a pharmaceutically acceptable salt thereof.

23. The combination for use, the method, the MetAP2 inhibitor for use, the CDK4/6 inhibitor for use, the pharmaceutical composition or the kit of any of the preceding claims, wherein the CDK4/6 inhibitor is ribociclib, or a pharmaceutically acceptable salt thereof.

24. The combination for use, the method, the MetAP2 inhibitor for use, the CDK4/6 inhibitor for use, the pharmaceutical composition or the kit of any of the preceding claims, wherein the MetAP2 inhibitor is for administration by subcutaneous injection.

25. The combination for use, the method, the MetAP2 inhibitor for use, the CDK4/6 inhibitor for use, the pharmaceutical composition or the kit of any of the preceding claims, wherein the CDK4/6 inhibitor is for oral administration.

26. The combination for use, the method, the MetAP2 inhibitor for use, the CDK4/6 inhibitor for use, the pharmaceutical composition or the kit of any of the preceding claims, wherein the cancer is a carcinoma, a lymphoma, a blastoma, a sarcoma, a leukemia, a brain cancer, a breast cancer, a blood cancer, a bone cancer, a lung cancer, a skin cancer, a liver cancer, an ovarian cancer, a bladder cancer, a renal cancer, a kidney cancer, a gastric cancer, a thyroid cancer, a pancreatic cancer, an esophageal cancer, a prostate cancer, a cervical cancer, a uterine cancer, a stomach cancer, a soft tissue cancer, a laryngeal cancer, a small intestine cancer, a testicular cancer, an anal cancer, a vulvar cancer, a joint cancer, an oral cancer, a pharynx cancer or a colorectal cancer.

27. The combination for use, the method, the MetAP2 inhibitor for use, the CDK4/6 inhibitor for use of any of the preceding claims, wherein the cancer is a breast cancer.

28. The combination for use, the method, the MetAP2 inhibitor for use, the CDK4/6 inhibitor for use of any of the preceding claims, wherein the breast cancer is HR+HER2− breast cancer or ER+ breast cancer.
